(19) United States
(12) Patent Application Publication
(10) Pub. No.: US 2011/0166455 A1
(43) Pub. Date: Jul. 7, 2011

(54) CATHETER

(76) Inventors: Edward H. Cully, Flagstaff, AZ (US); Dennis R. Dietz, Littleton, CO (US); Curtis J. Franklin, Flagstaff, AZ (US); Craig T. Nordhausen, Parker, CO (US); Clyde G. Oakley, Centennial, CO (US); Ryan C. Patterson, Farmington, UT (US); Jim H. Polenske, Bellemont, AZ (US); Thomas W. Shilling, Lakewood, CO (US); Thomas L. Tolt, Centennial, CO (US)

(21) Appl. No.: 12/684,079

(22) Filed: Jan. 7, 2010

(51) Int. Cl. A61B 8/14

(52) U.S. Cl. 600/463

(57) ABSTRACT

An improved catheter is provided. The catheter may include a deflectable member located at a distal end of the catheter. The deflectable member may comprise an ultrasound transducer array. In embodiments where the deflectable member includes an ultrasound transducer array, the ultrasound transducer array may be operable to image both when aligned with the catheter and when pivoted relative to the catheter. When pivoted relative to the catheter, the ultrasound transducer array may have a field of view distal to the distal end of the catheter. The ultrasound array may be interconnected to a motor to effectuate pivotal reciprocal motion of the ultrasound transducer array such that the catheter may be operable to produce real-time or near real-time three dimensional images.

### FIELD OF THE INVENTION

The invention relates to improved catheters, and is particularly apt to catheters for imaging and/or interventional device delivery at desired locations in the body of a patient.

### BACKGROUND OF THE INVENTION

Catheters are tubular medical devices that may be inserted into a body vessel, cavity or duct, and manipulated utilizing a portion that extends out of the body. Typically, catheters are relatively thin and flexible to facilitate advancement/retraction along non-linear paths. Catheters may be employed for a wide variety of purposes, including the internal bodily positioning of diagnostic and/or therapeutic devices. For example, catheters may be employed to position internal imaging devices, deploy implantable devices (e.g., stents, stent grafts, vena cava filters), and/or deliver energy (e.g., ablation catheters).

In this regard, use of ultrasonic imaging techniques to obtain visible images of structures is increasingly common, particularly in medical applications. Broadly stated, an ultrasonic transducer, typically comprising a number of individually actuated piezoelectric elements, is provided with suitable drive signals such that a pulse of ultrasonic energy travels into the body of the patient. The ultrasonic energy is reflected at interfaces between structures of varying acoustic impedance. The same or a different transducer detects the receipt of the return energy and provides a corresponding output signal. This signal can be processed in a known manner to yield an image, visible on a display screen, of the interfaces between the structures and hence of the structures themselves.

Numerous prior art patents discuss the use of ultrasonic imaging in combination with specialized surgical equipment in order to perform very precise surgical procedures. For example, a number of patents show use of ultrasonic techniques for guiding a “biopsy gun”, i.e., an instrument for taking a tissue sample from a particular area for pathological examination, for example, to determine whether a particular structure is a malignant tumor or the like. Similarly, other prior art patents discuss use of ultrasonic imaging techniques to assist in other delicate operations, e.g., removal of viable eggs for in vitro fertilization, and for related purposes.

In the past few decades, there have been significant breakthroughs in the development and application of interventional medical devices including inferior vena cava filters, vascular stents, aortic aneurysm stent grafts, vascular occluders, cardiac occluders, prosthetic cardiac valves, and catheter and needle delivery of radio frequency ablation. However, imaging modalities have not kept pace as these procedures are typically performed under fluoroscopic guidance and make use of X-ray contrast agents. Fluoroscopy has draw backs including its inability to image soft tissues and the inherent radiation exposure for both the patient and the clinician. Furthermore, conventional fluoroscopic imaging provides only a planar two dimensional (2D) view.

Intracardiac Echocardiography (ICE) catheters have become the preferred imaging modality for use in structural heart intervention because they provide high resolution 2D ultrasound images of the soft tissue structure of the heart. Additionally, ICE imaging does not contribute ionizing radiation to the procedure. ICE catheters can be used by the interventional cardiologist and staff within the context of their normal procedural flow and without the addition of other hospital staff. Current ICE catheter technology does have limitations though. The conventional ICE catheters are limited to generating only 2D images. Furthermore, the clinician must steer and reposition the catheter in order to capture multiple image planes within the anatomy. The catheter manipulation needed to obtain specific 2D image planes requires that a user spend a significant amount of time becoming facile with the catheter steering mechanisms.

Visualizing the three dimensional (3D) architecture of the heart, for example, on a real-time basis during intervention is highly desirable from a clinical perspective as it facilitates more complex procedures such as left atrial appendage occlusion, mitral valve repair, and ablation for atrial fibrillation. 3D imaging also allows the clinician to fully determine the relative position of structures. This capability is of particular import in cases of structural abnormalities in the heart where typical anatomy is not present. Two dimensional transducer arrays provide a means to generate 3D images, but currently available 2D arrays require a high number of elements in order to provide sufficient aperture size and corresponding image resolution. This high element count results in a 2D transducer that is prohibitive with respect to clinically acceptable catheter profiles.

The Philips iE33 echocardiography system running the new 3D transesophageal (TEE) probe (available from Philips Healthcare, Andover, Mass., USA) represents the first commercially-available real-time 3D (four dimensional (4D)) TEE ultrasound imaging device. This system provides the clinician with the 4D imaging capabilities needed for more complex interventions, but there are several significant disadvantages associated with this system. Due to the large size of the TEE probe (50 mm circumference and 16.6 mm width), patients need to be anesthetized or heavily sedated prior to probe introduction (G. Hamilton Baker, MD et al., Usefulness of Live Three-Dimensional Transesophageal Echocardiography in a Congenital Heart Disease Center, Am J Cardiol 2009; 103: 1025-1028). This requires that an anesthesiologist be present to induce and monitor the patient on anesthesia. In addition any hemodynamic information relevant to the procedure must be gathered prior to the induction of general anesthesia due to the effects of anesthetic on the hemodynamic status of the patient. Furthermore, minor and major complications from TEE probe use do occur including complications ranging from sore throat to esophageal perforation. The complexity of the Phillips TEE system and probe require the participation of additional staff such as an anesthesiologist, echocardiographer and ultrasound technician. This increases procedure time and cost.

Interventional clinicians desire an imaging system that is catheter-based and small enough for percutaneous access with three dimensional imaging in real-time (4D) capabilities. Rather than steering the catheter within the anatomy to capture various views, as is the case with conventional ICE catheters, it is desirable that such a catheter system be capable of obtaining multiple image planes or volumes from a single, stable catheter position within the anatomy. A catheter that would allow the clinician to guide or steer the catheter to a position within the heart, vasculature, or other body cavities, lock the catheter in a stable position, and yet still allow the selection of a range of image planes or volumes within the anatomy would facilitate more complex procedures. Due to the size constraints of some anatomical locations, e.g., that in the heart, it is desirable that the viewing angles necessary be obtainable within a small anatomical volume of for example less than about 3 cm.

As internal diagnostic and therapeutic procedures continue to evolve, the desirability of enhanced procedure imaging via compact and maneuverable catheters has been recognized. More particularly, the present inventors have recognized the desirability of providing catheter features that facilitate selective positioning and control of componentry located at a distal end of a catheter, while maintaining a relatively small profile, thereby yielding enhanced functionality for various clinical applications.

### SUMMARY OF THE INVENTION

The present invention relates to improved catheter designs. For purposes hereof, a catheter is defined as a device which is capable of being inserted into a body vessel, cavity or duct, wherein at least a portion of the catheter extends out of the body and the catheter is capable of being manipulated and/or removed from the body by manipulating/pulling on the portion of the catheter extending out of the body. Embodiments of catheters disclosed herein may include a catheter body. A catheter body may, for example, include an outer tubular body, an inner tubular body, a catheter shaft, or any combination thereof. Catheter bodies disclosed herein may or may not include a lumen. Such lumens may be conveyance lumens for the conveyance of a device and/or material. For example, such lumens may be used for the delivery of an interventional device, the delivery of a diagnostic device, the implantation and/or retrieval of an object, the delivery of drugs, or any combination thereof.

Embodiments of catheters designs disclosed herein may include a deflectable member. The deflectable member may be disposed at a distal end of a catheter body and may be operable to deflect relative to the catheter body. “Deflectable” is defined as the ability to move a member interconnected to the catheter body, or a portion of the catheter body, away from the longitudinal axis of the catheter body, preferably such that the member or portion of the catheter body is fully or partially forward-facing. Deflectable may also include the ability to move the member, or the portion of the catheter body, away from the longitudinal axis of the catheter body, preferably such that the member or portion of the catheter body is fully or partially rearward-facing. Deflectable may include the ability to move the member away from the longitudinal axis of the catheter body at a distal end of the catheter body. For example, a deflectable member may be operable to be deflected plus or minus 180 degrees from a position where the deflectable member is aligned with a distal end of the catheter body (e.g., where the deflectable member is disposed distal to the distal end of the catheter body). In another example, a deflectable member may be deflectable such that a distal port of a conveyance lumen of the catheter body may be opened. The deflectable member may be operable to move relative to the catheter body along a predetermined path that is defined by the structure of the interconnection between the deflectable member and catheter body. For example, the deflectable member and catheter body may each be directly connected to a hinge (e.g., the deflectable member and catheter body may each be in contact with and/or fixed to the hinge) disposed between the deflectable member and catheter body, and the hinge may determine the predetermined path of movement that the deflectable member may move through relative to the catheter body. The deflectable member may be selectively deflectable relative to the catheter body to facilitate operation of componentry comprising the deflectable member.

The deflectable member may include a motor for selective driven movement of a component or components within the deflectable member. The motor may be any device or mechanism that creates motion that may be used for the aforementioned selective driven movement.

The selectively driven component or components may, for example, include a diagnostic device (e.g., an imaging device), a therapeutic device, or any combination thereof. For example, the selectively driven component may be a transducer array such as an ultrasound transducer array that may be used for imaging. Further, the ultrasound transducer array may, for example, be a one dimensional array, one and a half dimensional array, or a two dimensional array. In additional examples, the selectively driven component may be an ablation device such as a Radio Frequency (RF) ablation applicator or a high frequency ultrasonic (HIFU) ablation applicator.

As used herein, “imaging” may include ultrasonic imaging, be it one dimensional, two dimensional, three dimensional, or real-time three dimensional imaging (4D). Two dimensional images may be generated by one dimensional transducer arrays (e.g., linear arrays or arrays having a single row of elements). Three dimensional images may be produced by two dimensional arrays (e.g., those arrays with elements arranged in an n by n planar configuration) or by mechanically reciprocated, one dimensional transducer arrays. The term “imaging” also includes optical imaging, tomography, including optical coherence tomography (OCT), radiographic imaging, photoacoustic imaging, and thermography.

In an aspect, a catheter may include a catheter body having a proximal end and a distal end. The catheter may further include a deflectable member interconnected to the distal end. The deflectable member may include a motor.

In certain embodiments, the deflectable member may be hingedly connected to the distal end of the catheter body and operable for positioning across a range of angles relative to the catheter body. For example, the deflectable member may be connected to the distal end of the catheter body and operable for positioning across a range of angles relative to a longitudinal axis of the catheter body at the distal end. The deflectable member may further include a component, wherein the motor may effectuate movement of the component.

In certain embodiments, the movement may, for example, be rotational, pivotal, reciprocal, or any combination thereof (e.g., reciprocally pivotal). The component may be an ultrasound transducer array. The ultrasound transducer array may be configured for at least one of two dimensional imaging, three dimensional imaging and real-time three dimensional imaging. The catheter may have a minimum presentation width of less than about 3 cm. A length of a region of the catheter body in which deflection occurs when the deflectable member is deflected 90 degrees relative to the catheter body may be less than a maximum cross dimension of the catheter body.

The catheter body may comprise at least one steerable segment. For example, the steerable segment may be proximate to the distal end.

The catheter body may comprise a lumen. Such lumen may be for conveyance of a device (e.g., an interventional device) and/or material. In one embodiment, the lumen may extend form the proximal end to the distal end.

The catheter may include a hinge interconnecting the deflectable member and the catheter body. In one approach, the deflectable member may be supportably connected to the hinge. In certain embodiments, the hinge may, for example, be a living hinge or an ideal hinge, and the hinge may include a non-tubular bendable portion.

In another aspect, a catheter may include an outer tubular body, a deflectable member, and a hinge interconnecting the deflectable member and the outer tubular body. The deflectable member may include a motor. In an approach, the deflectable member may further include an ultrasound transducer array. The outer tubular body may comprise at least one steerable segment. The catheter may include an actuation device operable for active deflection of the deflectable member. The actuation device may, for example, include balloons, tether lines, wires (e.g., pull wires), rods, bars, tubes, hypotubes, stylets (including pre-shaped stylets), electro-thermally activated shape memory materials, electro-active materials, fluid, permanent magnets, electromagnets, or any combination thereof. The catheter may include a handle disposed at the proximal end. The handle may include a movable member to control the deflection of the deflectable member. The handle may include a mechanism, such as a worm gear arrangement or an active brake, capable of maintaining a selected deflection of the deflectable member.

In an arrangement, a catheter may include a catheter body having at least one steerable segment and a deflectable member. The deflectable member may include a component and a motor to effectuate movement of the component. In an embodiment, the catheter may include a hinge interconnecting the deflectable member and the catheter body.

In another aspect, a catheter may include a catheter body with at least one steerable segment, a deflectable member, a component supportably disposed on the deflectable member, and a motor supportably disposed on the deflectable member and operable for selective movement of the component. The deflectable member may be supportably disposed at a distal end of the catheter body and operable for selective deflectable positioning across a range of angles relative to the longitudinal axis of the catheter body at the distal end. In an approach, the component may be an ultrasound transducer array. The catheter may be configured such that a plane that may be perpendicular to a longitudinal axis of the deflectable member intersects both the component and the motor.

In yet another aspect, a catheter may include a catheter body and a deflectable member supportably disposed at a distal end of the catheter body and operable for selective deflectable positioning across a range of angles relative to the longitudinal axis of the catheter body. The catheter may further include a component disposed in the deflectable member. The component may be operable to move independently of the deflectable member, and the deflectable member may be operable to move independently from the catheter body.

In certain arrangements, a catheter may include a catheter body, a lumen, a deflectable member, and an electrical conductor member. The lumen may be for conveyance of a device and/or material, and may extend through at least a portion of the catheter body to a port located distal to a proximal end of the catheter body. The deflectable member may be located at a distal end of the catheter body and may include a motor and a component. The electrical conductor member may include a plurality of electrical conductors in an arrangement extending from the component to the catheter body. The arrangement may be bendable in response to deflection of the deflectable member. In an embodiment, the arrangement may comprise a flexboard arrangement. Such a flexboard arrangement may be bendable in response to oscillatory movement of the ultrasound transducer array. The flexboard arrangement may comprise a plurality of electrically conductive traces supportably disposed on a flexible, non-conductive substrate. In an approach, the flexboard arrangement may electrically interface with a plurality of conductors that extend from a proximal end to a distal end of the catheter body.

In an aspect, a catheter may include a catheter body, a lumen, and a deflectable member. The lumen may be configured for conveyance of a device and/or material and may extend through at least a portion of the catheter body to a port located distal to a proximal end of the catheter body. The deflectable member may be located at a distal end of the catheter body and may comprise a motor operable to effectuate movement of a component of the deflectable member. In an approach, the catheter may include a first electrical conductor portion and a second electrical conductor portion. The first electrical conductor portion may include a plurality of electrical conductors arranged with electrically non-conductive material therebetween, and may extend from the proximal end to the distal end. The second electrical conductor portion may be electrically interconnected to the first electrical conductor portion at the distal end and to an ultrasound transducer array. The second electrical conductor portion may be bendable in response to deflection of the deflectable member. The second electrical conductor portion may be bendable in response to oscillatory movement of the component.

In another arrangement, a catheter may include an outer tubular body, an inner tubular body, and a deflectable member. The inner tubular body may define a lumen therethrough for conveyance of a device and/or material. The outer tubular body and the inner tubular body may be disposed for selective relative movement therebetween. At least a portion the deflectable member may be permanently located outside of the outer tubular body at a distal end of the outer tubular body. The deflectable member may be supportability interconnected to the inner tubular body or the outer tubular body. Upon the selective relative movement, the deflectable member may be selectively deflectable in a predetermined manner. The deflectable member may include a component (e.g., an ultrasound transducer array) and a motor operable for movement of the component. In an embodiment, the deflectable member may be supportably interconnected to a hinge. The hinge may be supportably interconnected to the inner tubular body and restrainably interconnected to the outer tubular body. The catheter may further include a restraining member interconnected to the deflectable member and the outer tubular body. Upon advancement of the inner tubular body relative to the outer tubular body, a deflection force may be communicated to the deflectable member by the restraining member. The restraining member may be also a flexible electrical interconnection member.

In another aspect, a catheter may include a catheter body and a deflectable member. The catheter body may have at least one steerable segment. The deflectable member may be located at, and interconnected to, a distal end of the catheter body and may be selectively deflectable from a first position to a second position. The deflectable member may comprise a motor. In an example, the deflectable member may further comprise an ultrasound transducer array. The deflectable member may be interconnected to the catheter body by a tether, wherein the tether restrainably interconnects the deflectable member to the catheter body. A tether may be disposed between the deflectable member and the catheter body, and the tether may include a flexible electrical interconnection member.

In still another aspect, a catheter may include a catheter body, a deflectable member, and an ultrasound transducer array disposed on the deflectable member (e.g., within the deflectable member) for pivotal movement about a pivot axis. The catheter may further include a first electrical interconnection member having a first portion coiled and electrically interconnected to the ultrasound transducer array, a motor operable to produce the pivotal movement, and a hinge disposed between the catheter body and the deflectable member. In an approach, the catheter may include an enclosed volume. The first portion of the first electrical interconnection member may be disposed in a clock spring arrangement. The deflectable member may comprise a distal end and a proximal end, and the ultrasound transducer array may be disposed closer to the distal end than the first portion of the first electrical interconnection member, and the motor may be operable to pivot the ultrasound transducer array through at least about 360 degrees. A fluid may be disposed within the enclosed volume. A midline of the first portion of the first electrical interconnection member may be disposed within a single plane that may be disposed perpendicular to the pivot axis.

In an aspect, a catheter may include a catheter body, a deflectable member, an ultrasound transducer array, and a first electrical interconnection member. The catheter body may include a proximal end and a distal end. The deflectable member may be supportably disposed on the distal end of the catheter body and may have a portion having a first volume. The deflectable member may be deflectable relative to a longitudinal axis of the catheter body at the distal end. The ultrasound transducer array may be disposed for pivotal movement about a pivot axis within the first volume. The first electrical interconnection member may have a first portion coiled within the first volume and electrically interconnected to the ultrasound transducer array. In an embodiment, upon the pivotal movement, the coiled first portion of the first electrical interconnection member may tighten or loosen (e.g., the diameter of the coiled first portion may decrease or increase upon the pivotal movement). The coiled first portion may be configured such that pivoting in either direction (e.g., tightening or loosening) relative to a predetermined position requires force to overcome a resistance to such pivoting from the coiled first portion. The first electrical interconnection member may be ribbon-shaped and comprise a plurality of conductors arranged with electrically non-conductive material therebetween.

In an aspect, a catheter may include a deflectable member having a portion having an enclosed volume, a fluid disposed within the enclosed volume, an ultrasound transducer array, a first electrical interconnection member, and a hinge. The ultrasound transducer array may be disposed for reciprocal pivotal movement within the enclosed volume. The first electrical interconnection member may have at least a portion helically disposed within the enclosed volume and fixedly interconnected to the ultrasound transducer array. Upon the reciprocal movement, the helically disposed portion may loosen and tighten along a length thereof. The hinge may be disposed between the deflectable member and the catheter body.

In an arrangement, a catheter may include a catheter body, a deflectable member having a portion having an enclosed volume, a fluid disposed within the enclosed volume, a hinge, and a bubble-trap member. The hinge may be disposed between the deflectable member and the catheter body. The bubble-trap member may be fixedly positioned within the enclosed volume and have a distal-facing, concave surface. A distal portion of the enclosed volume may be defined distal to the bubble-trap member and a proximal portion of the enclosed volume may be defined proximal to the bubble-trap member. An aperture may be provided through the bubble-trap member to fluidly interconnect from the distal portion of the enclosed volume to the proximal portion of the enclosed volume.

In another arrangement, a catheter may include a deflectable member having a portion having an enclosed volume, a fluid disposed within the enclosed volume, an ultrasound transducer array disposed for movement within the enclosed volume, a hinge, and a bellows member. The bellows member may have a flexible, closed-end portion located in the fluid disposed within the enclosed volume and an open-end portion isolated from the fluid. The bellows member may be collapsible and expansible in response to volumetric variations in the fluid.

In yet another arrangement, a method for operating a catheter may include advancing a catheter body through a natural or otherwise-formed passageway in a patient, steering a distal end of the catheter body to a desired position, selectively deflecting a deflectable member hingedly connected to the distal end of the catheter body to one or more angles relative to the catheter body with the distal end of the catheter body maintained in the desired position, and operating a motor of the deflectable member to effectuate movement of an ultrasound transducer array to obtain at least two unique 2D images (i.e., images obtained with the ultrasound transducer array in two different orientations). The selective deflection may be achieved through an actuation device operable for selective deflection of the deflectable member. In an approach, the selective deflection step may be completed within a volume having a cross-dimension of about 3 cm or less.

In an aspect, a method for operating a catheter that includes a catheter body may include advancing the catheter through a passageway in a patient to a desired position such that a distal end of the catheter body is located at a first position. The catheter body may have at least one independently steerable segment and a deflectable member supportably disposed at the distal end of the catheter body. The method may further include deflecting the deflectable member to a desired angular position within a range of viewing angles relative to the distal end of the catheter body with the distal end maintained in the first position. The method may further include operating a motor supportably disposed on the deflectable member with the deflectable member in the desired angular position, for driven movement of an ultrasound transducer array supportably disposed on the deflectable member. In an embodiment, the method may further include steering the catheter body by flexure along a length thereof. The deflecting step may comprise deforming a hinge (which interconnects the distal end of the catheter body and the deflectable member) from a first configuration to a second configuration. In an embodiment, the method may further include advancing or retrieving a device or material through a port at the distal end of the catheter body and into an imaging volume of the ultrasound transducer array during the operating step.

The deflectable member may have a round cross-sectional profile. The deflectable member may include an enclosed volume and a sealable port. In one aspect, the deflectable member may include at least one sealable fluid filling port that allows the enclosed volume to be filled with a fluid, e.g., one that will facilitate acoustic coupling. The sealable port may be used to fill the enclosed volume of the deflectable member with fluid and then it may be sealed. Filling of the enclosed volume through the sealable port may be achieved by the temporary insertion of a syringe needle. At least one additional sealable port may be included for the exit of enclosed air during the fluid filling step.

In an embodiment, the deflectable member may include a motor disposed within the enclosed volume and operatively interconnected to an imaging device, e.g., an ultrasound transducer array. The motor drives the array for the reciprocal pivotal movement.

In an embodiment, the deflectable member may include a portion having an enclosed volume and an ultrasound transducer array disposed within the enclosed volume. In certain embodiments the deflectable member may further include a fluid (e.g., a liquid) disposed within the enclosed volume. In such embodiments, an ultrasound transducer array may be surrounded by the fluid to facilitate acoustic coupling. In certain embodiments the ultrasound transducer array may be disposed for reciprocal pivotal movement within the enclosed volume, thereby yielding three-dimensional images of internal body anatomy.

In one aspect, the deflectable member may include a bellows member having a flexible, closed-end portion located within the fluid in the enclosed volume and an open-end isolated from the fluid, wherein the bellows member is collapsible and expansible in response to volumetric variations in the fluid. As may be appreciated, the provision of a bellows member may maintain operational integrity of the deflectable member when exposed to conditions that may cause a volumetric change in the contained fluid.

At least the closed end portion of the bellows member may be elastically deformable. In this regard, the closed end portion of the bellows member may be elastically expandable in response to volumetric variations in the fluid. The bellows member may be operable to maintain operational integrity of the deflectable member despite fluid volume changes that may occur due to exposure of the deflectable member to relatively warm or cool temperatures during, for example, transport and/or storage. Such an elastically expandable bellows member may be particularly advantageous with respect to low temperatures where the fluid typically contracts more than the deflectable member.

In another aspect, the deflectable member may include a bubble-trap member fixedly positioned relative to the enclosed volume and a fluid disposed within the enclosed volume. The bubble-trap member may have a distal-facing concave surface, wherein a distal portion of the enclosed volume is defined distal to the bubble-trap member and a proximal portion of the enclosed volume is defined proximal to the bubble-trap member. The ultrasound transducer array may be located in the distal portion and an aperture may be provided through the bubble-trap member to fluidly connect the distal portion of the enclosed volume to the proximal portion of the enclosed volume.

As may be appreciated, bubbles present in the contained fluid can negatively affect images obtained by the ultrasound transducer array and are undesired. In the described arrangement, the deflectable member may be oriented with the proximal end upwards, wherein bubbles may be directed by the concave surface through the aperture of the bubble-trap, and effectively isolated from the ultrasound transducer array by virtue of the bubbles being trapped in the proximal portion of the enclosed volume by the bubble-trap. In another method of controlling bubble location, a user may grasp the catheter at a point proximal to the enclosed volume and swing around the portion with the enclosed volume to impart centrifugal force on the fluid within the enclosed volume thereby causing the fluid to move toward the distal end and any bubbles within the fluid to move towards the proximal portion of the enclosed volume.

In an arrangement, a filter may be disposed across the aperture. The filter may be configured such that air may pass through the aperture while the fluid may be unable to pass through the aperture. The filter may include expanded polytetrafluoroethylene (ePTFE).

In an embodiment, the ultrasound transducer array may be disposed for reciprocal pivotal movement within the enclosed volume, and a gap between the ultrasound transducer array and an inner wall of the enclosed volume may be sized such that fluid is drawn into the gap via capillary forces. To achieve such a gap, the ultrasound transducer array may include a cylindrical enclosure disposed about the array and the gap may exist between the outer diameter of the cylindrical enclosure and the inner wall of the enclosed volume.

In an aspect, the deflectable member may include a catheter having a portion having an enclosed volume, an imaging device such as an ultrasound transducer array disposed for reciprocal pivotal movement about a pivot axis within the enclosed volume, and an electrical interconnection member having a first portion coiled (e.g., coiled in a single plane in a clock spring arrangement, coiled along an axis in a helical arrangement) within the enclosed volume and electrically interconnected to the imaging device. In an arrangement, the first portion of the electrical interconnection member may be helically disposed within the enclosed volume about a helix axis. As the imaging device is pivoted, the helically wrapped first portion may tighten and loosen about the helix axis. The pivot axis may be coincident with the helix axis. The enclosed volume may be disposed at a distal end of the deflectable member. A fluid may be disposed within the enclosed volume.

In another further aspect, the imaging device, e.g., an ultrasound transducer array may be disposed for reciprocal movement about a pivot axis within the enclosed volume. The deflectable member may further include at least a first electrical interconnection member (e.g. for conveying imaging signals to/from the imaging device). The first electrical interconnection member may include a first portion coiled about the pivot axis and interconnected to the ultrasound transducer array.

In an embodiment, the first electrical interconnection member may include a second portion adjoining the first portion, wherein the second portion is fixedly positioned relative to a catheter body, and wherein upon reciprocal movement of the imaging device, the coiled first portion of the first electrical interconnection member tightens and loosens about the pivot axis. The second portion of the first electrical interconnection member may be helically and fixedly positioned about an inner core member disposed within the catheter body.

In one approach, the first electrical interconnection member may be ribbon-shaped and may comprise a plurality of conductors arranged side-by-side with electrically non-conductive material disposed therebetween across the width of the member. By way of example, the first electrical interconnection member may comprise a GORE™ Micro-Miniature Ribbon Cable available from WL Gore & Associates, Newark, Del., U.S.A, wherein the first portion of the first electrical interconnection member may be disposed so that a top or bottom side thereof faces and wraps about a pivot axis of an ultrasound transducer array.

In another embodiment, the first portion of the electrical interconnection member may be coiled a plurality of times about the pivot axis. More particularly, the first portion of the first electrical interconnection member may be helically disposed about the pivot axis a plurality of times. In one approach, the first electrical interconnection member may be helically disposed about the pivot axis in a non-overlapping manner, i.e. where no portion of the first electrical interconnection member overlies another portion thereof.

In another approach, the first electrical interconnection member may be ribbon-shaped and may be helically disposed about the pivot axis a plurality of times. Upon reciprocal pivotal movement of the ultrasound transducer array, the helically wrapped, ribbon shaped portion may tighten and loosen about the helix axis. The deflectable member may further include a motor operable to produce the reciprocal pivotal movement. A flexboard may be electrically interconnected to the imaging device and the flexboard may electrically interconnect to the first electrical interconnection member at a location between the motor and an outer wall of the catheter. The interconnection between the flexboard and the first electrical interconnection member may be supported by a cylindrical interconnection support.

The deflectable member may be configured such that the imaging device is disposed distally along the deflectable member relative to the first portion of the first electrical interconnection member. In an alternate arrangement, the deflectable member may be configured such that the first portion of the first electrical interconnection member is disposed distally relative to the imaging device. In such an alternate arrangement, a portion of the first electrical interconnection member may be fixed relative to a tip case of the deflectable member where the first electrical interconnection member passes the imaging device. In either arrangement, the first portion may be coiled within the enclosed volume.

In an arrangement, the deflectable member may include a driveshaft operatively interconnected to the imaging device. The driveshaft may be operable to drive the imaging device for the reciprocal pivotal movement. The driveshaft may extend from the proximal end of the deflectable member to the imaging device. The driveshaft may be driven by a motor.

In an embodiment, the first portion of the first electrical interconnection member may be disposed in a clock spring arrangement. A center line of the first portion of the first electrical interconnection member may be disposed within a single plane that is in turn disposed perpendicular to the pivot axis. The deflectable member includes a distal end and a proximal end, and in an arrangement, the first portion (the clock spring) may be disposed closer to the distal end of the deflectable member than the imaging device. The first portion may comprise a flexboard.

In an aspect, the catheter may include a deflectable member, an imaging device, and at least a first electrical interconnection member. The deflectable member may have a portion having a first volume that may be open to an environment surrounding at least a portion of the deflectable member. The imaging device may be disposed for reciprocal pivotal movement about a pivot axis within the first volume. In this regard, the imaging device may be exposed to fluid (e.g., blood) present in the environment surrounding the deflectable member. The first electrical interconnection member may have a first portion coiled within the first volume and electrically interconnected to the imaging device. In an embodiment, the first portion of the first electrical interconnection member may be helically disposed within the first volume about a helix axis. The first electrical interconnection member may further include a second portion adjoining the first portion. The second portion may be fixedly positioned relative to a case partially surrounding the first volume. Upon the reciprocal pivotal movement, the coiled first portion of the first electrical interconnection member may tighten and loosen. The first electrical interconnection member may be ribbon-shaped and include a plurality of conductors arranged side-by-side with electrically non-conductive material therebetween. The first portion of the first electrical interconnection member may be disposed in a clock spring arrangement. The clock spring arrangement may be disposed within the first volume that may be open to the environment surrounding at least a portion of the deflectable member. A structure may surround the imaging device. For example, an acoustically-transmissive structure, capable of focusing, defocusing, or transmitting without altering, acoustic energy may fully or partially surround an ultrasound transducer array. The structure may have a round cross-sectional profile. Such a profile, especially if rounded, may reduce turbulence in the surrounding blood, reduce damage to the surrounding blood cells, and aid in avoiding thrombus formation while the imaging device is undergoing reciprocal pivotal movement.

In another aspect, a method is provided for operating a catheter having a deflectable imaging device located at a distal end thereof. A deflectable imaging device may be in the form of a deflectable member that includes componentry for the generation of images. The method may include moving the distal end of the catheter from an initial position to a desired position and obtaining image data from the deflectable imaging device during at least a portion of the moving step. The deflectable imaging device may be located in a first position during the moving step. Moving to the desired position may include the utilization of steering controls in the catheter to direct the catheter orientation within the anatomy. The method may further include utilizing the image data to determine when the catheter is located at the desired position, deflecting the deflectable imaging device relative to the distal end of the catheter from the first position to a second position after the moving step; and optionally advancing an interventional device through an optional port at the distal end of the catheter and into an imaging field of view of the deflectable imaging device in the second position.

In an arrangement, the deflecting step may further include translating a proximal end of at least one of an outer tubular body of the catheter and actuation device of the catheter relative to a proximal end of the other one of the outer tubular body and actuation device.

A deflection force may be applied to a hinge in response to the translating step. The deflectable imaging device may be supportably interconnected by the hinge to one of the catheter body and the actuation device. The deflection force may be initiated in response to the translating step. The deflection force may be communicated in a balanced and distributed manner about a central axis of the outer tubular body. Communicating the deflection force in such a manner may reduce undesirable bending and/or whipping of the catheter.

In an arrangement, the position of the deflectable imaging device may be maintained relative to the distal end of the catheter during the moving and obtaining steps. In an embodiment, the deflectable imaging device may be side-looking in the first position and forward-looking or rearward-looking in the second position. In an embodiment, the imaging field of view may be maintained in a substantially fixed registration relative to the distal end of the catheter during the advancing step.

The following aspects describe catheters including a deflectable member. Although not mentioned, such deflectable members may include motors for selective driven movement of a component or components within the deflectable member. For example, where appropriate, the deflectable members described hereinafter may each include a motor for selective driven movement of the ultrasound transducer arrays.

In an additional aspect, at least a portion of the deflectable member may be permanently located outside of the outer tubular body. In this regard, the deflectable member may be selectively deflectable away from a central axis of the outer tubular body. In certain embodiments, such deflectability may be at least partially or entirely distal to the distal end of the outer tubular body.

In one aspect, the catheter may also include a lumen for conveyance of a device and/or material such as delivering an interventional device extending through the outer tubular body from the proximal end of the outer tubular body to a point distal thereto. For purposes hereof, “interventional device” includes without limitation diagnostic devices (e.g., pressure transducers, conductivity measurement devices, temperature measurement devices, flow measurement devices, electro- and neuro-physiology mapping devices, material detection devices, imaging devices, central venous pressure (CVP) monitoring devices, intracardiac echocardiography (ICE) catheters, balloon sizing catheters, needles, biopsy tools), therapeutic devices (e.g., ablation catheters (e.g., radio-frequency, ultrasonic, optical), patent foramen ovale (PFO) closure devices, cryotherapy catheters, vena cava filters, stents, stent-grafts, septostomy tools), and agent delivery devices (e.g., needles, cannulae, catheters, elongated members). For purposes hereof, “agent” includes without limitation therapeutic agents, pharmaceuticals, chemical compounds, biologic compounds, genetic materials, dyes, saline, and contrast agents. The agent may be liquid, gel, solid, or any other appropriate form. Furthermore, the lumen may be used to deliver agents therethrough without the use of an interventional device. The combinative inclusion of a deflectable member and lumen for conveyance of a device and/or material therethrough facilitates multi-functionality of the catheter. This is advantageous because it reduces the number of catheters and access sites required during the procedure, provides the potential to limit the interventional procedure time, and enhances ease of use.

In this regard, in certain embodiments the lumen may be defined by an inside surface of the wall of the outer tubular body. In other embodiments, the lumen may be defined by an inside surface of an inner tubular body located within the outer tubular body and extending from the proximal end to the distal end thereof.

In another aspect, a deflectable member may be selectively deflectable through an arc of at least about 45 degrees, and in various implementations at least about 90 degrees, and in other embodiments an arc of at least about 180, about 200, about 260, or about 270 degrees. For example, the deflectable member may be deflectable in a pivot-like manner about a pivot, or hinge, axis through an arc of at least about 90 degrees or at least about 200 degrees. Further, the deflectable member may be selectively deflectable and maintainable at a plurality of positions across a range of different angled positions. Such embodiments are particularly apt for implementing a deflectable member comprising an imaging device.

In certain embodiments, a deflectable member in the form of a deflectable imaging device may be selectively deflectable from an exposed (e.g., where at least a portion of the aperture of the deflectable imaging device is free from interference from the outer tubular body) side-looking first position to an exposed forward-looking, second position. “Side-looking” as used herein is defined as the position of the deflectable imaging device where the field of view of the deflectable imaging device is oriented substantially perpendicular to the distal end of the outer tubular body center axis, i.e., central axis. “Forward-looking” includes where the imaging field of view of the deflectable imaging device is at least partially deflected to enable imaging of a volume that includes regions distal to the distal end of the catheter. For example, a deflectable imaging device (e.g., an ultrasound transducer array) may be aligned with (e.g., disposed parallel to or coaxially with) a central axis of the outer tubular body in a first position. Such an approach accommodates introduction into a vessel or body cavity and imaging of anatomical landmarks during catheter positioning (e.g., during insertion and advancement of the catheter into a vascular passageway or bodily cavity), wherein anatomical landmark images may be employed to precisely position a port of a lumen comprising the catheter. In turn, the ultrasound transducer array may be deflected from the side-looking, first position to a forward-looking, second position (e.g., angled at least about 45 degrees, or in some applications at least about 90 degrees) relative to a central axis of the catheter. An interventional device may then be selectively advanced through a lumen of the catheter and into a work area located adjacent to a lumen port and within an imaging field of view of the ultrasound transducer array, wherein imaged internal procedures may be completed utilizing the interventional device with imaging from the ultrasound transducer array alone or in combination with other imaging modalities (e.g., fluoroscopy). The deflectable imaging device may be deflected such that no part of the deflectable imaging device occupies a volume with the same cross section as the port and extending distally from the port. As such, the imaging field of view of the deflectable imaging device may be maintained in a fixed registration relative to the outer tubular body while the interventional device is being advanced through the outer tubular body, through the port, and into the imaging field of view of the deflectable imaging device.

In certain embodiments, a deflectable imaging device may be selectively deflectable from a side-looking first position to a rearward-looking, second position. “Rearward-looking” includes where the imaging field of view of the deflectable imaging device is at least partially deflected to enable imaging of a volume that includes regions proximal to the distal end of the catheter.

In other embodiments, a deflectable imaging device may be selectively deflectable from a side-looking first position to a variety of selected forward-looking, side-looking and rearward-looking positions thereby enabling the acquisition of multiple imaging planes or volumes within the patient anatomy while preferably maintaining a relatively-fixed or stable catheter position. An ultrasound transducer array may be configured to obtain volumetric imaging and color flow information in which the center beam of the volume can be redirected by such deflection of the transducer. This is particularly beneficial for embodiments for real-time rendering of sequential three dimensional images using a deflectable imaging device with an oscillating one dimensional array or stationary two-dimensional array. In such embodiments, the angle of orientation of the ultrasound transducer array, and deflectable member, relative to the longitudinal axis of the catheter body can be any angle between about +180 degrees to about −180 degrees or an arc of at least about 180, about 200, about 260, or about 270 degrees. Angles contemplated include about +180, +170, +160, +150, +140, +130, +120, +110, +100, +90, +80, +70, +60, +50, +40, +30, +20, +10, 0, −10, −20, −30, −40, −50, −60, −70, −80, −90, −100, −110, −120, −130, −140, −150, −160, −170, and −180 degrees or can fall within or outside of any two of these values.

In a related aspect, a deflectable member may comprise an ultrasound transducer array having an aperture length at least as large as a maximum cross-dimension of the outer tubular body. Correspondingly, the deflectable ultrasound transducer array may be provided for selective deflection from a first position that accommodates advancement of the catheter through a vascular passageway to a second position that is angled relative to the first position. Again, in certain embodiments the second position may be selectively established by a user.

In a related aspect, deflectable member may be deflectable from a first position aligned with the central axis of the catheter (e.g., parallel thereto) to a second position angled relative to the central axis, wherein when in the second position the deflectable member is disposed outside of a working area located adjacent to a lumen port. As such, an interventional device may be advanceable through the port free from interference with the deflectable member.

In certain embodiments, the deflectable member may be provided so that the cross-sectional configuration thereof generally coincides with the cross-sectional configuration of the outer tubular body at the distal end thereof. For example, when a cylindrically-shaped outer tubular body is employed, a deflectable member may be located beyond the distal end of the outer tubular body and configured to coincide with (e.g., slightly exceed, occupy, or fit within) an imaginary cylindrical volume defined by and adjacent to such distal end, wherein the deflectable member is selectively deflectable out of such volume. Such an approach facilitates initial advancement and positioning of the catheter through vascular passageways.

In certain embodiments, a deflectable member may be provided to deflect along an arc path that extends away from a central axis of the outer tubular body. By way of example, in various implementations the deflectable member may be disposed to deflect from a first position that is located distal to a lumen port, to a second position that is lateral to the outer tubular body (e.g., to one side of the outer tubular body).

In another aspect, a deflectable member may be provided to deflect from a longitudinal axis, e.g., the central axis of the catheter. Upon a deflection of 90 degrees from the longitudinal axis, a displacement arc is defined. The displacement arc is the minimum constant-radius arc that is tangent to a face of the deflectable member and tangent to a straight line collinear with the central axis of the catheter at the most distal point of the catheter. The displacement arc associated with a particular embodiment of a deflectable member may be used to compare the deflection performance of that particular embodiment to other deflectable member embodiments and to a minimum bend radius of a steered catheter (in cases where the rigid tip is positioned using only conventional steering). In an aspect, the radius of the displacement arc may be less than about 1 cm. In an aspect, a deflectable member may be provided wherein a ratio of a maximum cross-dimension of the distal end of the outer tubular body to the radius of the displacement arc is at least about 1. By way of example, for a cylindrical outer tubular body, the ratio may be defined by the outer diameter of the distal end of the outer tubular body over the displacement arc radius, wherein such ratio may be advantageously established to be at least about 1.

In an aspect, a catheter with a deflectable member may be provided where the deflectable member may deflect from a longitudinal axis, and where upon a deflection of 90 degrees from the longitudinal axis, a region over which deflection occurs is defined. The region over which deflection occurs is the region along the length of the catheter in which a curvature or other change is introduced in order to achieve the 90 degree deflection. In the case of an ideal hinge, the region over which deflection occurs would be a point. In the case of a living hinge, the region over which deflection occurs approximates a point. In certain embodiments, the region over which deflection occurs may be less than a maximum cross dimension of a catheter body.

In another aspect, a deflectable member may be interconnected to the catheter body wall at the distal end of the outer tubular body. As will be further described, such interconnection may provide support functionality and/or selective deflection functionality. In the latter regard, the deflectable member may be deflectable about a deflection axis that is offset from a central axis of the outer tubular body. For example, the deflection axis may lie in a plane that extends transverse to the central axis of an outer tubular body and/or in a plane that extends parallel to the central axis. In the former regard, in one embodiment the deflection axis may lie in a plane that extends orthogonal to the central axis. In certain implementations, the deflection axis may lie in a plane that extends tangent to a port of a lumen that extends through the outer tubular body of the catheter.

In yet another aspect, the catheter may comprise a lumen (e.g., for delivering an interventional device) extending from the proximal end to an port located at the distal end of the outer tubular body, wherein the port has a central axis coaxially aligned with a central axis of the outer tubular body. Such an arrangement facilitates the realization of relatively small catheter cross-dimensions, thereby enhancing catheter positioning (e.g., within small and/or tortuous vascular passageways). The deflectable member may also be disposed for deflection away from the coaxial central axes, thereby facilitating angled lateral positioning away from the initial catheter introduction (e.g., 0 degree) position of the deflectable member. In certain embodiments, the deflectable member may be deflectable through an arc of at least about 90 degrees or at least about 200 degrees.

In a further aspect, the catheter may include an actuation device, extending from the proximal end to the distal end of the outer tubular body, wherein the actuation device may be interconnected to the deflectable member. Actuation devices may, for example, include balloons, tether lines, wires (e.g., pull wires), rods, bars, tubes, hypotubes, stylets (including pre-shaped stylets), electro-thermally activated shape memory materials, electro-active materials, fluid, permanent magnets, electromagnets, or any combination thereof. The actuation device and outer tubular body may be disposed for relative movement such that the deflectable member is deflectable through an arc of at least about 45 degrees in response to 0.5 cm or less relative movement between the actuation device and the outer tubular body. By way of example, in certain embodiments the deflectable member may be deflectable through an arc of at least about 90 degrees in response to 1.0 cm or less relative movement of the actuation device and outer tubular body.

In a further aspect, the deflectable member may be interconnected to the outer tubular body. In one approach, the deflectable member may be supportably interconnected to the outer tubular body at the distal end thereof. In turn, an actuation device comprising one or more elongate members (e.g., of wire-like construction) may be disposed along the outer tubular body and interconnected at a distal end to the deflectable member, wherein upon applying a tensile or compressive force (e.g., a pull or push force) to a proximal end of the elongate member(s) the distal end of the elongate member(s) may cause the deflectable member to deflect. In this approach, the outer tubular body may define a lumen therethrough (e.g., for delivering an interventional device) extending from the proximal end of the outer tubular body to a port located distal to the proximal end.

In another approach, a deflectable member may be supportably interconnected to one of the outer tubular body and an actuation device, and restrainably interconnected by a restraining member (e.g., a ligature) to the other one of the outer tubular body and actuation device, wherein upon relative movement of the outer tubular body and actuation device the restraining member restrains movement of the deflectable member to affect deflection thereof.

For example, the deflectable member may be supportably interconnected to an actuation device and restrainably interconnected to the outer tubular body at the distal end thereof. In this approach, the actuation device may comprise an inner tubular body defining a lumen therethrough (e.g., for delivering an interventional device) extending from the proximal end of the catheter body to a port located distal to the proximal end.

More particularly, and in a further aspect, the catheter may comprise an inner tubular body, disposed within the outer tubular body for relative movement therebetween (e.g., relative slidable movement). A deflectable member located at the distal end may be supportably interconnected to the inner tubular body. In certain embodiments, the deflectable member may be disposed so that upon selective relative movement of the outer tubular body and inner tubular body the deflectable member is selectively deflectable and maintainable in a desired angular orientation.

For example, in one implementation an inner tubular body may be slidably advanced and retracted relative to an outer tubular body, wherein engagement between surfaces of the two components provides a mechanism interface sufficient to maintain a selected relative position of the two components and corresponding deflected position of the deflectable member. A proximal handle may also be provided to facilitate the maintenance of selected relative positioning of the two components.

In an additional aspect, the catheter may include an actuation device, extending from a proximal end to a distal end of the outer tubular body and moveable relative to the outer tubular body to apply a deflection force to the deflectable member. In this regard, the actuation device may be provided so that deflection force is communicated by the actuation device from the proximal end to the distal end in a balanced and distributed manner about a central axis of the outer tubular body. As may be appreciated, such balanced and distributed force communication facilitates the realization of a non-biased catheter yielding enhanced control and positioning attributes.

In an embodiment, the deflectable member may be operable by the actuation device for selective positioning. In another embodiment, the operation of the actuation device may be independent from steering of the catheter body. In a further embodiment, the operation of the actuation device may operate independently from steering of the catheter and independently from the operation of a motor for driven oscillatory movement of the ultrasound transducer array as described below.

In conjunction with one or more of the above-noted aspects, the catheter may include a hinge that is supportably interconnected to the outer tubular body or, in certain embodiments, to an included actuation device (e.g., an inner tubular body). The hinge may be structurally separate from and fixedly interconnected to the catheter body (e.g., the outer tubular body or the inner tubular body). The hinge may be further fixedly interconnected to the deflectable member, wherein the deflectable member is deflectable in a pivot-like manner. In certain embodiments the hinge may be constructed from the catheter body (e.g., the catheter body may have a portion removed and the remaining portion maybe used as a hinge). The hinge member may be at least partially elastically deformable to deform from a first configuration to a second configuration upon the application of a predetermined actuation force, and to at least partially return from the second configuration to the first configuration upon removal of the predetermined actuation force. Such functionality facilitates the provision of a deflectable member that may be selectively actuated via an actuation device to move from an initial first position to a desired second position upon the application of a predetermined actuation force (e.g., a tensile or pulling force, or a compressive pushing force applied thereto), wherein upon selective release of the actuation force the deflectable member may automatically at least partially retract to its initial first position. In turn, successive deflectable positioning/retraction of the deflectable member may be realized during a given procedure, thereby yielding enhanced functionality in various clinical applications.

In certain embodiments, the hinge member may be provided to have a column strength sufficient to reduce unintended deflection of the deflectable member during positioning of the catheter (e.g., due to mechanical resistance associated with advancement of the catheter). By way of example, the hinge member may exhibit a column strength at least equivalent to that of the outer tubular body.

In certain implementations the hinge may be a portion of a one-piece, integrally defined member. For example, the hinge may comprise a shape memory material (e.g., Nitinol). In one approach, the hinge member may include a curved first portion and a second portion interconnected thereto, wherein the second portion is deflectable about a deflection axis defined by the curved first portion. By way of example, the curved first portion may comprise a cylindrically-shaped surface. In one embodiment, the curved first portion may include two cylindrically-shaped surfaces having corresponding central axes that extend in a common plane and intersect at an angle, wherein a shallow, saddle-like configuration is defined by the two cylindrically-shaped surfaces. In an approach, the hinge member may include a pintle. In an approach, the hinge member may include a membrane that is bendable such that the deflectable member is operable to move through a predefined path at least partially controlled by the membrane.

In yet a further aspect, the outer tubular body may be constructed to facilitate the inclusion of electrical componentry at the distal end thereof. More particularly, the outer tubular body may comprise a plurality of interconnected electrical conductors extending from the proximal end to the distal end. For example, in certain embodiments the electrical conductors may be interconnected in a ribbon-shaped member that is helically disposed about and along all or at least a portion of a catheter central axis, thereby yielding enhanced structurally qualities to the wall of the outer tubular body and avoiding excessive strain on the electrical conductors during flexure of the outer tubular body. For example, in certain embodiments the electrical conductors may be braided along at least a portion of the catheter central axis, thereby yielding enhanced structurally qualities to the wall of the outer tubular body. The outer tubular body may further include a first layer disposed inside of the first plurality of electrical conductors and extending from the proximal end to the distal end, and a second layer disposed on the outside of the first plurality of electrical conductors, extending from the proximal end to the distal end. The first tubular layer and second tubular layer may each be provided to have a dielectric constant of about 2.1 or less, wherein capacitive coupling may be advantageously reduced between the plurality of electrical conductors and bodily fluids present outside of the catheter and within a lumen extending through the outer tubular body.

In yet another aspect, a catheter may include a tubular body. The tubular body may include a wall with a proximal end and a distal end. The wall may include first and second layers extending from the proximal end to the distal end. The second layer may be disposed outside of the first layer. The first and second layers may each have a withstand voltage of at least about 2,500 volts AC. The wall may further include at least one electrical conductor extending from the proximal end to the distal end and disposed between the first and second layers. A lumen may extend through the tubular body. Combined, the first and second layers may provide an elongation resistance such that a tensile load of about 3 pound-force (lbf) (13 Newton (N)) results in no more than a 1 percent elongation of the tubular body.

In an arrangement, the tubular body may provide an elongation resistance such that a tensile load of about 3 lbf (13 N) applied to the tubular body results in no more than a 1 percent elongation of the tubular body, and in such an arrangement at least about 80 percent of the elongation resistance may be provided by the first and second layers.

In an embodiment, the first and second layers may have a combined thickness of at most about 0.002 inches (0.05 millimeters (mm)). Moreover, the first and second layers may have a combined elastic modulus of at least about 345,000 pounds per square inch (psi) (2,379 megapascal (MPa)). The first and second layers may exhibit a substantially uniform tensile profile about the circumference and along the length of the tubular body when a tensile load is applied to the tubular body. The first and second layers may each include helically wound material (e.g., film). For example, the first layer may include a plurality of helically wound films. A first portion of the plurality of films may be wound in a first direction, and a second portion of the films may be wound in a second direction that is opposite from the first direction. One or more of the plurality of films may include a high-strength tensilized film. One or more of the plurality of films may include non-porous fluoropolymer. The non-porous fluoropolymer may comprise non-porous ePTFE. The second layer may be constructed similarly to the first layer. The at least one electrical conductor may be in the form of a multiple conductor ribbon and/or conductive thin film and may be helically wrapped along at least a portion of the tubular body.

As will be appreciated, the construction of the tubular body of the current aspect may be utilized in other aspects described herein such as, for example, aspects where a tubular body is disposed within another tubular body and relative motion between the tubular bodies is used to deflect a deflectable member.

In an embodiment of the current aspect the first and second layers may have a combined thickness of at most about 0.010 inches (0.25 mm). Moreover, the first and second layers may have a combined elastic modulus of at least about 69,000 psi (475.7 MPa). In the present embodiment, the first layer may comprise a first sub-layer of the first layer and a second sub-layer of the first layer. The first sub-layer of the first layer is disposed inside the second sub-layer of the first layer. The second layer may comprise a first sub-layer of the second layer and a second sub-layer of the second layer. The first sub-layer of the second layer is disposed outside the second sub-layer of the first layer. The first sub-layer of the first layer and the first sub-layer of the second layer may include a first type of helically wound film. The second sub-layer of the first layer and the second sub-layer of the second layer may include a second type of helically wound film. The first type of helically wound film may include non-porous fluoropolymer and the second type of helically wound film may include porous fluoropolymer.

In another embodiment, the first layer may have a thickness of at most about 0.001 inches (0.025 mm) and the second layer may have a thickness of at most about 0.005 inches (0.13 mm). Moreover, the first layer may have an elastic modulus of at least about 172,500 psi (1,189 MPa) and the second layer may have an elastic modulus of at least about 34,500 psi (237.9 MPa).

In another aspect, the outer tubular body may comprise a plurality of electrical conductors extending from a proximal end to the distal end and a set of tubular layers inside and/or outside of the first plurality of electrical conductors. The set of tubular layers may comprise a low dielectric constant layer (e.g., located closest to the electrical conductors), and a high withstand voltage layer. In this regard, the low dielectric constant layer may have a dielectric constant of 2.1 or less, and the high withstand voltage layer may be provided to yield a withstand voltage of at least about 2500 volts AC. In certain embodiments, a set of low dielectric and high withstand voltage layers may be provided both inside and outside of the plurality of electrical conductors along the length of the outer tubular body.

In certain embodiments tie layers may be interposed between the electrical conductors and one or more inner and/or outer layers. By way of example, such tie layers may comprise a film material that may have a melt temperature that is lower than other components of the outer tubular body, wherein the noted layers of components may be assembled and the tie layers selectively melted to yield an interconnected structure. Such selectively melted tie layers may prevent other layers of the outer tubular body from migrating relative to each other during manipulation of the outer tubular body (e.g., during insertion into a patient).

For some arrangements, the outer tubular body may further include a shielding layer disposed outside of the electrical conductors. By way example, the shielding layer may be provided to reduce electromagnetic interference (EMI) emissions from the catheter as well as shield the catheter from external EMI.

In certain embodiments, lubricious inside and outside layers and/or coatings may also be included. That is, an inner layer may be disposed within the first tubular layer and an outer layer may be disposed outside of the second tubular layer.

In yet a further aspect, the catheter may be provided to comprise a first electrical conductor portion extending from a proximal end to a distal end of the catheter, and a second electrical conductor portion electrically interconnected to the first electrical conductive portion at the distal end. The first electrical conductor portion may comprise a plurality of interconnected electrical conductors arranged side-by-side with electrically non-conductive material therebetween. In certain implementations, the first electrical conductor portion may be helically disposed about a catheter central axis from the proximal end to the distal end thereof. In conjunction with such implementations, the second electrical conductor portion may comprise a plurality of electrical conductors interconnected to the plurality of interconnected electrical conductors of the first electrical conductor portion, and extending parallel to a central axis of the outer tubular body at the distal end. In certain embodiments, the first electrical conductor portion may be defined by a ribbon-shaped member included within the wall of the outer tubular body, thereby contributing to the structural integrity thereof.

In conjunction with the noted aspect, the first electrical conductor portion may define a first width across the interconnected plurality of electrical conductors, and the second electrical conductor portion may define a second width across the corresponding plurality of electrical conductors. In this regard, the second electrical conductor portion may be defined by electrically conductive traces disposed on a substrate. By way of example, the substrate may extend between the end of the first electrical conductor portion and electrical componentry provided at the distal end of a catheter, including for example an ultrasound transducer array.

In various embodiments, the second electrical conductor portion may be interconnected to a deflectable member and may be of a bendable construction, wherein at least a portion of the second electrical conductor portion is bendable with and in response to deflection of the deflectable member. More particularly, the second electrical conductor portion may be defined by electrically conductive traces on a substrate that is bendable in tandem with a deflectable member through an arc of at least about 90, 180, 200, 260, or 270 degrees.

In a further aspect, the catheter may comprise a deflectable member that includes an ultrasound transducer array, wherein at least a portion of the deflectable ultrasound transducer array may be located within the outer tubular body wall at the distal end. Further, the catheter may include steering means whereby the catheter body can be directed within the anatomy to a preferred location within a cavity, chamber of the heart or for access to a vascular lumen. Still further, the catheter may include a lumen (e.g., for delivering an interventional device) extending from the proximal end to a point distal thereto.

In yet another aspect, the catheter may comprise a motor to effectuate oscillatory or rotary movement of an imaging device, e.g., an ultrasound transducer array. The ultrasound transducer array may be disposed for reciprocal pivotal movement (i.e., rotating back and forth, rather than continuously around, for example, the catheter body central axis, or an axis parallel thereto, with the motor operable for driving the movement. As used herein, the term “rotating” refers to oscillatory or angular motion or movement between a selected +/− degrees of angular range. Oscillatory or angular motion includes but is not limited to partial motion in a clock-wise or counter-clockwise direction or motion between a positive and negative range of angular degrees. A motor includes micro-motors, actuators, microactuators, such as electromagnetic motors including stepper motors, inductive motors or synchronous motor (e.g., Faulhaber Series 0206 B available from MicroMo Electronics, Inc., Clearwater, Fla., U.S.A.); shape memory material actuator mechanisms, such as disclosed in US 2007/0016063 by Park et al.; active and passive or active magnetic actuators; ultrasonic motors (e.g., Squiggle® motors available from New Scale Technologies, Victor, N.Y., U.S.A.); hydraulic or pneumatic drives such as or any combination thereof. The motor may reside in a member that may be moved relative to the catheter body, or may be external from the catheter body, or in the catheter body. The motor may be located in a liquid environment or a non-liquid environment. The motor may be sealed in that it may be capable of being operated in a liquid environment without modification, or the motor may be non-sealed such that it would not be capable of operating in a liquid environment without modification. For example, it may be desired that a particular electromagnetic motor not be operated within a liquid-filled environment. In such an arrangement, a liquid or fluid tight barrier may be used between the electromagnetic motor and the ultrasound transducer array. Motor dimensions are selected to be compatible with the desired application, for example, to fit within components sized for a particular intra-cavity or intravascular clinical application. For example in ICE applications, the components contained therein, such as the motor, may fit in a volume of about 1 mm to about 4 mm in diameter.

In a still further aspect, the catheter may comprise a steerable or pre-curved catheter segment located near the distal end of the outer tubular body and the deflectable member may comprise an ultrasound transducer array. Further, the catheter may include a lumen (e.g., for delivering an interventional device) extending from the proximal end to a point distal thereto.

In another aspect, the catheter may comprise an outer tubular body having a wall, a proximal end and a distal end. The catheter may further include a lumen (e.g., for delivering an interventional device) extending through the outer tubular body from the proximal end to a port located distal to the proximal end. The catheter may further include a first electrical conductor portion comprising a plurality of interconnected electrical conductors arranged side-by-side with electrically non-conductive material therebetween. The first electrical conductor portion may extend from the proximal end to the distal end. The catheter may further include a second electrical conductor portion electrically interconnected to the first electrical conductor portion at the distal end. The second electrical conductor portion may comprise a plurality of electrical conductors. The catheter may further include a deflectable member located at the distal end. The second electrical conductor portion may be electrically interconnected to the deflectable member and may be bendable in response to deflection of the deflectable member.

In another aspect, the catheter may comprise an outer tubular body having a wall, a proximal end and a distal end. The catheter may further include a lumen (e.g., for delivering an interventional device or agent delivery device) extending through the outer tubular body from the proximal end to a port located distal to the proximal end. The catheter may further include a deflectable member, at least a portion of which is permanently located outside of the outer tubular body at the distal end, selectively deflectable relative to the outer tubular body and distal to the port. In an embodiment, the catheter may further include a hinge located at the distal end where the deflectable member may be supportably interconnected to the hinge. In such an embodiment, the deflectable member may be selectively deflectable relative to the outer tubular body about a hinge axis defined by the hinge.

Numerous aspects described hereinabove comprise a selectively deflectable imaging device disposed at a distal end of an outer tubular body of a catheter. Additional aspects of the present invention may include deflectable members in place of such deflectable imaging devices. Such deflectable members may include imaging devices, diagnostic devices, therapeutic devices, or any combination thereof.

The various features discussed above in relation to each aforementioned aspect may be utilized by any of the aforementioned aspects. Additional aspects and corresponding advantages will be apparent to those skilled in the art upon consideration of the further description that follows.

The use herein of terms such as first, second, third, etc. are used herein to distinguish between elements in a particular embodiment and should be interpreted in light of the particular embodiment.

### BRIEF DESCRIPTION OF THE DRAWINGS

FIG. 1A shows a catheter embodiment having a catheter body and a deflectable member.

FIGS. 1B and 1C illustrate the concept of a minimum presentation width for a catheter.

FIG. 2A shows a catheter embodiment having a deflectable ultrasound transducer array located at an end of the catheter.

FIG. 2B shows a cross-sectional view of the catheter embodiment of FIG. 2A.

FIG. 2C shows a catheter embodiment having a deflectable ultrasound transducer array located at a distal end of the catheter.

FIGS. 2D and 2E show the catheter embodiment of FIGS. 2B and 2C, wherein the catheter further includes an optional steerable segment.

FIGS. 3A through 3D show further catheter embodiments having a deflectable ultrasound transducer array located at a distal end of the catheter.

FIG. 4 shows a catheter embodiment having electrically conductive wires attached to an ultrasound transducer array located near the distal end of the catheter, wherein the electrically conductive wires helically extend to the proximal end of the catheter and are embedded in the catheter wall.

FIG. 4A shows an exemplary conductive wire assembly.

FIG. 5A shows an embodiment of a catheter that includes a deflectable member.

FIGS. 5B through 5E show an embodiment of a catheter that includes a deflectable member wherein the deflectable member is deflectable by moving an inner tubular body relative to an outer tubular body.

FIG. 5F shows an embodiment of an electrical interconnection between a helically disposed electrical interconnection member and a flexible electrical member.

FIGS. 6A through 6D show an embodiment of a catheter that includes a deflectable member wherein the deflectable member is deflectable by moving an elongate member relative to a catheter body.

FIGS. 7A and 7B show a further aspect wherein an ultrasound transducer array is located near the distal end of the catheter. The array can be manipulated between side-looking and forward-looking by utilizing an actuation device attached to the array and extending to the proximal end of the catheter.

FIGS. 8A through 8D show various exemplary variations of the catheter of FIGS. 7A and 7B.

FIGS. 9, 9A and 9B demonstrate further embodiments wherein an ultrasound array is deflectable.

FIGS. 10A and 10B demonstrate further alternative embodiments.

FIGS. 11, 11A and 11B demonstrate further embodiments.

FIG. 12 demonstrates a still further embodiment.

FIG. 13 is a flow chart for an embodiment of a method of operating a catheter.

FIGS. 14A, 14B, 14C, 14D and 15 illustrate alternative support designs.

FIG. 16 illustrates a further embodiment of a catheter.

FIG. 17 illustrates a further embodiment of a catheter.

FIGS. 18A and 18B demonstrate a further embodiment wherein an ultrasound array is deflectable.

FIGS. 19A, 19B and 19C demonstrate a further embodiment wherein an ultrasound array is deflectable.

FIGS. 20A and 20B demonstrate a further embodiment wherein an ultrasound array is deflectable.

FIG. 21 illustrates an alternative support design.

FIGS. 22A and 22B demonstrate a further embodiment wherein an ultrasound array is deflectable.

FIGS. 23A and 23B demonstrate a further embodiment wherein an ultrasound array is deflectable.

FIGS. 24A, 24B and 24C demonstrate a further embodiment of a catheter wherein an ultrasound array is deployable from within the catheter.

FIGS. 25A and 25B demonstrate a further embodiment of a catheter wherein an ultrasound array is deployable from within the catheter.

FIG. 25C demonstrates a further embodiment of a catheter wherein an ultrasound array is deployable from within the catheter to a rearward-looking position.

FIGS. 26A and 26B demonstrate a further embodiment of a catheter wherein a tip portion is temporarily bonded to a tubular body.

FIGS. 27A, 27B and 27C illustrate a further embodiment of a catheter wherein an ultrasound array is movable via a pair of cables.

FIGS. 28A and 28B demonstrate a further embodiment of a catheter that is pivotably interconnected to an inner tubular body.

FIGS. 29A and 29B demonstrate another embodiment of a catheter that is pivotably interconnected to an inner tubular body.

FIGS. 30A and 30B demonstrate yet another embodiment of a catheter that is pivotably interconnected to an inner tubular body.

FIGS. 31A and 31B illustrate the embodiment of FIGS. 30A and 30B with the addition of a resilient tube.

FIGS. 32A and 32B demonstrate a further embodiment of a catheter that includes a buckling initiator.

FIGS. 33A and 33B demonstrate a further embodiment of a catheter that includes two tethers.

FIGS. 34A and 34B demonstrate a further embodiment of a catheter that includes two tethers partially wrapped about an inner tubular body.

FIGS. 35A and 35B demonstrate a further embodiment of a catheter that is secured in an introductory configuration by a tether wound about an inner tubular body.

FIGS. 36A through 36C demonstrate a further embodiment of a catheter attached to a pivoting arm and deployable with a push wire.

FIGS. 37A and 37B demonstrate a further embodiment of a catheter deployable with a push wire.

FIGS. 38A and 39B demonstrate two further embodiments of catheters with ultrasound imaging arrays deployed on a plurality of arms.

FIGS. 40A and 40B demonstrate a further embodiment of a catheter with ultrasound imaging arrays deployed on a plurality of arms.

FIGS. 41A through 41C demonstrate a further embodiment of a catheter with an ultrasound imaging array deployed on a deflectable portion of an inner tubular body.

FIGS. 42A through 42C illustrate a spring element that may be disposed within a catheter.

FIGS. 43A through 43C illustrate a catheter with a collapsible lumen that may be used to pivot an ultrasound imaging array.

FIGS. 44A and 44B illustrate a catheter with a collapsible lumen.

FIGS. 45A and 45B illustrate a catheter with an expandable lumen.

FIGS. 46A and 46B illustrate a catheter that includes an inner tubular body that includes a hinge portion and a tip support portion.

FIGS. 47A and 47B illustrate a catheter that includes tubular portion that includes a hinge.

FIGS. 48A through 48D illustrate a catheter that includes a snare.

FIGS. 49A and 49B illustrate a catheter that includes an electrical interconnection member that connects to a distal end of an ultrasound imaging array.

FIG. 50 illustrates a method of electrically interconnecting a spirally wound portion of a conductor to an ultrasound imaging array.

FIGS. 51A and 51B illustrate catheters with pull wires that transition from a first side of a catheter to a second side of the catheter.

FIGS. 52A and 52B illustrate an electrical interconnection member wrapped about a substrate.

FIG. 53 is a partial cross-sectional view of an ultrasound catheter probe assembly.

FIG. 54 is another partial cross-sectional view the ultrasound catheter probe assembly of FIG. 53.

FIG. 55 is a partial cross-sectional view of an ultrasound catheter probe assembly.

FIG. 56A is a partial cross-sectional view of an ultrasound catheter probe assembly.

FIG. 56B is a partial cross-sectional end view of the ultrasound catheter probe assembly of FIG. 56A.

FIG. 57 illustrates an ultrasound imaging system with a handle, a catheter, and a deflectable member.

FIG. 58 illustrates a transverse cross section of a catheter that may be used in the ultrasound imaging system of FIG. 57.

FIG. 59 illustrates a transverse cross section of another embodiment of a catheter.

FIGS. 60 and 61 illustrate a distal end of a catheter body connected by a hinge to a deflectable member.

FIG. 62 illustrates a distal end of a catheter body connected by a hinge to a deflectable member.

FIGS. 63A through 63D illustrate an embodiment of a living hinge.

FIGS. 64A through 64C illustrate a deflectable member connected to a catheter body by a living hinge.

FIG. 64D illustrates another deflectable member connected to a catheter body by a living hinge.

FIGS. 65A through 65E illustrate a deflectable member connected to a catheter body by a hinge.

FIG. 65F illustrates a deflectable member connected to a catheter body with two living hinges.

FIGS. 66A through 66E illustrate a deflectable member connected to a catheter body by a hinge having a pivot pin.

FIG. 67 illustrates another embodiment of a hinge.

FIG. 68 illustrates a deflectable member connected to a catheter body by a hinge and electrical interconnections between the deflectable member and the catheter body.

FIGS. 69A through 69C illustrate another deflectable member having a motor and an electrical interconnection member in a clock spring formation around the motor.

FIGS. 70A and 70B illustrate a deflectable member having a motor and a transducer array.

FIGS. 71A and 71B illustrate a deflectable member having a transducer array, motor, and electrical interconnection member connected to a catheter body by a living hinge.

FIG. 72 illustrates another deflectable member having a motor and a transducer array.

FIG. 73A illustrates another deflectable member having a transducer array, motor, and electrical interconnection member connected to a catheter body by a living hinge.

FIG. 73B illustrates another deflectable member having a transducer array, motor, and electrical interconnection member connected to a catheter body by a living hinge.

FIG. 74 illustrates another deflectable member connected, by a living hinge, to a catheter body, where the deflectable member includes a transducer array and the catheter body includes a motor.

FIGS. 75 and 76 show placement of a steerable catheter embodiment for intracardiac echocardiography within the right atrium of the heart.

FIG. 77 shows placement of the embodiment of FIG. 75 in the right atrium of the heart with a deflectable member deflected to a second position.

FIG. 78 shows placement of the embodiment of FIG. 75 in the right atrium of the heart with the deflectable member deflected to a third position

### DETAILED DESCRIPTION OF THE DRAWINGS

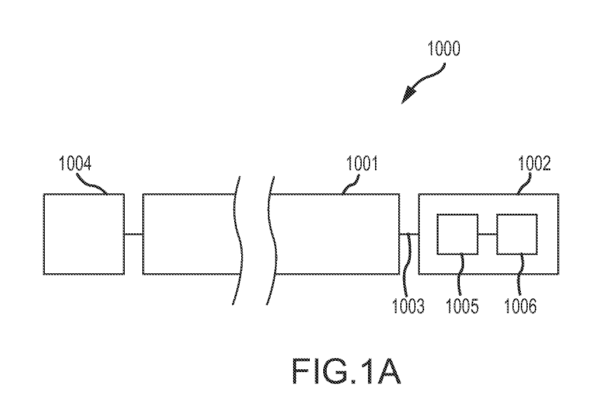

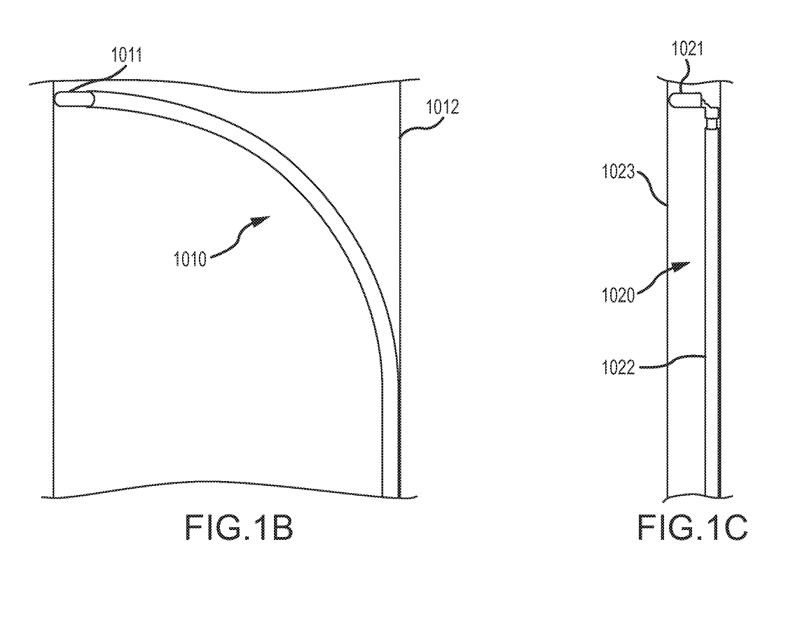

FIG. 1A schematically illustrates an embodiment of a catheter 1000. The catheter 1000 may be inserted into a body of a patient, and portions of the catheter 1000 within the body may be manipulated utilizing another portion of the catheter 1000 such as a portion located outside of the body. Thus, when the catheter 1000 is inserted into a body, a proximal end of the catheter 1000 remains outside of the body and accessible to a clinician for control of distal portions of the catheter 1000 positioned within the body. The catheter 1000 may be employed for a wide variety of purposes, including: the positioning and/or delivery of electronic devices such as diagnostic devices (e.g., imaging devices) and devices which delivery therapies such as therapeutic compounds or energy (e.g., ablation catheters); the deployment and/or retrieval of implantable devices (e.g., stents, stent grafts, vena cava filters); or any combination thereof.

The catheter 1000 includes a catheter body 1001. The catheter body 1001 is an elongate member with a proximal end and a distal end. The catheter body 1001 may comprise, for example, a shaft (e.g., a solid shaft, a shaft comprising at least one lumen), an outer tubular body, an inner tubular body, or any combination thereof. The catheter body 1001 may include a steerable segment or a plurality of steerable segments along a length thereof. At least portions of the catheter body 1001 may be flexible and capable of bending to follow the contours of passageways within the body of the patient into which it is being inserted.

The catheter body 1001 may optionally include a lumen. Such a lumen may run all or a portion of the length of the catheter body 1001 and may have a port at or near the distal end of the catheter body 1001. Such a lumen may be used to convey a device and/or material therethrough (e.g., deliver a device and/or material to or near to the distal end of the catheter body 1001). In another example, the lumen may be used to deliver a therapeutic device, an imaging device, an implantable device, a dosage of a therapeutic compound, or any combination thereof to or proximate to the distal end of the catheter body 1001. In another example, the lumen may be used to retrieve a device such as a vena cava filter.

The catheter 1000 includes a deflectable member 1002. As illustrated, the deflectable member 1002 may be disposed at the distal end of the catheter body 1001. The deflectable member may be operable to deflect relative to the distal end of the catheter body 1001. For example, the deflectable member 1001 may be operable for positioning across a range of angles relative to the longitudinal axis of the catheter body 1001 at the distal end of the catheter body 1001. The deflectable member 1002 may have a smooth, rounded exterior profile that may help in reducing thrombus formation and/or tissue damage as the deflectable member 1002 is moved (e.g., advanced, retracted, rotated, repositioned, deflected) within the body.

The deflectable member 1002 is interconnected to the catheter body 1001 through an interconnection 1003 that allows the deflectable member 1002 to deflect relative to the distal end of the catheter body 1001. The interconnection 1003 may comprise a, component or material that connects two objects, typically allowing relative rotation between them, e.g., one or more joints or hinges of appropriate type such as a living hinge or an ideal hinge (which may be referred to as an real hinge). Such hinges may be made of flexible material or of components that may move relative to each other. Such hinges may include a pintle. In the case of a single ideal hinge, the deflectable member 1002 may rotate relative to the catheter body 1001 about a fixed axis of rotation. In the case of a single living hinge, the deflectable member 1002 may rotate relative to the catheter body 1001 about a substantially fixed axis of rotation. The interconnection 1003 may comprise linking members, such as bars pivotably interconnected to the catheter body 1001 and/or deflectable member 1002, to control the motion of the deflectable member 1002 relative to the catheter body 1001. The interconnection 1003 may comprise a biasing member (e.g., a spring) to bias the deflectable member 1002 to a desired position relative to the catheter body 1001 (e.g., aligned with the distal end of the catheter body 1001). The interconnection 1003 may comprise a shape memory material.

The deflection of the deflectable member 1002 may be controlled by a deflection control member 1004. The deflection control member 1004 may be disposed along the catheter body 1001 at a point outside of the body (e.g., at the proximal end of the catheter body 1001). The deflection control member 1004 may, for example, include a knob, slider, or any other appropriate device interconnected to one or more control wires that are in turn interconnected to the deflectable member 1002, such that rotation of the knob or movement of the slider produces a corresponding deflection of the deflectable member 1002. In such an embodiment, the control wire or wires may run along the catheter body 1001 from the deflection control member 1004 to the deflectable member 1002. In another embodiment, the deflection control member 1004 may be an electronic controller operable to control an electrically deflected deflectable member 1002. In such an embodiment, electrical conductors for deflection control may run along the catheter body 1001 from the deflection control member 1004 to the components for deflecting the deflectable member 1002.

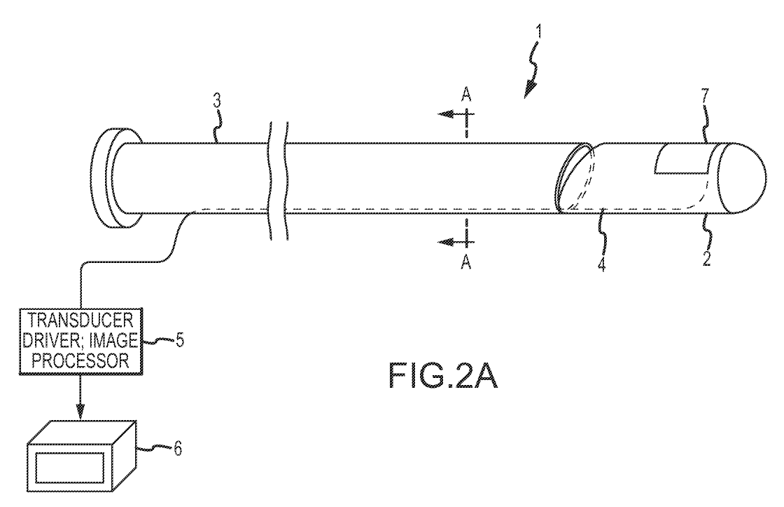

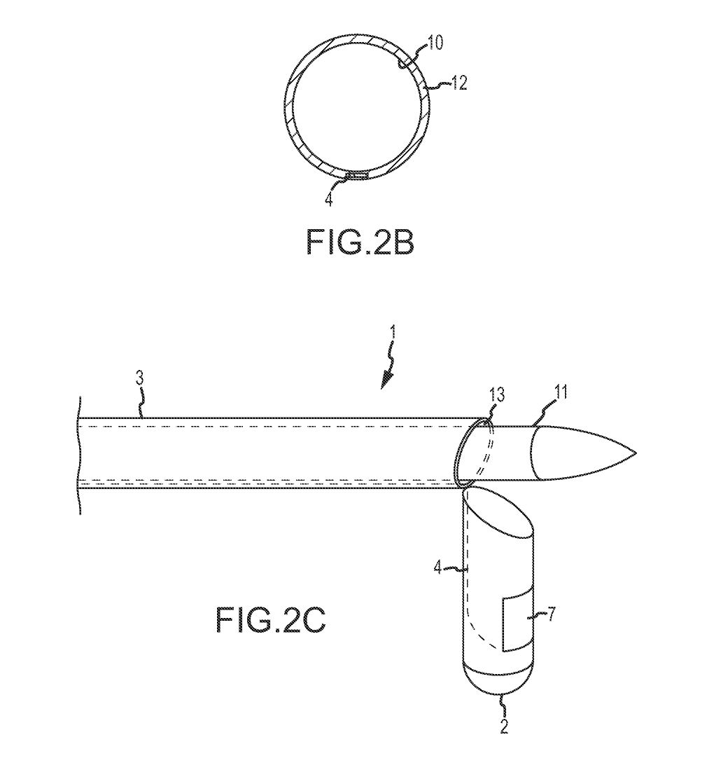

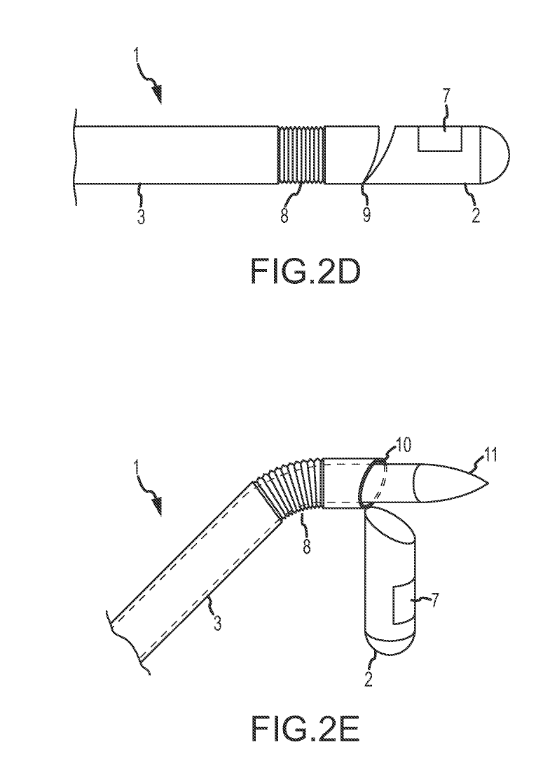

The deflectable member 1002 may optionally include a motor 1005 for driving a driven member 1006. The motor 1005 may be operatively interconnected to the driven member 1006 to move the driven member 1006. For example, the motor 1005 may be operable to drive the driven member 1006 such that the driven member 1006 pivotally reciprocates about a pivot axis. The motor 1005 may be any appropriate device, including the devices discussed herein, for creating motion that may be used to drive the driven member 1006. Although FIG. 2A schematically shows the driven member 1006 disposed distal to the motor 1005, other configurations are contemplated. For example, the motor 1005 may be disposed distal to the driven member 1006. In another example, the motor 1005 and the driven member 1006 may be located in a side-by-side (e.g., stacked, piggy-back) arrangement such that portions of the motor 1005 and the driven member 1006 are co-located at the same point along a longitudinal axis of the deflectable member 1002 (e.g., both the motor 1005 and the driven member 1006 intersect a single plane disposed perpendicular to the longitudinal axis of the deflectable member).

The driven member 1006 may be an electrical device such as an imaging, diagnostic and/or therapeutic device. The driven member 1006 may include a transducer array. The driven member 1006 may include an ultrasound transducer. The driven member 1006 may include an ultrasound transducer array, such as a one dimensional array or a two dimensional array. In an example, the driven member 1006 may include a one dimensional ultrasound transducer array that may be reciprocally pivoted by the motor 1005 such that an imaging plane of the one dimensional ultrasound transducer array is swept through a volume, thus enabling the generation of 3D images and 4D image sequences.

The catheter body 1001 may include one or more members that run along the length of the catheter body 1001. For example, the catheter body 1001 may include electrical conductors running along the length of the catheter body 1001 that electrically connect the motor 1005 and the driven member 1006 to componentry located elsewhere on or apart from the catheter such as motor controllers, ultrasound transducer controllers, and ultrasound imaging equipment. The catheter body 1001 may include control wires or other control devices to steer a steerable portion of the catheter body 1001 and/or control the deflection of the deflectable member 1002.

The catheter 1000 may, for example, be employed for imaging a heart. In an exemplary use, the catheter 1000 may be introduced into the body and positioned within the heart. While within the heart, the motor 1005 may reciprocally drive the driven member 1006 in the form of an ultrasound transducer array to generate 3D images and/or 4D image sequences of the heart. Also while in the heart, the deflectable member 1002 may be deflected to reposition the field of view of the ultrasound transducer array.

Certain embodiments of the deflectable member 1002 may be deflectable such that a minimum presentation width of the catheter 1000 is less than about 3 cm. The minimum presentation width for a catheter is equal to the minimum diameter of a straight tube in which the entire catheter may fit (without kinking) while a tip of the catheter is oriented perpendicular to the axis of the tube. The concept of the minimum presentation width is illustrated in FIGS. 1B and 1C. FIG. 1B illustrates a catheter 1010 steered using conventional catheter steering techniques, such as control wires disposed within the wall of the catheter 1010. For catheter 1010 to fit into a tube 1012 with a tip 1011 of the catheter 1010 oriented perpendicular to the tube 1012, the tube 1012 must be sized to accommodate the length of the tip 1011 of the catheter 1010 and the radius of the portion of the catheter 1010 that must bend to orient the tip 1011 at 90 degrees. Typically, a conventionally steered catheter may have a minimum presentation width of about 6 cm or more. In contrast, embodiments of catheters described herein, such as catheter 1020 that includes a deflectable member 1021, may be operable to fit within a tube 1023 whose diameter is close to the sum of the length of the deflectable member 1021 plus the diameter of a catheter body 1022 of the catheter 1020.

The detailed description that follows in relation to FIGS. 2A through 52B is directed to various catheter embodiments that include a deflectable member that comprises an ultrasound transducer array, and a lumen (e.g., for delivering an interventional device). Such embodiments are for exemplarily purposes and are not intended to limit the scope of the present invention. In that regard, the deflectable member may comprise componentry other than or in addition to an ultrasound transducer array. Such componentry may include: mechanical devices such as needles, and biopsy probes, including cutters, graspers, and scrapers; electrical devices such as conductors, electrodes, sensors, controllers, and imaging componentry; and deliverable components such as stents, grafts, liners, filters, snares, and therapeutics.

Although not mentioned, the embodiments of FIGS. 2A through 52B may also include a motor for moving the ultrasound transducer array or other componentry. Further, additional embodiments may utilize inventive features described herein that do not necessitate the inclusion of a lumen.

An ultrasound transducer array built into a catheter presents unique design challenges. Two critical points include, for example, the resolution in the image plane and the ability to align that image plane with an interventional device.

The resolution in the imaging plane of an ultrasound array can be approximated by the following equation:

Lateral resolution=Constant*wavelength*Image Depth/Aperture Length

For catheters being described here, the wavelength is typically in the range of 0.2 mm (at 7.5 MHz). The constant is in the range of 2.0. The ratio of (Image Depth/Aperture Length) is a critical parameter. For ultrasound imaging in the range of 5-10 MHz for catheters presented here, acceptable resolution in the imaging plane can be achieved when this ratio is in the range of 10 or less.

For imaging with a catheter in the major vessels and the heart, it is desirable to image at depths of 70 to 100 mm. Catheters used in the heart and major vessels are typically 3 to 4 mm in diameter or smaller. Thus while conceptually a transducer array can be made of arbitrary size and placed at any position within the catheter body, this model shows that transducer arrays that readily fit within the catheter structure do not have sufficient width for acceptable imaging.

The ultrasound image plane produced by the array placed on the catheter typically has a narrow width normally referred to as the out of plane image width. For objects to be seen in the ultrasound image, it is important that they be in this image plane. When a flexible/bendable catheter is placed in a major vessel or heart, the image plane can be aligned to some degree. It is desirable to guide a second device placed in the body with the ultrasound image, but doing so requires placing that second device in the plane of the ultrasound image. If the imaging array and the interventional device are both on flexible/bendable catheters that are inserted into the body, it is extremely difficult to orient one interventional device into the ultrasound image plane of the imaging catheter.

Certain embodiments of the present invention utilize an ultrasound image to guide an interventional device. To accomplish this, a large enough aperture is needed to produce an image of acceptable resolution while being able to place the device in a known position that is stable relative to the imaging array and/or to be able to align and/or register the interventional device to the ultrasound image plane.

In certain implementations, the aperture length of the ultrasound array may be larger than the maximum cross dimension of the catheter. In certain implementations, the aperture length of the ultrasound array may be much larger (2 to 3 times larger) than the diameter of the catheter. This large transducer, however, may fit within the 3 to 4 mm maximum diameter of the catheter to be inserted into the body. Once in the body, the imaging array is deployed out of the catheter body leaving space to pass an interventional device through that same catheter that will then be located in a known position relative to the imaging array. In certain arrangements, the imaging array may be deployed in a way so that the interventional device can be readily kept within the ultrasound image plane.

The catheter may be configured for delivery through a skin puncture at a remote vascular access site (e.g., vessel in the leg). Through this vascular access site, the catheter may be introduced into regions of the cardiovascular system such as the inferior vena cava, heart chambers, abdominal aorta, and thoracic aorta.

Positioning the catheter in these anatomic locations provides a conduit for conveyance of devices or therapy to and/or from specific target tissues or structures. One example of this includes bedside delivery of inferior vena cava filters in patients for whom transport to the catheterization laboratory is either high risk or otherwise undesirable. The catheter with the ultrasound transducer array allows the clinician to not only identify the correct anatomical location for placement of the inferior vena cava filter, but also provides a lumen through which the vena cava filter can be delivered under direct ultrasound visualization. Both location identification and delivery of a device can occur without withdrawal or exchange of the catheter and/or imaging device. In addition, post-delivery visualization of the device allows the clinician to verify placement location and function(s) prior to removal of the catheter.

Another application of such a catheter is as a conduit through which ablation catheters can be delivered within the atria of the heart. Although ultrasound imaging catheters are utilized today in many of these cardiac ablation procedures, it is very difficult to achieve proper orientation of the ablation catheters and ultrasound catheter so as to attain adequate visualization of the ablation site. The catheter described herein provides a lumen through which the ablation catheter can be directed and the position of the ablation catheter tip monitored under direct ultrasound visualization. As described, the coaxial registration of this catheter and other interventional devices and therapy delivery systems provides the means by which direct visualization and control can be achieved.

Turning back now to the figures, FIG. 2A shows a catheter embodiment having an ultrasound transducer array 7 located on a deflectable distal end of the catheter 1. Specifically, catheter 1 comprises a proximal end 3 and a distal end 2. Located on the distal end 2 is the ultrasound transducer array 7. Attached to ultrasound transducer array 7 is at least one electrically conductive wire 4 (such as a GORE™ Micro-Miniature Ribbon Cable) that extends from the array 7 to the proximal end 3 of catheter 1. The at least one electrically conductive wire 4 exits the catheter proximal end 3 through a port or other opening in the catheter wall and is electrically connected to transducer driver; image processor 5 which provides a visual image via device 6. Such an electrical connection may include a continuous conduction path through a conductor or series of conductors. Such an electrical connection may include an inductive element, such as an isolation transformer. Where appropriate, other electrical interconnections discussed herein may include such inductive elements.

FIG. 2B is a cross-section of FIG. 2A taken along lines A-A. As can be seen in FIG. 2B, the catheter 1 includes a catheter wall portion 12 that extends at least the length of proximal end 3 and further defines lumen 10 that extends at least the length of proximal end 3. Catheter wall 12 can be any suitable material or materials, such as extruded polymers, and can comprise one or more layers of materials. Further shown is the at least one electrically conductive wire 4 located at the bottom portion of catheter wall 12.

Operation of the catheter 1 can be understood with reference to FIGS. 2A and 2C. Specifically, the catheter distal end 2 can be introduced into the desired body lumen and advanced to a desired treatment site with ultrasound transducer array 7 in a side-looking configuration (as shown in FIG. 2A). Once the target area is reached, interventional device 11 can be advanced through the lumen 10 of the catheter 1 and out the distal port 13 and advanced in a distal direction. As can be seen, the catheter 1 can be configured such that advancing interventional device 11 in a distal direction out distal port 13 can deflect distal end 2 and thus result in ultrasound transducer array 7 being converted from side-looking to forward-looking. Thus, the physician can advance interventional device 11 into the field of view of ultrasound transducer array 7.

Deflectable can include 1) “actively deflectable” meaning that, in embodiments with an array, the array or catheter portion containing the array can be moved by remote application of force (e.g., electrical (e.g., wired or wireless), mechanical, hydraulic, pneumatic, magnetic, etc.), transmission of that force by various means including pull wires, hydraulic lines, air lines, magnetic coupling, or electrical conductors; and 2) “passively deflectable” meaning that, in embodiments with an array, the array or catheter portion containing the array when in the resting, unstrained condition, tends to be in alignment with the catheter longitudinal axis and may be moved by local forces imparted by the introduction of interventional device 11.

In certain embodiments, the ultrasound transducer array may be deflected up to 90 degrees from the longitudinal axis of the catheter, as shown in FIG. 2C. Moreover, the deflectable ultrasound transducer array 7 can be attached to the catheter by a hinge 9 as shown in FIG. 2D. In an embodiment, hinge 9 can be a spring-loaded hinged device. Such a spring-loaded hinge can be actuated from the proximal end of the catheter by any suitable means. In an embodiment, the spring-loaded hinge is a shape memory material actuated by withdrawal of an outer sheath.

With reference to FIGS. 2D and 2E, the catheter 1 can further comprise a steerable segment 8. FIG. 2E shows the steerable segment 8 deflected at an angle with respect to the catheter proximal to the steerable segment 8.

“Steerable” is defined as the ability to direct the orientation of a portion of a catheter distal to a steerable segment at an angle with respect to a portion of a catheter proximal to the steerable segment. “Steering” may include any known method of steering that may be utilized to direct the orientation of the portion of the catheter distal to the steerable segment at an angle with respect to the portion of the catheter proximal to the steerable segment, including methods that utilize more than one steerable segment. Such methods may include, without limitation, use of remote application of force (e.g., electrical (e.g., wired or wireless), mechanical, hydraulic, pneumatic, magnetic, etc.) with transmission of that force by various means including pull and/or push wires, hydraulic lines, air lines, magnetic coupling, or electrical conductors including without limitation transmission by manipulation of push and/or pull wires, filaments, tubes, and/or cables. In addition, the catheter body may be constructed to have segments with differing flexibility or compression properties from the other segments of the catheter body. In an embodiment having an inner tubular body and an outer tubular body, the outer tubular body may have one or more steerable segments with push/pull wires anchored to the distal end of the steerable segments and extending through one or more lumens of the outer tubular wall to attachment to the steering control in the handle. Steering of the outer tubular body may steer the inner tubular body as well. In a variation, the inner tubular body may be steerable and steering of the inner tubular body may steer the outer tubular body as well.

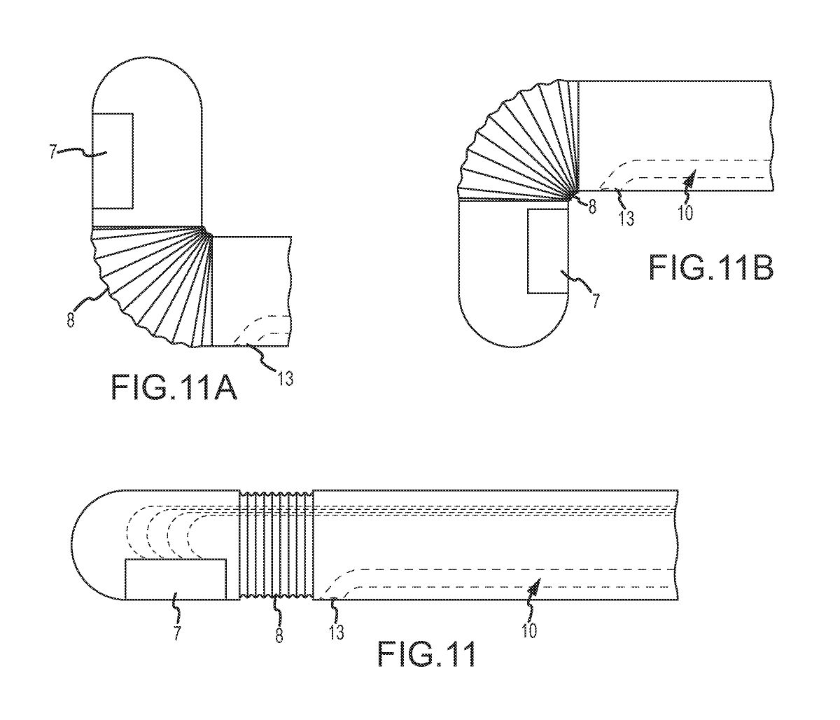

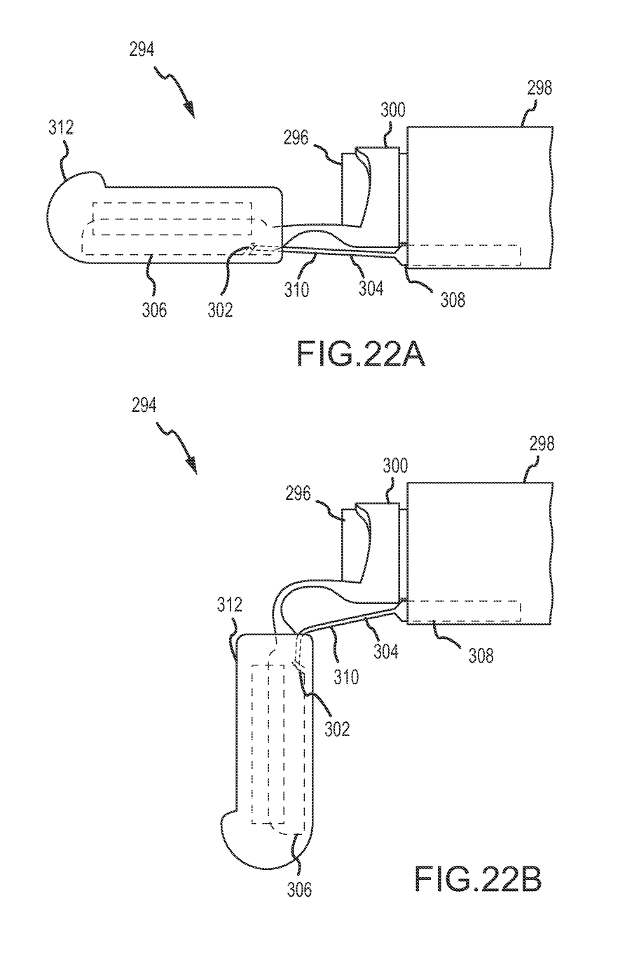

Steering with reference to FIG. 2E allows a clinician to guide or navigate a catheter to the appropriate anatomical position. Subsequently the clinician can utilize the actuation device as in reference to FIG. 22B to deflect the deflectable member to aim the imaging device at desired devices or anatomical features. Micro-steering as in reference to FIGS. 11A and 11B may be used to aim the imaging device at the anatomical features. Aiming may also be used to follow the trajectory of an interventional device as it is being advanced. In an embodiment, steering the catheter and then aiming the imaging device by deflection are operated independently.

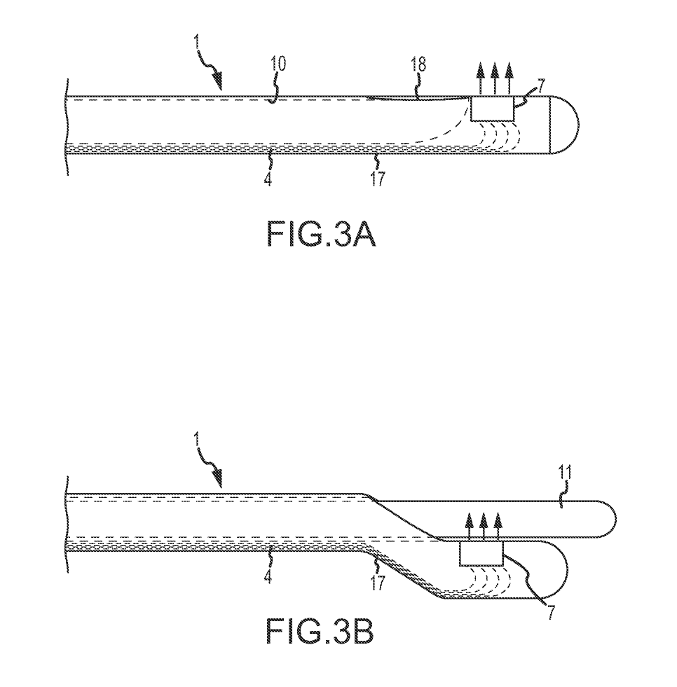

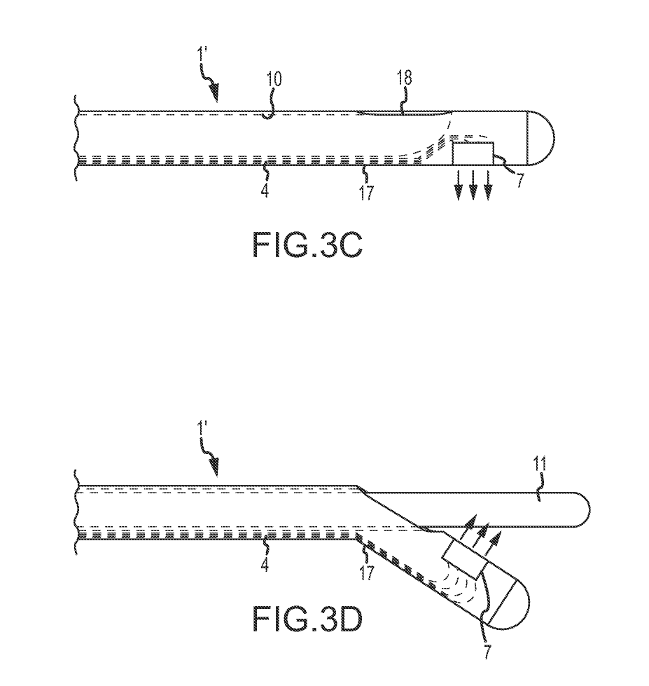

In a further embodiment, FIGS. 3A and 3B demonstrate a catheter 1 including an ultrasound transducer array 7 on a deflectable distal end 17 of the catheter 1. The catheter 1 comprises a proximal end (not shown) and a deflectable distal end 17. Ultrasound transducer array 7 is located at the deflectable distal end 17. Conductive wires 4 are attached to the ultrasound transducer array 7 and extend in a proximal direction to the proximal end of catheter 1. The catheter 1 also includes a generally centrally located lumen 10 that extends from the proximal end to the distal tip of the catheter. At distal end 17, the generally centrally located lumen 10 is essentially blocked or closed off by ultrasound transducer array 7. Finally, the catheter 1 also includes at least one longitudinally extending slit 18 that extends through a region proximal to the ultrasound transducer array 7.

As can be seen in FIG. 3B, once interventional device 11 is advanced distally through lumen 10, the interventional device 11 deflects deflectable distal end 17 and ultrasound transducer array 7 in a downward motion, thus opening lumen 10 so that interventional device 11 may be advanced distally past the ultrasound transducer array 7.

FIG. 3C illustrates a catheter 1′ that is an alternate configuration of the catheter 1 of FIGS. 3A and 3B. The catheter 1′ is configured the same as the catheter 1 with an exception that the ultrasound imaging array 7 is oriented such that it is operable to image a volume on a side of the catheter 1′ opposite from the longitudinally extending slit 18 (e.g., in a direction opposite from the ultrasound imaging array 7 of FIGS. 3A and 3B). This may be beneficial, for example, to maintain registration with a fixed anatomical landmark as the interventional device 11 is deployed.

FIG. 3D illustrates a catheter 1″ that is a variation of the catheter 1 of FIGS. 3A and 3B. The catheter 1″ is configured such that the ultrasound imaging array 7 pivots to a partially forward-looking position when the interventional device 11 is advanced through the longitudinally extending slit 18. The ultrasound imaging array 7 of catheter 1″ may be oriented as illustrated or it may be oriented to image in an opposite direction (similar to the ultrasound imaging array 7 of catheter 1′). In additional embodiments (not shown), a catheter similar to catheter 1 may include multiple imaging arrays (e.g., occupying the positions shown in both FIGS. 3A and 3C).

In various embodiments described herein, catheters may be provided having an ultrasound transducer array located near the distal end thereof. The catheter body may comprise a tube having a proximal end and a distal end. Moreover, the catheter may have at least one lumen extending from the proximal end to at least near the ultrasound transducer array. The catheter may comprise electrically conductive wires (e.g., a GORE™ Micro-Miniature Ribbon Cable) attached to the ultrasound transducer array and being imbedded in the catheter wall and helically extending from the ultrasound transducer array to the proximal end of the catheter.

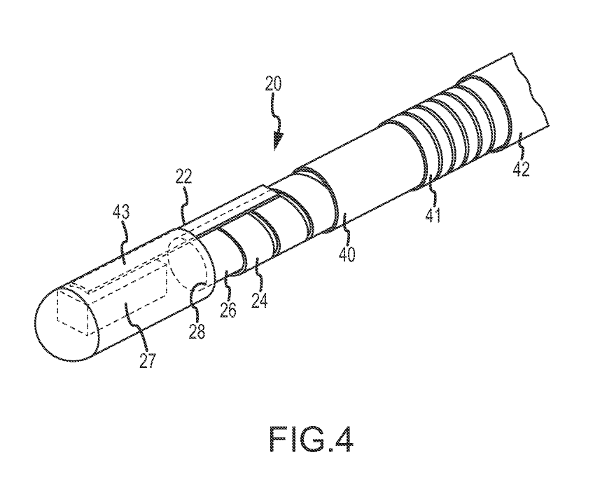

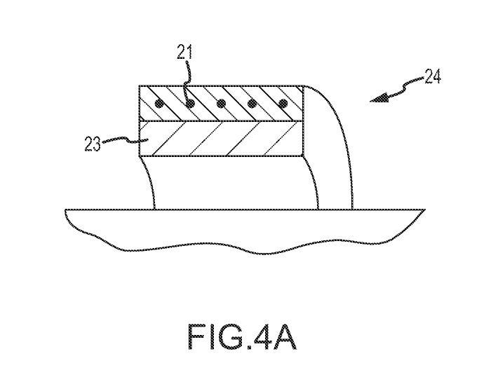

Such a catheter is depicted, for example, in FIGS. 4 and 4A. Specifically, FIGS. 4 and 4A demonstrate catheter 20 having a proximal end (not shown) and a distal end 22 with ultrasound transducer array 27 located at the distal end 22 of catheter 20. As can be seen, lumen 28 is defined by the inner surface of polymer tube 26, which can be formed from a suitable lubricious polymer (such as, for example, PEBAX® 72D, PEBAX® 63D, PEBAX® 55D, high density polyethylene, polytetrafluoroethylene, and expanded polytetrafluoroethylene, and combinations thereof) and extends from the proximal end to the distal end 22 near the ultrasound transducer array 27. The electrically conductive wires (e.g., GORE™ Micro-Miniature Ribbon Cable) 24 are helically wrapped about polymer tube 26 and extend from near the ultrasound transducer array 27 proximally to the proximal end. An example of a suitable microminiature flat cable is shown in FIG. 4A where microminiature flat cable 24 includes electrically conductive wires 21 and suitable ground, such as copper 23. A conductive circuit element 43 (such as a flexboard) is attached to ultrasound transducer array 27 and to the electrically conductive wires 24. A suitable polymer film layer 40 (such as a lubricious polymer and or shrink wrap polymer) can be located over electrically conductive wires 24 to act as an insulating layer between the electrically conductive wires 24 and a shielding layer 41. Shielding layer 41 may comprise any suitable conductor that can be helically wrapped over polymer film 40, for example, in the opposing direction of the electrically conductive wires 21. Finally, outer jacket 42 can be provided over shielding layer 41 and can be of any suitable material, such as a lubricious polymer. Suitable polymers include, for example, PEBAX® 70D, PEBAX® 55D, PEBAX®40D, and PEBAX® film 23D. The catheter depicted in FIGS. 4 and 4A can include the deflectable distal end and steerable segments discussed above.

The above catheter provides a means to electrically interface with an ultrasound probe at the distal end of a catheter while providing a working lumen to facilitate conveyance of a device and/or material (e.g., for delivery of interventional devices to the imaged area). The construction of the catheter utilizes the conductors both to power the array as well as to provide mechanical properties that enhance kink resistance and torqueability. The novel construction presented provides a means to package the conductors and necessary shielding in a thin wall, thus providing a sheath profile that is suited for interventional procedures, with an OD targeted at or below 14 French (Fr) and an ID targeted at above 8 Fr, thus facilitating delivery of typical ablation catheters, filter delivery systems, needles, and other common interventional devices designed for vascular and other procedures.

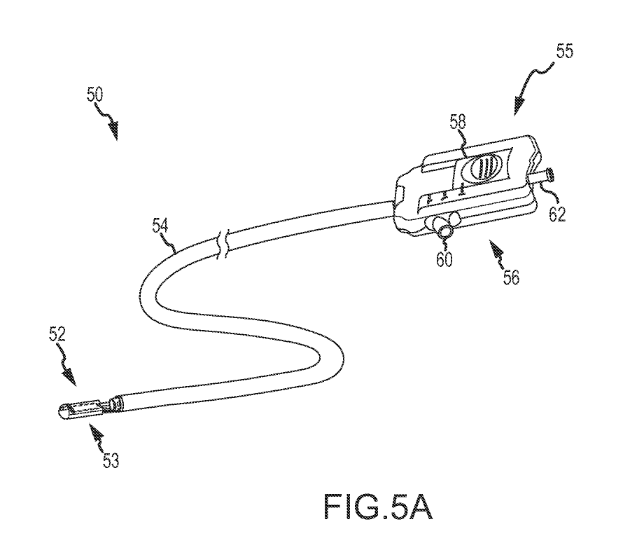

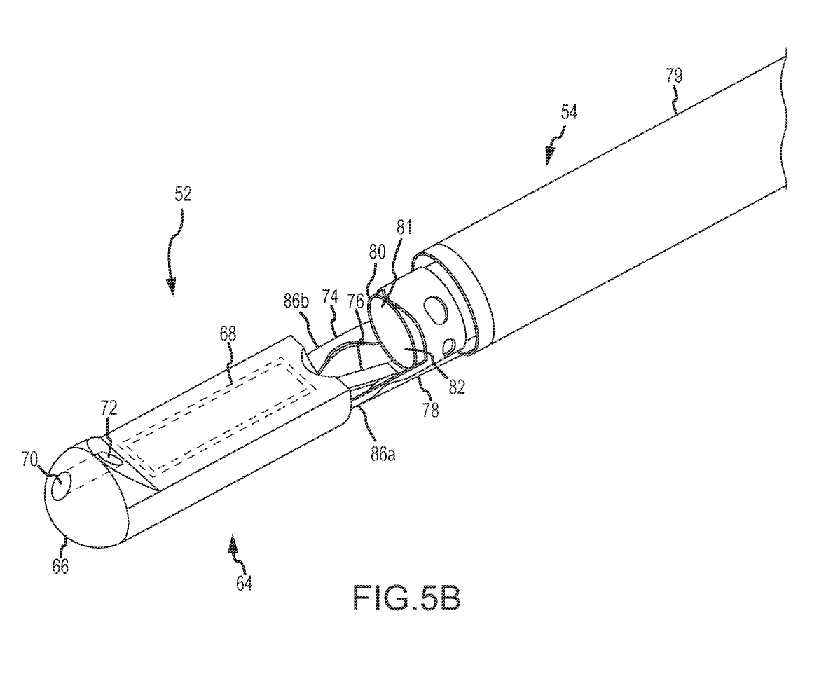

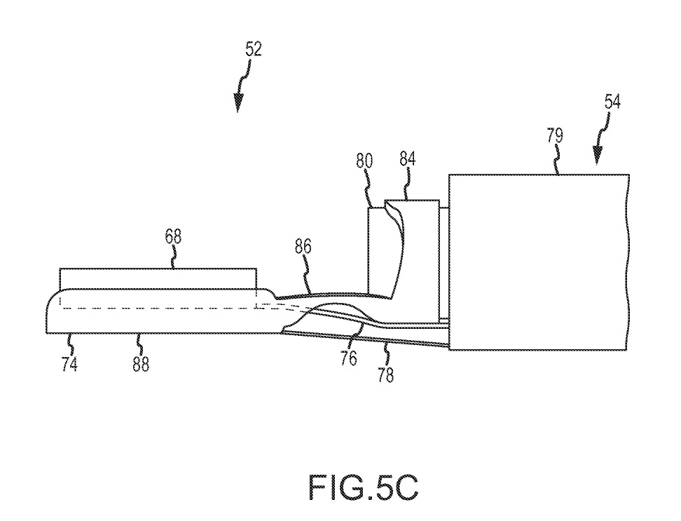

FIG. 5A shows an embodiment of a catheter 50 that includes a deflectable member 52 and a catheter body 54. The catheter body 54 may be flexible and capable of bending to follow the contours of a body vessel into which it is being inserted. The deflectable member 52 may be disposed at a distal end 53 of the catheter 50. The catheter 50 includes a handle 56 that may be disposed at a proximal end 55 of the catheter 50. During a procedure where the deflectable member 52 is inserted into the body of a patient, the handle 56 and a portion of the catheter body 54 remain outside of the body. The user (e.g., physician, technician, interventionalist) of the catheter 50 may control the position and various functions of the catheter 50. For example, the user may hold the handle 56 and manipulate a slide 58 to control a deflection of the deflectable member 52. In this regard, the deflectable member 52 may be selectively deflectable. The handle 56 and slide 58 may be configured such that the position of the slide 58 relative to the handle 56 may be maintained, thereby maintaining the selected deflection of the deflectable member 52. Such maintenance of position may at least partially be achieved by, for example, friction (e.g., friction between the slide 58 and a stationary portion of the handle 56), detents, and/or any other appropriate means. The catheter 50 may be removed from the body by pulling (e.g., pulling the handle 56).

Furthermore, the user may insert an interventional device (e.g., a diagnostic device and/or therapeutic device) through an interventional device inlet 62. The user may then feed the interventional device through the catheter 50 to move the interventional device to the distal end 53 of the catheter 50. Electrical interconnections between an image processor and the deflectable member may be routed through an electronics port 60 and through the catheter body 54 as described below.

FIGS. 5B through 5E show an embodiment of a catheter that includes a deflectable member 52 wherein the deflectable member 52 is deflectable by moving an inner tubular body 80 relative to an outer tubular body 79 of the catheter body 54. As shown in FIG. 5B, the illustrated deflectable member 52 includes a tip 64. The tip 64 may encase various components and members.

The tip 64 may have a cross section that corresponds to the cross section of the outer tubular body 79. For example, and as illustrated in FIG. 5B, the tip 64 may have a rounded distal end 66 that corresponds to the outer surface of the outer tubular body 79. The portion of the tip 64 that houses the ultrasound transducer array 68 may be shaped to at least partially correspond (e.g., along the lower outer surface of the tip 64 as viewed in FIG. 5B) to the outer surface of the outer tubular body 79. At least a portion of the tip 64 may be shaped to promote transport through internal structures of the patient such as the vasculature. In this regard, the rounded distal end 66 that may aid in moving the deflectable member 52 through the vasculature. Other appropriate end shapes may be used for the shape of the distal end 66 of the tip 64.

In an embodiment, such as the one illustrated in FIGS. 5B through 5D, the tip 64 may hold an ultrasound transducer array 68. As will be appreciated, as illustrated in FIG. 5B, the ultrasound transducer array 68 may be side-looking when the deflectable member 52 is aligned with the outer tubular body 79. The field of view of the ultrasound transducer array 68 may be located perpendicular to the flat upper face (as oriented in FIG. 5B) of the ultrasound transducer array 68. As illustrated in FIG. 5B, the field of view of the ultrasound transducer array 68 may be unobstructed by the outer tubular body 79 when the ultrasound transducer array 68 is side-looking. In this regard, the ultrasound transducer array 68 may be operable to image during catheter body 54 positioning, thereby enabling imaging of anatomical landmarks to aid in positioning the distal end of a lumen 82. The ultrasound transducer array 68 may have an aperture length. The aperture length may be greater than a maximum cross dimension of the outer tubular body 79. At least a portion of the deflectable member 52 may be permanently positioned distal to the distal end of the outer tubular body 79. In an embodiment, the entirety of the deflectable member 52 may be permanently positioned distal to the distal end of the outer tubular body 79. In such an embodiment, the deflectable member may be incapable of being positioned within the outer tubular body 79.

The tip 64 may further include a feature to enable the catheter to track a guidewire. For example, as illustrated in FIG. 5B, the tip 64 may include a distal guidewire aperture 70 functionally connected to a proximal guidewire aperture 72. In this regard, the catheter may be operable to travel along the length of a guidewire threaded through the distal 70 and proximal 72 guidewire apertures.

As noted, the deflectable member 52 may be deflectable relative to the outer tubular body 79. In this regard, the deflectable member 52 may be interconnected to one or more members to control the motion of the deflectable member 52 as it is being deflected. A tether 78 may interconnect the deflectable member 52 to the catheter body 54. The tether 78 may be anchored to the deflectable member 52 on one end and to the catheter body 54 on the other end. The tether 78 may be configured as a tensile member operable to prevent the anchor points from moving a distance away from each other greater than the length of the tether 78. In this regard, through the tether 78, the deflectable member 52 may be restrainably interconnected to the outer tubular body 79.

An inner tubular body 80 may be disposed within the outer tubular body 79. The inner tubular body 80 may include the lumen 82 passing through the length of the inner tubular body 80. The inner tubular body 80 may be movable relative to the outer tubular body 79. This movement may be actuated by movement of the slide 58 of FIG. 5A. A support 74 may interconnect the deflectable member 52 to the inner tubular body 80. The support 74 may be structurally separate from the inner tubular body 80 and the outer tubular body 79. A flexboard 76 may contain electrical interconnections operable to electrically connect the ultrasound transducer array 68 to an electrical interconnection member 104 (shown in FIG. 5E) disposed within the outer tubular body 79. The exposed portion of flexboard 76 between the tip 64 and the outer tubular body 79 may be encapsulated to isolate it from possible contact with fluids (e.g., blood) when the deflectable member 52 is disposed within a patient. In this regard, the flexboard 76 may be encapsulated with an adhesive, a film wrap, or any appropriate component operable to isolate the electrical conductors of the flexboard 76 from the surrounding environment. In an embodiment, the tether 78 may be wrapped around the portion of the flexboard 76 between the tip 64 and the outer tubular body 79.

Deflection of the deflectable member 52 will now be discussed with reference to FIGS. 5C and 5D. FIGS. 5C and 5D illustrate the deflectable member 52 with the portion of the tip 64 surrounding the ultrasound image array 68 and support 74 removed. As illustrated in FIG. 5C, the support 74 may include a tubular body interface portion 84 operable to fix the support 74 to the inner tubular body 80. The tubular body interface portion 84 may be fixed to the inner tubular body 80 in any appropriate manner. For example, the tubular body interface portion 84 may be secured to the inner tubular body 80 with an external shrink wrap. In such a configuration, the tubular body interface portion 84 may be placed over the inner tubular body 80 and then a shrink-wrap member may be placed over the tubular body interface portion 84. Heat may then be applied causing the shrink wrap material to shrink and fix the tubular body interface portion 84 to the inner tubular body 80. An additional wrap may then be applied over the shrink wrap to further fix the tubular body interface portion 84 to the inner tubular body 80. In another example, the tubular body interface portion 84 may be secured to the inner tubular body 80 with an adhesive, a weld, fasteners, or any combination thereof. In another example, the tubular body interface portion 84 may be secured to the inner tubular body 80 as part of the assembly process used to build the inner tubular body 80. For example, the inner tubular body 80 may be partially assembled, the tubular body interface portion 84 may be positioned around the partially assembled inner tubular body 80, and then the inner tubular body 80 may be completed, thus capturing the tubular body interface portion 84 within a portion of the inner tubular body 80.

The support 74 may comprise, for example, a shape memory material (e.g., a shape memory alloy such as Nitinol). The support 74 may further include a hinge portion 86. The hinge portion 86 may comprise one or more members interconnecting the tubular body interface portion 84 with a cradle portion 88. The hinge portion 86, as illustrated in FIGS. 5B through 5C, may comprise two members. The cradle portion 88 may support the ultrasound transducer array 68. The support 74, including the hinge portion 86, may possess a column strength adequate to keep the deflectable member 52 substantially aligned with the outer tubular body 79 in the absence of any advancement of the inner tubular body 80 relative to the outer tubular body 79. In this regard, the deflectable member 52 may be operable to remain substantially aligned with the outer tubular body 79 when the outer tubular body 79 is being inserted into and guided through the patient.

The hinge portion 86 may be shaped such that upon application of an actuation force, the hinge portion 86 elastically deforms along a predetermined path about a deflection axis 92. The predetermined path may be such that the tip 64 and the hinge portion 86 each are moved to a position where they do not interfere with an interventional device emerging from the distal end of the lumen 82. An imaging field of view of the ultrasound transducer array 68 may be substantially maintained in a position relative to the outer tubular body 79 when the interventional device is advanced through the port 81 at the distal end of the lumen 82 and into the field of view. As illustrated in FIGS. 5B through 5D, the hinge portion may comprise two generally parallel sections 86a and 86b, where the ends of each of the generally parallel sections 86a and 86b (e.g., where the hinge portion 86 meets the cradle portion 88 and where the hinge portion 86 meets the tubular body interface portion 84) may be generally shaped to coincide with a cylinder oriented along a center axis 91 of the inner tubular body 80. A central portion of each of the generally parallel sections 86a and 86b may be twisted toward the center axis 91 of the outer tubular body 79 such that the central portions are generally aligned with the deflection axis 92. The hinge portion 86 is disposed such that it is disposed about less than the entirety of the circumference of the inner tubular body 80.

To deflect the deflectable member 52 relative to the outer tubular body 79, the inner tubular body 80 may be moved relative to the outer tubular body 79. Such relative movement is illustrated in FIG. 5D. As shown in FIG. 5D, movement of the inner tubular body 80 in an actuation direction 90 (e.g., in the direction of the ultrasound transducer array 68 when the deflectable member 52 is aligned with the outer tubular body 79) may impart a force on the support 74 in the actuation direction 90. However, since the cradle portion 88 is restrainably connected to the outer tubular body 79 by the tether 78, the cradle portion 88 is prevented from moving substantially in the actuation direction 90. In this regard, the movement of the inner tubular body 80 in the actuation direction 90 may result in the cradle portion 88 pivoting about its interface with the tether 78 and also in the hinge portion 86 bending as illustrated in FIG. 5D. Thus the movement of the inner tubular body 80 in the actuation direction 90 may result in the cradle portion 88 (and the ultrasound transducer array 68 attached to the cradle portion 80) rotating 90 degrees as illustrated in FIG. 5D. Accordingly, movement of the inner tubular body 80 may cause a controlled deflection of the deflectable member 52. As illustrated, the deflectable member 52 may be selectively deflectable away from the center axis 91 of the outer tubular body 79.

In an exemplary embodiment, a movement of the inner tubular body 80 of about 0.1 cm may result in the deflectable member 52 deflecting through an arc of about 9 degrees. In this regard, movement of the inner tubular body 80 of about 1 cm may result in the deflectable member 52 deflecting about 90 degrees. Thusly, the deflectable member 52 may be selectively deflected from a side-looking position to a forward-looking position. Intermediate positions of the deflectable member 52 may be achieved by moving the inner tubular body 80 a predeterminable distance. For example, in the current exemplary embodiment, the deflectable member 52 may be deflected 45 degrees from the side-looking position by moving the inner tubular body 80 about 0.5 cm relative to the outer tubular body 79 in the actuation direction 90. Other appropriate member geometries may be incorporated to produce other relationships between inner tubular body 80 and deflectable member 52 deflection. Moreover, deflections of greater than 90 degrees may be obtained (e.g., such that the deflectable member 52 is at least partially side-looking to a side of the catheter body 54 opposite from that illustrated in FIG. 5C). Moreover, an embodiment of the catheter 50 may be configured such that a predeterminable maximum deflection of the deflectable member 52 may be achieved. For example, the handle 56 may be configured to limit the movement of the slide 58 such that the full range of movement of the slide 58 corresponds to a 45 degree deflection (or any other appropriate deflection) of the deflectable member 52.

The slide 58 and handle 56 may be configured such that substantially any relative motion of the slide 58 to the handle 56 results in a deflection of the deflectable member 52. In this regard, there may be substantially no dead zone of the slide 58 where slide 58 movement does not result in deflection of the deflectable member 52. Furthermore, the relationship between movement of the slide 58 (e.g., relative to the handle 56) and the amount of corresponding deflection of the deflectable member 52 may be substantially linear.

When the deflectable member 52 is deflected from the position illustrated in FIG. 5C so that no part of the tip 64 occupies a cylinder the same diameter as and extending distally from the port 81, an interventional device may be advanced through the port 81 without contacting the tip 64. As such, the imaging field of view of the ultrasound transducer array 68 may be maintained in a fixed registration relative to the catheter body 54 while the interventional device is being advanced into the catheter body 54, through the port 81, and into the imaging field of view of the ultrasound transducer array 68.

When in a forward-looking position, the field of view of the ultrasound transducer array 68 may encompass an area in which an interventional device may be inserted through the lumen 82. In this regard, the ultrasound transducer array 68 may be operable to aid in the positioning and operation of the interventional device.

The deflectable member 52 may deflect about the deflection axis 92 (deflection axis 92 is aligned with the view of FIG. 5D and therefore is represented by a point). The deflection axis 92 may be defined as a point fixed relative to the tubular body interface portion 84 about which the cradle portion 88 rotates. As illustrated in FIG. 5D, the deflection axis 92 may be offset from the center axis 91 of the outer tubular body 79. For any given deflection of the deflectable member 52, a displacement arc 93 may be defined as the minimum constant-radius arc that is tangent to a face of the deflectable member 52 and tangent to a straight line collinear with the center axis 91 of the catheter at the most distal point of the catheter. In an embodiment of the catheter 50, the ratio of a maximum cross-dimension of the distal end of the outer tubular body 79 to the radius of the displacement arc 93 upon a deflection of 90 degrees from the central axis 91 may be at least about 1.

The deflectable member 52 may deflect about the deflection axis 92 such that the ultrasound transducer array 68 is positioned proximate to the port 81. Such positioning, in conjunction with a small displacement arc 93, reduces the distance an interventional device must travel between emerging from the port 81 and entering the field of view of the ultrasound transducer array 68. For example, upon deflection of 90 degrees as shown in FIG. 5D, the ultrasound transducer array 68 may be positioned such that the acoustical face of the ultrasound transducer array 68 is a distance from the port 81 (as measured along the central axis 91) that is less than the maximum cross dimension of the distal end of the outer tubular body 79.

As illustrated in FIGS. 5C and 5D, the flexboard 76 may remain interconnected to the catheter body 54 and the deflectable member 52 independent of the deflection of the deflectable member 52.

FIG. 5E illustrates an embodiment of the catheter body 54. The catheter body 54 as illustrated comprises the inner tubular body 80 and the outer tubular body 79. In the illustrated embodiment, the outer tubular body 79 comprises all of the components illustrated in FIG. 5E except for the inner tubular body 80. For the illustration of FIG. 5E, portions of various layers have been removed to reveal the construction of the catheter body 54. The outer tubular body 79 may include an outer covering 94. The outer covering 94 may, for example, be a high voltage breakdown material. In an exemplary configuration the outer covering 94 may comprise a substantially non-porous composite film including expanded polytetrafluoroethylene (ePTFE) with a thermal adhesive layer of ethylene fluoroethylene perfluoride on one side. The exemplary configuration may have a width of about 25 mm, a thickness of about 0.0025 mm, an isopropyl alcohol bubble point of greater than about 0.6 MPa, and a tensile strength of about 309 MPa in the length direction (e.g., the strongest direction). The outer covering 94 may be lubricious to aid in the passage of the outer tubular body 79 through the patient. The outer covering 94 may provide a high voltage breakdown (e.g., the outer covering 94 may have a withstand voltage of at least about 2,500 volts AC).

In an exemplary arrangement, the outer covering 94 may include a plurality of helically wound films. A first portion of the plurality of films may be wound in a first direction, and a second portion of the films may be wound in a second direction that is opposite from the first direction. Where each film of the plurality of films has a longitudinal modulus of at least about 1,000,000 psi (6,895 MPa) and a transverse modulus of at least about 20,000 psi (137.9 MPa), each film of the plurality of films may be wound about a central axis of the tubular body at an angle of less than about 20 degrees relative to the central axis of the tubular body 79.

Within the outer covering 94 may be disposed an outer low-dielectric constant layer 96. The outer low-dielectric constant layer 96 may reduce capacitance between the electrical interconnection member 104 and materials (e.g., blood) outside of the outer covering 94. The outer low-dielectric constant layer 96 may have a dielectric constant of less than about 2.2. In an embodiment, the outer low-dielectric constant layer 96 may be about 0.07-0.15 mm thick. In an embodiment, the outer low-dielectric constant layer 96 may comprise a porous material, such as ePTFE. The voids in the porous material may be filled with a low-dielectric material such as air.

In an exemplary arrangement, the combinative properties of the outer covering 94 and the outer low-dielectric constant layer 96 may include a maximum thickness of 0.005 inches (0.13 mm) and an elastic modulus of 34,500 psi (237.9 MPa). In this regard, the outer covering 94 and the outer low-dielectric constant layer 96 may be viewed as a single composite layer including two sub-layers (the outer covering 94 and the outer low-dielectric constant layer 96).

Moving toward the center of the outer tubular body 79, the next layer may be first tie layer 97. The first tie layer 97 may comprise a film material that may have a melt temperature that is lower then other components of the outer tubular body 79. During fabrication of the outer tubular body 79, the first tie layer 97 may be selectively melted to yield an interconnected structure. For example, selectively melting the first tie layer 97 may serve to secure the outer low-dielectric constant layer 96, the first tie layer 97, and a shield layer 98 (discussed below) to each other.

Moving toward the center of the outer tubular body 79, the next layer may be the shield layer 98. The shield layer 98 may be used to reduce electrical emissions from the outer tubular body 79. The shield layer 98 may be used to shield components internal to the shield layer 98 (e.g., the electrical interconnection member 104) from external electrical noise. The shield layer 98 may be in the form of a double served wire shield or braid. In an exemplary embodiment, the shield layer 98 may be about 0.05-0.08 mm thick. Moving toward the center of the outer tubular body 79, the next layer may be a second tie layer 100. The second tie layer 100 may comprise a film material that may have a melt temperature that is lower then other components of the outer tubular body 79. During fabrication of the outer tubular body 79, the second tie layer 100 may be selectively melted to yield an interconnected structure.

Interior to the second tie layer 100 may be the electrical interconnection member 104. The electrical interconnection member 104 may comprise a plurality of conductors arranged in a side-by-side fashion with an insulative (e.g., non-conductive) material between the conductors. The electrical interconnection member 104 may comprise one or more microminiature flat cables. The electrical interconnection member 104 may contain any appropriate number of conductors arranged in a side-by-side fashion. By way of example, the electrical interconnection member 104 may contain 32 or 64 conductors arranged in a side-by-side fashion. The electrical interconnection member 104 may be helically disposed within the outer tubular body 79. In this regard, the electrical interconnection member 104 may be helically disposed within the wall of the outer tubular body 79. The electrical interconnection member 104 may be helically disposed such that no part of the electrical interconnection member 104 overlies itself. The electrical interconnection member 104 may extend from the proximal end 55 of the catheter 50 to the distal end 53 of the outer tubular body 79. In an embodiment, the electrical interconnection member 104 may be disposed parallel to and along the central axis of the outer tubular body 79.

As illustrated in FIG. 5E, there may be a gap of width Y between the coils of the helically wound electrical interconnection member 104. In addition, the electrical interconnection member 104 may have a width of X as illustrated in FIG. 5E. The electrical interconnection member 104 may be helically disposed such that the ratio of the width X to the width Y is greater than 1. In such an arrangement, the helically disposed electrical interconnection member 104 may provide significant mechanical strength and flexural properties to the outer tubular body 79. This may, in certain embodiments, obviate or reduce the need for a separate reinforcing layer within the outer tubular body 79. Moreover, the gap Y may vary along the length of the outer tubular body 79 (e.g., continuously or in one or more discrete steps). For example, it may be beneficial to have a greater stiffness to the outer tubular body 79 toward the proximal end of the outer tubular body 79. Accordingly, the gap Y may be made smaller toward the proximal end of the outer tubular body 79.

An inner tie layer 102 may be disposed interior to the electrical interconnection member 104. The inner tie layer 102 may be configured similar to and serve a similar function as the second tie layer 100. The inner tie layer 102 may have a melting point of, for example, 160 degrees Celsius. Moving toward the center of the outer tubular body 79, the next layer may be an inner low-dielectric constant layer 106. The inner low-dielectric constant layer 106 may be configured similar to and serve a similar function as the outer low-dielectric constant layer 96.

The inner low-dielectric constant layer 106 may be operable to reduce capacitance between the electrical interconnection member 104 and materials (e.g., blood, interventional device) within the outer tubular body 79. Moving toward the center of the outer tubular body 79, the next layer may be an inner covering 108. The inner covering 108 may be configured similar to and serve a similar function as the outer covering 94. The inner covering 108 and the outer covering 94 may have a combined thickness of at most about 0.002 inches (0.05 mm). Moreover, the inner covering 108 and outer covering 94 may have a combined elastic modulus of at least about 345,000 psi (2,379 MPa). Combined, the inner covering 108 and the outer covering 94 may provide an elongation resistance such that a tensile load, applied to the inner covering 108 and the outer covering 94, of about 3 lbf (13 N) results in no more than a 1 percent elongation of the tubular body 79. In an arrangement, the tubular body 79 may provide an elongation resistance such that a tensile load, applied to the tubular body 79, of about 3 lbf (13 N) results in no more than a 1 percent elongation of the tubular body 79, and in such an arrangement at least about 80 percent of the elongation resistance may be provided by the inner covering 108 and outer covering 94.

The inner covering 108 and outer covering 94 may exhibit a substantially uniform tensile profile about their circumferences and along the length of the tubular body 79 when a tensile load is applied to the tubular body 79. Such a uniform response to an applied tensile load may, inter alia, help to reduce undesirable directional biasing of the catheter body 54 during positioning (e.g., insertion into a patient) and use (e.g., while deflecting the deflectable member 52).

As with the outer covering 94 and the outer low-dielectric constant layer 96, the inner low-dielectric constant layer 106 and the inner covering 108 may be viewed as sub-layers to a single composite layer.

The tie layers (first tie layer 97, second tie layer 100, and inner tie layer 102) may each have substantially the same melting point. In this regard, during construction, the catheter body 54 may be subjected to an elevated temperature that may melt each of the tie layers simultaneously and fix various layers of the catheter body 54 relative to each other. Alternatively, the tie layers may have different melting points allowing selective melting of one or two of the tie layers while leaving the other tie layer or tie layers unmelted. Accordingly, embodiments of catheter bodies 54 may comprise zero, one, two, three, or more tie layers that have been melted to secure various layers of the catheter body 54 to other layers of the catheter body 54.

The aforementioned layers (from the outer covering 94 through the inner covering 108) may each be fixed relative to each other. Together these layers may form the outer tubular body 79. Interior to these layers and movable relative to these layers may be the inner tubular body 80. The inner tubular body 80 may be disposed such that there is an amount of clearance between the outside surface of the inner tubular body 80 and the interior surface of the inner covering 108. The inner tubular body 80 may be a braid reinforced polyether block amide (e.g., the polyether block amide may comprise a PEBAX® material available from Arkema Inc., Philadelphia, Pa.) tube. The inner tubular body 80 may be reinforced with a braided or coiled reinforcing member. The inner tubular body 80 may possess a column strength adequate that it may be capable of translating a lateral motion of the slide 58 along the length of the inner tubular body 80 such that the deflectable member 52 may be actuated by the relative movement of the inner tubular body 80 where it interfaces with the support 74 at the tubular body interface portion 84. The inner tubular body 80 may also be operable to maintain the shape of the lumen 82 passing through the length of the inner tubular body 80 during deflection of the deflectable member 52. Accordingly, a user of the catheter 50 may be capable of selecting and controlling the amount of deflection of the deflectable member 52 through manipulation of the handle 56. The lumen 82 may have a center axis aligned with the center axis 91 of the outer tubular body 79.

To assist in reducing actuation forces (e.g., the force to move the inner tubular body 80 relative to the outer tubular body 79), the inner surface of the inner covering 108, the outer surface of the inner tubular body 80, or both may include a friction reduction layer. The friction reduction layer may be in the form of one or more lubricious coatings and/or additional layers.

In a variation of the embodiment illustrated in FIG. 5E, the inner tubular body 80 may be replaced with an external tubular body that is disposed outside of the outer covering 94. In such an embodiment, the components of the outer tubular body 79 (from the outer covering 94 to the inner covering 108) may remain substantially unchanged from as illustrated in FIG. 5E (the diameters of the components may be reduced slightly to maintain similar overall inner and outer diameters of the catheter body 54). The external tubular body may be fitted outside of the outer covering 94 and may be movable relative to the outer covering 94. Such relative movement may facilitate deflection of the deflectable member 52 in a manner similar to as described with reference to FIGS. 5A through 5D. In such an embodiment, the electrical interconnection member 104 would be a part of the outer tubular body 79 that would be located inside of the external tubular body. The external tubular body may be constructed similarly to the inner tubular body 80 described above.

In an exemplary embodiment, the catheter body 54 may have a capacitance of less than 2,000 picofarads. In an embodiment, the catheter body 54 may have a capacitance of about 1,600 picofarads. In the above-described embodiment of FIG. 5E, the outer covering 94 and outer low-dielectric constant layer 96 may, in combination, have a withstand voltage of at least about 2,500 volts AC. Similarly, the inner covering 108 and inner low-dielectric constant layer 106 may, in combination, have a withstand voltage of at least about 2,500 volts AC. Other embodiments may achieve different withstand voltages by, for example, varying the thicknesses of the covering and/or low-dielectric constant layers. In an exemplary embodiment, the outer diameter of the outer tubular body 79 may, for example, be about 12.25 Fr. The inner diameter of the inner tubular body may, for example, be about 8.4 Fr.

The catheter body 54 may have a kink diameter (the diameter of bend in the catheter body 54 below which the catheter body 54 will kink) that is less than ten times the diameter of the catheter body 54. Such a configuration is appropriate for anatomical placement of the catheter body 54.

As used herein, the term “outer tubular body” refers to the outermost layer of a catheter body and all layers of that catheter body disposed to move with the outermost layer. For example, in the catheter body 54 as illustrated in FIG. 5E, the outer tubular body 79 includes all illustrated layers of the catheter body 54 except the inner tubular body 80. Generally, in embodiments where there is no inner tubular body present, the outer tubular body may coincide with the catheter body.

The various layers of the outer tubular body 79 described with reference to FIG. 5E may, where appropriate, be fabricated by helically winding strips of material along the length of the catheter body 54. In an embodiment, selected layers may be wrapped in a direction opposite of other layers. By selectively winding layers in appropriate directions, some physical properties of the catheter body 54 (e.g., stiffness) may be selectively altered.

FIG. 5F shows an embodiment of an electrical interconnection between the helically disposed electrical interconnection member 104 and the flexboard 76 (a flexible/bendable electrical member). For explanatory purposes, all the parts of the catheter body 54 except the electrical interconnection member 104 and the flexboard 76 are not illustrated in FIG. 5F. The flexboard 76 may have a curved section 109. The curved section 109 may be curved to correspond with the curvature of the outer tubular body 79. The curved section 109 of the flexboard 76 may be disposed within the outer tubular body 79 at the end of the outer tubular body 79 proximate to the deflectable member 52 in the same position with respect to the layers of the outer tubular body 79 as the electrical interconnection member 104. Accordingly, the curved section 109 of the flexboard 76 may come into contact with the electrical interconnection member 104. In this regard, the distal end of the electrical interconnection member 104 may interconnect to the flexboard 76 in an interconnect region 110.

Within the interconnect region 110, the electrically conductive portions (e.g., wires) of the electrical interconnection member 104 may be interconnected to electrically conductive portions (e.g., traces, conductive paths) of the flexboard 76. This electrical interconnection may be achieved by peeling back or removing some of the insulative material of the electrical interconnection member 104 and contacting the exposed electrically conductive portions to corresponding exposed electrically conductive portions on the flexboard 76. The end of the electrical interconnection member 104 and the exposed conductive portions of the electrical interconnection member 104 may be disposed at an angle relative to the width of the electrical interconnection member 104. In this regard, the pitch (e.g., the distance between the centers of the electrically conductive portions) between the exposed electrically conductive portions of the flexboard 76 may be greater than the pitch (as measured across the width) of the electrical interconnection member 104, while maintaining an electrical interconnection between each conductor of both the electrical interconnection member 104 and the flexboard 76.

As illustrated in FIG. 5F, the flexboard 76 may comprise a flexing or bending region 112 that has a width narrower than the width of the electrical interconnection member 104. As will be appreciated, the width of each individual electrically conductive path through the flexing region 112 may be smaller than the width of each electrically conductive member within the electrical interconnection member 104. Furthermore, the pitch between each electrically conductive member within the flexing region 112 may be smaller than the pitch of the electrical interconnection member 104.

The flexing region 112 may be interconnected to an array interface region 114 of the flexboard 76 through which the electrically conductive paths of the electrical interconnection member 104 and the flexboard 76 may be electrically interconnected to individual transducers of the ultrasound transducer array 68.

As illustrated in FIGS. 5C and 5D, the flexing region 112 of the flexboard 76 may be operable to flex during deflection of the deflectable member 52. In this regard, the flexing region 112 may be bendable in response to deflection of the deflectable member 52. The individual conductors of the electrical interconnection member 104 may remain in electrical communication with the individual transducers of the ultrasound transducer array 68 during deflection of the deflectable member 52.

In an embodiment, the electrical interconnection member 104 may comprises two or more separate sets of conductors (e.g., two or more microminiature flat cables). In such an embodiment, each of the separate sets of conductors may be interconnected to the flexboard 76 in a manner similar to as illustrated in FIG. 5F. Furthermore, the electrical interconnection member 104 (either a unitary electrical interconnection member 104 as illustrated in FIG. 5F or an electrical interconnection member 104 comprising a plurality of generally parallel distinct cables) may comprise members that extend from the distal end 53 to the proximal end 55 of the catheter body 54 or the electrical interconnection member 104 may comprise a plurality of discrete, serially interconnected members that together extend from the distal end 53 to the proximal end 55 of the catheter body 54. In an embodiment, the flexboard 76 may include the electrical interconnection member 104. In such an embodiment, the flexboard 76 may have a helically wrapped portion extending from the distal end 53 to the proximal end 55 of the catheter body 54. In such an embodiment, no electrical conductor interconnections (e.g., between the flexboard 76 and a microminiature flat cable) may be required between the array interface region 114 and the proximal end of the catheter body 54.

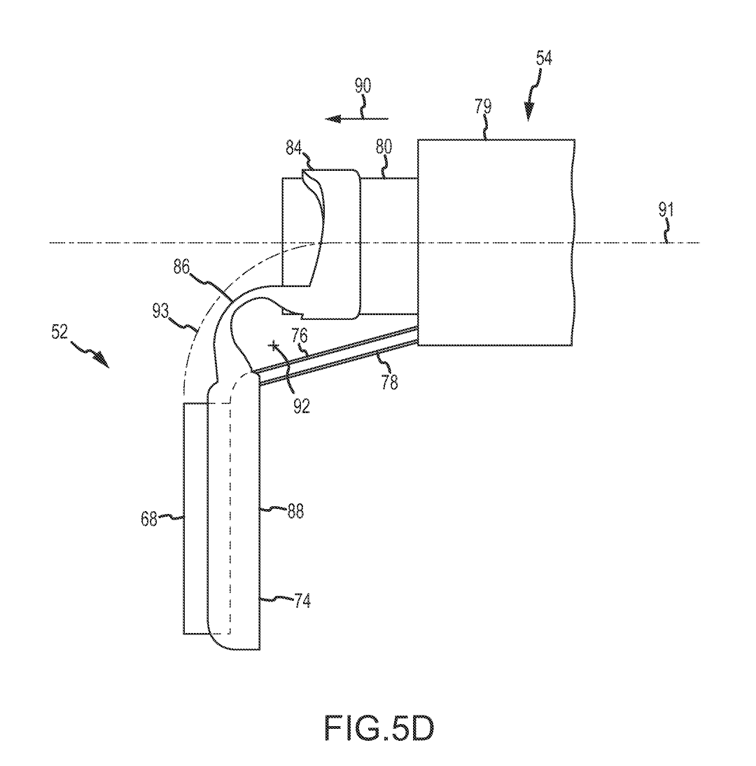

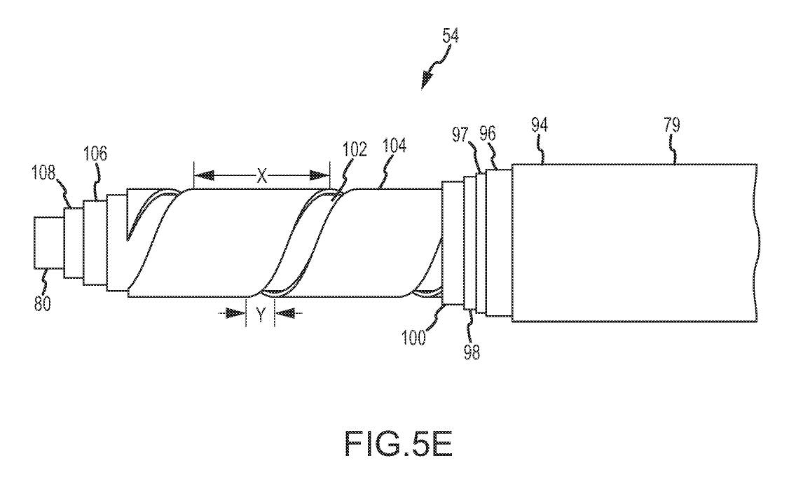

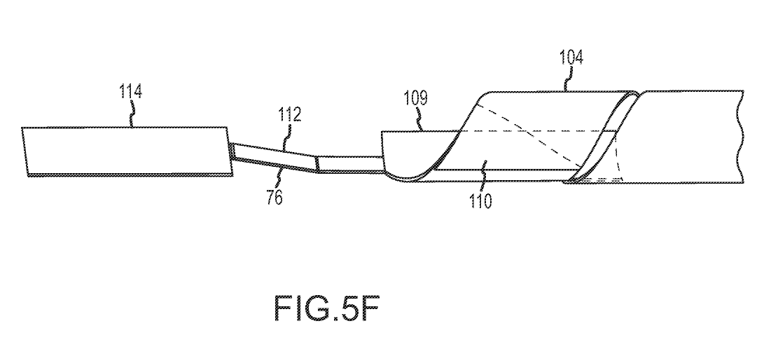

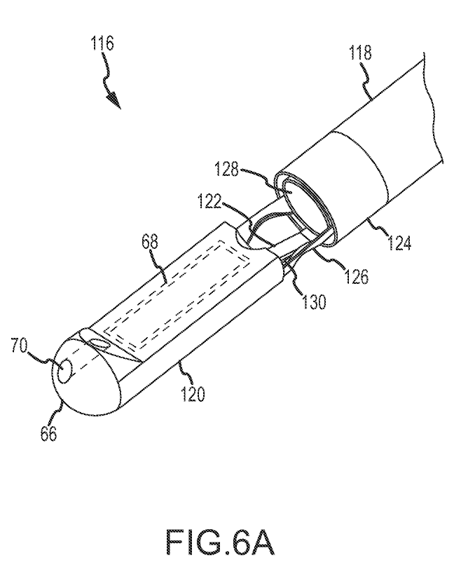

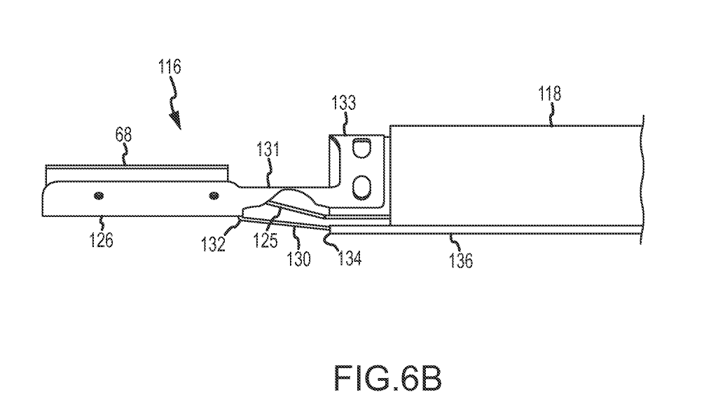

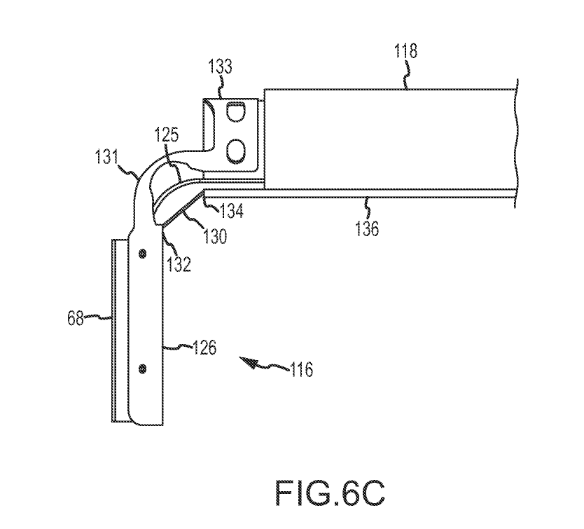

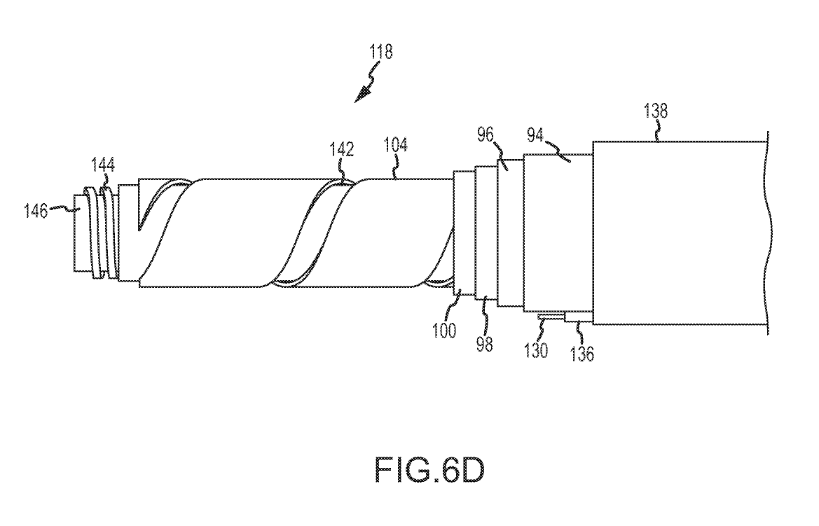

FIGS. 6A through 6D show an embodiment of a catheter that includes a deflectable member 116 wherein the deflectable member 116 is deflectable by moving an elongate member relative to an outer tubular body 118. It will be appreciated that the embodiment illustrated in FIGS. 6A through 6D does not include an inner tubular body and the outer tubular body 118 may also be characterized as a catheter body.

The deflectable member 116 may be selectively deflectable. As shown in FIG. 6A, the illustrated deflectable member 116 includes a tip 120. The tip 120 may include the ultrasound transducer array 68 and may include a rounded distal end 66 and guidewire aperture 70 similar to the tip 64 described with reference to FIG. 5B. As with the tip 64 of FIG. 5B, the ultrasound transducer array 68 may be side-looking when the deflectable member 116 is aligned with the outer tubular body 118. In this regard, the ultrasound transducer array 68 may be operable to image anatomical landmarks during catheter insertion to aid in guiding and/or positioning the outer tubular body 118.

The outer tubular body 118 may include a lumen 128 operable to allow an interventional device to pass therethrough. At least a portion of the deflectable member 116 may be permanently positioned distal to the distal end of with the outer tubular body 118. In an embodiment, the entirety of the deflectable member 116 may be permanently positioned distal to the distal end of the outer tubular body 118.

The deflectable member 116 may be deflectable relative to the outer tubular body 118. In this regard, the deflectable member 116 may be interconnected to one or more elongate members to control the motion of the deflectable member 116 as it is being deflected. The elongate member may take the form of a pull wire 130. The pull wire 130 may be a round wire. Alternatively, for example, the pull wire 130 may be rectangular in cross-section. For example, the pull wire may be rectangular in cross-section with a width-to-thickness ratio of about 5 to 1.

As with the catheter embodiment illustrated in FIGS. 5B through 5E, the catheter of FIGS. 6A through 6D may include a support 126 that supports the ultrasound transducer array 68. The support 126 may interconnect the deflectable member 116 to the outer tubular body 118. A flexboard 122 may contain electrical interconnections operable to electrically connect the ultrasound transducer array 68 to an electrical interconnection member 104 (shown in FIG. 6D) disposed within the outer tubular body 118. The exposed portion of flexboard 122 may be encapsulated similarly to the flexboard 76 discussed above.

The outer tubular body 118 may include a distal portion 124. The distal portion 124 may comprise a plurality of wrapped layers disposed about a securement portion 133 (shown in FIGS. 6B and 6C) of the support 126. The wrapped layers may serve to secure the securement portion 133 to an inner portion of the outer tubular body 118 as discussed below with reference to FIG. 6D.

Deflection of the deflectable member 116 will now be discussed with reference to FIGS. 6B and 6C. FIGS. 6B and 6C illustrate the deflectable member 116 with the portion of the tip 120 surrounding the ultrasound image array 68 and support 126 removed. Also, the distal portion 124 of the outer tubular body 118 wrapped around the securement portion 133 has been removed. The support 126 may be configured similarly to the support 74 discussed above. The support 126 may further include a hinge portion 131 similar to the hinge portion 86.

To deflect the deflectable member 116 relative to the outer tubular body 118, the pull wire 130 may be moved relative to the outer tubular body 118. As shown in FIG. 6C, pulling the pull wire 130 (e.g., toward the handle 56) may impart a force on the support 126 at a pull wire anchor point 132 directed along the pull wire 130 toward a pull wire outlet 134. The pull wire outlet 134 is the point where the pull wire 130 emerges from a pull wire housing 136. The pull wire housing 136 may be fixed to the outer tubular body 118. Such a force may result in the deflectable member 116 bending toward the pull wire outlet 134. As in the embodiment illustrated in FIGS. 5C and 5D, the deflection of the deflectable member will be constrained by the hinge portion 131 of the support 126. As illustrated in FIG. 6C, the resultant deflection of the deflectable member 116 may result in the ultrasound transducer array 68 being pivoted to a forward-looking position. It will be appreciated that varying amounts of deflection of the deflectable member 116 may be achieved through controlled movement of the pull wire 130. In this regard, any deflection angle between 0 degrees and 90 degrees may be achievable by displacing the pull wire 130 a lesser amount than as illustrated in FIG. 6C. Furthermore, deflections of greater than 90 degrees may be obtainable by displacing the pull wire 130 a greater amount than as illustrated in FIG. 6C. As illustrated in FIGS. 6B and 6C, the flexboard 122 may remain interconnected to the outer tubular body 118 and the deflectable member 116 independent of the deflection of the deflectable member 116.

FIG. 6D illustrates an embodiment of the outer tubular body 118. For the illustration of FIG. 6D, portions of various layers have been removed to reveal the construction of the outer tubular body 118. Layers similar to those of the embodiment of FIG. 5E are labeled with the same reference numbers as in FIG. 5E and will not be discussed at length here. The pull wire housing 136 housing the pull wire 130 may be disposed proximate to the outer covering 94. An external wrap 138 may then be disposed over the outer covering 94 and pull wire housing 136 to secure the pull wire housing 136 to the outer covering 94. Alternatively, the pull wire housing 136 and pull wire 130 may, for example, be disposed between the outer covering 94 and the outer low-dielectric constant layer 96. In such an embodiment, the outer wrap 138 may not be needed. Other appropriate locations for the pull wire housing 136 and pull wire 130 may be utilized.

Disposed interior to the outer low-dielectric constant layer 96 may be the shield layer 98. A first tie layer (not shown in FIG. 6D), similar to first tie layer 97, may be disposed between the outer low-dielectric constant layer 96 and the shield layer 98. Disposed interior to the shield layer may be the second tie layer 100. Disposed interior to the second tie layer 100 may be the electrical interconnection member 104. Disposed interior to the electrical interconnection member 104 may be an inner low-dielectric constant layer 142. In this regard, the electrical interconnection member 104 may be helically disposed within the wall of the outer tubular body 118.

Moving toward the center of the outer tubular body 118, the next layer may be a coiled reinforcement layer 144. The coiled reinforcement layer 144 may, for example, comprise a stainless steel coil. In an exemplary embodiment, the coiled reinforcement layer 144 may be about 0.05-0.08 mm thick. Moving toward the center of the outer tubular body 118, the next layer may be an inner covering 146. The inner covering 146 may be configured similar to and serve a similar function as the outer covering 94. The lumen 128 may have a central axis aligned with the central axis of the outer tubular body 118.

As noted above, the wrapped layers of the distal portion 124 of the outer tubular body 118 may serve to secure the securement portion 133 of the support 126 to an inner portion of the outer tubular body 118. For example, each layer outboard of the electrical interconnection member 104 may be removed in the distal portion 124. Furthermore, the electrical interconnection member 104 may be electrically interconnected to the flexboard 122 proximal to the distal portion 124 in a manner similar to as described with reference to FIG. 5F. Accordingly, the securement portion 133 of the support 126 may be positioned over the remaining inner layers (e.g., the inner low-dielectric constant layer 142, the coiled reinforcement layer 144 and the inner covering 146) and a plurality of layers of material may be wrapped about the distal portion 124 to secure the securement portion 133 to the outer tubular body 118.

The outer diameter of the outer tubular body 118 may, for example, be about 12.25 Fr. The inner diameter of the outer tubular body 118 may, for example, be about 8.4 Fr.

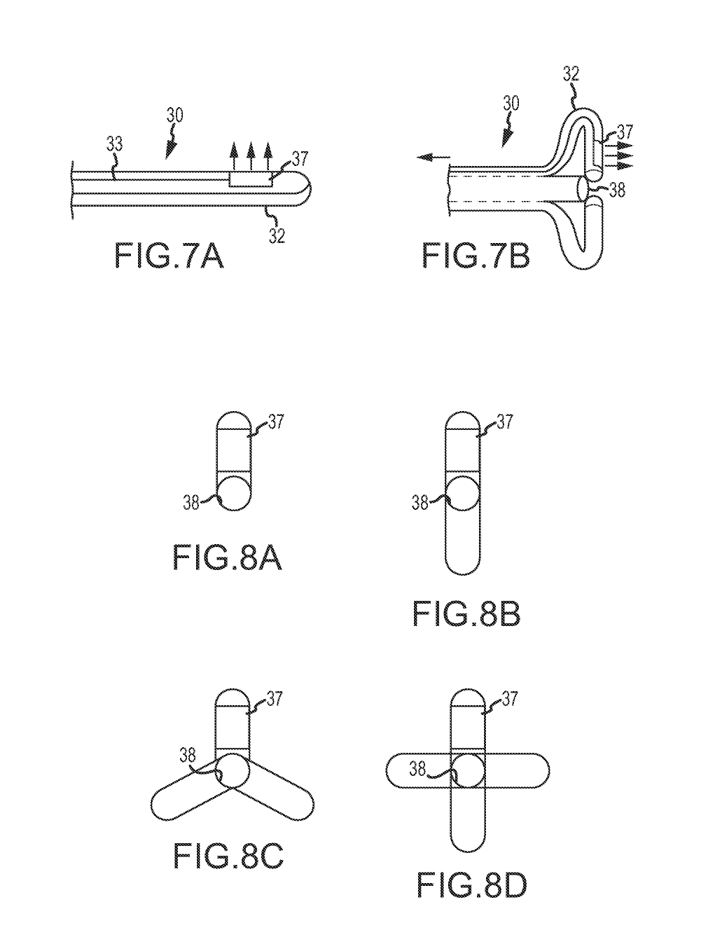

FIGS. 7A and 7B demonstrate further embodiments. As shown, the catheter 30 comprises a deflectable distal end 32. Located at deflectable distal end 32 is ultrasound transducer array 37. The catheter also includes wire 33 attached to the ultrasound transducer array 37 and extending to the proximal end of catheter 30 where it exits through a port or other opening at the proximal end of catheter 30. As shown in FIG. 7A, ultrasound transducer array 37 is in a side-looking configuration. The catheter can be delivered to the treatment site with the ultrasound transducer array 37 in the side-looking configuration, as shown in FIG. 7A. Once the treatment site is reached, wire 33 can be pulled in a proximal direction to deflect deflectable distal end 32 to result in ultrasound transducer array 37 being moved to a forward-looking configuration, as shown in FIG. 7B. As shown in FIG. 7B, once ultrasound transducer array 37 is positioned in the forward-looking position and deflectable distal end 32 is deflected as shown, generally centrally located lumen 38 is then available for delivery of a suitable interventional device to a point distal to the catheter distal end 32. Alternatively, a tube containing lumen 38 and movable relative to the outer surface of the catheter 30 may be used to deflect the deflectable distal end 32 to the forward-looking configuration.

FIG. 8A is a front view of a single lobe configuration of the device shown in FIGS. 7A and 7B. FIG. 8B shows a dual-lobe configuration of the catheter shown in FIGS. 7A and 7B. FIG. 8C shows a tri-lobe configuration and FIG. 8D shows a quad-lobe configuration. As will be understood, any suitable number of lobes can be constructed as desired. Moreover, in multiple-lobe configurations, ultrasound transducer arrays 37 may be disposed on one or more of the lobes.

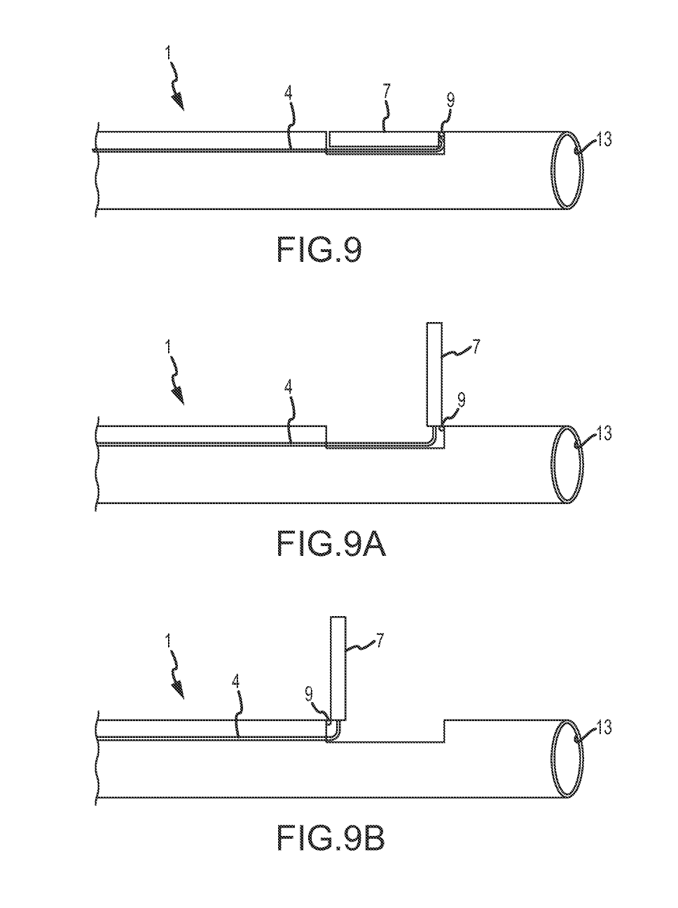

Further embodiments are shown in FIGS. 9, 9A and 9B. FIG. 9 shows catheter 1 having an ultrasound transducer array 7 near the distal end thereof. The ultrasound transducer array 7 is attached to catheter 1 by hinge 9. Electrically conductive wires 4 are connected to ultrasound transducer array 7 and extend proximally to the proximal end of the catheter 1. The catheter 1 includes distal port 13. The hinge 9 can be located at the distal end of ultrasound transducer array 7, as shown in FIG. 9A, or at the proximal end of ultrasound transducer array 7, as shown in FIG. 9B. In any event, the ultrasound transducer array 7 can be either passively or actively deflectable, as discussed above. Ultrasound transducer array 7 can be deflected up to the forward-looking configuration (as shown in FIGS. 9A and 9B) and an interventional device can be advanced at least partially out of distal port 13, such that at least a portion of the interventional device will be in the field of view of the ultrasound transducer array 7.

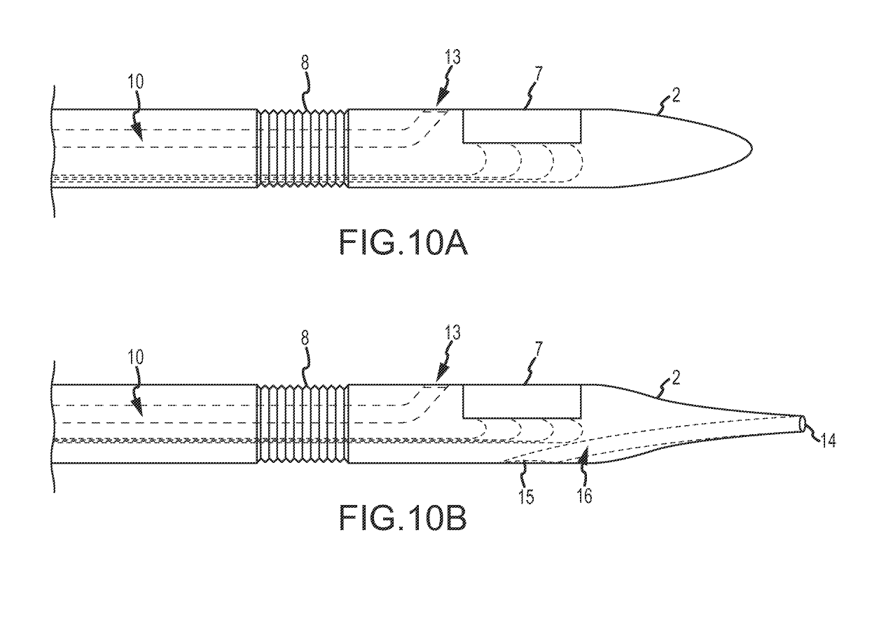

FIGS. 10A and 10B demonstrate a further embodiment where the catheter includes ultrasound transducer array 7 near the catheter distal end 2 of the catheter. The catheter further includes steerable segment 8 and lumen 10. Lumen 10 can be sized to accept a suitable interventional device that can be inserted at the proximal end of the catheter and advanced through lumen 10 and out port 13. The catheter can further include guidewire receiving lumen 16. Guidewire receiving lumen 16 can include proximal port 15 and distal port 14, thus allowing for the well known “rapid exchange” of suitable guidewires.

As further demonstrated in FIGS. 11 and 11A and 11B, the catheter steerable segment 8 can be bent in any suitable direction. For example, as shown in FIG. 11A the steerable segment is bent away from port 13 and as shown in FIG. 11B the steerable segment is bent toward port 13.

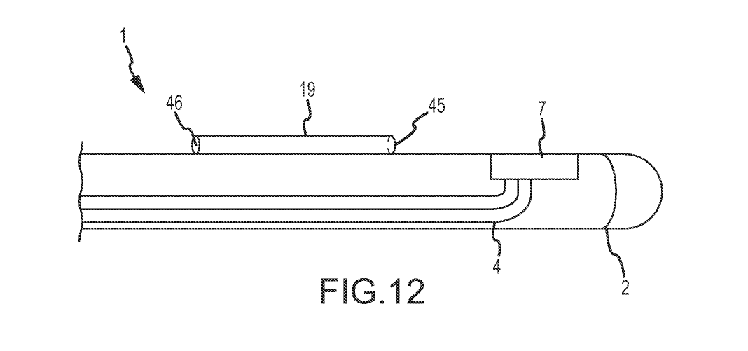

FIG. 12 demonstrates yet another embodiment. Specifically, catheter 1 can include ultrasound transducer array 7 located at the distal end 2 of the catheter 1. Electrically conductive wires 4 are attached to the ultrasound transducer array 7 and extend to the proximal end of the catheter 1. Lumen 19 is located proximal to the ultrasound transducer array 7 and includes proximal port 46 and distal port 45. The lumen 19 can be sized to accept a suitable guidewire and/or interventional device. Lumen 19 can be constructed of a suitable polymer tube material, such as ePTFE. The electrically conductive wires 4 can be located at or near the center of the catheter 1.

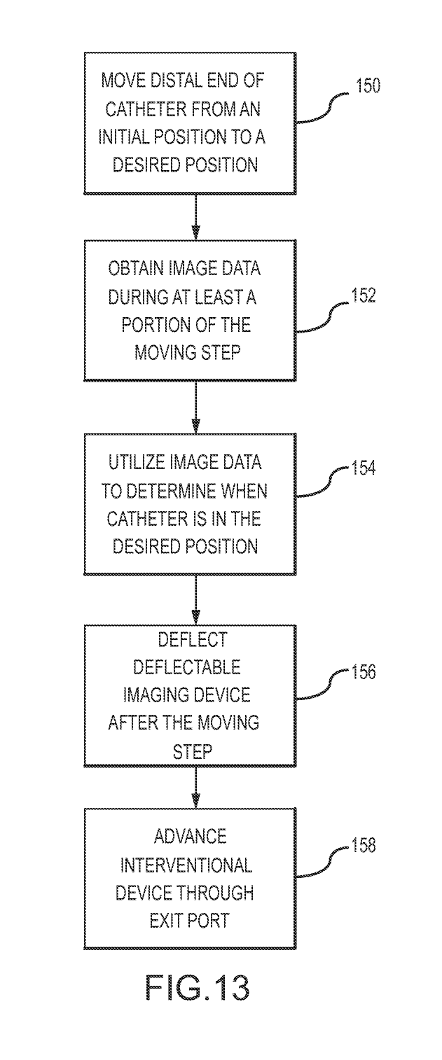

FIG. 13 is a flow chart for an embodiment of a method of operating a catheter having a deflectable imaging device located at a distal end thereof. The first step 150 in the method may be to move the distal end of the catheter from an initial position to a desired position, wherein the deflectable imaging device is located in a first position during the moving step. The deflectable imaging device may be side-looking when in the first position. The moving step may include introducing the catheter into a body through an entry site that is smaller than the aperture of the deflectable imaging device. The moving step may include rotating the catheter relative to its surroundings.

The next step 152 may be to obtain image data from the deflectable imaging device during at least a portion of the moving step. The obtaining step may be performed with the deflectable imaging device located in the first position. During the moving and obtaining steps, a position of the deflectable imaging device relative to the distal end of the catheter may be maintained. Thus the deflectable imaging device may be moved and images may be obtained without moving the deflectable imaging device relative to the distal end of the catheter. During the moving step, the catheter, and therefore the deflectable imaging device, may be rotated relative to its surroundings. Such rotation may allow the deflectable imaging device to obtain images in a plurality of different directions transverse to the path traveled by the catheter during the moving step.

The next step 154 may be to utilize the image data to determine when the catheter is located at the desired position. For example, the image data may indicate the position of the deflectable imaging device, and therefore the distal end of the catheter, relative to a landmark (e.g., an anatomical landmark).

The next step 156 may be to deflect the deflectable imaging device from the first position to a second position. The deflecting step may follow the moving step. The deflectable imaging device may be forward-looking in the second position. The deflectable imaging device may be angled at least about 45 degrees relative to a central axis of the catheter when in the second position. Optionally, after the deflecting step, the deflectable imaging device may be returned to the first position and the catheter repositioned (e.g., repeating the moving step 150, the obtaining step 152, and the utilizing step 154). Once repositioned, the deflecting step 156 may be repeated and the method may be continued.

In an embodiment, the catheter may comprise an outer tubular body and an activation device, each extending from a proximal end to the distal end of the catheter. In such an embodiment, the deflecting step may include translating a proximal end of at least one of the outer tubular body and actuation device relative to a proximal end of the other one of the outer tubular body and actuation device. The deflectable imaging device may be supportably interconnected by a hinge to one of the outer tubular body and the actuation device, and the deflecting step may further comprise applying a deflection force to the hinge in response to the translating step. Furthermore, the deflecting step may further include initiating the application of the deflection force to the hinge in response to the translating step. The deflection force may be applied and then maintained by manipulating a handle interconnected to the proximal end of the catheter. Moreover, the applying step may comprise communicating the deflection force by the actuation device from the proximal end to the distal end of the catheter in a balanced and distributed manner about a central axis of the outer tubular body.

The next step 158 may be to advance an interventional device through a port at the distal end of the catheter and into an imaging field of view of the deflectable imaging device in the second position. The imaging field of view may be maintained in substantially fixed registration to the distal end of the catheter during the advancing step.

After advancing and using the interventional device (e.g., to perform a procedure, to install or retrieve a device, to make a measurement), the interventional device may be withdrawn through the port. The deflectable imaging device may then be returned to the first position. The return to the first position may be facilitated by an elastic deformation quality of the hinge. For example, the hinge may be biased toward positioning the deflectable imaging device in the first position. As such, when the deflectable imaging device is in the second position and the deflection force is removed, the deflectable imaging device may return to the first position. After withdrawal of the interventional device through the port (and optionally from the entire catheter) and return of the deflectable imaging device to the first position, the catheter may then be repositioned and/or removed.

As with the supports 74, 126 above, the supports described below may be made from any appropriate material, such as, for example, a shape memory material (e.g., Nitinol). Any appropriate tubular body discussed herein may be configured to include any suitable electrical configuration member. For example, where appropriate in the embodiments discussed below, the outer tubular bodies may contain electrical interconnection members similar to the electrical interconnection member 104 of FIG. 5E.

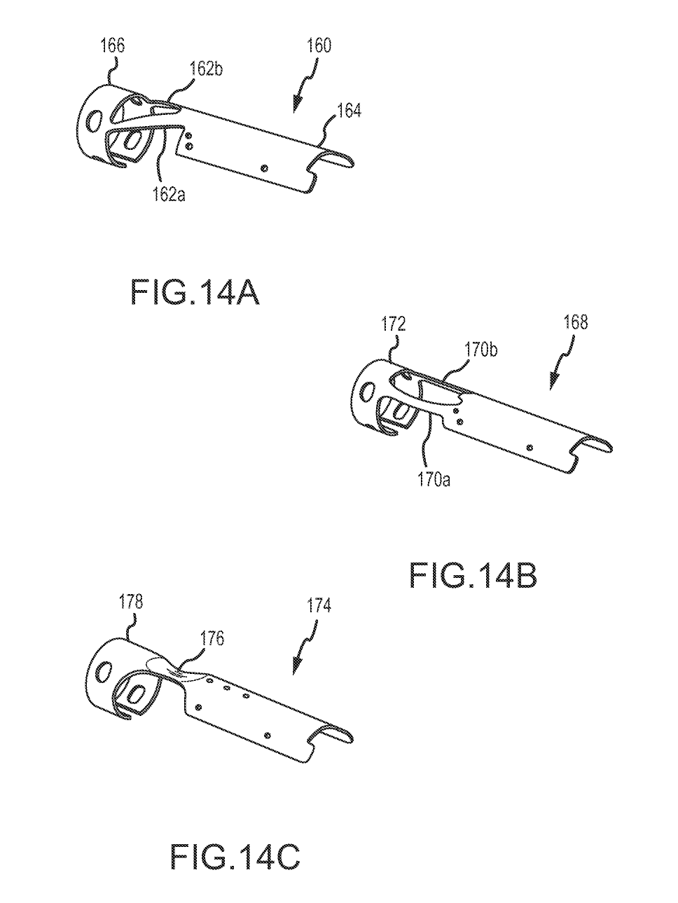

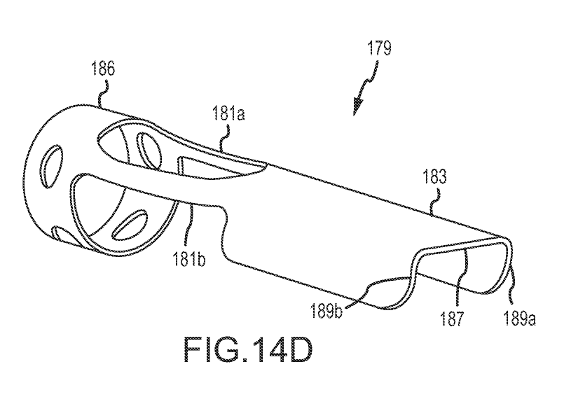

The support 74 of FIGS. 5B through 5D, the support 126 of FIGS. 6A through 6C, and any similarly configured support disclosed herein may contain variations of the hinge portion 86 described with reference to FIGS. 5B through 5D and hinge portion 131 described with reference to FIGS. 6A through 6C. For example, FIGS. 14A through 14C illustrate three alternative hinge portion designs. FIG. 14A illustrates a support 160 that includes hinge portions 162a, 162b that are tapered—the hinge portions 162 a/b become thinner as the distance from a cradle portion 164 increases in the direction of a tubular body interface portion 166.

FIG. 14B illustrates a support 168 that includes hinge portions 170a, 170b that are scalloped and disposed within a curved plane of a tubular body interface portion 172. FIG. 14C illustrates a support 174 that includes a unitary hinge portion 176. The unitary hinge portion 176 is a scalloped with a narrow portion disposed proximate to its midpoint. Furthermore, the unitary hinge portion 176 is curved such that a portion of the unitary hinge portion 176 is disposed within the interior of a tube defined by and extending from a tubular body interface portion 178. FIG. 14D illustrates a support 179 that includes hinge portions 181a, 181b, a tubular body interface portion 185 and a cradle portion 183. The cradle portion 183 includes a flat section 187 and two side sections 189a, 189b oriented generally perpendicular to the flat section 187. Such design variations as those illustrated in FIGS. 14A through 14D may provide satisfactory cycles to failure (e.g., bending cycles), lateral stiffness and angular bending stiffness, while maintaining strain and plastic deformation within acceptable levels.

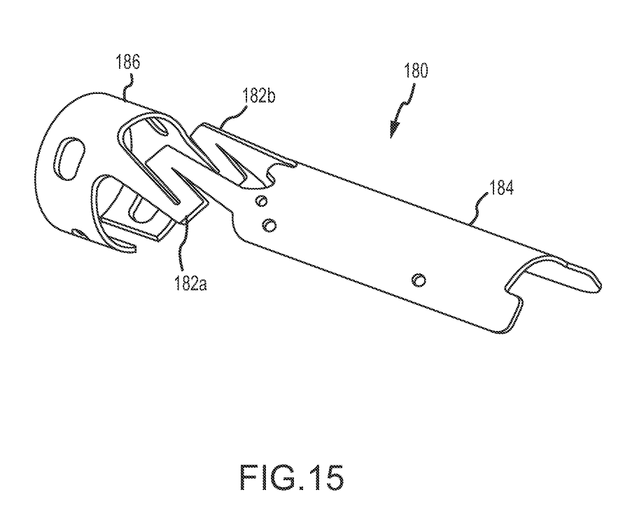

FIG. 15 illustrates a support 180 that incorporates a pair of zigzagging hinge portions 182a, 182b. Such a design allows for the maintenance of adequate hinge portion 182a, 182b width and thickness while allowing for a longer effective cantilever bend length, thus decreasing the level of force required to deflect a cradle portion 184 relative to a tubular body interface portion 186. Other appropriate configurations where the effective cantilever bend length may be increased (as compared to a straight hinge portion) may also be utilized.

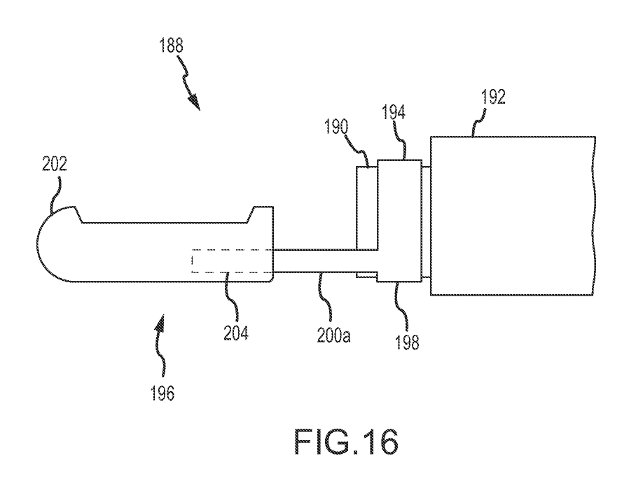

FIG. 16 illustrates a catheter 188 that includes an inner tubular body 190 and an outer tubular body 192. Attached to the inner tubular body 190 is a support 194 that supports a deflectable member 196. The support 194 includes a tubular body interface portion 198 that is attached to the inner tubular body 190 using any appropriate method of attachment such as, for example, clamping and/or gluing. The support 194 further includes two hinge portions: a first hinge portion 200a and a second hinge portion (not visible in FIG. 16 due to its position parallel to and directly behind the first hinge portion 200a). The deflectable member 196 includes a tip portion 202 that may, for example, be molded over an end portion 204 of the first hinge portion 200a and the second hinge portion. The tip portion 202 may also contain an ultrasound imaging array, appropriate electrical connections, and any other appropriate component. Any appropriate electrical interconnection scheme and any appropriate deflection actuation scheme, such as those described herein, may be used with the support 194 of FIG. 16.

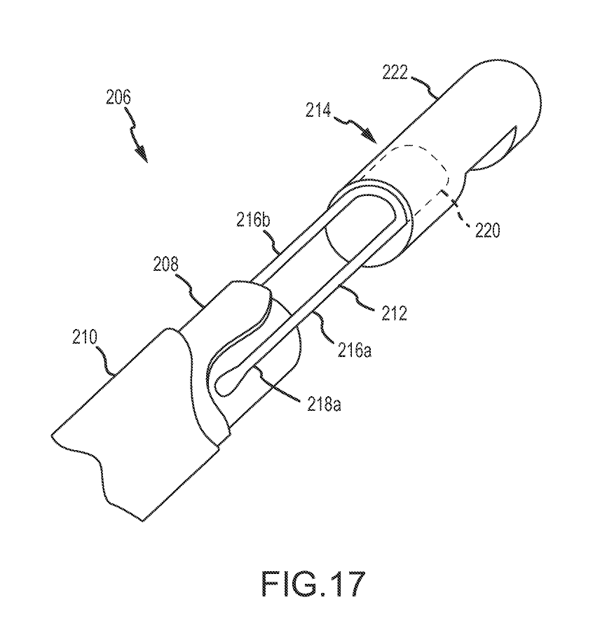

FIG. 17 illustrates a catheter 206 that includes an inner tubular body 208 and an outer tubular body 210. Attached to the inner tubular body 208 is a support 212 that supports a deflectable member 214. The support 212 includes first and second hinge portions 216a, 216b that allow for deflection of the deflectable member 214 relative to the inner and outer tubular bodies 208, 210. The outer tubular body 210 has been cut away in FIG. 17 to aid this description. The support 212 further includes a first inner tubular body interface region 218a. The first inner tubular body interface region 218a may be disposed between layers of the inner tubular body 208 to secure the support 212 to the inner tubular body 208. To illustrate this attachment in FIG. 17, a portion of the inner tubular body 208 disposed over the first inner tubular body interface region 218a has been cut away. A second inner tubular body interface region is attached to the second hinge portion 216b and is disposed within the layers of the inner tubular body 208 and is therefore not visible in FIG. 17. The inner tubular body interface regions may be attached to the inner tubular body 208 using any appropriate attachment method (e.g., glued, tacked). The support 212 may further include an end portion 220. The deflectable member may include a tip portion 222 that may be molded over the end portion 220 to secure the deflectable member 214 to the support 212 (similar to as described with reference to FIG. 16). The tip portion 222 may also contain an ultrasound imaging array, appropriate electrical connections, and any other appropriate component. Any appropriate electrical interconnection scheme and any appropriate deflection actuation scheme, such as those described herein, may be used with the support 212 of FIG. 17. In an alternate configuration, the support 212 may include a single hinge portion.

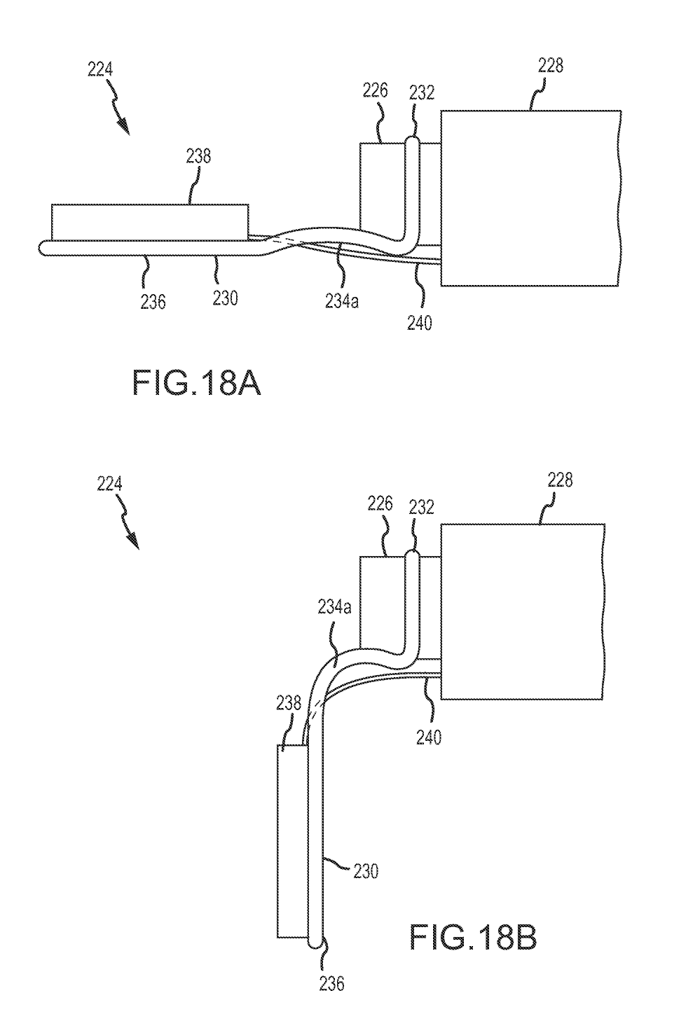

FIGS. 18A and 18B illustrate a catheter 224 that includes an inner tubular body 226 and an outer tubular body 228. Attached to the inner tubular body 226 is a support 230. The support 230 is constructed from a strand of wire bent into a shape to perform the functions described below. The support 230 may be constructed such that it is made from a continuous loop of wire (e.g., during formation, the ends of the wire strand used to make the support 230 may be attached to each other). The support 230 includes a tubular body interface portion 232 that is operable to be secured to the inner tubular body 226 in any appropriate way (e.g., clamped and/or bonded). The support 230 further includes two hinge portions: a first hinge portion 234a and a second hinge portion (not visible in FIGS. 18A and 18B due to its position parallel to and directly behind the first hinge portion 234a). The support 230 further includes an array support portion 236 operable to support an ultrasound imaging array 238. The hinge portions allow for deflection of the ultrasound imaging array 238 relative to the inner and outer tubular bodies 226, 228. The catheter 224 may further include a tether and/or electrical interconnection member 240. The catheter 224 may also further include a second tether and/or electrical interconnection member (not shown). As illustrated in FIGS. 18A and 18B, an extension (a leftward movement in FIGS. 18A and 18B) of the inner tubular body 226 relative to the outer tubular body 228 may result in the deflection of the ultrasound imaging array 238 relative to the outer tubular body 228. The catheter 224 may also include a tip portion (not shown) that may be molded over the ultrasound imaging array 238, array support portion 236, and any other appropriate components. Any appropriate electrical interconnection scheme and any appropriate deflection actuation scheme, such as those described herein, may be used with the support 230 of FIGS. 18A and 18B.

Returning briefly to FIGS. 5C and 5D, the tether 78 and flexboard 76 are illustrated interconnected between the outer tubular body 79 and the cradle portion 88. In an alternate arrangement of FIGS. 5C and 5D, the functions of the tether 78 and flexboard 76 may be combined. In such an arrangement, the flexboard 76 may also act as a tether. The flexboard 76 that also serves as a tether may be a typical flexboard, or it may be specially adapted (e.g., reinforced) to serve as a tether. Where appropriate, a flexboard or other electrical interconnection member between a deflectable member and a catheter body may also serve as a tether (e.g., such an arrangement could be employed in catheter 224 of FIGS. 18A and 18B).

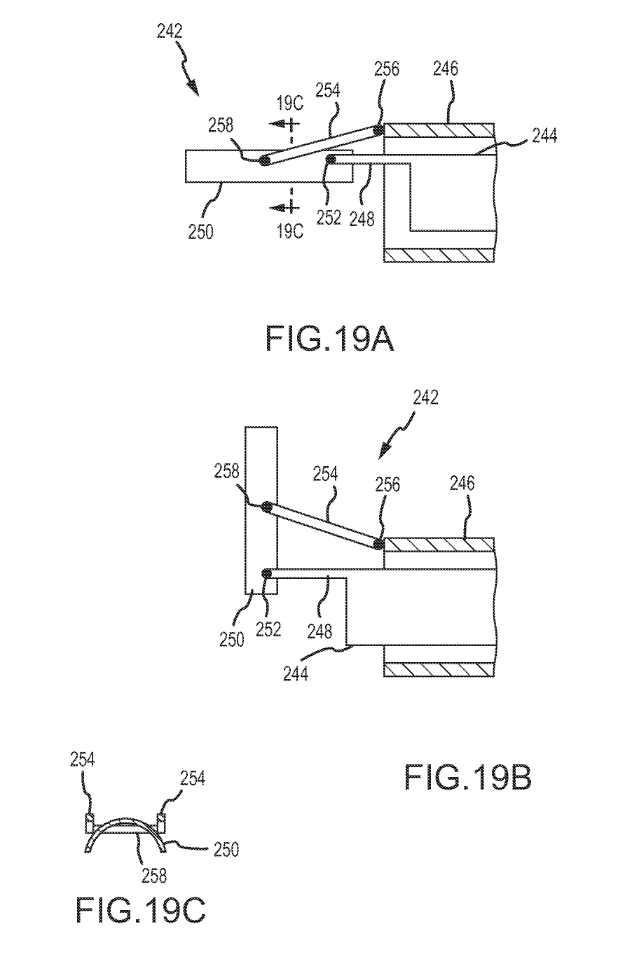

FIGS. 19A-19C illustrate a catheter 242 that includes an inner tubular body 244 and an outer tubular body 246. An inner tubular body extension 248 extends from a distal end of the inner tubular body 244. The inner tubular body extension 248 is pivotably interconnected to an array support 250 via an inner body to array support pivot 252. The inner tubular body extension 248 is generally rigid enough to be able to pivot the array support 250 as described below. The array support 250 may support an ultrasound imaging array (not shown in FIGS. 19A-19C). The array support 250 may be operable to pivot relative to the inner tubular body extension 248 about the inner body to array support pivot 252. The catheter 242 may also include a tether 254. The tether may be of sufficient rigidity to not substantially buckle as the array support 250 is pivoted. The tether 254 may include two individual members (only one of the members is visible in FIGS. 19A and 19B due to one of the members position parallel to and directly behind the other member). On a first end, the tether 254 may be pivotably interconnected to the outer tubular body 246 via an outer body to tether pivot 256. On a second end, the tether 254 may be pivotably interconnected to the array support 250 via a tether to array support 258. As shown in FIG. 19C (a cross sectional view of FIG. 19A along section lines 19C), the two members of the tether 254 may be disposed on each end of the tether to array support 258. The array support 250 may be curved and the tether to array support 258 may pass through corresponding holes in the array support 250. The other pivots 252, 256 may be similarly configured. The inner tubular body extension 248 may be configured similarly to the tether 254 in that it may also be made up of two members that straddle the array support 250 and interconnect to two ends of the inner body to array support pivot 252.

To pivot the array support 250 relative to the inner and outer tubular bodies 244, 246, the inner tubular body 244 is moved along a common central axis relative to the outer tubular body 246. As illustrated in FIGS. 19A and 19B, this relative motion, in combination with the tether's 254 maintenance of a fixed distance between the pivot 258 on the array support 250 and the pivot 256 on the outer tubular body 246, causes the array support 250 to rotate about the inner body to array support pivot 252 until, as shown in FIG. 19B, the array support is substantially perpendicular to the common central axis of the inner and outer tubular bodies 244, 246. Moving the inner tubular body 244 in the opposite direction causes the array support 250 to pivot back into the position shown in FIG. 19A. It will be appreciated that the inner tubular body 244 may be extended beyond the position illustrated in FIG. 19B such that the array support 250 is pivoted through an angle greater than 90 degrees. In an embodiment, the array support 250 may be pivotable through an angle approaching 180 degrees such that the open portion of the array support 250 is generally pointing upwards (e.g., in a direction opposite to that shown in FIG. 19A).

The catheter 242 may also include a tip portion (not shown) that may be molded over the array support 250, an ultrasound imaging array, and any other appropriate components. Any appropriate electrical interconnection, such as those described herein, may be used with the catheter 242 of FIGS. 19A through 19C.

In a variation of the embodiment of FIG. 19A, the inner tubular body extension 248 may be replaced with an outer tubular body extension of a similar configuration but part of the outer tubular body 246 instead of the inner tubular body 244. In such a variation, the outer tubular body extension may be rigidly fixed to the outer tubular body 246 and permanently positioned similar to the tether 254. In such a variation, the outer tubular body extension may be pivotably interconnected to the array support 250 in any appropriate manner. Such a pivotable interconnection may be disposed toward the proximate end of the array support 250 (e.g., the end closest to the inner tubular body 244). A link may be disposed between the proximate end of the array support 250 and the inner tubular body 244 such that when the inner tubular body 244 is advanced relative to the outer tubular body 246, the array support 250 pivots about the pivotable interface between the outer tubular body extension and the array support 250.

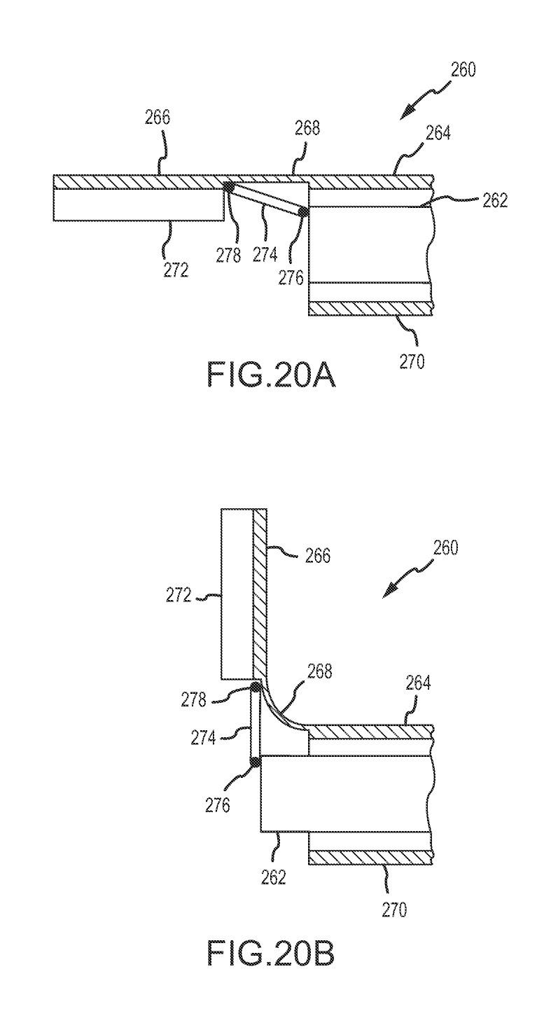

FIGS. 20A and 20B illustrate a catheter 260 that includes an inner tubular body 262 and an outer tubular body 264. The outer tubular body 264 includes a support portion 266 and a hinge portion 268 disposed between the support portion 266 and a tubular portion 270 of the outer tubular body 264. The hinge portion 268 may generally position the support portion 266 such that the support portion 266 is aligned with the tubular portion 270 as shown in FIG. 20A. The hinge portion 268 may be resilient in that it may impart a return force when deflected from the aligned position. For example, the hinge portion 268 may urge the support portion 266 back to the position shown in FIG. 20A when it is disposed in the position shown in FIG. 20B. The hinge portion 268 may be an appropriately sized portion of the outer tubular body 264 and/or it may include additional material such as a support member (e.g., to increase stiffness). An ultrasound imaging array 270 may be interconnected to the support portion 266. A link 274 may be disposed between the inner tubular body 262 and the support portion 266. The link 274 may be adequately rigid to resist buckling. The link 274 may be attached to the inner tubular body 262 via an inner tubular body to link pivot 276. The link 274 may be attached to the support portion 266 via a support portion to link pivot 278.

To pivot the support portion 266 and its attached ultrasound imaging array 272 relative to the inner and outer tubular bodies 262, 264, the inner tubular body 262 is moved along a common central axis relative to the outer tubular body 264. As illustrated in FIGS. 20A and 20B, this relative motion, in combination with the link's 274 maintenance of a fixed distance between the pivots 276, 278 causes the support portion 266 to rotate until, as shown in FIG. 20B, the array support is substantially perpendicular to the common central axis of the inner and outer tubular bodies 262, 264. Moving the inner tubular body 262 in the opposite direction causes the support portion 266 to pivot back into the position shown in FIG. 20A.

The catheter 260 may also include a tip portion (not shown) that may be molded over the support portion 266 and the ultrasound imaging array 272, and any other appropriate components. Any appropriate electrical interconnection, such as those described herein, may be used with the catheter 260 of FIGS. 20A and 20B.

In a first variation of the embodiment of FIG. 20A, link 274 may be replaced with bendable member fixedly attached to the support portion 266 on one end and the inner tubular body 262 on the other end. Such a bendable member may bend when the inner tubular body 244 is advanced relative to the outer tubular body 246 and allow for the support portion to be pivoted as shown in FIG. 20B. In a second variation of the embodiment of FIG. 20A, the support portion 266 and hinge portion 268 may be replaced by a separate member that may be configured similarly to, for example, supports 160, 168, 174 and/or 180, with the modification that the respective tubular body interface portion be sized and configured to be attached to the outer tubular body 264. The first and second variations may be incorporated singularly or both may be incorporated into an embodiment.

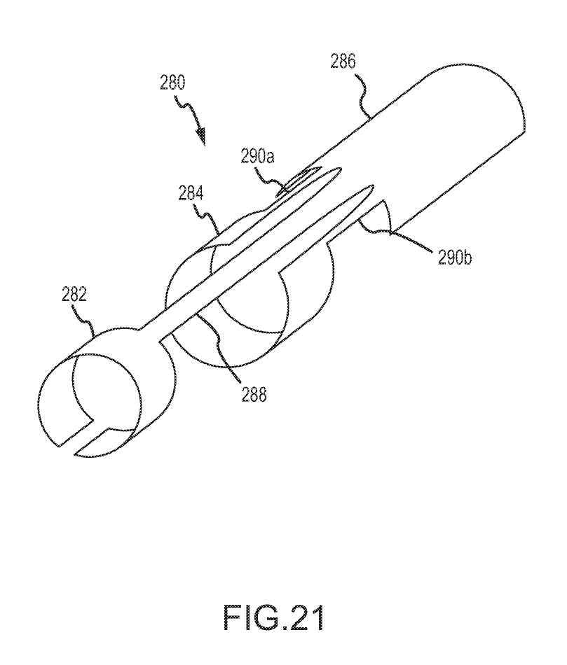

FIG. 21 illustrates a support 280 that may be used in a catheter, where the catheter includes an inner tubular body, an outer tubular body and an ultrasound imaging array. The support 280 includes a proximal tubular body interface portion 282 that is capable of being attached to an inner tubular body using any appropriate method of attachment such as, for example, clamping and/or gluing. The support 280 further includes a distal tubular body interface portion 284 that is capable of being attached to an outer tubular body using any appropriate method of attachment. The support 280 further includes an array support portion 286 for supporting an ultrasonic imaging array. The support 280 further includes two links: a first link 288 and a second link. The second link includes two parts, link 290a and link 290b. The support 280 may be configured such that when the proximal tubular body interface portion 282 is moved relative to the distal tubular body interface portion 284, the array support portion 286 may pivot relative to a common axis of the proximal tubular body interface portion 282 and the distal tubular body interface portion 284. Such action may be achieved by selecting appropriate relative widths and/or shapes of the links 288, 290a, 290b. In an alternate arrangement of the support 280, the proximal tubular body interface portion 282 may be attached to an outer tubular body and the distal tubular body interface portion 284 may attached to an inner tubular body. In such an embodiment, the proximal tubular body interface portion 282 and the distal tubular body interface portion 284 would be sized to attach to the outer and inner tubular bodies, respectively.

FIGS. 22A and 22B illustrate a catheter 294 that includes an inner tubular body 296 and an outer tubular body 298. Attached to the inner tubular body 296 is a support 300. The support 300 may be configured similarly to the support 74 of FIGS. 5B-5D with the addition of a notch 302. The catheter 294 may further include a tether 304 that interconnects the outer tubular body 298 to a cradle portion 306 of the support 300. Functionally, the tether 304 may perform a similar function to the tether 78 of FIGS. 5B-5D. The tether 304 may, for example, be formed from a flat ribbon (e.g., a flattened tube) including high strength toughened fluoropolymer (HSTF) and expanded fluorinated ethylene propylene (EFEP). The tether 304 may be configured such that it includes a flat portion 308 and a densified portion 310. The densified portion 310 of the tether 304 may be formed by twisting the tether 304 in the area to be densified and then heating the tether 304. The densified portion 310 may be generally round in cross section. Alternatively, the densified portion 310 may have a generally rectangular cross section, or a cross section having any other appropriate shape. In this regard, the flat portion 308 may be disposed between appropriate layers of the outer tubular body 298 without unacceptably affecting the diameter and/or shape of the outer tubular body 298, while the densified portion 310 may be generally round, which may, for example, aid in insertion and positioning within the notch 302 and help to avoid interference with other components (e.g., an electrical interconnection member and/or the support 300).

The notch 302 may be configured to accept the densified portion 310 of the tether 304 such that the densified portion 310 is hooked on to the notch 302. Accordingly, the notch 302 may be configured such that its opening is generally further away from the outer tubular body 298 than the deepest portion of the notch 302 where the tether 304 may tend to occupy. Since the tether 304 will generally be in tension during deflection of the cradle portion 306, the tether 304 may tend to remain within the notch 302. A tip 312 may be formed over the cradle portion 306 and as such may aid in retention of the densified portion 310 within the notch 302. As noted, the support 300 may be configured similarly to the support 74 of FIGS. 5B-5D and as such may be actuated in a similar manner (e.g., by motion of the inner tubular body 296 relative to the outer tubular body 298 and a corresponding bend of the support 300 as shown in FIG. 22B). The catheter 294 may also include any other appropriate components. Any appropriate electrical interconnection scheme, such as those described herein, may be used with the catheter 294 of FIGS. 22A and 22B.

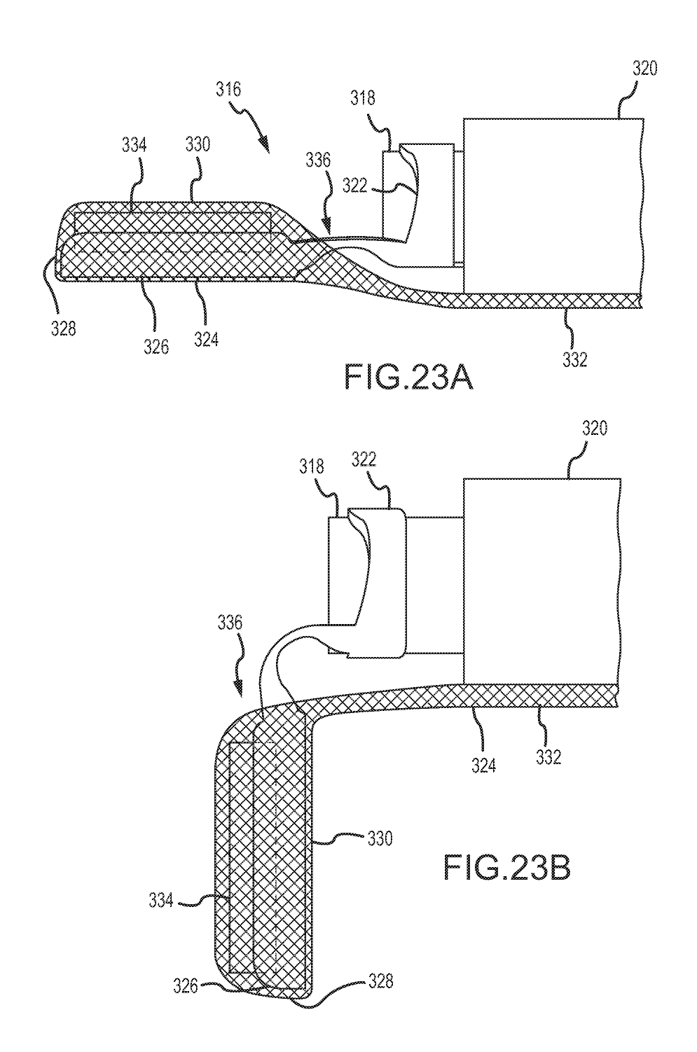

FIGS. 23A and 23B illustrate a catheter 316 that includes an inner tubular body 318 and an outer tubular body 320. Attached to the inner tubular body 318 is a support 322. The support 322 may be configured similarly to the support 74 of FIGS. 5B-5D. The catheter 316 may further include a tether sock 324 that functions to cause a cradle portion 326 of the support 322 to deflect (as shown in FIG. 23B) relative to the inner tubular body 318 when the inner tubular body 318 is moved relative to the outer tubular body 320. In this regard, the tether sock 324 performs a similar function as tether 78 of FIGS. 5B-5D. The tether sock may 324 may be generally tubular with a closed end 328. Once installed in the catheter 316, the tether sock 324 may include a tubular portion 330 and a collapsed portion 332. The tubular portion 330 may envelop the cradle portion 326 and an ultrasound imaging array 334. Alternatively, the tubular portion 330 may envelop the cradle portion 326 without covering the ultrasound imaging array 334. The collapsed portion 332 may generally be in the form of a collapsed tube and may be secured to the outer tubular body 320 in any appropriate manner. Between the tubular portion 330 and the collapsed portion 332, the tether sock 324 may include an opening 336. The opening 334 may be formed by, for example, cutting a slit into the tubular tether sock 324 prior to installation in the catheter 316. Such installation may include passing the cradle portion 326 through the opening 336 to dispose the cradle portion 326 within the closed end 328 of the tether sock 324. The remaining tether sock 324 (the portion of the tether sock 326 not disposed around the cradle portion 326) may be collapsed to form the collapsed portion 332 and attached to the outer tubular body 320 in any appropriate manner. The tether 324 may, for example, be formed from a material that includes a layer of HSTF sandwiched between two EFEP layers. The catheter 316 may also include any other appropriate components. Any appropriate electrical interconnection scheme, such as those described herein, may be used with the catheter 316 of FIGS. 23A and 23B.

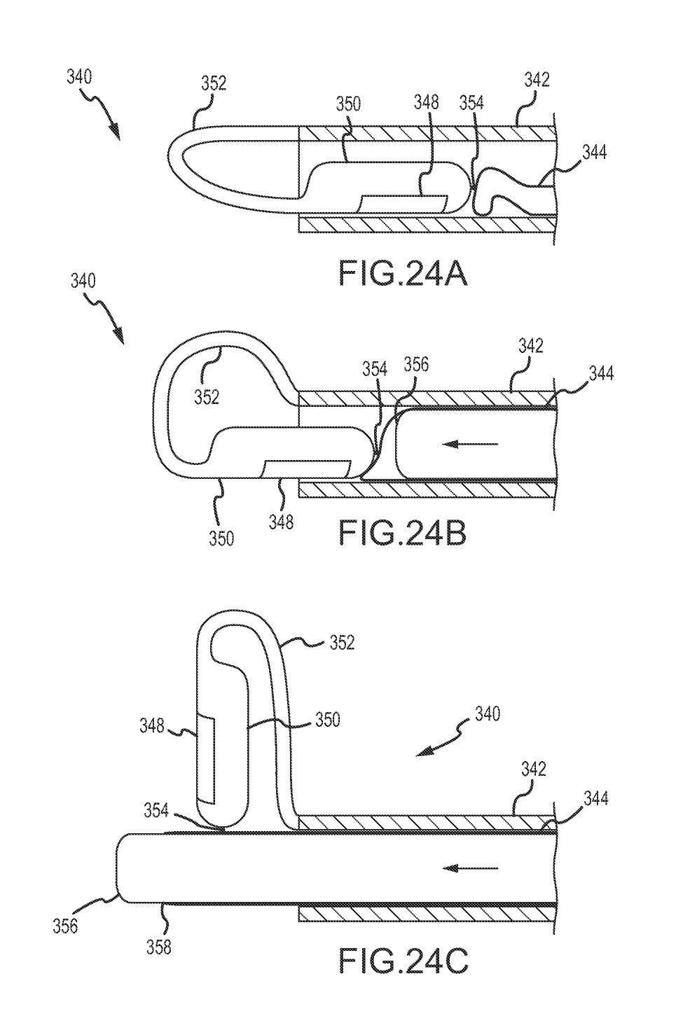

FIGS. 24A-24C illustrate a catheter 340 that includes an outer tubular body 342 and a collapsible inner lumen 344. In FIGS. 24A-24C, the collapsible inner lumen 344 and the outer tubular body 342 are shown in cross section. All other illustrated components of the catheter 340 are not shown in cross section.

While being inserted into a patient, the catheter 340 may be configured as shown in FIG. 24A with an ultrasound imaging array 348 disposed within the outer tubular body 342. The ultrasound imaging array 348 may be disposed within a tip portion 350. The ultrasound imaging array 348 may be electrically and mechanically interconnected to the outer tubular body 342 via a loop 352. The collapsible inner lumen 344 may be in a collapsed state while the tip portion 350 is disposed within the outer tubular body 342 as illustrated in FIG. 24A. The collapsible inner lumen 344 may be interconnected to the tip portion 350 by a joint 354. While in the position illustrated in FIG. 24A, the ultrasound imaging array 348 may be operable and thus images may be generated to aid in positioning of the catheter 340 before and/or during insertion of an interventional device 356.

FIG. 24B illustrates the catheter 340 as the interventional device 356 is displacing the tip portion 350. In this regard, as the interventional device 356 is advanced through the collapsible inner lumen 344, the interventional device 356 may push the tip portion 350 out of the outer tubular body 342.

FIG. 24C illustrates the catheter 340 after the interventional device 356 has been pushed through an opening 358 at the end of the collapsible inner lumen 344. The tip portion 350 may remain interconnected to the collapsible inner lumen 344 by virtue of the joint 354 between the two components. Once the interventional device 356 is extended through the opening 358, the ultrasonic imaging array 348 may be generally forward facing (e.g., facing in a distal direction relative to the catheter 340). Such positioning may be facilitated by an appropriately configured loop 352. The ultrasound imaging array 348 may remain electrically interconnected through appropriate cabling in the loop 352. The catheter 340 may also include any other appropriate components

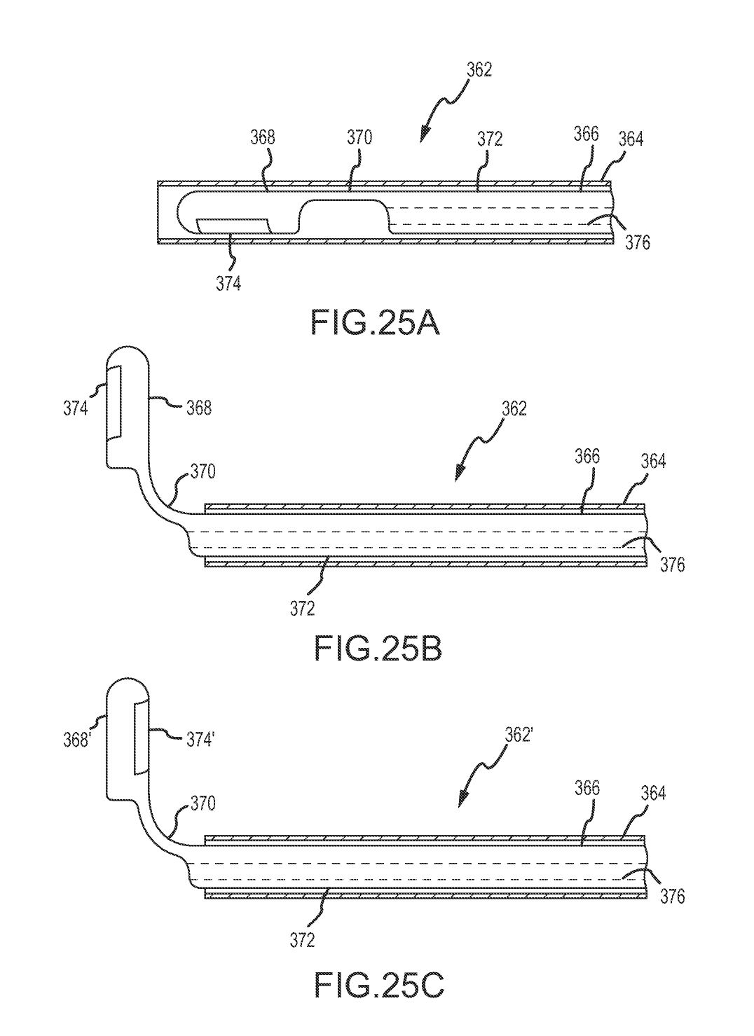

FIGS. 25A and 25B illustrate a catheter 362 that includes an outer tubular body 364 and an inner member 366. In FIGS. 25A and 25B, the outer tubular body 364 is shown in cross section. All other illustrated components of the catheter 362 are not shown in cross section. The inner member 366 may include a tip portion 368 and an intermediate portion 370 disposed between the tip portion 368 and a tube portion 372 of the inner member 366. The intermediate portion 370 may be configured such that it positions the tip portion 368 at about a right angle relative to the tube portion 372 (as illustrated in FIG. 25B) in the substantial absence of externally applied forces. In this regard, when the tip portion 368 is disposed within the outer tubular body 364, the outer tubular body 364 may contain the tip portion 368 such that the tip portion 368 remains aligned with the tube portion 372 as illustrated in FIG. 25A. In certain embodiments, the end of the outer tubular body 364 may be structurally reinforced to aid in retaining the tip portion 368 in alignment with the tube portion 372 while the tip portion 368 is disposed therein. The tip potion 368 may include an ultrasound imaging array 374. The tip portion 368 may also house an electrical interconnection member (not shown) electrically interconnected to the ultrasound imaging array 374. The electrical interconnection member may continue through the intermediate portion 370 and then along the inner member 366. The inner member 366 may also include a lumen 376 therethrough. Although illustrated as a single element, the tip portion 368, the intermediate portion 370, and the tube portion 372 may be discrete portions that are interconnected during an assembly process. In this regard, the intermediate portion 370 may be constructed from a shape memory material (e.g., Nitinol) with the memorized configuration including a 90 degree bend to position the tip portion 368 as shown in FIG. 25B.

In use, the catheter 362 may be inserted into a patient with the tip portion 368 disposed within the outer tubular body 364. Once the catheter 362 is in a desired position, the inner member 366 may be advanced relative to the outer tubular body 364 and/or the outer tubular body 364 may be retracted such that the tip portion 368 is no longer disposed within the outer tubular body 364. Accordingly, the tip portion 368 may move to the deployed position (illustrated in FIG. 25B) and the ultrasound imaging array 374 may be used to generate images of a volume distal to the catheter 362. An interventional device (not shown) may be advanced through the lumen 376.

FIG. 25C illustrates a catheter 362′ similar to catheter 362 of FIGS. 25A and 25B with a differently positioned ultrasound imaging array 374′. The ultrasound imaging array 374′ is disposed on the tip portion 368′ such that upon deflection of the tip portion 368′, the ultrasound imaging array 374′ may be pivoted into an at least partially rearward-looking position. The rearward-looking ultrasound imaging array 374′ may be in place of the ultrasound imaging array 374 of FIGS. 25A and 25B, or it may be in addition to the ultrasound imaging array 374 of FIGS. 25A and 25B.

Where appropriate, other embodiments described herein may include ultrasound imaging arrays that may be displaced into rearward-looking positions. These may be in place of or in addition to the disclosed ultrasound imaging arrays. For example, the embodiment illustrated in FIG. 2A may include an ultrasound imaging array that may be displaced into an at least partially rearward-looking position.

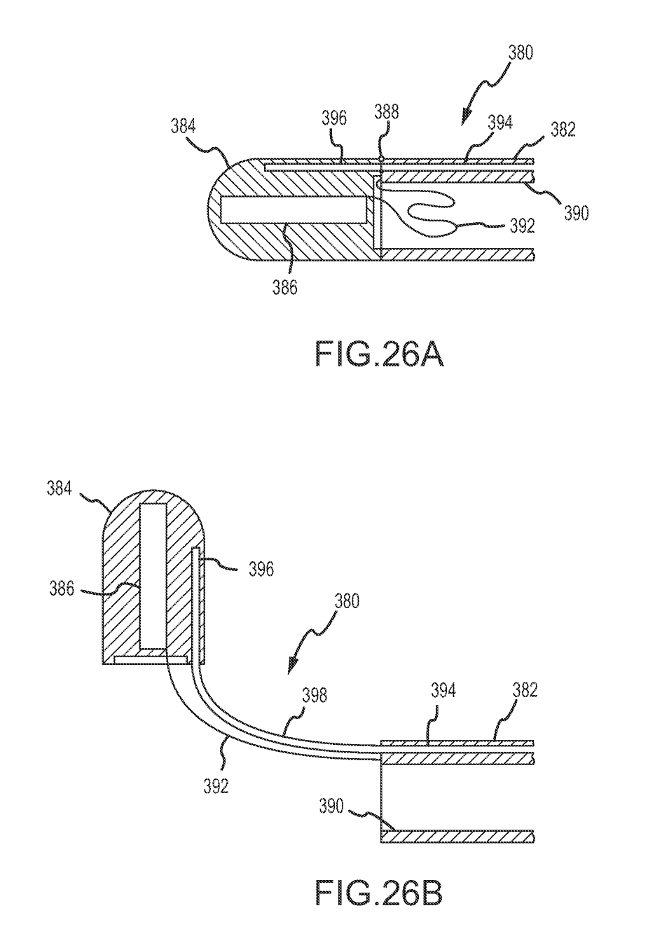

FIGS. 26A and 26B illustrate a catheter 380 that includes a tubular body 382 and a tip 384. In FIGS. 26A and 26B, the tubular body 382 and tip are shown in cross section. All other illustrated components of the catheter 380 are not shown in cross section. The tip 384 may include an ultrasound imaging array 386. The tip 384 may, for example, be fabricated by overmolding the tip 384 over the ultrasound imaging array 386. The tip 384 may be temporarily interconnected to the tubular body 382 by a temporary bond 388 to keep the tip 384 secured while the catheter 380 is inserted into a patient. The temporary bond 388 may, for example, be achieved by an adhesive or a severable mechanical link. Any other appropriate method of achieving a severable bond may be used for the temporary bond. To aid in insertion, the tip 384 may have a rounded distal end. The tubular body 382 includes a lumen 390 for the introduction of an interventional device or other appropriate device (not shown). The catheter 380 also includes a cable 392 that electrically interconnects the ultrasound imaging array 386 in the tip 384 to an electrical interconnection member (not shown) within the wall of the tubular body 382. While the tip is temporarily attached to the tubular body 382, the cable 392 may be disposed within a portion of the lumen 390, as illustrated in FIG. 26A. The tubular body 382 may include a tubular body channel 394 running along the length of the tubular body 382. A corresponding tip channel 396 may be disposed within the tip 384. Together, the tubular body channel 394 and the tip channel 396 may be configured to accept an actuation member, such as a flat wire 398. The flat wire 398 may be configured such that it positions the tip 384 at about a right angle relative to the tubular body 382 (as illustrated in FIG. 26B) in the substantial absence of externally applied forces. In this regard, the flat wire 398 may be constructed from a shape memory material (e.g., Nitinol) with the memorized configuration including a 90 degree bend as shown in FIG. 25B. Moreover, the flat wire 398 may be configured such that it is operable to be advanced through the tubular body channel 394 and the tip channel 396.

In use, the catheter 380 may be inserted into a patient with the tip 384 temporarily bonded to the tubular body 382. While in the position illustrated in FIG. 26A, the ultrasound imaging array 386 may be operable and thus images may be generated to aid in positioning of the catheter 380 during catheter 380 insertion. Once the catheter 380 is in a desired position, the flat wire 398 may be advanced relative to the tubular body 382 and into the tip through the tubular body channel 394 and the tip channel 396. Once the flat wire 398 contacts the end of the tip channel 396 (and/or once friction between the flat wire 398 and the tip 384 reaches a predeterminable threshold), additional insertion force applied to the flat wire 398 may cause the temporary bond 388 to fail and release the tip 384 from the tubular body 382. Once released, further advancement of the flat wire 398 relative to the tubular body 382 may result in pushing the tip 384 away from the tubular body 382. Once free from the tubular body 382, the section of flat wire 398 between the tip 384 and the tubular body 382 may return to a memorized shape which may cause the tip 384 to displaced as illustrated in FIG. 26B. In such a position, the ultrasound imaging array 386 may be used to generate images of a volume distal to the catheter 380. An interventional device (not shown) may be advanced through the lumen 376. Furthermore, the force required to break the temporary bond 388 may be selected such that the flat wire 398 ends up being press fit into the tip channel 396 to a degree that allows a subsequent retraction of the flat wire 398 to draw the tip 384 proximate to the end of the tubular body 382 for further positioning and/or removal of the catheter 380 from the patient.

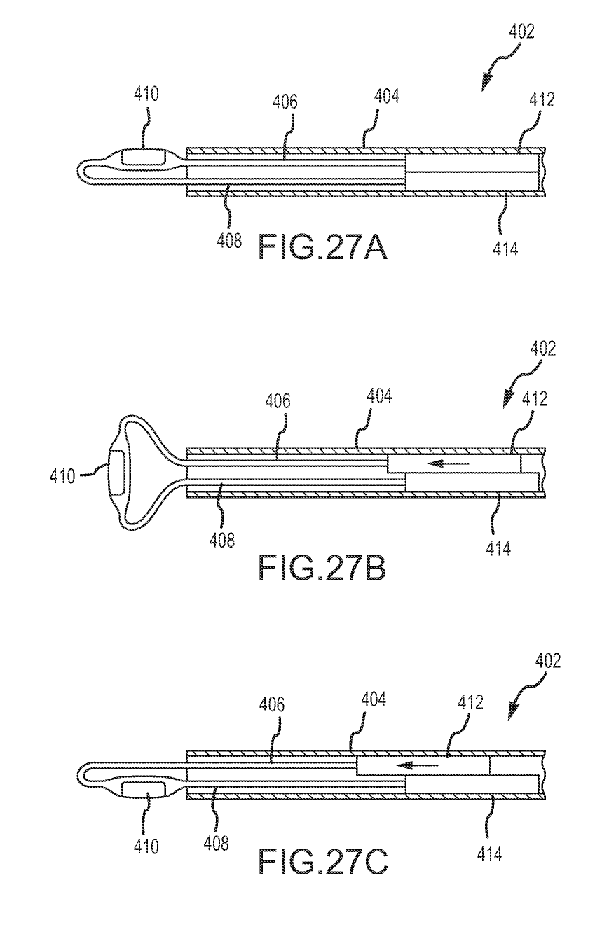

FIGS. 27A through 27C illustrate a catheter 402 that includes a tubular body 404. In FIGS. 27A through 27C, the tubular body 404 is shown in cross section. All other illustrated components of the catheter 402 are not shown in cross section. Disposed within a portion of the tubular body 404 are a first control cable 406 and a second control cable 408. The first and second control cables 406, 408 are operatively interconnected to opposite ends of an ultrasound imaging array 410. The control cables 406, 408 each have an appropriate level of stiffness such that, by moving the first control cable 406 relative to the second control cable 408, the position of the ultrasound imaging array 410 relative to the tubular body 404 may be manipulated. As shown in FIG. 27A, the control cables 406, 408 may be disposed such that the ultrasound imaging array 410 is pointed in a first direction (upward as shown in FIG. 27A). By moving the first control cable 406 in a distal direction relative to the second control cable 408, the ultrasound imaging array 410 may be adjusted to point in a distal direction (as shown in FIG. 27B). By moving the first control cable 406 still further in a distal direction relative to the second control cable 408, the ultrasound imaging array 410 may be adjusted to point in direction opposite form the first direction (downward as shown in FIG. 27C). It will be appreciated that any position between the illustrated positions may also be achieved. It will also be appreciated that the above described positions of the ultrasound imaging array 410 may be achieved by relative movement of the control cables 406, 408 and as such, may be achieved by anchoring either control cable 406, 408 relative to the tubular body 404 and moving the other of the control cables or by moving both control cables 406, 408 simultaneously. At least one of the control cables 406, 408 may contain electrical conductors to electrically interconnect to the ultrasound imaging array 410.

The first control cable 406 may be attached to a first half rod 412. The second control cable 408 may be attached to a second half rod 414. The half rods 412, 414 may each be half cylinders configured such that when proximate to each other, they form a cylinder about equal in diameter to the inner diameter of the tubular body 404. The half rods 412, 414 may be made of flexible and/or lubricious material (e.g., PTFE) and may be operable to flex along with the tubular body 404 (e.g., while the catheter 402 is disposed within the patient). The half rods 412, 414 may be disposed proximate to the distal end of the catheter 402, and the second half rod 414 may be fixed relative to the tubular body 404, while the first half rod 412 remains movable relative to the tubular body 404. Moreover, an actuator (not shown), such as a flat wire or the like, may be attached to the first half rod 412 and run along the length of the tubular body 404 to enable a user move the first half rod 412 relative to the second half rod 414 and thus manipulate the position of the ultrasound imaging array 410.

The repositioning of the ultrasound imaging array 410 has been described as a result of moving the first half rod 412 while the second half rod 414 remains stationary relative to the tubular body 404. In alternate embodiments, the ultrasound imaging array 410 may be repositioned by moving the second half rod 414 while the first half rod 412 remains stationary or by moving both the first half rod 412 and the second half rod 414 simultaneously, sequentially or a combination of simultaneously and sequentially.

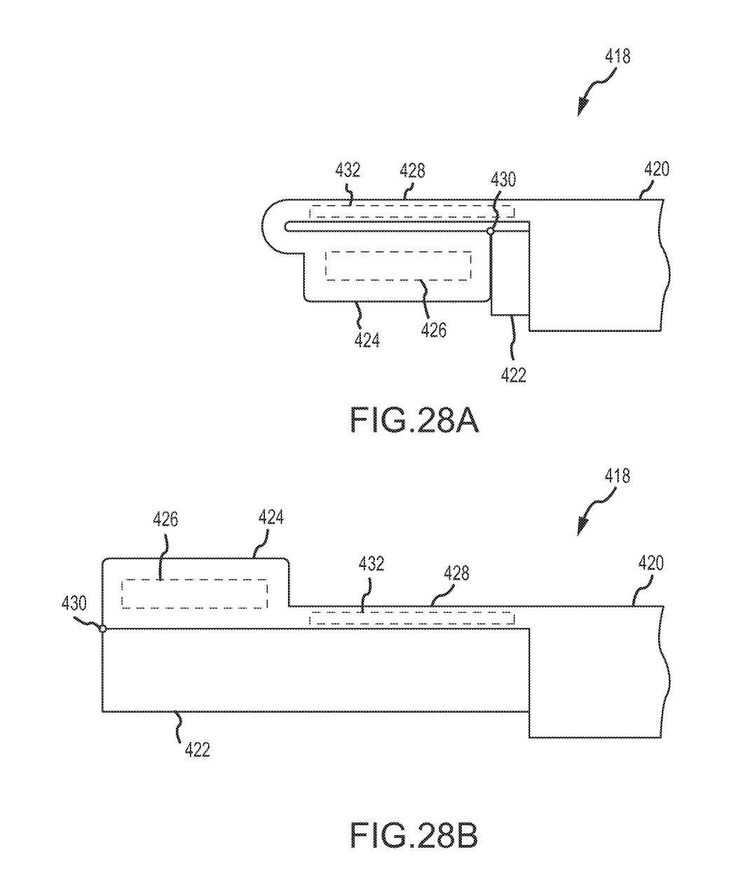

FIGS. 28A and 28B illustrate a catheter 418 that includes an outer tubular body 420 and an inner tubular body 422. The inner tubular body 422 may include a lumen therethrough. The catheter 418 also includes a tip portion 424 that includes an ultrasound imaging array 426. The tip portion 424 is interconnected to the outer tubular body 420 by a tip support 428. The tip support 428 may include an electrical interconnection member (e.g., flexboard, cable) to electrically interconnect to the ultrasound imaging array 426. Although illustrated as a single piece, the outer tubular body 420, the tip support 428, and the tip portion 424 may each be separate components that are joined together in an assembly process. One end of the tip portion 424 may be joined to the tip support 428 and the other end may be joined to the distal end of the inner tubular body 422 at a hinge 430. The hinge 430 may allow the tip portion 424 to rotate about the hinge 430 relative to the inner tubular body 422. The tip support 428 may be of a uniform or non-uniform predetermined stiffness to facilitate the positioning as illustrated in FIG. 28A (e.g., axial alignment of the tip portion 424 with the inner tubular body 422). The tip support 428 may include a shape memory material.

In the embodiment of FIGS. 28A and 28B and all other appropriate embodiments described herein, the hinge 430 or other appropriate hinge may be a live hinge (also known in the art as a “living” hinge) or any other appropriate type of hinge, and may be constructed from any appropriate material (e.g., the hinge may be a polymeric hinge). The hinge 430 or other appropriate hinge may be an ideal hinge and may include multiple components such as pins and corresponding holes and/or loops.

During insertion into a patient, the catheter 418 may be arranged as in FIG. 28A with the tip portion 424 in axial alignment with the inner tubular body 422 and a field of view of the ultrasound imaging array 426 pointing perpendicular to the longitudinal axis of the catheter 418 (downward as illustrated in FIG. 28A). In this regard, the catheter 418 may be substantially contained within a diameter equal to the outer diameter of the outer tubular body 420. As desired, the tip portion 424 may be pivoted relative to the inner tubular body 422 to vary the direction of the field of view of the ultrasound imaging array 426. For example, by moving the inner tubular body 422 distally relative to the outer tubular body 420, the tip portion 424 may be pivoted to the position illustrated in FIG. 28B such that the field of view of the ultrasound imaging array 426 is pointing upward. It will be appreciated that positions between those illustrated in FIGS. 28A and 28B may be achieved during rotation, including a position where the tip portion 424 is disposed vertically (relative to the position illustrated in FIGS. 28A and 28B) and the field of view of the ultrasound imaging array 426 is pointing distally. It will also be appreciated that once the tip portion 424 is disposed vertically, the distal end of the lumen of the inner tubular body 422 will be clear from obstruction by the tip portion 424 and an interventional device may then be inserted through the lumen.

In a variation of the embodiment of FIGS. 28A and 28B, the inner tubular body may be a collapsible lumen. In such an embodiment, introduction of the interventional device may be used to deploy the tip portion 424 to a distally looking position and subsequent retraction of the collapsible lumen may be used to return the tip portion 424 to the position of FIG. 28A.

In another variation of the embodiment of FIGS. 28A and 28B, the tip support 428 may include a stiffening member 432. The stiffening member 432 may be configured such that it remains straight during deployment of the catheter 418. As such, during pivoting of the tip portion 424, the tip support 428 may substantially only bend in the regions between the stiffening member 432 and the tip portion 424 and between the stiffening member 432 and the outer tubular body 420.

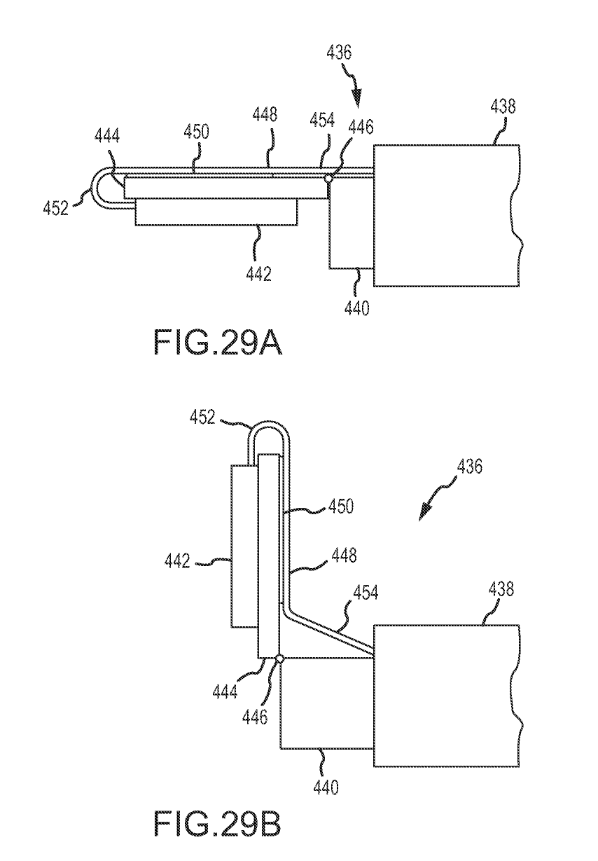

FIGS. 29A and 29B illustrate a catheter 436 that includes an outer tubular body 438 and an inner tubular body 440. The inner tubular body 440 may include a lumen therethrough. The catheter 436 also includes an ultrasound imaging array 442 interconnected to a tip support 444. The tip support 444 is interconnected to the distal end of the inner tubular body 440 at a hinge 446. The hinge 446 may allow the tip support 444 to rotate about the hinge 446 relative to the inner tubular body 440. An electrical interconnection member 448 may electrically interconnect to the ultrasound imaging array 442. The electrical interconnection member 448 is connected to a distal end of the ultrasound imaging array 442. The electrical interconnection member 448 may be bonded or otherwise fixed to a portion 450 of the tip support 444 on an opposite side of the tip support from the ultrasound imaging array 442. The electrical interconnection member 448 may include a loop 452 between the connection to the ultrasound imaging array 442 and the bonded portion 450. The bonded portion 450, by virtue of its fixed position relative to the tip support 444 may serve as a strain relief preventing strain associated with pivoting of the ultrasound imaging array 442 from being translated to the loop 452 and array 442 through the electrical interconnection member 448. A tether portion 454 of the electrical interconnection member 448 may be disposed between the bonded portion 450 and the point where the electrical interconnection member 448 enters into the outer tubular body 436. The tether portion 454 may be an unmodified portion of the electrical interconnection member 448 or it may be modified (e.g., structurally reinforced) to accommodate additional forces due to its serving as a tether. The tip support 444 and the ultrasound imaging array 442 may be encased or otherwise disposed within a tip (not shown).

During insertion into a patient, the catheter 436 may be arranged as in FIG. 29A with the ultrasound imaging array 442 in axial alignment with the inner tubular body 440 and a field of view of the ultrasound imaging array 442 pointing perpendicular to the longitudinal axis of the catheter 436 (downward as illustrated in FIG. 29A). In this regard, the catheter 436 may be substantially contained within a diameter equal to the outer diameter of the outer tubular body 438. As desired, the ultrasound imaging array 442 may be pivoted relative to the inner tubular body 440 by moving the inner tubular body 440 distally relative to the outer tubular body 438. Such relative motion will cause the ultrasound imaging array 442 to pivot about the hinge 446 due to the restraint of motion of the ultrasound imaging array 442 by the tether portion 454. The ultrasound imaging array 442 may be returned to the position illustrated in FIG. 29A by moving the inner tubular body 440 proximally relative to the outer tubular body 438.

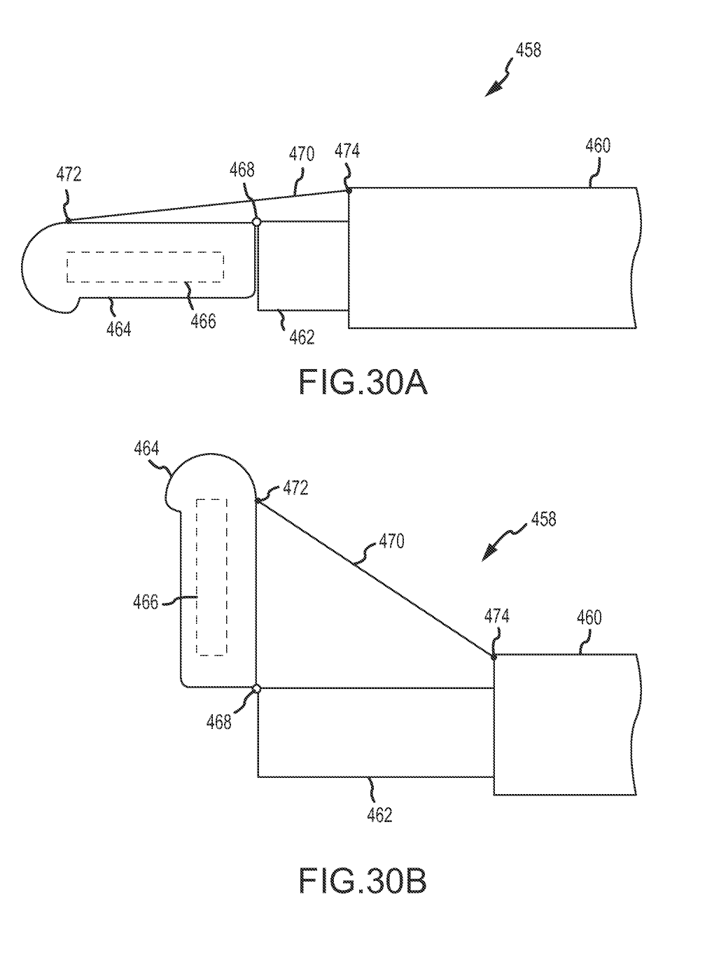

FIGS. 30A and 30B illustrate a catheter 458 that includes an outer tubular body 460 and an inner tubular body 462. The inner tubular body 462 may include a lumen therethrough. The catheter 458 also includes an ultrasound imaging array 466 disposed within a tip portion 464. The tip portion 464 is interconnected to the distal end of the inner tubular body 462 at a hinge 468. The hinge 468 may allow the tip portion 464 to rotate about the hinge 468 relative to the inner tubular body 462. The catheter 458 may further include a tether 470. The tether 470 may be anchored to a distal region of the tip portion 464 at tip anchor point 472. The tether 470 may be anchored to a distal end of the outer tubular body 460 at an outer tubular body anchor point 474. Any appropriate electrical interconnection scheme, such as those described herein, may be used with the catheter 458 of FIGS. 30A and 30B.

During insertion into a patient, the catheter 458 may be arranged as in FIG. 30A with the tip portion 464 in axial alignment with the inner tubular body 462 and a field of view of the ultrasound imaging array 466 pointing at a right angle to the longitudinal axis of the catheter 458 (downward as illustrated in FIG. 30A). Such positioning of the tip portion 464 may be facilitated by a spring or other appropriate mechanism or component biasing the tip portion 464 toward the position illustrated in FIG. 30A. In this regard, the catheter 458 may be substantially contained within a diameter equal to the outer diameter of the outer tubular body 460. As desired, the tip portion 464 may be pivoted relative to the inner tubular body 462 by moving the outer tubular body 460 proximally relative to the inner tubular body 462. Such relative motion will cause the tip portion 464 to pivot about the hinge 468 due to the restraint of motion of the tip portion 464 by the hinge 468. The tip portion 464 may be returned to the position illustrated in FIG. 30A by moving the outer tubular body 460 distally relative to the inner tubular body 462 and allowing the biasing mechanism or component to return the tip portion 464 to the position illustrated in FIG. 30A. In an alternate embodiment, the tether 470 may possess enough rigidity such that substantially no biasing of the tip portion 464 to the position illustrated in FIG. 30A is needed.

It will be appreciated that the hinges 446, 468 of FIGS. 29A and 30A, respectively (along with, where appropriate, any other hinge discussed herein), may be in the form of live hinges such as the live hinge that is part of the support 174 illustrated in FIG. 14C. It will also be appreciated that the hinges 446, 468 of FIGS. 29A and 30A, respectively, may be in the form of live hinges and array supports that are parts of the inner tubular bodies 440, 462, respectively. Such inner tubular bodies that also serve as supports for the arrays would be similar in configuration to the outer tubular body 264 with support portion 266 illustrated in FIG. 20B.

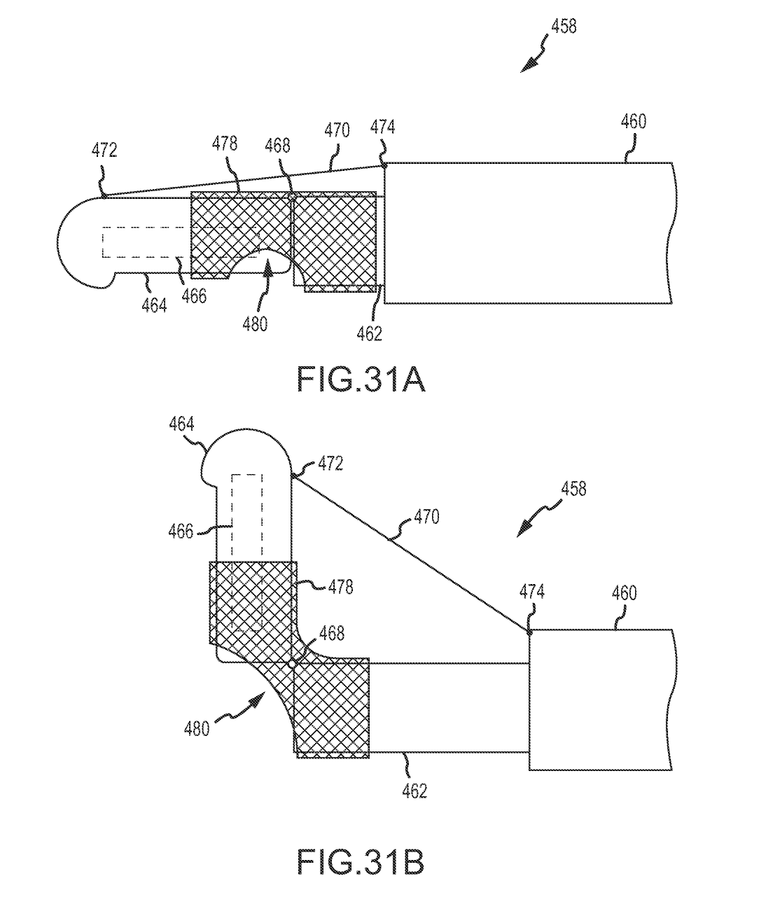

FIGS. 31A and 31B illustrate the catheter 458 and components thereof of FIGS. 30A and 30B with the addition of a resilient tube 478. The resilient tube 478 may act as a biasing mechanism to bias the tip portion 464 toward the position illustrated in FIG. 31A. The resilient tube 478 may also assist in making the catheter 458 more atraumatic to a vessel into which it has been inserted. The resilient tube 478 may include, for example, an elastic material capable of being deformed as shown in FIG. 31B when the tip portion 464 is deflected and returning toward the state illustrated in FIG. 31A once the deflection force has been removed or reduced (e.g., when the outer tubular body 460 is returned to the position relative to the inner tubular body 462 illustrated in FIG. 31A). To preserve the ability to introduce an interventional device through the lumen of the inner tubular body 462, the resilient tube 478 may include an opening 480. When in the position illustrated in FIG. 31B, the opening 480 may align with the lumen and therefore not interfere with an interventional device deployed through the lumen. The resilient tube 478 may be interconnected to the inner tubular body 462 and the tip portion 464 in any appropriate manner, such as for example, shrink fit, bonding, welding, or with an adhesive. Although illustrated as occupying the field of view of the ultrasound imaging array 466, alternatively, the resilient member 478 may be disposed such that it is not within the field of view of the ultrasound imaging array 466. This may be accomplished by reconfiguring the resilient member 478 relative to as illustrated and/or by repositioning the ultrasound imaging array 466 relative to as illustrated. The resilient member 478, or a similar, appropriately modified resilient member, may be used in any suitable embodiment disclosed herein.

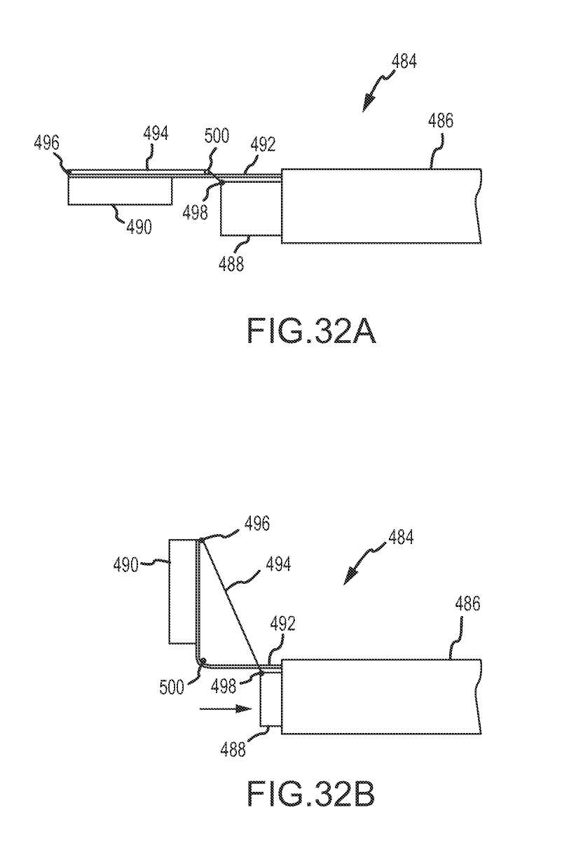

FIGS. 32A and 32B illustrate a catheter 484 that includes an outer tubular body 486 and an inner tubular body 488. The inner tubular body 488 may include a lumen therethrough. The catheter 484 also includes an ultrasound imaging array 490 interconnected to an electrical interconnection member 492. The electrical interconnection member 492 may, for example, be in the form of a flexboard interconnected to a spirally wound electrical interconnection member within the outer tubular body 486 on one end and interconnected to the ultrasound imaging array 490 on the other end. The catheter 484 also includes a tether 494 anchored on one end to a distal end of the electrical interconnection member 492 and/or ultrasound imaging array 490 at a tether to array anchor 496. On the other end, the tether 494 may be anchored to the inner tubular body 488 at a tether to inner tubular body anchor 498. As shown in FIG. 32A, the tether 494 may be disposed such that it bends around a buckling initiator 500 when the ultrasound imaging array 490 is aligned with the inner tubular body 488. The electrical interconnection member 492 may serve both to provide an electrical connection to the ultrasound imaging array 490 and act as a spring member to bias the ultrasound imaging array 490 toward the position illustrated in FIG. 32A (e.g., aligned with the inner tubular body 488). To achieve this, the electrical interconnection member 492 may include a stiffener and/or spring element interconnected to the electrical interconnection member 492 in the region between the ultrasound imaging array 490 and the outer tubular body 486. A tip (not shown) may be molded over the ultrasound imaging array 490.

During insertion into a patient, the catheter 484, with an appropriately configured tip (not shown), may be arranged as in FIG. 32A with the ultrasound imaging array 490 in axial alignment with the inner tubular body 488 and a field of view of the ultrasound imaging array 490 pointing generally perpendicularly from the longitudinal axis of the catheter 484 (illustrated as downward in FIG. 32A). In this regard, the catheter 484 may be substantially contained within a diameter equal to the outer diameter of the outer tubular body 486. As desired, the ultrasound imaging array 490 may be pivoted relative to the inner tubular body 488 by moving the inner tubular body 440 proximally relative to the outer tubular body 486. Such relative motion will place the tether 494 in tension, resulting in a downward force by the tether 494 on the buckling element 500. The downward force may cause the electrical interconnection member 492 to buckle in a controlled manner such that the electrical interconnection member 492 pivots in a clockwise direction (relative to the view of FIG. 32A). Once the buckling has been initiated, continued relative movement of the inner tubular body 488 may result in the ultrasound imaging array 490 pivoting to the forward-looking position shown in FIG. 32B. The ultrasound imaging array 490 may be returned to the position illustrated in FIG. 32A by moving the inner tubular body 488 distally relative to the outer tubular body 438. In such a case, the aforementioned biasing of the electrical interconnection member 492 may result in the ultrasound imaging array 490 returning to the position illustrated in FIG. 32A.

It will be appreciated that, where appropriate, the electrical interconnection members described herein that are disposed between tubular bodies and ultrasound imaging arrays that move relative to those tubular bodies, may be configured to additionally serve as biasing members (such as described above with respect to FIGS. 32A and 32B).

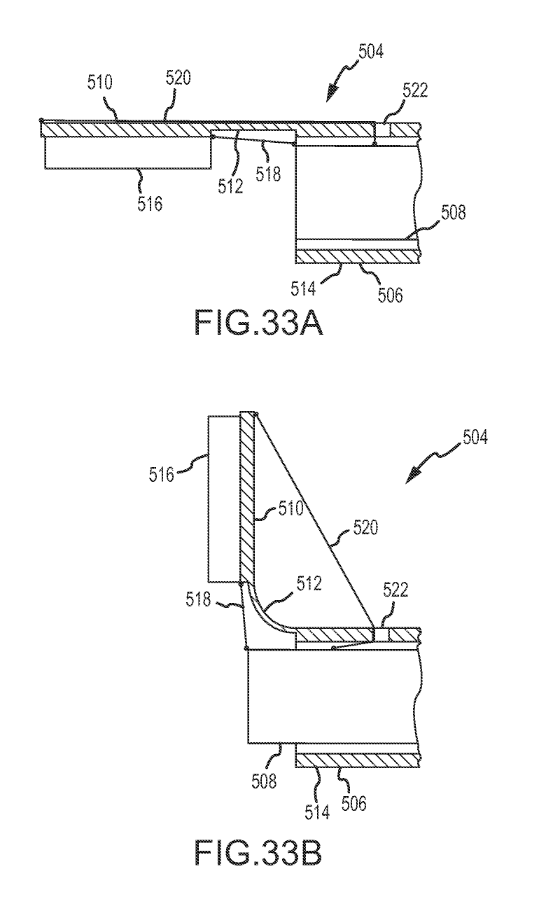

FIGS. 33A and 33B illustrate a catheter 504 that includes an outer tubular body 506 and an inner tubular body 508. The inner tubular body 508 may include a lumen therethrough. In FIGS. 33A and 33B, the outer tubular body 506 is shown in cross section. All other illustrated components of the catheter 504 are not shown in cross section. The outer tubular body 506 includes a support portion 510 and a hinge portion 512 disposed between the support portion 510 and a tubular portion 514 of the outer tubular body 506. The hinge portion 512 may generally restrict the motion of the support portion 510 to pivoting relative to the tubular portion 514 (e.g., pivoting between the position shown in FIG. 33A and the position shown in 33B).

The hinge portion 512 may, as illustrated in FIGS. 33A and 33B, be an appropriately sized portion of the outer tubular body 506 and/or it may include additional material such as a support member (e.g., to increase stiffness). In a variation of the embodiment of FIGS. 33A and 33B, the support portion 510 and hinge portion 512 may be replaced by a separate member that may be configured similarly to, for example, supports 160, 168, 174 and/or 180, with the modification that the respective tubular body interface portion be sized and configured to be attached to the outer tubular body 506.

An ultrasound imaging array 516 may be interconnected to the support portion 510. A first end of a first tether 518 may be interconnected to a distal end of the inner tubular body 508 and a second end of the first tether 518 may be interconnected to a proximal end of the support portion 510. A first end of a second tether 520 may be interconnected to the inner tubular body 508 and a second end of the second tether 520 may be interconnected to a distal end of the support portion 510. The second tether may be threaded through a through hole 522 in the outer tubular body 506.

To pivot the support portion 510 and its attached ultrasound imaging array 516 from the position illustrated in FIG. 33a (e.g., aligned with the inner tubular body 508) to the position illustrated in FIG. 33B (e.g., perpendicular to a longitudinal axis of the catheter 504 and forward looking), the inner tubular body 508 is moved distally relative to the outer tubular body 506. Such movement results in the second tether 520 being drawn into the interior of the outer tubular body 506 through the through hole 522. As the second tether is drawn through the through hole 522, the effective length of the tether between the through hole 522 and the distal end of the support portion 510 is shortened, causing the support portion 510 to pivot. To return the support portion 510 to the position illustrated in FIG. 33A from the position illustrated in FIG. 33B, the inner tubular body 508 is moved proximally relative to the outer tubular body 506. Such movement results in the inner tubular body 508 pulling (by virtue of their interconnection via the first tether 518) the support portion 510 back toward a position where the support portion 510 is aligned with the inner tubular body 508. It will be appreciated that when causing one of the tethers 518, 520 to be in tension due to movement of the inner tubular body 508 relative to the outer tubular body 506, tension will be relieved in the other one of the tethers 518, 520. In an alternative configuration of catheter 504, the first and second tethers 518, 520 may be combined into a single tether anchored along the inner tubular body 508 as shown and threaded along the support portion 510. Such a tether may be anchored to the support portion 510 at a single point.

The catheter 504 may also include a tip portion (not shown) that may be molded over the support portion 510, the ultrasound imaging array 516, and/or any other appropriate components. Any appropriate electrical interconnection, such as those described herein, may be used with the catheter 504 of FIGS. 33A and 33B.

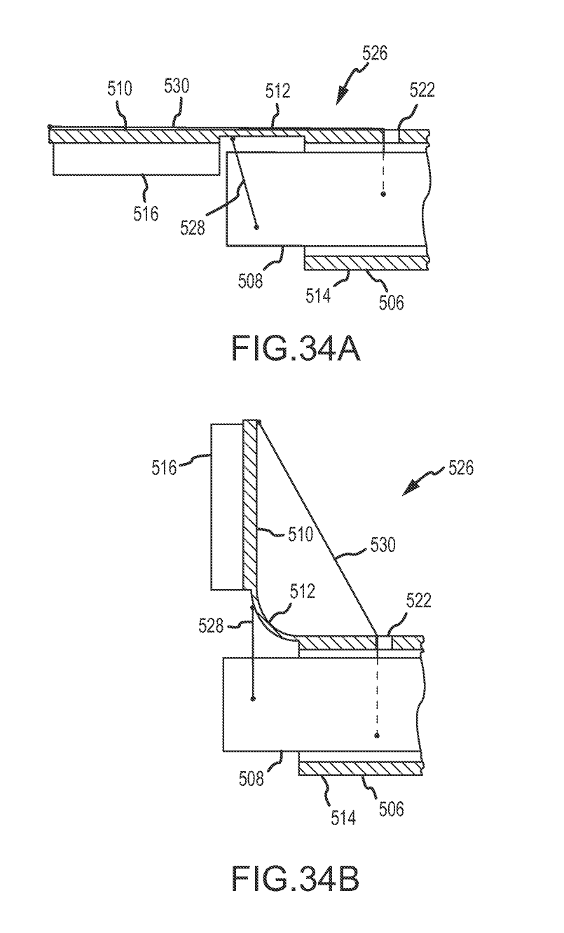

FIGS. 34A and 34B present catheter 526 that is a variation of the catheter 504 of FIGS. 33A and 33B. As such, similar components are similarly numbered and will not be discussed with reference to FIGS. 34A and 34B. A first end of a first tether 528 may be interconnected to a sidewall of the inner tubular body 508 and a second end of the first tether 528 may be interconnected to a distal point on the hinge portion 512. A first end of a second tether 530 may be interconnected to the sidewall of the inner tubular body 508 at a point along the length of the inner tubular body 508 that corresponds to the position of the through hole 522 and a second end of the second tether 520 may be interconnected to a distal end of the support portion 510. The second tether may be threaded through the through hole 522 in the outer tubular body 506. The inner tubular body 508 may be disposed such that a distal portion of it extends distally from the distal end of the outer tubular body 506. The inner tubular body 508 is rotatable relative to the outer tubular body 506.

With the support portion 510 aligned with the tubular portion 514 as shown in FIG. 34A, the tethers 528, 530 may be disposed as follows. The first tether 528 may be at least partially wrapped about and anchored to the outer circumference of the inner tubular body 508. The second tether 530 may be at least partially wrapped about, in a direction opposite from that of the first tether 528, and anchored to the outer circumference of the inner tubular body 508. As illustrated in FIG. 34A, when seen from the perspective of a point distal to the distal end of the inner tubular body 508 and looking toward the distal end of the inner tubular body 508 (hereinafter referred to as an end view), the first tether 528 is partially wrapped about the inner tubular body 508 in a clockwise direction and the second tether 530 is partially wrapped about the inner tubular body 508 in a counterclockwise direction. The tethers 528, 530 may be in the form of cord like members able to transmit tensile forces along their length and to conformally wrap about the inner tubular body 508. In an arrangement, the tethers 528, 530 may be in the form of a spring wound about the inner tubular body 508.

To pivot the support portion 510 and its attached ultrasound imaging array 516 from the position illustrated in FIG. 34a (e.g., aligned with the inner tubular body 508) to the position illustrated in FIG. 34B (e.g., perpendicular to a longitudinal axis of the catheter 526 and forward looking), the inner tubular body 508 is rotated counterclockwise (as seen in an end view) relative to the outer tubular body 506. Such rotation results in the second tether 530 being drawn into the interior of the outer tubular body 506 through the through hole 522 due to its wrapping about the inner tubular body 508. As the second tether is drawn through the through hole 522, the effective length of the tether between the through hole 522 and the distal end of the support portion 510 is shortened, causing the support portion 510 to pivot. Simultaneously, the first tether 528 is being unwrapped from the inner tubular body 508. To return the support portion 510 to the position illustrated in FIG. 34A from the position illustrated in FIG. 34B, the inner tubular body 508 is rotated in a clockwise direction (as seen in an end view) relative to the outer tubular body 506. Such rotation results in the first tether 528 being wrapped about the inner tubular body 508, thus pulling the support portion 510 back toward the position illustrated in FIG. 34A. Simultaneously, the second tether 530 is being unwrapped from the inner tubular body 508. Where the catheter 526 is configured such that the support portion 510 is biased toward the position illustrated in FIG. 34A, the first tether 528 may be unnecessary (e.g., the biasing may be adequate to return the support portion 510 to the position illustrated in FIG. 34A by unwrapping the second tether 530). Along the same lines, where the catheter 526 is configured such that the support portion 510 is biased toward the position illustrated in FIG. 34B, the second tether 530 may be unnecessary (e.g., the biasing may be adequate to move the support portion 510 to the position illustrated in FIG. 34B by unwrapping the first tether 528). Similarly, the first tether 518 of the catheter 504 of FIGS. 33A and 33B may be unnecessary where the support portion 510 is biased toward the position illustrated in FIG. 33A, and the second tether 520 of the catheter 504 of FIGS. 33A and 33B may be unnecessary where the support portion 510 is biased toward the position illustrated in FIG. 33B.

The catheter 526 may also include a tip portion (not shown) that may be molded over the support portion 510, the ultrasound imaging array 516, and/or any other appropriate components. Any appropriate electrical interconnection, such as those described herein, may be used with the catheter 526 of FIGS. 34A and 34B.

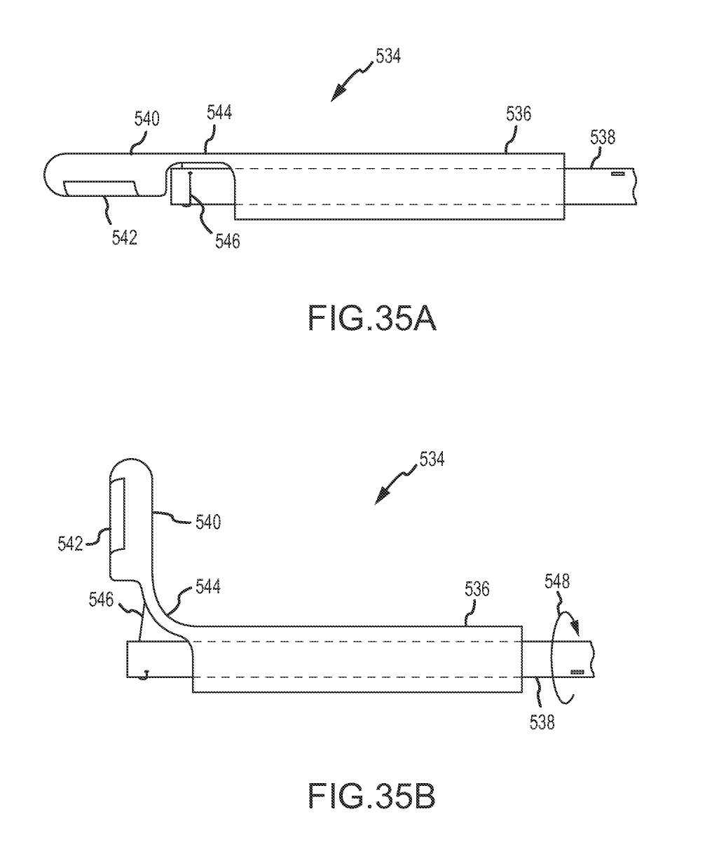

FIGS. 35A and 35B illustrate a catheter 534 that includes an outer tubular body 536 and an inner tubular body 538. The inner tubular body 538 may include a lumen therethrough. The outer tubular body 536 includes a support portion 540 and a hinge portion 544. The hinge portion 544 may be biased such that it generally positions the support portion 540 such that the support portion 540 is at about a right angle relative to the inner tubular body 538 (as illustrated in FIG. 35B) in the substantial absence of externally applied forces. An ultrasound imaging array 542 may be interconnected to the support portion 540. The hinge portion 544 may be an appropriately sized portion of the outer tubular body 536 and/or it may include additional material (e.g., to increase stiffness).

The catheter 534 includes a tether 546 disposed between a distal portion of the hinge portion 544 and the inner tubular body 538. The tether 546 may be at least partially wrapped about and anchored to the outer circumference of the inner tubular body 538. The tether 546 may be in the form of a cord like member able to transmit tensile forces along its length and to conformally wrap about the inner tubular body 538.

To pivot the support portion 540 and its attached ultrasound imaging array 542 from the position illustrated in FIG. 35A (e.g., aligned with the inner tubular body 538) to the position illustrated in FIG. 35B (e.g., perpendicular to a longitudinal axis of the catheter 534 and forward looking), the inner tubular body 538 may be rotated clockwise (as seen in an end view) relative to the outer tubular body 536. Such rotation results in the tether 546 being unwrapped from the inner tubular body 538 and the support portion 540 moving toward the position illustrated in FIG. 35B due to the aforementioned biasing of the hinge portion 544.

To return the support portion 540 to the position illustrated in FIG. 35A from the position illustrated in FIG. 35B, the inner tubular body 538 may be rotated in a counterclockwise direction (as seen in an end view) relative to the outer tubular body 536. Such rotation results in the tether 546 wrapping about the inner tubular body 538, thus pulling the support portion 540 back toward the position illustrated in FIG. 35A.

The catheter 534 may also include any appropriate electrical interconnection to the ultrasound imaging array 542, including appropriate connection schemes described herein. In a variation of the embodiment of FIG. 35A, the support portion 540 and hinge portion 544 may be replaced by a separate member that may be configured similarly to, for example, supports 160, 168, 174 and/or 180, with the modification that the respective tubular body interface portion be sized and configured to be attached to the outer tubular body 536.

In use, the catheter 534 may be inserted into a patient with the support portion 540 aligned with the outer tubular body 536. Once the catheter 534 is in a desired position, the inner tubular body 538 may be rotated relative to the outer tubular body to allow the hinge portion 544 to move the support portion 540 to a desired angle relative to the longitudinal axis of the catheter 534. An interventional device (not shown) may be advanced through the lumen within the inner tubular body 538.

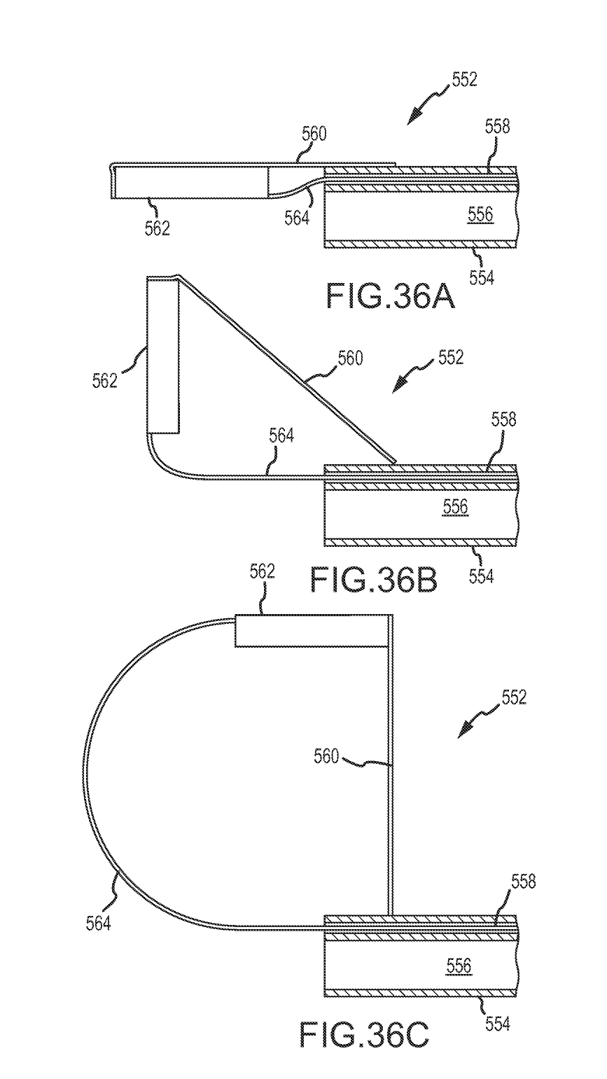

FIGS. 36A through 36C illustrate a catheter 552 that includes a tubular body 554. The tubular body 554 includes a lumen 556 therethrough. The tubular body 554 further includes a channel 558 running through a sidewall of the tubular body 554. A proximal end of an arm 560 is attached to the tubular body 554 in a manner such that the arm 560 may pivot relative to the tubular body 554. The arm 560 may be of sufficient rigidity to allow for the pivoting of an ultrasound imaging array 562 as described below. A distal end of the ultrasound imaging array 562 may be interconnected to a distal end of the arm 560 such that when the ultrasound imaging array 562 is aligned with the tubular body 554, a rear face (pointing upward in the orientation shown in FIG. 36A) of the ultrasound imaging array 562 may be generally parallel to the arm 560. The catheter 552 further includes a push wire 564 running along the channel 558. A distal end of the push wire 564 is interconnected to a proximal end of the ultrasound imaging array 562. The interconnection between the distal end of the push wire 564 and the proximal end of the ultrasound imaging array 562 may be a rigid connection as illustrated in FIGS. 36A through 36C, or it may be a hinged connection or any other appropriate type of connection. The interconnection point between the push wire 564 and the ultrasound imaging array 562 may be disposed closer a front face (pointing downward in the orientation shown in FIG. 36A) of the ultrasound imaging array 562 than to the rear face of the ultrasound imaging array 562. Such disposition may aid in initial displacement of the ultrasound imaging array 562 away from the position illustrated in FIG. 36A by imparting a larger torque on the ultrasound imaging array 562 than would be achieved if the push wire 564 were closer to being collinear with the arm 560.

To pivot the ultrasound imaging array 562 from the position illustrated in FIG. 36A (e.g., aligned with the tubular body 554) to the position illustrated in FIG. 36B (e.g., perpendicular to a longitudinal axis of the catheter 552 and forward looking), the push wire 564 may be advanced relative to the tubular body 554. As illustrated in FIGS. 36A and 36B, this relative motion, in combination with the arm's 560 maintenance of a fixed distance between its attachment point to the tubular body 554 and the distal end of the ultrasound imaging array 562 may result in the ultrasound imaging array 562 pivoting to the forward-looking position of FIG. 36B. It will be appreciated that the push wire 564 should have appropriate column strength to transfer the necessary degree of force to move the ultrasound imaging array 562 as illustrated. To return the ultrasound imaging array 562 to the position illustrated in FIG. 36A from the position illustrated in FIG. 36B, the push wire 564 may be withdrawn.

The catheter 552 may also include any appropriate electrical interconnection to the ultrasound imaging array 562, including appropriate connection schemes described herein. For example, an electrical interconnection member may be disposed along the arm 560 and may electrically interconnect the ultrasound imaging array 562 to an electrical interconnection member disposed within a wall of the tubular body 554. A tip (not shown) may be molded over the ultrasound imaging array 562.

The catheter 552 may be further operable to deploy the ultrasound imaging array 562 to the position illustrated in FIG. 36C where the ultrasound imaging array 562 is facing in a direction substantially opposite from the insertion position illustrated in FIG. 36A. This may be achieved by continuing to advance the push wire 564 relative to the tubular body 554 beyond the position shown in FIG. 36B. It will be appreciated that further advancement of the push wire 564 may yield further pivoting of the ultrasound imaging array 562 beyond that illustrated in FIG. 36C. It will also be appreciated that the ultrasound imaging array 562 may be positioned in any intermediate position between the discussed positions.

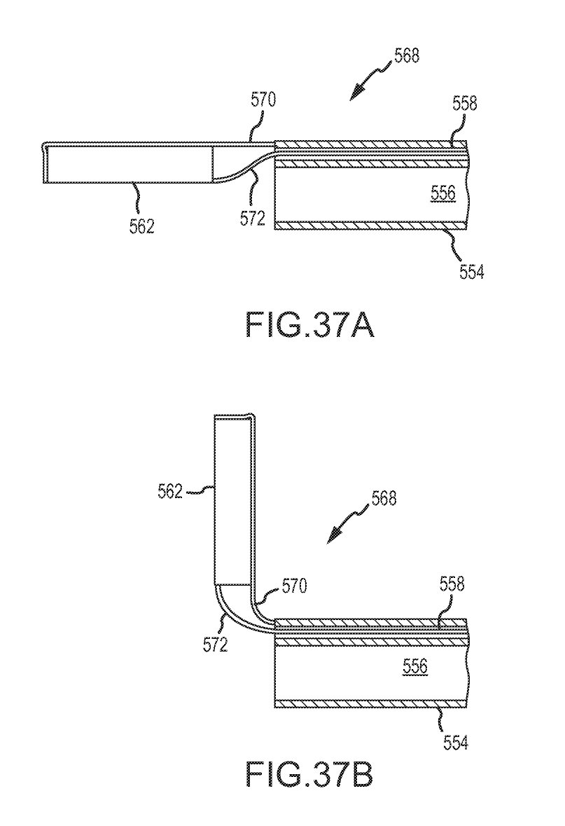

FIGS. 37A and 37B present a catheter 568 that is a variation of the catheter 552 of FIGS. 36A and 36B. As such, similar components are similarly numbered and will not be discussed with reference to FIGS. 37A and 37B. An arm 570 is attached to the distal end of the tubular body 554. The arm 570 may, for example, be in the form of a flexboard that includes electrical conductors for interconnection to the ultrasound imaging array 562. In embodiments where the arm 570 includes a flexboard, the flexboard may include reinforcing or other members to facilitate the use of the flexboard as described below (e.g., use as a hinge). The arm 570 may be of sufficient flexibility to allow for the pivoting of an ultrasound imaging array 562 as described below. The arm 570 may be connected to the ultrasound imaging array 562 along the rear face of the ultrasound imaging array 562. The catheter 568 further includes a push wire 572 running along the channel 558. A distal end of the push wire 572 is interconnected to a proximal end of the ultrasound imaging array 562 as in catheter 552 of FIGS. 36A and 36B.

To pivot the ultrasound imaging array 562 from the position illustrated in FIG. 37A to the position illustrated in FIG. 37B, the push wire 572 may be advanced relative to the tubular body 554. As illustrated in FIGS. 37A and 37B, this relative motion, in combination with the arm's 570 flexibility may result in the ultrasound imaging array 562 pivoting to the forward-looking position of FIG. 37B. To return the ultrasound imaging array 562 to the position illustrated in FIG. 37A from the position illustrated in FIG. 37B, the push wire 572 may be withdrawn. A tip (not shown) may be molded over the ultrasound imaging array 562.

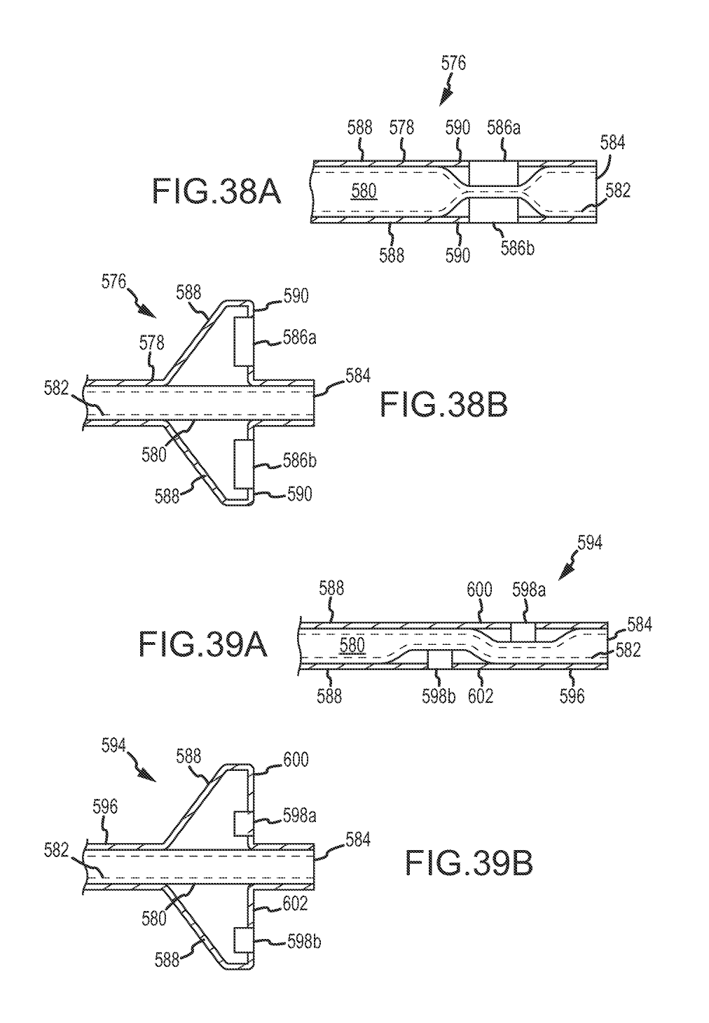

FIGS. 38A and 38B present a catheter 576 that is configured somewhat similarly to the catheters of FIGS. 7A through 8D in that relative movement of components can cause a deflectable portion of an outer tubular body 578 to deflect an ultrasound imaging array to a forward-looking position. In the case of the catheter 576, the ultrasound imaging array may include a first imaging array 586a and a second imaging array 586b. As illustrated in FIG. 38A, an introductory configuration (e.g., the configuration of the catheter 576 as it is introduced into a patient) of the catheter 576 includes the first and second imaging arrays 586a, 586b in a back-to-back relationship, with an at least partially collapsed inner tubular body 580 between the imaging arrays 586a, 586b. The inner tubular body 580 may include a lumen 582 therethrough. The outer tubular body 578 and the inner tubular body 580 may be fixed relative to each other at a single point at a distal end 584 of the catheter 576.

To move the imaging arrays 586a, 586b from the positions illustrated in FIG. 38A (e.g., side-looking) to the positions illustrated in FIG. 38B (e.g., forward-looking), a proximal end of the outer tubular body 578 may be pushed distally while maintaining the position of the inner tubular body 580 (and/or a proximal end of the inner tubular body 580 may be drawn proximally while maintaining the position of the outer tubular body 578). Such relative motion may cause portions of the outer tubular body 578 containing the imaging arrays 586a, 586b to be displaced outward, thus pivoting the imaging arrays 586a, 586b to forward-looking positions as illustrated in FIG. 38B. To aid in controlling the motion of the imaging arrays 586a, 586b, the outer tubular body 578 may include first rigid portions 588 (e.g., of sufficient rigidity to perform the functions as described herein) that remain substantially straight as the imaging arrays 586a, 586b are pivoted. The first rigid portions 588 may be formed by adding appropriate stiffening members to the outer tubular body 578. Furthermore, the outer tubular body 578 may include second rigid portions 590 disposed proximate to the imaging arrays 586a, 586b. The second rigid portions 590 may serve to reduce or eliminate bending forces from being transmitted to the imaging arrays 586a, 586b during pivoting and to aid in alignment of the imaging arrays 586a, 586b. As shown in FIG. 38B, once the imaging arrays 586a, 586b are positioned in the forward-looking position, the lumen 582 is available for delivery of a suitable interventional device to a point distal to the catheter distal end 584.

The catheter 576 may also include any appropriate electrical interconnection to the imaging arrays 586a, 586b, including appropriate connection schemes described herein. For example, an electrical interconnection member may be disposed along the outer tubular body 578 and first and second rigid portions 588, 590.

FIGS. 39A and 39B present a catheter 594 that is a variation of the catheter 576 of FIGS. 38A and 38B. As such, similar components are similarly numbered and will not be discussed with reference to FIGS. 39A and 39B. As illustrated in FIG. 39A, an introductory configuration of the catheter 594 includes a first imaging array 598a and a second imaging array 598b arranged in an offset (e.g., they occupy different positions along the length of the catheter 594) back-to-back arrangement, with an at least partially collapsed inner tubular body 580 proximate to the imaging arrays 598a, 598b. The inner tubular body 580 may include a lumen 582 therethrough. An outer tubular body 596 and the inner tubular body 580 may be fixed relative to each other at a distal end 584 of the catheter 594.

The imaging arrays 598a and 598b may be pivoted in a manner similar to as discussed above with reference to FIGS. 38A and 38B. The outer tubular body 596 may include second rigid portions 600, 602 disposed proximate to the imaging arrays 598a, 598b. The second rigid portions 600, 602 may serve to reduce or eliminate bending forces from being transmitted to the imaging arrays 598a, 598b during pivoting and to aid in alignment of the imaging arrays 598a, 598b. As shown in FIG. 38B, the second rigid portions 600, 602 may each position the imaging arrays 598a, 598b at unique distances from a central axis of the catheter 594.

The imaging arrays 586a, 586b, 598a, 598b of FIGS. 38A through 39B are illustrated as proximate to distal ends 584 of the catheters 576, 594. In alternate configurations, the imaging arrays 586a, 586b, 598a, 598b may be disposed at a predetermined distance form the distal ends 584. In this regard, the imaging arrays 586a, 586b, 598a, 598b may be disposed at any appropriate point along the catheters 576, 594.

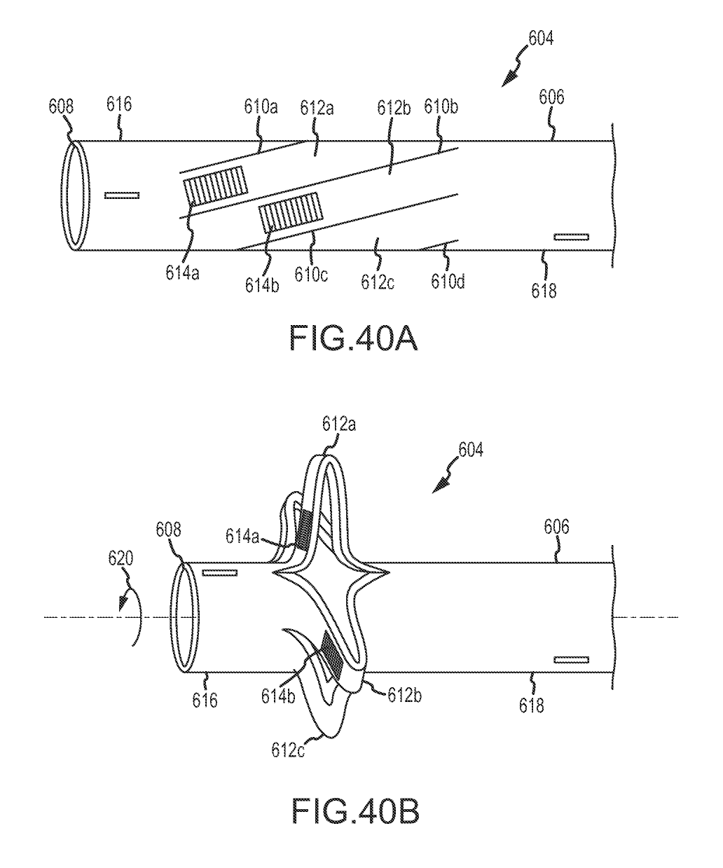

FIGS. 40A and 40B present a catheter 604 that includes a tubular body 606 with a lumen 608 therethrough. The tubular body 606 includes a plurality of spirally disposed slits (slits 610a, 610b, 610c and 610d are visible in FIG. 40A) defining a plurality of arms such as arms 612a, 612b and 612c. Any appropriate number of slits to define any appropriate number of arms may be included in the tubular body 606. At least one of the arms may include an ultrasound imaging array. For example, in the embodiment illustrated in FIGS. 40A and 40B, arms 612a and 612b include ultrasound imaging arrays 614a and 614b, respectively. A relative rotation (e.g., in the direction of directional arrow 620) of a distal portion 616 (distal to the arms 612a-612c) of the tubular body 606 to a proximal portion 618 (proximal to the arms 612a-612c) of the tubular body 606 may cause the arms to deflect outwardly as illustrated in FIG. 40B, moving the ultrasound imaging arrays 614a and 614b to generally forward-looking positions. An interventional device may be advanced through the lumen 608.

The relative rotation between the distal portion 616 and the proximal portion 618 may be achieved in any appropriate manner. For example, the catheter 604 may include an inner tubular body (not shown) similar to the inner tubular body of catheter 576 of FIGS. 38A and 38B. Such an inner tubular body may be secured to the tubular body 606 in the distal portion 616. In such an embodiment, rotation of the inner tubular body relative to the tubular body 616 may cause the distal portion 616 (by virtue of its securement to the inner tubular body) to rotate relative to the proximal portion 618, thereby causing the arms to deflect outwardly as illustrated in FIG. 40B. Moreover, the inner tubular body may include a lumen therethrough (e.g., for deployment of an interventional device).

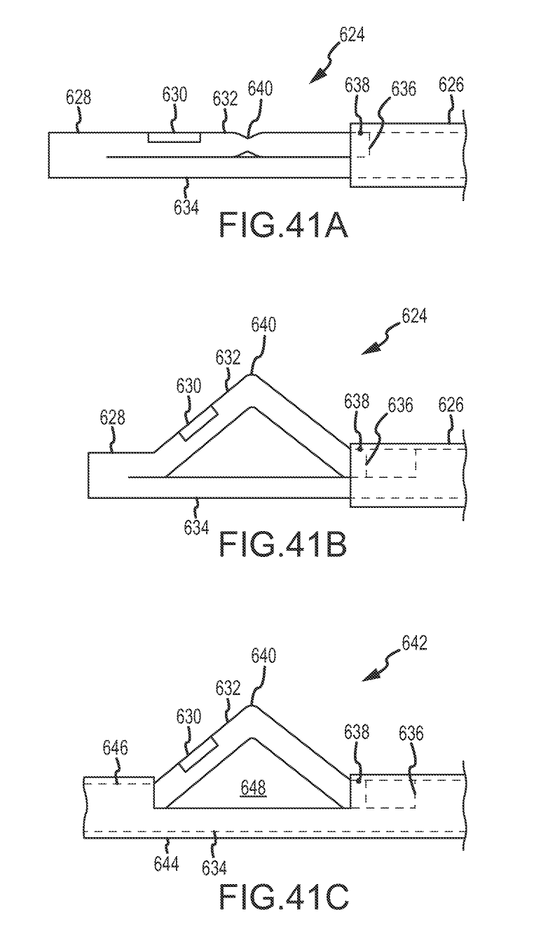

FIGS. 41A and 41B present a catheter 624 that includes an outer tubular body 626 and an inner tubular body 628. The inner tubular body 628 includes a lumen therethrough. An ultrasound imaging array 630 is interconnected to the inner tubular body 628. In the vicinity of the ultrasound imaging array 630, the inner tubular body 628 may be cut along the longitudinal axis of the inner tubular body 628, thus dividing the inner tubular body 628 into a first longitudinal portion 632 and a second longitudinal portion 634. The ultrasound imaging array 630 is disposed on the distal half of the first longitudinal portion 632. Distal ends of the first and second longitudinal portions 632, 634 may remain interconnected to each other and to a distal portion of the inner tubular body 628. A proximal end of the first longitudinal portion 632 may be severed from the remainder of the inner tubular body 628 along a transverse cut 636. The second longitudinal portion 634 remains connected to the inner tubular body 628. The proximal end of the first longitudinal portion 632 may be bonded or otherwise attached to the outer tubular body 626 at a bond 638. The first longitudinal portion 632 may include a hinge 640. The hinge 640 may be a portion of the first longitudinal portion 632 modified such that the first longitudinal portion 632 preferentially buckles and/or bends at the hinge 640 when the outer tubular body 626 is advanced distally relative to the inner tubular body 628 (and/or the inner tubular body 628 is retracted proximally relative to the outer tubular body 626).

To move the ultrasound imaging array 630 from the position illustrated in FIG. 41A (e.g., side-looking) to the position illustrated in FIG. 41B (e.g., at least partially forward-looking), the outer tubular body 626 is advanced distally relative to the inner tubular body 628. Since the proximal end of the first longitudinal portion 632 is bonded to the outer tubular body 626 and the distal end is connected of the inner tubular body 628, advancement of the outer tubular body 626 will cause the first longitudinal portion 632 to buckle at the hinge 640, thus pivoting the ultrasound imaging array 630 such that a field of view of the ultrasound imaging array 630 is at least partially forward-looking, as shown in FIG. 41B. The first longitudinal portion 632 may be returned to the position illustrated in FIG. 41A by proximally retracting the outer tubular body 626 relative to the inner tubular body 628.

FIG. 41C presents a catheter 642 that is a variation of the catheter 624 of FIGS. 41A and 41B. As such, similar components are similarly numbered and will not be discussed with reference to FIG. 41C. As illustrated in FIG. 41C, an inner tubular body 646 may include first and second longitudinal portions 632, 634. However, as opposed to the embodiment of FIGS. 41A and 41B, where the first and second longitudinal portions 632, 634 are located proximate to the distal end of the catheter 642, the first and second longitudinal portions 632, 634 of the catheter 642 may be disposed at any appropriate point along the catheter 642. An outer tubular body 644 may include a window 648 to accommodate the deployment of the first longitudinal portion 632. The ultrasound imaging array 630 of FIG. 41C may be pivoted in a manner similar to as discussed above with reference to FIGS. 41A and 41B.

Catheter 642 also includes a second ultrasound imaging array 650 that is oriented to image in an at least partially rearward-looking direction. Ultrasound imaging array 650 may be in addition to the ultrasound imaging array 630 or it may be the only imaging array of catheter 642.

FIG. 41C illustrates a catheter with a section (e.g., the first longitudinal portion 632) that has a length and is configured such that when deployed, the ends of the length remain along the body of the catheter while a central section buckles outwardly from the body of the catheter. In this regard an ultrasound imaging array disposed on the central section may be deployed. Several other similarly configured embodiments are disclosed herein. These include, for example, the embodiments of FIGS. 7A through 8D, 38A through 39B, and 40A through 41B. In each of these embodiments, and in other appropriate embodiments disclosed herein, one or more ultrasound imaging arrays may be disposed at any appropriate location on the central section. Thusly, in these embodiments, ultrasound imaging arrays may be disposed such that they move to forward-looking positions, rearward-looking positions, or both when deployed.

The catheters 624, 642 may also include any appropriate electrical interconnection to the ultrasound imaging array 630, including appropriate connection schemes described herein. For example, electrical interconnection members may be disposed along the inner tubular bodies 628, 646.

In addition to deployment of an ultrasound imaging array to obtain images of an area of interest, deployment of ultrasound imaging arrays may also aid in positioning a lumen (e.g., for introduction of an interventional device or other appropriate device). For example, the deployment of the ultrasound transducer array 37 of FIG. 8C (tri-lobe configuration) may result in each of the three lobes of the catheter moving against, for example, the walls of the blood vessel in which the catheter has been deployed. As a result, the end of the lumen 38 may be generally disposed in the center of the blood vessel. Other embodiments described herein, such as, for example, those associated with FIGS. 38A through 40B may also dispose the lumen generally at the center of a channel (e.g., blood vessel) during ultrasound imaging array deployment (e.g., if the channel is of a size that generally corresponds to the size of the catheter when the ultrasound imaging array is deployed).

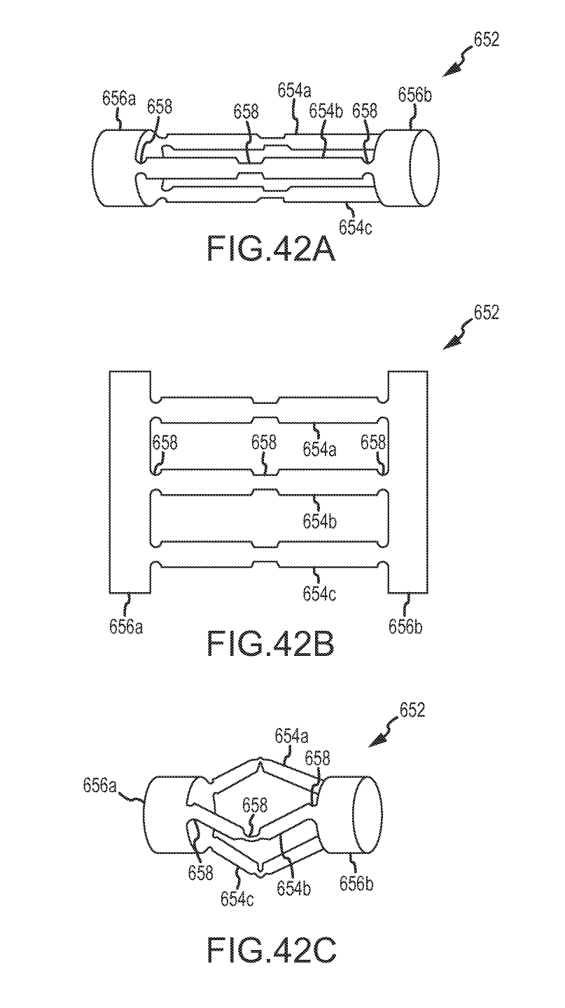

FIGS. 42A through 42C illustrate an exemplary spring element 652 that may be employed to generate a return force to aid in the return of a deployed ultrasound imaging array toward a pre-deployment position. The spring element 652 may include any appropriate number of springs. For instance and as illustrated in FIGS. 42A through 42C, the spring element 652 may include three springs 654a, 654b, 654c disposed between two end section 656a, 656b. The spring element 652 may, for example, be made from a blank, such as illustrated in FIG. 42B. The blank may be rolled to form the cylindrical configuration of FIG. 42A. The ends of the end sections 656a, 656b may be joined to maintain the cylindrical configuration of FIG. 42A. The springs 654a, 654b, 654c may include narrow regions, such as narrow regions 658 disposed along spring 654b, disposed at about the mid-point of the springs 654a, 654b, 654c and at each end of each spring 654a, 654b, 654c. The narrow regions may act as hinges, providing preferential bending points for the springs 654a, 654b, 654c. Accordingly, if a compressive force is applied to the spring element 652 (e.g., to end sections 656a, 656b), each of the springs 654a, 654b, 654c may buckle outwardly as illustrated in FIG. 42C. One or more ultrasound imaging arrays associated with one or more of the springs 654a, 654b, 654c would be consequently pivoted.

The configuration of spring element 652 may, for example, be disposed within the sidewall of the catheter body of the embodiment of FIG. 8C. Each of the springs 654a, 654b, 654c may be disposed within one of the lobes of the three lobe design of FIG. 8C. When integrated into the catheter of FIG. 8C, the spring element 652 may provide a return force biasing the catheter toward a straight, non-deployed position (e.g., for catheter insertion, positioning and removal). In another example, a spring element similar to the spring element 652 (e.g., with the appropriate number of appropriately shaped springs) may be deployed within the tubular body 606 of the catheter 604 of FIGS. 40A and 40B to provide a biasing force toward the straight configuration as illustrated in FIG. 40A.

In still another example, spring elements similar to the spring element 652 (e.g., but with two springs) may be deployed within the outer tubular bodies 578, 596 of the catheters 576, 594 of FIGS. 38A through 39B to provide a biasing force toward the straight configurations as illustrated in FIGS. 38A and 39A. In yet another example, an appropriately modified spring element similar to the spring element 652 (e.g., but with one spring) may be deployed within the inner tubular body 628 of the catheter 624 of FIG. 41A to provide a biasing force toward the straight configuration as illustrated in FIG. 41A.

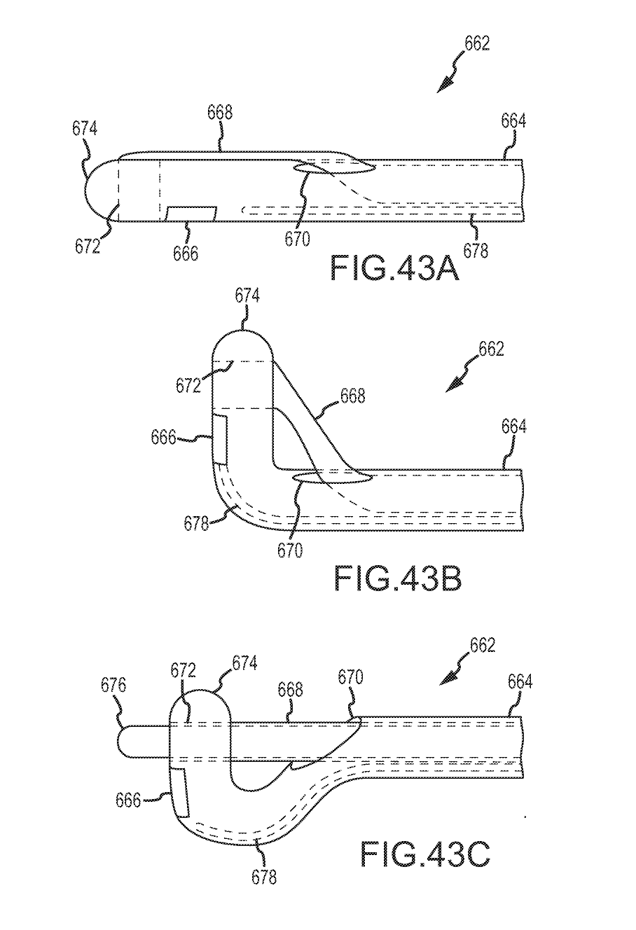

FIGS. 43A through 43C illustrate a catheter 662 that includes an outer tubular body 664. An ultrasound imaging array 666 is interconnected to the outer tubular body 664. The catheter 662 includes a collapsible lumen 668. The collapsible lumen 668 generally runs along the length of the catheter 662 in a central cavity of the outer tubular body 664. However, near the distal end of the catheter 662, the collapsible lumen 668 is routed through a side port 670 of the outer tubular body 664. For a predetermined distance, the collapsible lumen 668 runs along an exterior surface of the outer tubular body 664. Close to a distal end of the catheter 662 (at a point distal to the side port 670), the collapsible lumen 668 is interconnected to an end port 672. The end port 672 is a transverse through-hole proximate to a tip 674 of the catheter 662. The end port 672 may be configured such that an opening of the end port 672 is on the same side of the outer tubular body 664 as the front face of the ultrasound imaging array 666.

During insertion of the catheter 662 into a patient, the catheter 662 may be configured as illustrated in FIG. 43A with the tip 674 generally pointing along the longitudinal axis of the catheter 662. Furthermore, the portion of the collapsible lumen 668 external to the outer tubular body 664 (e.g., the portion of the collapsible lumen between the side port 670 and the end port 672) may be collapsed and generally positioned against the outside wall of the outer tubular body 664.

When it is desired to obtain images of a region distal to the tip 674, the collapsible lumen 668 may be pulled proximally relative to the outer tubular body 664. The result may be for the distal end of the catheter 662 to bend (upward when in the orientation shown in FIG. 43B) such that the ultrasound imaging array 666 is pivoted to a forward-looking position. To achieve such a bending motion, the distal end of the catheter 662 may be designed such that a region between the ultrasound imaging array 666 and the side port 670 is relatively flexible, while a region including the ultrasound imaging array 666 and distal to the ultrasound imaging array is relatively rigid. Accordingly, pulling the collapsible lumen 668 proximally may result in the relatively flexible region bending causing the ultrasound imaging array 666 front face and the opening of the end port 672 to pivot to a forward-looking configuration as illustrated in FIG. 43B.

When it is desired to insert an interventional device 676 into the patient, the interventional device 676 may be advanced distally through the collapsible lumen 668. As the interventional device 676 is advanced through the side port 670, the opening of the side port 670 may be displaced such that it is in line with the central cavity of the outer tubular body 664. As the interventional device 676 is advanced through the section of the collapsible lumen 668 external to the outer tubular body 664, that portion of the collapsible lumen 668 may also be moved such that it is aligned with the central cavity of the outer tubular body 664. As the interventional device 676 is advanced through the end port 672, the end port 672 may also be moved such that it too is aligned with the central cavity of the outer tubular body 664 and the section of the collapsible lumen 668 external to the outer tubular body 664. As the interventional device 676 is advanced, the ultrasound imaging array 666 may be displaced perpendicularly (e.g., downward when in the orientation illustrated in FIG. 43C) relative to the longitudinal axis of the catheter 662. It will be appreciated that the ultrasound imaging array 666 may remain operable to generate images distal to the tip 674 while the interventional device 676 is deployed distal to the tip 674.

Upon retraction of the interventional device 676, the catheter 662 may be returned to an aligned position (e.g., the configuration of FIG. 43A) for subsequent repositioning or removal. In an embodiment, the distal end of the catheter 662 may include a spring element that may return the catheter 662 to an aligned position once the external displacement forces (e.g., retraction force on the collapsible lumen 668 and/or displacement force due to the presence of the interventional device 676) have been removed. In another embodiment, a stylet (e.g., a relatively stiff wire, not shown) may be advanced through a stylet channel 678. The stylet may have sufficient stiffness to return the end of the catheter 662 toward an aligned position (e.g., the position of FIG. 43A).

The catheter 662 may also include any appropriate electrical interconnection to the ultrasound imaging array 666, including appropriate connection schemes described herein. For example, electrical interconnection members may be disposed along the outer tubular body 664.

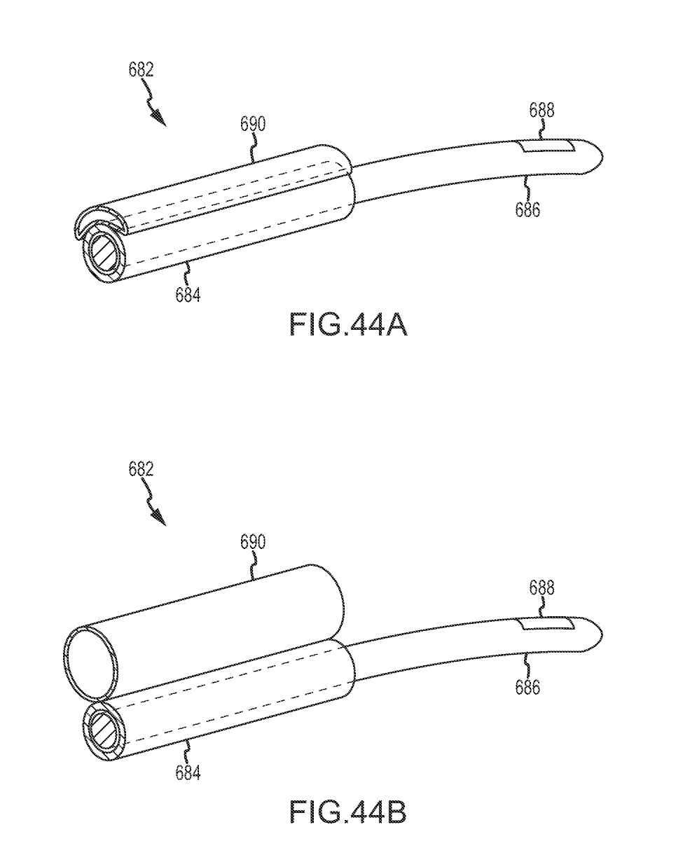

FIGS. 44A and 44B illustrate a catheter 682 that includes a tubular body 684. The tubular body may be sized and configured to deliver a steerable imaging catheter 686 to a selected site within a patient. The steerable imaging catheter 686 may include an ultrasound imaging array 688 disposed at a distal end thereof. Interconnected to an outer surface of the tubular body 684 may be a distensible channel 690. As illustrated in FIG. 44A, the distensible channel 690 may be inserted in a collapsed state, thereby reducing the cross section of the catheter 682 during insertion. Once the catheter 682 is satisfactorily positioned, an interventional device (not shown) may be delivered through the distensible channel 690. The distensible channel 690 may expand as the interventional device is advanced through the distensible channel 690. The distensible channel 690 may be made from any appropriate catheter material, including by way of example, ePTFE, silicone, urethane, PEBAX®, Latex, and/or any combination thereof. The distensible channel 690 may be elastic and may stretch to the diameter of the interventional device as the interventional device is introduced. In another arrangement, the distensible channel 690 may be inelastic and may unfold as the interventional device is introduced. For example, the distensible channel 690 may include a film tube. In another arrangement, the distensible channel 690 may include elastic and inelastic materials.

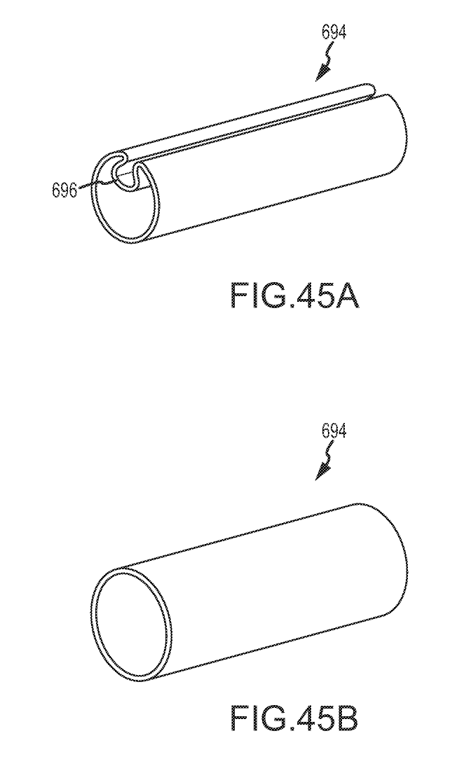

FIGS. 45A and 45B illustrate a catheter body 694. An introductory configuration is illustrated in FIG. 45A. The introductory configuration may include an invaginated portion 696. Once the catheter body 694 is satisfactorily positioned, an interventional device (not shown) may be delivered therethrough. The catheter body 694 may expand as the interventional device is advanced. Expansion of the catheter body 694 may comprise pushing the invaginated portion 696 outward until it forms part of a generally tubular catheter body as illustrated in FIG. 45B. In this regard, the catheter body 694 may be introduced into a patient while in a configuration with a first cross sectional area. Then, at a selected point, an interventional device may be inserted through the catheter body 694 and the catheter body 694 may expand to a second cross sectional area, where the second cross sectional area is larger than the first cross sectional area. The deformation of the catheter body 694 from the introductory configuration (FIG. 45A) to the expanded configuration (FIG. 45B) may be an elastic deformation, where after removal of the interventional device, the catheter body 694 is able to return toward its original profile, or it may be an at least partially plastic deformation.

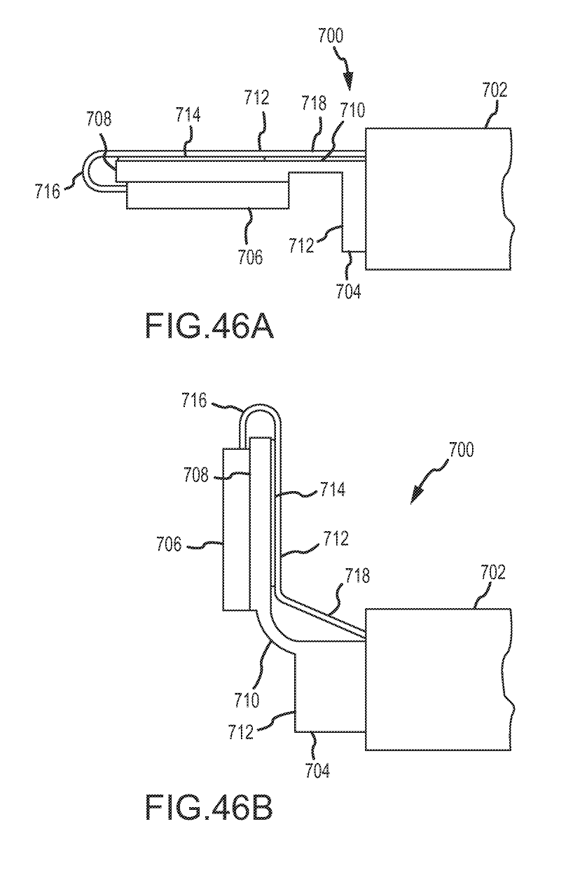

FIGS. 46A and 46B illustrate a catheter 700 that includes an outer tubular body 702 and an inner tubular body 704. The inner tubular body 704 may include a lumen therethrough. The catheter 700 also includes an ultrasound imaging array 706 interconnected to a tip support portion 708 of the inner tubular body 704. The tip support portion 708 of the inner tubular body 704 is interconnected to the distal end of the inner tubular body 704 by a hinge portion 710 of the inner tubular body 704. The tip support portion 708 and the hinge portion 710 of the inner tubular body 704 may be formed by, for example, cutting away a portion of the distal end of the inner tubular body 704, leaving a section (tip support portion 708) to which the ultrasound imaging array 706 may be interconnected and a section (hinge portion 710) that may act a hinge between the tip support portion 708 and a tubular end 711 of the inner tubular body 704. The inner tubular body 704 may be of any appropriate construction. For example, the inner tubular body 704 may be constructed similarly to the inner tubular body 80 of FIG. 5E, with the addition of a braided mesh to reinforce the inner tubular body 704. The braided mesh may serve to provide a return force to return the ultrasound imaging array 706 to an introductory position (as illustrated in FIG. 46A) from a deployed position (as illustrated in FIG. 46B).

The hinge portion 710 may allow the tip support portion 708 to pivot about the hinge portion 710 relative to the inner tubular body 704. An electrical interconnection member 712 may electrically interconnect to the ultrasound imaging array 706. The electrical interconnection member 712 is connected to a distal end of the ultrasound imaging array 706. The electrical interconnection member 712 may be bonded or otherwise fixed to a portion 714 of the tip support portion 708 on an opposite side of the tip support from the ultrasound imaging array 706. The electrical interconnection member 712 may include a loop 716 between the connection to the ultrasound imaging array 706 and the portion 714. The portion 714, by virtue of its fixed position relative to the tip support portion 708 may serve as a strain relief preventing strain associated with pivoting of the ultrasound imaging array 706 from being translated to the loop 716 and array 706 through the electrical interconnection member 712. A tether portion 718 of the electrical interconnection member 712 may be disposed between the bonded portion 714 and the point where the electrical interconnection member 712 enters into the outer tubular body 702. The tether portion 718 may be an unmodified portion of the electrical interconnection member 712 or it may be modified (e.g., structurally reinforced) to accommodate additional forces due to its serving as a tether. The tip support portion 708 and the ultrasound imaging array 706 may be encased or otherwise disposed within a tip (not shown).

During insertion into a patient, the catheter 700 may be arranged as in FIG. 46A with the ultrasound imaging array 706 in axial alignment with the inner tubular body 704 and a field of view of the ultrasound imaging array 706 pointing perpendicular to the longitudinal axis of the catheter 700 (downward as illustrated in FIG. 46A). In this regard, the catheter 700 may be substantially contained within a diameter equal to the outer diameter of the outer tubular body 702. As desired, the ultrasound imaging array 706 may be pivoted relative to the inner tubular body 704 by moving the inner tubular body 704 distally relative to the outer tubular body 702. Such relative motion will cause the ultrasound imaging array 706 to pivot about the hinge portion 710 due to the restraint of motion of the ultrasound imaging array 706 by the tether portion 718. The ultrasound imaging array 706 may be returned to the position illustrated in FIG. 46A by moving the inner tubular body 704 proximally relative to the outer tubular body 702.

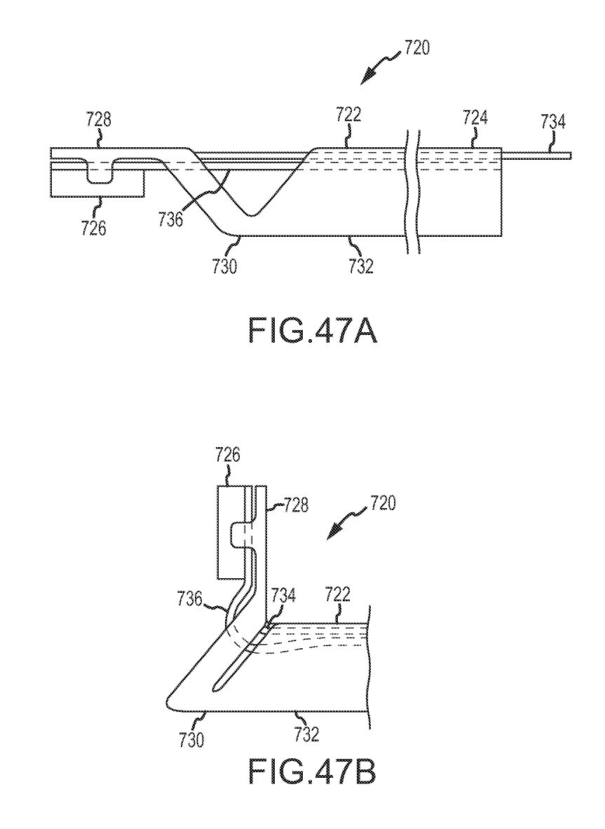

FIGS. 47A and 47B illustrate a catheter 720 that includes a tubular hinge 722 interconnected to a distal end of a tubular body 724. The tubular hinge 722 and tubular body 724 may include a lumen therethrough for the introduction of an interventional device. The catheter 720 also includes an ultrasound imaging array 726 interconnected to a support portion 728 of the tubular hinge 722. A hinge portion 730 of the tubular hinge 722 is disposed between the support portion 728 of the tubular hinge 722 and a tubular portion 732 of the tubular hinge 722. The catheter 720 further includes a wire 734 connected to the support portion 728 and running along the tubular hinge 722 and the tubular body 724. Pulling on a proximal end of the wire 732 may cause the support portion 728 to pivot relative to the tubular portion 732 about the hinge portion 730 as shown in FIG. 47B. Releasing the pulling force on the wire 734 and/or pushing on the proximal end of the wire 734 may result in the support portion 728 returning to the position shown in FIG. 47A. The tubular hinge 722 may include a shape memory material (e.g., Nitinol) and/or a spring material, such that the tubular hinge 722 may return toward the position illustrated in FIG. 47A once the pulling force is released. An electrical interconnection member 736 may electrically interconnect to the ultrasound imaging array 726. The electrical interconnection member 736 may be in the form of a flexboard or other flexible conductive member. The electrical interconnection member 736 may be routed through the tubular hinge 722 as shown in FIGS. 47A and 47B and then interconnect to a spirally wound electrical interconnection member disposed within the tubular body 724 (e.g., similar to the electrical interconnection member 104 of FIG. 5E). The support portion 728 and the ultrasound imaging array 726 may be encased or otherwise disposed within a tip (not shown).

During insertion into a patient, the catheter 720 may be arranged as in FIG. 47A with the ultrasound imaging array 726 in axial alignment with the tubular body 724 and a field of view of the ultrasound imaging array 726 pointing perpendicular to the longitudinal axis of the catheter 720 (downward as illustrated in FIG. 47A). In this regard, the catheter 720 may be substantially contained within a diameter equal to the outer diameter of the tubular body 724. As desired, the ultrasound imaging array 726 may be pivoted relative to the tubular body 724 by moving the wire 734 distally relative to the tubular body 724. Such relative motion will cause the ultrasound imaging array 726 to pivot about the hinge portion 730 due to the restraint of motion of the ultrasound imaging array 726 by the tubular hinge 722.

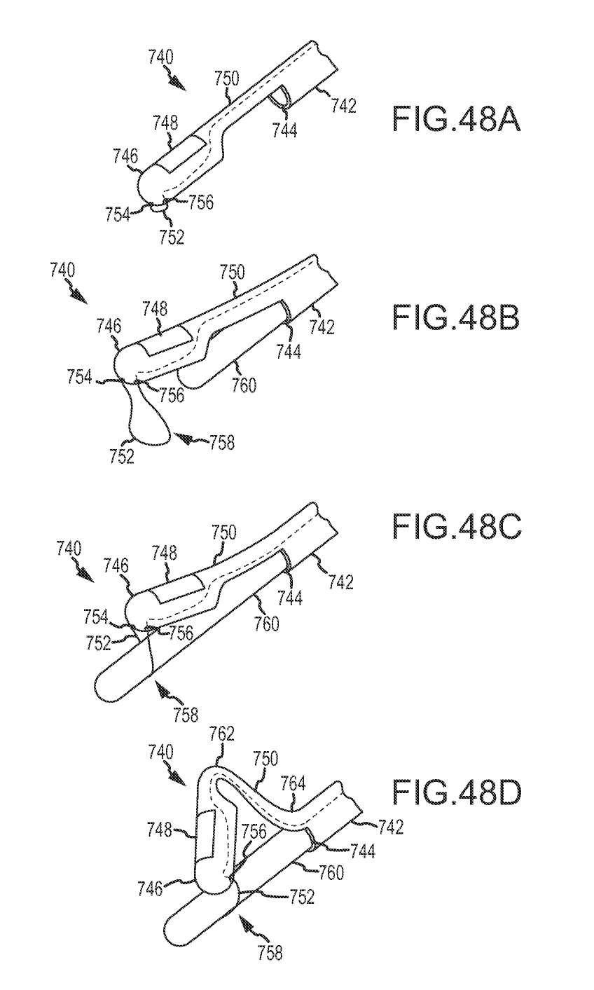

FIGS. 48A through 48D illustrate a catheter 740 that includes a tubular body 742 that includes a lumen 744 therethrough. The catheter 740 also includes a tip portion 746 that in turn includes an ultrasound imaging array 748. The tip portion 746 may be interconnected to the tubular body 742 by an intermediate portion 750. A wire 752 is attached to a distal portion of the tip portion 746 at a wire anchor 754. The wire 752 may be made from any appropriate material or group of materials, including, but not limited to, metals and polymers. The wire 752 is externally (relative to the tip portion 746) routed from the wire anchor 754 to a wire feed hole 756 on the distal portion of the tip portion 746. The wire 752 passes through the wire feed hole 756 and enters the interior of the tip portion 746. Thereafter, the wire 752 runs internally along the tip portion 746, intermediate portion 750, and at least a portion of the tubular body 742. A proximal end of the wire 752 (not shown) may be accessible to an operator of the catheter 740. The catheter 740 may be configured such that in the absence of externally applied forces, the tip portion 746 and intermediate portion 750 are axially aligned with the tubular body 742 as illustrated in FIG. 48A. In this regard, a shape memory material (e.g., Nitinol) or a spring material may be incorporated into the catheter 740 such that the tip portion 746 and intermediate portion 750 may return to the position illustrated in FIG. 48A once any external forces are released.

During insertion into a patient, the catheter 740 may be arranged as in FIG. 48A with the tip portion 746 and intermediate portion 750 in axial alignment with the tubular body 742 and a field of view of the ultrasound imaging array 748 pointing perpendicular to the longitudinal axis of the catheter 740 (generally upward as illustrated in FIG. 48A). In this regard, the tip portion 746 may be substantially contained within a diameter equal to the outer diameter of the tubular body 742.

As desired, the tip portion 746 that includes the ultrasound imaging array 748 may be pivoted relative to the tubular body 742 to a forward-looking position where the ultrasound imaging array 748 may be used to generate images of a volume distal to the catheter 740. To pivot the tip portion 746, a first step may be to feed a portion of the wire 752 through the wire feed hole 756 to form a snare 758 (a loop of the wire 752 external to the tip portion 746) illustrated in FIG. 48B. The wire feed hole 756 and corresponding passages within the tip portion 746 may be configured such that, upon such feeding, the wire 752 generally forms the snare 758 in a plane perpendicular to the longitudinal axis of the catheter 740 and encircling a cylindrical distal extension of the lumen 744. Accordingly, when an interventional device 760 is fed distally from the lumen 744, it will pass through the snare 758 as illustrated in FIG. 48C. Once the interventional device 760 is fed through the snare 758, the wire 752 may be drawn into the tip portion 746 through the wire feed hole 756 such that the snare 758 captures the interventional device 760 such that the distal end of the tip portion 746 and the interventional device 760 move in tandem. One captured, the interventional device 760 may be moved proximally relative to the tubular body 742, causing the tip portion 746 to pivot such that the ultrasound imaging array 748 is in an at least partially forward-looking position as illustrated in FIG. 48D. The intermediate portion 750 may be configured such that it bends in a first bend area 762 and a second bend area 764 to facilitate the pivoting of the tip portion 746 as illustrated in FIG. 48D. To return the tip portion 746 toward it positioning of FIG. 48A, the interventional device 760 may, while captured by the snare 758, be advanced distally and/or the snare 758 may loosened, thereby decoupling the distal end of the tip portion 746 and the interventional device 760 (thus allowing the shape memory material and/or spring material to move the tip portion 746).

The catheter 740 may also include any appropriate electrical interconnection to the ultrasound imaging array 748, including appropriate connection schemes described herein. For example, electrical interconnection members may be disposed along the tubular body 742 and the intermediate portion 750.

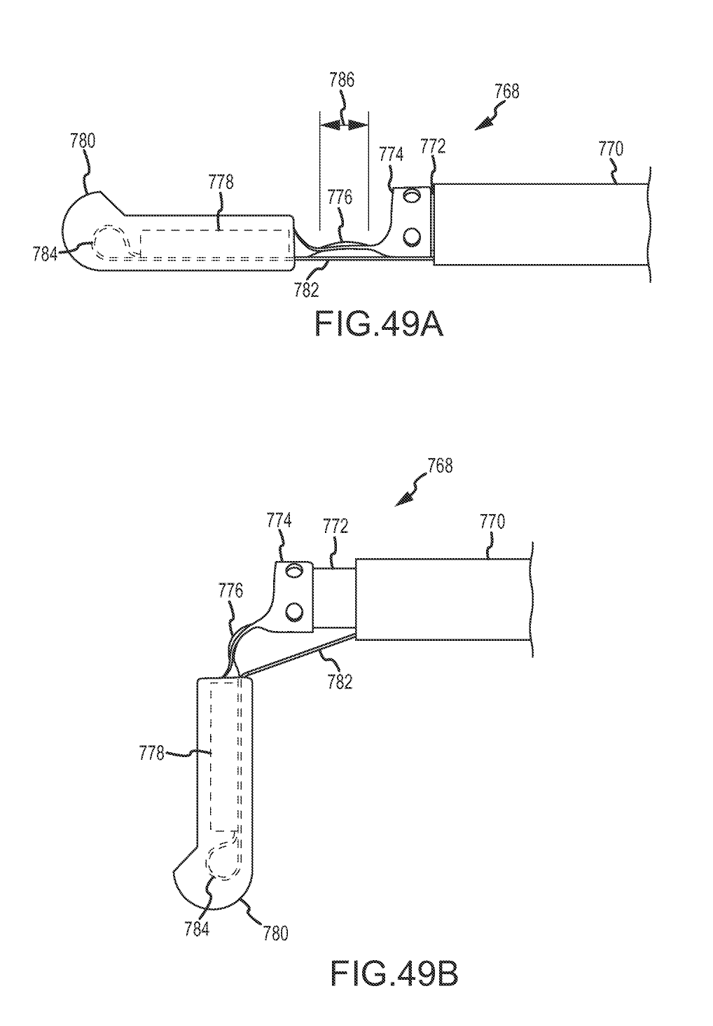

FIGS. 49A and 49B illustrate a catheter 768 that includes an outer tubular body 770 and an inner tubular body 772. The catheter 768 also includes an ultrasound imaging array 778 and a support 774 and with a hinge portion 776. The support 774 and the ultrasound imaging array 778 may be disposed within a tip 780. The catheter 768 is somewhat similar to the catheter 54 of FIGS. 5B through 5D and therefore similar traits will not be discussed. An exemplary difference between the catheter 768 and the catheter 54 is that a flexboard 782 of catheter 768 is disposed along an outside bottom (as viewed in FIG. 49A) surface of the support 774 and includes an end loop 784 where the flexboard 782 is connected to the distal end of the ultrasound imaging array 778. Such a design may reduce forces (e.g., act as a strain relief) translated to the junction between the flexboard 782 and the ultrasound imaging array 778 due to pivoting of the ultrasound imaging array 778. Such a design also obviates the need for the flexboard 782 to be threaded through or around the support 774 to enable interconnection to the ultrasound imaging array 778 at the proximal end of the ultrasound imaging array 778. In turn, this allows for a unitary hinge portion 776 (as opposed to the dual hinge portions 86a, 86b of the catheter 54 of FIG. 5B) such as illustrated in FIGS. 49A and 49B. Moreover, the strain relief of the ultrasound imaging array 778 to flexboard 782 connection provided by the configuration of FIGS. 49A and 49B may be beneficial in enabling the flexboard 782 to also serve the function of a tether (similar to the tether 78 of FIG. 5B). In an alternate embodiment, the catheter 768 of FIGS. 49A and 49B may include a tether similar to tether 78 of FIG. 5B.

FIG. 49A illustrates a region over which deflection occurs 786. The region over which deflection occurs 786 is the region along the length of the catheter 768 where the hinge portion 776 bends to produce the deflection illustrated in FIG. 49B. The region over which deflection occurs 786 is shorter than the diameter of the outer tubular body 770.

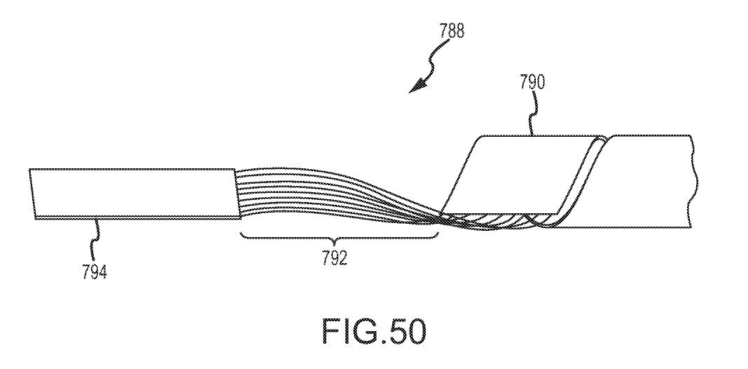

FIG. 50 depicts an embodiment of an electrical interconnection member 788. The electrical interconnection member 788 may, for example, take the place of the assembly illustrated in FIG. 5F in the catheter 50 illustrated in FIGS. 5A through 5E. Moreover, electrical interconnection member 788 or features thereof may be used in any appropriate embodiment disclosed herein. The electrical interconnection member 788 includes a helically disposed portion 790 that may be disposed in a tubular body of a catheter (e.g., similar to the electrical interconnection member 104 of FIG. 5F). The helically disposed portion 790 of the electrical interconnection member 788 may include a plurality of individual conductors bound together in a side-by-side arrangement. The electrical interconnection member 788 may include a non-bonded portion 792 where the individual conductors of the electrical interconnection member 788 are not bonded together. The individual conductors of the non-bonded portion 792 may each be individually insulated to help prevent shorting between the conductors. The non-bonded portion 792 may provide a portion of the electrical interconnection member 788 that is relatively more flexible than the helically disposed portion 790. In this regard, the non-bonded portion 792 may have sufficient flexibility to provide an electrical connection between members that are hinged relative to each other. Therefore, in appropriate embodiments described herein, the non-bonded portion 792 of the electrical interconnection member 788 may replace a flexboard or other flexible electrical interconnections.

The electrical interconnection member 788 may further include an array connection portion 794 configured to electrically connect to an ultrasound imaging array (not shown in FIG. 50). The array connection portion 794 may, for example, include the plurality of individual conductors bound together in the same side-by-side arrangement as in the helically disposed portion. In this regard, the electrical interconnection member 788 may be configured by removing the bonding structure between conductors in the non-bonded portion 792, while leaving the bonding in tact in the helically disposed portion 790 and the array connection portion 794. The conductors of the array connection portion 794 may be selectively exposed such that they may be electrically interconnected to appropriate members of an ultrasound imaging array. In another embodiment, the array connection portion 794 may interconnect to an intermediate member that may be arranged to provide electrical connections from the individual conductors of the array connection portion 794 to the appropriate members of an ultrasound imaging array.

An alternate embodiment of the electrical interconnection member 788 may be configured without the array connection portion 794. Such a configuration may utilize “flying leads” where each conductor of the non-bonded portion 792 remains electrically interconnected to the helically disposed portion 790 on one end and unconnected on the other end. These unconnected flying leads may then, for example, be individually bonded to corresponding conductors on an ultrasound imaging array.

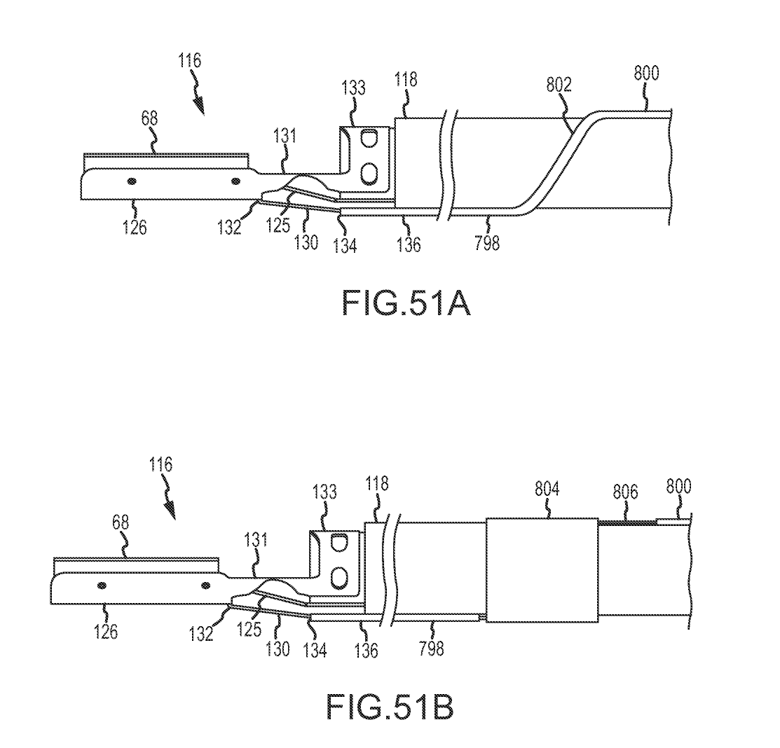

In embodiments described herein wherein a movable elongate member (e.g., pull wire) is employed to cause a deflection of an ultrasound imaging array, the elongate member is generally routed along one side of a catheter body. In a variation of such embodiments, the elongate member may be configured such that a first portion of it is disposed along a first side of the catheter body, and a second portion of the elongate member is disposed along a second side of the catheter body. For example, FIGS. 51A and 51B illustrate the embodiment of FIG. 6B with a first portion 798 of the pull wire housing 136 and pull wire 130 disposed along a first side of the catheter body 118 and a second portion 800 of the pull wire housing and pull wire disposed along a second side of the catheter body 118. Other components of FIG. 6B are as previously described and will not be described further. Such configurations may help to reduce the level of non-symmetrical forces imparted onto the catheter body 118 (e.g., during catheter placement and/or operation) by the pull wire housing 136 and pull wire 130. This may lead to an increased ability to maintain catheter stability during tip deployment.

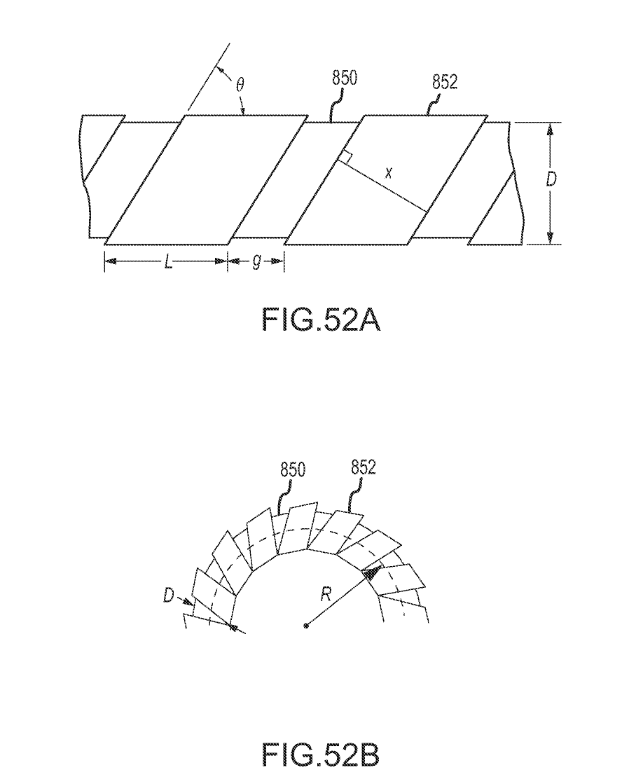

FIG. 51A illustrates an embodiment where the first portion 798 of the pull wire housing 136 and pull wire 130 is connected to the second portion 800 of the pull wire housing 136 and pull wire 130 by a transition section 802. The transition section 802 is a section of the pull wire housing 136 and pull wire 130 that is spirally wound about the catheter body 118. FIG. 52A illustrates en embodiment where the first portion 798 of the pull wire housing 136 and pull wire 130 is connected to the second portion 800 of the pull wire housing 136 and a second pull wire 806 via a coupling 804. The coupling 804 may be cylindrically disposed about a portion of the length of the catheter body 118 and may be operable to slide along that portion of the length of the catheter body 118 in response to forces imparted on the pull wires 130, 806. The second pull wire 806 may be disposed on the second side of the catheter body 118 and is attached to the coupling 804. The pull wire 130 is also attached to the coupling 804. When an operator pulls the second pull wire 806 proximally, the coupling 804 is displaced proximally, and the pull wire 130, by virtue of its connection to the coupling 804, is also pulled proximally. Both of the illustrated pull wire configurations of FIGS. 51A and 51B may also operate as push wires.

FIGS. 52A and 52B illustrate a portion of a catheter body that includes a substrate 850 and a helically wound electrical interconnection member 852. The substrate 850 and electrical interconnection member 852 may be incorporated into any appropriate embodiment disclosed herein, including embodiments where an inner tubular body contains the electrical interconnection member 852 and embodiments where an outer tubular body contains the electrical interconnection member 852. The substrate 850 is the layer about which the electrical interconnection member 852 is wound. For example, the substrate 850 would be the inner tie layer 102 in the embodiment of FIG. 5E.

Turning to FIG. 52A, the electrical interconnection member 852 may have a width of (x) and the substrate may have a diameter of (D). The electrical interconnection member 852 may be wrapped about the substrate 850 such that there exists a gap (g) between subsequent coils of the electrical interconnection member 852. The electrical interconnection member 852 may be wound at an angle of (θ), thereby resulting in a length (L) of each winding of the electrical interconnection member 852 along the longitudinal axis of the catheter. Accordingly, the length (L) is related to the angle (θ) as follows:

L=x/sin(θ) Equation 1

Furthermore, the angle (θ) is related to (D), (L) and (g) as follows:

tan(θ)=(π(D))/(z(L+g)) Equation 2

Where (z) is the number of unique electrical interconnection members 852 wound about the substrate 850 (in the catheter of FIGS. 52A and 52B, (z)=1). For a particular electrical interconnection member 852, (x) is known. Also, for a particular substrate 850, (D) will be known. And for a particular catheter, (z) and (g) may be known. Accordingly, Equations 1 and 2 may have two unknown variables, (9) and (L). Therefore, for given values of (D), (z), (g) and (x), (θ) and (L) may be determined. In an exemplary catheter where the diameter (D) of the substrate was 0.130 inches (3.3 mm), the number (z) of electrical interconnection members 852 was 1, the desired gap (g) was 0.030 inches (0.76 mm), and the electrical interconnection member 852 width (x) was 0.189 inches (4.8 mm), (θ) was found to be 58 degrees and (L) was found to be 0.222 inches (5.64 mm).

Turning to FIG. 52B, for a given catheter, there may be a minimum desired bend radius (R). To ensure that subsequent coils of the electrical interconnection member 852 do not overlap each other when the catheter is bent to the minimum desired bend radius (R), the gap (g) should equal or exceed a minimum gap (gₘ). The minimum gap (gₘ) is the gap size where subsequent coils of the electrical interconnection member 852 come into contact with each other when the catheter is bent to the minimum desired bend radius (R) as illustrated in FIG. 52B. The minimum desired bend radius (R) is related to the length (L) and minimum gap (gₘ) as follows:

(L+gₘ)/L=R/(R−(D/2)) Equation 3

Plugging the values for (L) (0.222 inches (5.64 mm)) and (D) (0.130 inches (3.3 mm)) into Equation 3 and using a minimum desired bend radius (R) of 1.0 inch (25.4 mm), yields a minimum gap (gₘ) of 0.015 inches (0.38 mm). Accordingly, the gap (g) of 0.030 inches (0.76 mm) used above in Equations 1 and 2 exceeds the minimum gap (gₘ) of 0.015 inches (0.38 mm) for a bend radius (R) of 1.0 inch (25.4 mm) from Equation 3. Therefore the gap (g) of 0.030 (0.76 mm) inches should not result in subsequent coils of the electrical interconnection member 852 coming into contact with each other when the catheter is bent to a bend radius (R) of 1.0 inch (25.4 mm).

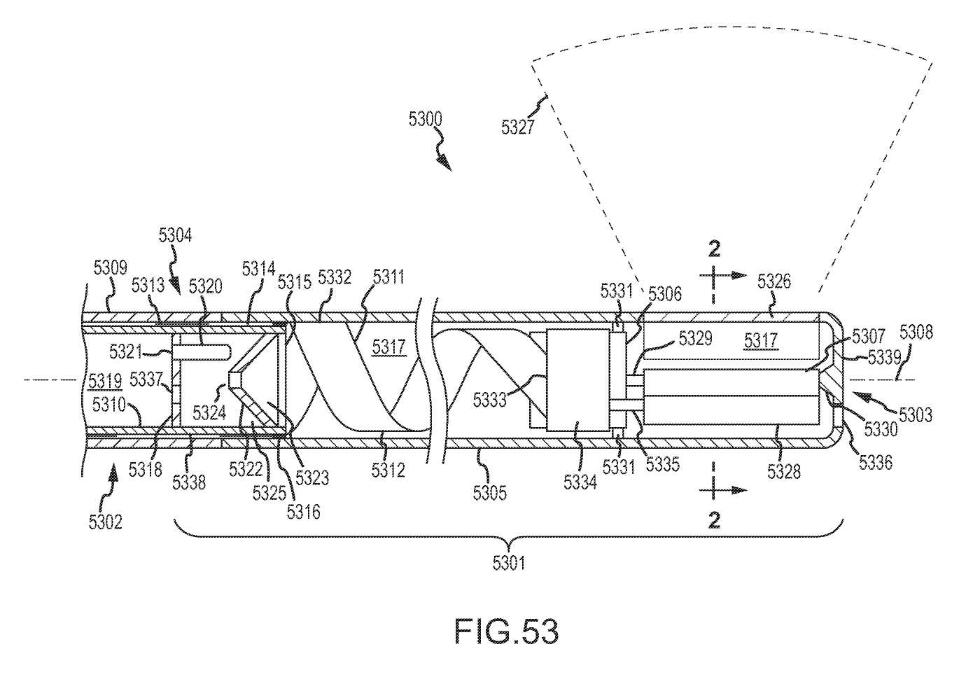

FIGS. 53 through 56B illustrate embodiments of catheter probe assemblies that include catheter tips, transducer arrays and associated componentry to reciprocally pivot the transducer arrays within the catheter tips. Although not illustrated, the catheter tips may be deflectable and the illustrated embodiments may further include hinges and associated componentry to selectively deflect the catheter tips (e.g., relative to the longitudinal axis of the catheter shafts at the distal ends of the catheter shafts). Also, the embodiments of FIGS. 53 through 56B may further include lumens.

FIG. 53 is a partial cross-sectional view an ultrasound catheter probe assembly 5300. The catheter probe assembly 5300 includes a catheter tip 5301 attached to a catheter shaft 5302. The catheter probe assembly 5300 may generally be sized and shaped for insertion into a patient and subsequent imaging of an internal portion of the patient. The catheter probe assembly 5300 may generally include a distal end 5303 and a proximal end (not shown). The catheter probe assembly 5300 proximal end may include a control device operable to be hand-held by a user (e.g., a clinician). The user may manipulate the movement of the catheter probe assembly 5300 by manipulating the control device. During imaging, the distal end 5303 of the catheter probe assembly 5300 may be disposed within the body of a patient while the control device and the proximal end of the catheter probe assembly remain external to the patient.

The catheter tip 5301 may be disposed between the distal end 5303 and a proximal end 5304 of the catheter tip 5301. The catheter tip 5301 may include a catheter tip case 5305. The catheter tip case 5305 may be a relatively rigid (as compared to the catheter shaft 5302) member housing a motor 5306 and a transducer array 5307, both of which are discussed below. Alternatively, as noted below, a portion of the catheter tip case 5305 may be steerable and/or flexible. The catheter tip 5301 may include a central axis 5308.

The catheter shaft 5302 may be operable to be guided into the patient. The catheter shaft 5302 may use any appropriate guidance method such as, but not limited to, a set of control wires and associated controls. In this regard, the catheter shaft 5302 may be steerable. The catheter shaft 5302 may be flexible and therefore be operable to be guided through and follow contours of the structure of the patient, such as the contours of the vasculature system. The catheter shaft 5302 may include an outer layer 5309 and an inner layer 5310. The outer layer 5309 may be constructed from a single layer of material or it may be constructed from a plurality of distinct layers of materials. Similarly, the inner layer 5310 may be constructed from a single layer of material or it may be constructed from a plurality of distinct layers of materials. The inner layer 5310 includes a distal section 5338 that is disposed at the distal end of the inner layer 5315. The distal section 5338 may be an integral part of the inner layer 5310. Alternatively, the distal section 5338 may be separate from the remainder of the inner layer 5310 prior to assembly of the catheter probe assembly 5300, and during assembly the distal section 5338 may be interconnected to the remainder of the inner layer 5310. The inner layer 5310, the outer layer 5309, or both may be configured and/or reinforced to mitigate unwanted catheter rotation due to reciprocal motion described herein and/or to generally increase the strength of the catheter probe assembly. Such reinforcement may take the form of a braided member disposed on or adjacent to the inner layer 5310 and/or the outer layer 5309.

An electrical interconnection member 5311 may be disposed within the catheter probe assembly 5300. The electrical interconnection member 5311 may be comprised of a first portion 5312 and a second portion 5313. The second portion 5313 of the electrical interconnection member 5311 is illustrated in cross-section in FIG. 53. The first portion 5312 of the electrical interconnection member 5311 is not shown in cross-section in FIG. 53. The second portion 5313 of the electrical interconnection member 5311 may be disposed between the outer layer 5309 and inner layer 5310 along the catheter shaft 5302. As illustrated the second portion 5313 of the electrical interconnection member 5311 may be helically disposed around the inner layer 5310. The second portion 5313 may be disposed in the region 5314 between the inner layer 5310 and outer layer 5309. In another embodiment, the second portion 5313 may be wrapped about and bonded to an inner core (not shown) that may be disposed within an internal portion 5319 of the catheter shaft 5302. The second portion 5313 bonded to the inner core may be fixed relative to the inner layer 5310 or it may float free from the inner layer 5310. The second portion 5313 bonded to the inner core may improve kink resistance and torque response of the catheter probe assembly 5300. In such an embodiment, the second portion 5313 may be bonded to the inner core and the first portion 5312 may remain free from attachment to the inner core and the catheter tip case 5305.

A distal end 5315 of the inner layer 5310 may be sealed along its outer perimeter using a sealing material 5316. The sealing material 5316 may be disposed as illustrated between the outer perimeter of the distal end 5315 of the inner layer 5310 and an inner surface of the catheter tip case 5305. In another embodiment, the outer layer 5309 of the catheter shaft 5302 may extend to or beyond the distal end 5315 of the inner layer 5310 and in such an embodiment, the sealing material 5316 may be disposed between the outer perimeter of the distal end 5315 of the inner layer 5310 and an inner surface of the outer layer 5309. Alternatively, the region 5314 between the inner layer 5310 and the outer layer 5309 may, in addition to containing the helically disposed second portion 5313 of the electrical interconnection member 5311, be partially or completely filled with the sealing material 5316. The sealing material 5316 may include any appropriate material such as, for example, a thermoset or thermoplastic material or expanded polytetrafluoroethylene (ePTFE). The second portion 5313 of the electrical interconnection member 5311 may extend along an entire length of the catheter shaft 5302 from the proximal end 5304 of the catheter tip 5301 to an imaging system (not shown). In this regard, the electrical interconnection member 5311 may operatively connect the catheter tip 5301 with the imaging system.

An enclosed volume 5317 may be defined by the catheter tip case 5305, an end portion of the inner layer 5310 of the catheter shaft 5302 and an enclosed volume end wall 5318. The enclosed volume end wall 5318 may be sealably disposed within the inner layer 5310 near to the distal end 5315 of the inner layer 5310. The enclosed volume 5317 may also be sealed by the sealing material 5316 as discussed above.

The enclosed volume 5317 may be fluid-filled and sealed. The fluid may be a biocompatible oil selected, inter alia, for its acoustical properties. For example, the fluid may be chosen to match or approximate the acoustic impedance and/or the acoustic velocity of fluid within the region of the body that is to be imaged. The enclosed volume 5317 may be sealed such that the fluid within the enclosed volume 5317 is substantially unable to leak out of the enclosed volume 5317. Furthermore, the enclosed volume 5317 may be sealed to substantially prevent gasses (e.g., air) from entering into the enclosed volume 5317.

The catheter probe assembly 5300 may be filled using any appropriate method. During filling, the catheter probe assembly 5300 and the fluid may be at known temperatures to beneficially control the volume of fluid introduced and the size of the enclosed volume 5317. In one exemplary filling method, the catheter tip case 5305 may include a sealable port 5336. Gasses within the enclosed volume may be drawn by vacuum out of the enclosed volume 5317 through the sealable port 5336. Then, the fluid may be introduced through the sealable port 5336 until the desired amount of fluid is within the enclosed volume 5317. The sealable port 5336 may then be sealed. In another example, the catheter probe assembly 5300 may include the sealable port 5336 at the distal end 5303 and a sealable port 5337 at the proximal end 5304. The sealable port 5337 may be disposed along the enclosed volume proximal end wall 5318. One of the ports 5337, 5338 may be used as an inlet port for the fluid while the other port 5337, 5338 may be used as an outlet port for displaced gasses. In this regard, as fluid is passed through the inlet port, gasses may escape (or be pulled from using a vacuum) from the enclosed volume 5317 through the outlet port. Once the desired volume of fluid is within the enclosed volume 5317, the ports 5337, 5338 may be sealed. In the above described filling methods, a measured amount of fluid may be removed from the enclosed volume 5317 after it has been completely filled. The amount of fluid removed may correspond to the desired amount of expansion of a bellows member 5320 (described below).

The catheter tip 5301 may include a check valve (not shown) that may be operable to allow fluid to flow out of the enclosed volume 5317 if the pressure differential between the enclosed volume 5317 and the surrounding environment exceeds a predetermined level. The check valve may be in the form of a slit valve disposed along the catheter tip case 5305. In this regard, the check valve may operate to relieve excess pressure that may be created during the filling process, thereby reducing the possibility of the catheter probe assembly 5300 bursting during the filling procedure. Once the enclosed volume is filled, the check valve may be permanently sealed. For example, a clamp may be placed over the check valve to seal the check valve.

The internal portion 5319 of the catheter shaft 5302 may be sealably separated from the enclosed volume 5317. The internal portion 5319 of the catheter shaft 5302 may be disposed within an interior volume of the inner layer 5310. The internal portion 5319 of the catheter shaft 5302 may contain air and may be vented such that the pressure within the internal portion 5319 of the catheter shaft 5302 is equal or close to the local atmosphere pressure in which the catheter probe assembly 5300 is situated. Such venting may be accomplished through a dedicated vent mechanism (such as an opening in the catheter shaft 5302 at a point outside of the body of the patient) between the internal portion 5319 of the catheter shaft 5302 and the local atmosphere.

As may be appreciated, if the enclosed volume 5317 was completely surrounded by substantially rigid members and filled with fluid, temperature variations of the catheter probe assembly 5300 could result in unwanted changes in pressure within the enclosed volume 5317. For example, in such a configuration, if the catheter probe assembly 5300 was exposed to elevated temperatures, the pressure of the fluid within the enclosed volume 5317 may increase; possibly causing some of the fluid to leak out of the enclosed volume 5317. Likewise for example, if the catheter probe assembly 5300 was exposed to reduced temperatures, the pressure of the fluid within the enclosed volume 5317 may decrease, possibly causing some air or other fluid to leak into the enclosed volume 5317. Accordingly, it may be beneficial to prevent or reduce pressure variations within the enclosed volume 5317 relative to the environmental conditions in which the catheter probe assembly 5300 is located.

To assist in equalizing pressure between the fluid within the enclosed volume 5317 and surrounding conditions, the bellows member 5320 may be incorporated into the catheter probe assembly 5300. The bellows member 5320 may be a generally flexible member that is collapsible and expansible in response to volumetric changes in the fluid within the enclosed volume 5317, such as volumetric changes as a result of temperature changes. The bellows member 5320 may be configured to define an internal volume and have a single opening. The single opening may be an open end 5321 of the bellows member 5320 such that the open end 5321 may be disposed along the end wall 5318 and oriented such that the internal volume of the bellows member 5320 is in communication with the internal portion 5319 of the catheter shaft 5302. The remaining portion of the bellows member 5320 may be disposed within the enclosed volume 5317 and may include a closed end portion.

The initial configuration of the bellows member 5320 may be selected such that the bellows member 5320 is operable to compensate for (e.g., equalize pressure between the enclosed volume 5317 and the internal portion 5319 of the catheter shaft 5302) temperature variations across the operational range of temperatures for the catheter probe assembly 5300. Moreover, the bellows member 5320 may be configured to compensate for temperature variations greater than the operational range of temperatures for catheter probe assembly 5300, such as temperature variations that may be seen during catheter probe assembly 5300 storage and/or transportation. The bellows member 5320 may be curved or otherwise shaped to avoid other internal components within the enclosed volume 5317.

At the maximum fluid temperature for which the bellows member 5320 is designed to compensate, the bellows member 5320 may be totally collapsed or close to being totally collapsed. In this regard, the expansion of the fluid within the enclosed volume 5317 may not result in a pressure increase within the enclosed volume 5317 since the bellows member 5320 collapse may compensate for the expansion of the fluid. At the minimum fluid temperature for which the bellows member 5320 is designed to compensate, the bellows member 5320 may be expanded at or near its expansion limit. In this regard, the volumetric contraction of the fluid within the enclosed volume 5317 may not result in a pressure decrease within the enclosed volume 5317 since the bellows member 5320 expansion may compensate for the contraction of the fluid. Furthermore, by positioning the bellows member 5320 in the enclosed volume 5317, it is protected from movement of the catheter shaft 5302.

Although the bellows member 5320 is illustrated as having a cross dimension considerably smaller than a cross dimension of the inner layer of the catheter shaft 5310, the bellows member 5320 may be considerably larger. In this regard, the bellows member 5320 may have a cross dimension approaching that of the inner layer of the catheter shaft 5310. It will be appreciated that such a bellows member may be relatively less flexible than the bellows member 5320 illustrated in FIG. 53, but may be similarly capable of accommodating fluid volume changes due to its relatively larger size. Such a larger bellows member may be constructed similarly to the inner 5310 and/or outer 5309 layers of the catheter shaft.

In conjunction with, or in place of, the bellows member 5320, a portion of the sidewall of the catheter tip case 5305 (e.g., a portion an end wall 5339 of the catheter tip case 5305 and/or a portion of the sidewall of the of the catheter tip case 5305 proximate to the first portion of the electrical interconnect member 5312) may be configured such that the portion performs a function similar to that of the bellows member 5320 described above. For example, the portion may be pliable and may flex inward if the fluid and catheter probe assembly 5300 become cooler and outward if the fluid and catheter probe assembly 5300 become warmer, thereby accommodating temperature related volume changes of the fluid.

In an embodiment, the bellows member 5320, or at least a distal portion thereof, may be elastically-deformable. In particular, the bellows member 5320 may be operable to stretch or elastically expand beyond a neutral state (e.g., a state where there is no pressure differential between the inside of the bellows member 5320 and the outside of the bellows member 5320) in reaction to a pressure differential between the enclosed volume 5317 and the interior of the catheter 5319 where the pressure within the interior of the catheter 5319 is greater than the pressure within the enclosed volume 5317. Such stretching or elastic expansion may accommodate greater pressure differentials than would be attainable with a similarly sized bellows member 5320 that was substantially incapable of stretching or elastically expanding. Furthermore, such a stretchable or elastically expandable bellows member 5320 may result in a catheter probe assembly 5300 that is capable of tolerating temperature variations greater than the operational range of temperatures for the catheter probe assembly 5300, such as temperature variations that may be seen during catheter probe assembly 5300 storage and/or transportation. Such a stretchable or elastically expandable bellows member 5320 may be capable of withstanding a greater range of fluid volumes (e.g., the catheter probe assembly 5300 with a stretchable or elastically expandable bellows member 5320 may be more tolerant of a wider range of ambient temperatures, extending particularly the low temperature range where the fluid typically contracts more than the catheter tip case 5305). Such a stretchable or elastically expandable bellows member 5320 may be silicone based and may be produced using, for example, a liquid transfer molding process.

In one embodiment, a resilient, elastically-deformable bellows member 5320 may be provided so that in a neutral state the bellows member 5320 automatically assumes an initial configuration. Such initial configuration may correspond with a preformed configuration (e.g. a bulbous, dropper-shaped configuration), except as spatially restricted by other rigid componentry (e.g., bubble trap 5322 and/or enclosed volume proximal end wall 5318). In turn, the bellows member 5320 may collapse and automatically expand and stretch relative to such initial configuration in response to pressure variations.

The catheter probe assembly may include a bubble-trap 5322, shown in cross section in FIG. 53. The bubble-trap 5322 may be interconnected to the distal end 5315 of the inner layer 5310 of the catheter shaft 5302. The bubble-trap 5322 may be interconnected to the inner layer 5310 by any appropriate means. For example, the bubble-trap 5322 may be bonded to the inner layer 5310 using an adhesive. For example, the bubble trap 5322 may be press-fit into the inner layer 5310.

The bubble-trap 5322 may include a recess defined by a distal-facing concave surface 5323. Furthermore, a distal portion of the enclosed volume 5317 is defined as the portion of the enclosed volume 5317 distal to the bubble-trap 5322. Correspondingly, a proximal portion of the enclosed volume 5317 is defined as the portion of the enclosed volume 5317 proximal to the bubble-trap 5322. The bubble-trap 5322 may include an aperture 5324 that fluidly interconnects the distal portion to the proximal portion. The aperture 5324 may be disposed at or near the most proximal portion of the distal facing concave surface 5323.

During the life cycle of the catheter probe assembly 5300, bubbles may be formed in or enter into the enclosed volume 5317. The bubble-trap 5322 may be operable to trap these bubbles in the proximal portion of the enclosed volume 5317. For example, during normal operation of the catheter probe assembly 5300 the catheter probe assembly may be disposed in a variety of attitudes including attitudes where the distal end 5303 of the catheter probe assembly 5300 is facing downward. When the catheter probe assembly 5300 is in a downward facing attitude, a bubble within the distal portion may tend to naturally flow upward. Upon coming into contact with the concave face 5323, the bubble may continue to rise until it reaches the aperture 5324. The bubble may then pass through the aperture 5324, moving from the distal portion to the proximal portion. Once the bubble is in the proximal portion and the catheter probe assembly 5300 is placed in an attitude where the distal portion is facing upward, the bubble-trap 5322 will tend to direct any rising bubbles in the proximal portion away from the aperture 5324. Following the slope of the proximal surface of the bubble-trap 5322, the bubbles will tend to migrate to a trap region 5325 and be trapped therein.

The bubble-trap 5322 is beneficial since bubbles present between the transducer array 5307 and an acoustic window 5326 of the case 5305 may produce unwanted image artifacts when the catheter probe assembly 5300 is used to generate an image of an image volume 5327. This is due to the differing acoustical properties of an air bubble versus the acoustical properties of the fluid within the enclosed volume 5317. By keeping bubbles that may form during the lifetime of the catheter probe assembly 5300 away from the transducer array 5307, the operational life of the catheter probe assembly 5300 may be increased. In this regard, bubbles that may form within the enclosed volume 5317 or enter into the enclosed volume 5317 may not lead to a degradation of the images created using the catheter probe assembly 5300.

Prior to insertion of the catheter probe assembly 5300 into a patient, a user (e.g., a physician or technician) may manipulate the catheter probe assembly 5300 in a manner to help move any bubbles that may be present within the enclosed volume 5317 into the volume proximal to the bubble trap 5322. For example, the user may dispose the catheter probe assembly 5300 in an attitude where the distal end 5303 is pointing downward to allow bubbles within the enclosed volume 5317 to move upward into the volume proximal to the bubble trap 5322 thus trapping the bubbles. In another example, the user may grasp the catheter probe assembly 5300 at a point proximal to the catheter tip 5301 and swing the catheter tip 5301 around to impart centrifugal force on the fluid within the enclosed volume 5317 thereby causing the fluid to move toward the distal end 5303 and any bubbles within the fluid to move towards the proximal end 5304. In addition, the catheter probe assembly 5300 may be packaged such that the distal end 5303 is pointing downward so that any bubbles within the enclosed volume 5317 may migrate to the proximal end 5304 of the catheter tip 5301 while the catheter probe assembly 5300 is in storage or is being transported prior to use.

In another example, the catheter probe assembly 5300 may be packaged, shipped and stored in an unfilled state, and prior to use a user may fill the catheter probe assembly 5300 with a fluid. For example, the user may insert a needle of a syringe into the sealable port 5336 and inject a fluid (e.g., saline or bubble-free saline) into the catheter probe assembly 5300 to fill the catheter probe assembly 5300. The user may then manipulate the catheter probe assembly 5300 in any of the manners described above to help move any bubbles that may be present within the enclosed volume 5317 into the volume proximal to the bubble trap 5322. Such systems for packaging, shipping, storing and filling (both pre-filled and filled by the user) may be used by appropriate fluid filled arrangement discussed herein.

A filter may be disposed across the aperture 5324. The filter may be configured such that gasses (e.g., air) may pass through the filter while liquid (e.g., oil, saline) may not be able to pass through the filter. Such a configuration may allow air bubbles to pass from the distal end of the enclosed volume 5317 (the portion of the enclosed volume to the right of the bubble trap 5322 in FIG. 53), through the filter disposed across the aperture 5324, and into the proximal end of the enclosed volume 5317 (the portion of the enclosed volume to the left of the bubble trap 5322 in FIG. 53), while preventing fluid from passing through the filter disposed across the aperture 5324. The filter may include ePTFE.

The catheter probe assembly 5300 includes the transducer array 5307 and an array backing 5328. The transducer array 5307 may comprise an array of a plurality of individual transducer elements that may each be electrically connected to the ultrasound imaging apparatus via a signal connection and a ground connection. The transducer array 5307 may be a one-dimensional array that includes a single row of individual transducer elements. The transducer array 5307 may be a two-dimensional array that includes individual transducer elements arranged, for example, in multiple columns and multiple rows. Ground connections of the entire transducer array 5307 may be aggregated and may be electrically connected to the ultrasound imaging apparatus through a single ground connection. The transducer array 5307 may be a mechanically active layer operable to convert electrical energy to mechanical (e.g., acoustic) energy and/or convert mechanical energy into electrical energy. For example, the transducer array 5307 may comprise piezoelectric elements. For example, the transducer array 5307 may be operable to convert electrical signals from the ultrasound imaging apparatus into ultrasonic acoustic energy. Furthermore, the transducer array 5307 may be operable to convert received ultrasonic acoustic energy into electrical signals.

The transducer array may include a cylindrical enclosure disposed about the array 5307 and array backing 5328. The cylindrical enclosure may reciprocally pivot along with the array 5307 and array backing 5328. The cylindrical enclosure may be constructed of a material that has an acoustic speed similar to blood or other body fluid in which the catheter probe assembly 5300 is to be inserted. The cylindrical enclosure may be sized such that a gap exists between the outer diameter of the cylindrical enclosure and the inner diameter of the case 5305 and acoustic window 5326. The gap may be sized such that capillary forces draw the fluid into, and keep the fluid within, the gap. The fluid may be the aforementioned oil, saline, blood (e.g., where the enclosed volume 5317 is open to its surroundings), or any other appropriate fluid. In one embodiment, the fluid may be placed into the enclosed volume 5317 at the time the catheter probe assembly 5300 is manufactured. In a variation, the fluid may be added at the time of use of the catheter probe assembly 5300. In another embodiment, a high viscosity non-water soluble couplant may be used in place of the above discussed fluid. The couplant may be positioned between the outer diameter of the cylindrical enclosure and the inner diameter of the case 5305. The couplant may be selected such that any escape of the couplant into a patient would not be unacceptably injurious. The couplant may be a grease, such as a silicone grease, Krytox™ (available from E. I. Du Pont De Nemours and Company, Wilmington, Del., U.S.A.), or any other appropriate high viscosity non-water soluble couplant.

To generate an ultrasound image, the ultrasound imaging apparatus may send electrical signals to the transducer array 5307 which in turn may convert the electrical energy to ultrasonic acoustic energy that may be emitted toward the image volume 5327. Structure within the image volume 5327 may reflect a portion of the acoustic energy back toward the transducer array 5307. The reflected acoustic energy may be converted to electrical signals by the transducer array 5307. The electrical signals may be sent to the ultrasound imaging apparatus where they may be processed and an image of the image volume 5327 may be generated.

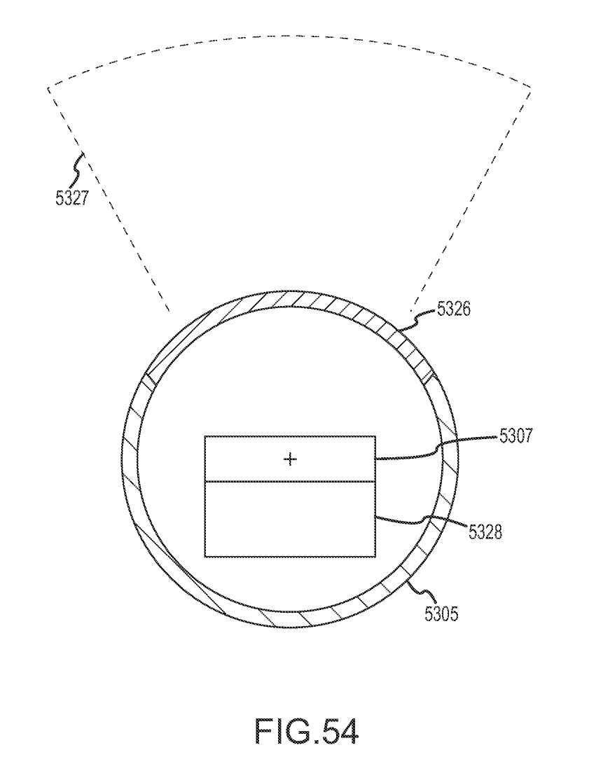

Generally, the transducer array 5307 is operable to transmit ultrasonic energy through the acoustic window 5326 of the catheter tip case 5305. In the catheter probe assembly 5300, the acoustic window 5326 forms part of the catheter tip case 5305 along a portion of the circumference of the case along a portion of the length of the case. FIG. 54 is a cross sectional view of the catheter probe assembly 5300 looking distally from section lines 2-2 of FIG. 53. As shown in FIG. 54, the acoustic window 5326 forms a portion of the circumference of the catheter tip case 5305 along section lines 2-2. The acoustic window 5326 may, for example, occupy 90 degrees or more of the circumference of the catheter tip case 5305. The acoustic window may comprise, for example, polyurethane, polyvinyl acetate, or polyester ether. The ultrasonic energy, in the form of acoustic waves, may be directed through the acoustic window 5326 and into the internal structure of the patient.

As shown in FIG. 54, the catheter tip case 5305 may have a generally circular cross section. Moreover, the outer surface of the catheter tip case 5305 and the acoustic window 5326 may be smooth. Such a smooth, circular exterior profile may help in reducing thrombus formation and/or tissue damage as the catheter probe assembly 5300 is moved (e.g., rotated, translated) within a patient.

In general, the images generated by the catheter probe assembly 5300 may be of a subject (e.g., internal structure of a patient) within the image volume 5327. The image volume 5327 extends outwardly from the catheter probe assembly 5300 perpendicular to the transducer array 5307. The entire image volume 5327 may be scanned by the transducer array 5307. The plurality of ultrasonic transducers may be disposed along the central axis 5308 and may be operable to scan an image plane with a width along the central axis 5308 and a depth perpendicular to the transducer array 5307. The transducer array 5307 may be disposed on a mechanism operable to reciprocally pivot the transducer array 5307 about the central axis 5308 such that the image plane is swept about the central axis 5308 to form the image volume as shown in FIGS. 53 and 54. The sweeping of the image plane about the central axis 5308 enables the transducer array 5307 to scan the entire image volume 5327 and thus a three dimensional image of the image volume 5327 may be generated. The catheter probe assembly 5300 may be operable to reciprocally pivot the transducer array 5307 at a rate sufficient enough to generate real-time or near real-time three-dimensional images of the image volume 5327. In this regard, the ultrasound imaging apparatus may be operable to display live or near-live video of the image volume. Imaging parameters within the image volume 5327, for example focal length and depth of field, may be controlled through electronic means known to those skilled in the art.

As noted above, the enclosed volume 5317 may be fluid-filled. The fluid may act to acoustically couple the transducer array 5307 to the acoustic window 5326 of the catheter tip case 5305. In this regard, the material of the acoustic window 5326 may be selected to correspond to the acoustic impedance and/or the acoustic velocity of the fluid of the body of the patient in the region where the catheter tip 5301 is to be disposed during imaging.

The transducer array 5307 may be interconnected to an output shaft 5329 of the motor 5306 at a proximal end of the transducer array 5307. Furthermore, the transducer array 5307 may be supported on a distal end of the transducer array 5307 by a pivot 5330. As illustrated in FIG. 53, the pivot 5330 may be a portion of the catheter tip case 5305 that extends toward the transducer array 5307 along the rotational axis (e.g., the central axis 5308) of the transducer array 5307. The transducer array 5307 may have a corresponding recess or pocket along its distal end to receive a portion of the pivot 5330. In this regard, the interface between the pivot 5330 and the transducer array 5307 may allow for the transducer array 5307 to reciprocally pivot about its rotational axis while substantially preventing any lateral movement of the transducer array 5307 relative to the catheter tip case 5305. Accordingly, the transducer array 5307 may be operable to be reciprocally pivoted about its rotational axis.

The motor 5306 may be disposed within the enclosed volume 5317. The motor 5306 may be an electrically powered motor operable to rotate the output shaft 5329 in both clockwise and counterclockwise directions. In this regard, the motor 5306 may be operable to reciprocally pivot the output shaft 5329 of the motor 5306 and therefore reciprocally pivot the transducer array 5307 interconnected to the output shaft 5329.

The motor 5306 may have an outer portion that has an outer diameter that is smaller than the inner diameter of the catheter tip case 5305 in the region of the catheter tip case 5305 where the motor 5306 is disposed. The outer portion of the motor 5306 may be fixedly mounted to the inner surface of the catheter tip case 5305 by one or more motor mounts 5331. The motor mounts 5331 may, for example, be comprised of beads of adhesive. The motor mounts 5331 may be disposed between the motor 5306 and inner surface of the catheter tip case 5305 in locations chosen to avoid interference with moving members (discussed below) associated with the reciprocal motion of the transducer array 5307. Motor mounts 5331 may be disposed along the distal end of the outer portion of the motor 5306. Motor mounts 5331 may also be disposed along the proximal end of the outer portion of the motor 5306 such as, for example, along the proximal end of the outer portion of the motor 5306 on the side of the motor 5306 opposite from the side visible in FIG. 53.

When output shaft 5329 position is known, the corresponding position of the transducer array 5307 will be known. Output shaft 5329 position may be tracked in any appropriate manner, such as through the use of an encoder and/or a magnetic position sensor. Output shaft 5329 position may also be tracked through the use of hard stops limiting the motion of the transducer array 5307. Such hard stops (not shown) may limit the range through which the transducer array 5307 may reciprocally pivot. By driving the motor 5306 in a clockwise or counterclockwise direction for a specific period of time, it may be assumed that the motor 5306 has driven the transducer array 5307 against one of the hard stops and therefore the position of the transducer array 5307 may be known.

Electrical interconnections to the motor 5306 from the ultrasound imaging apparatus may be achieved through a dedicated set of electrical interconnections (e.g., wires) separate from the electrical interconnection member 5311. Alternatively, electrical interconnections to the motor 5306 may be made using a portion of the conductors of the electrical interconnection member 5311. Where a dedicated set of electrical interconnections are used to communicate with and/or drive the motor 5306, such interconnections may be run from the motor 5306 to the ultrasound imaging apparatus in any appropriate manner including, for example, through the interior 5319 of the catheter shaft 5302 and/or through the gap 5314. Furthermore, electrical interconnections from the ultrasound imaging apparatus to other components, such as thermocouples, other sensors, or other members that may be disposed within the catheter tip 5301, may be achieved through a dedicated set of electrical interconnections or they may be made using a portion of the conductors of the electrical interconnection member 5311.

The electrical interconnection member 5311 may electrically interconnect the transducer array 5307 with the ultrasound imaging apparatus. The electrical interconnection member 5311 may be a multi-conductor cable comprising of a plurality of conductors arranged side-by-side with electrically nonconductive material between the conductors. The electrical interconnection member 5311 may be ribbon shaped. For example, the electrical interconnection member 5311 may comprise one or more GORE™ Micro-Miniature Ribbon Cables. For example, the electrical interconnection member 5311 may include 64 separate conductors.

The electrical interconnection member 5311 may be anchored such that a portion of it is fixed relative to the catheter tip case 5305. As noted above, the second portion 5313 of the electrical interconnection member 5311 may be secured between the inner layer 5310 and outer layer 5309 of the catheter shaft 5302. Within the enclosed volume 5317, a first end 5332 of the first portion 5312 of the electrical interconnection member 5311 may be secured to the inner surface of the catheter tip case 5305. In this regard, the securing of the first end 5332 may be configured such that the transition from a secured portion of the electrical interconnection member 5311 to a free floating portion may be disposed perpendicular to the orientation of the conductors (e.g., across the width of the electrical interconnection member 5311) at the first end 5332. In another embodiment, the electrical interconnection member may be secured to the inner surface of the case by virtue of its securement between the inner layer 5310 and outer layer 5309 of the catheter shaft 5302. In such an embodiment, the transition from secured to free floating may not be oriented perpendicular to the conductors of the electrical interconnection member 5311. Any appropriate method of anchoring the electrical interconnection member 5311 to the catheter tip case 5305 may be used. For example, adhesive may be used.

Since during scanning the transducer array 5307 may be pivoted about the central axis 5308 relative to the catheter tip case 5305, the electrical interconnection member 5311 must be operable to maintain an electrical connection to the transducer array 5307 while the transducer array 5307 is pivoting relative to the catheter tip case 5305 to which the electrical interconnection member 5311 is fixed at the first end 5332. This may be achieved by coiling the first portion 5312 of the electrical interconnection member 5311 within the enclosed volume 5317. The first end 5332 of the coil may be anchored as discussed. A second end 5333 of the coil may be anchored to an interconnection support 5334 that pivots along with the transducer array 5307 about the central axis 5308. Where the electrical interconnection member 5311 is ribbon shaped, the first portion 5312 of the electrical interconnection member 5311 may be disposed such that a top or bottom side of the ribbon faces and wraps about the central axis 5308.

FIG. 53 illustrates a configuration where the first portion 5312 of the electrical interconnection member 5311 is helically disposed within the enclosed volume 5317. The first portion 5312 of the electrical interconnection member 5311 may be coiled about the central axis 5308 a plurality of times. The first portion 5312 of the electrical interconnection member 5311 may be coiled about the central axis 5308 such that the first portion 5312 of the electrical interconnection member 5311 forms a helix about the central axis 5308. By coiling the electrical interconnection member 5311 about the central axis 5308 a plurality of times, undesirable counteracting torque on the pivoting of the transducer array 5307 may be significantly avoided. Pivoting of the transducer array 5307 about the central axis 5308 in such a configuration may result in a slight tightening, or slight loosening, of the turns of the coiled first portion 5312 of the electrical interconnection member 5311. Such a slight tightening and loosening may result in each coil (e.g., each individual rotation of the helix about the central axis 5308) producing only a small lateral displacement and corresponding displacement of fluid. Furthermore, the displacement may not be uniform for each coil of the helix. Furthermore, by distributing the movement of the first portion 5312 of the electrical interconnection member 5311 over a plurality of coils, the mechanical stresses of movement are distributed over the entire helically disposed first portion 5312. Distributing mechanical stresses may result in longer mechanical life for the electrical interconnection member 5311. The helically disposed first portion 5312 of the electrical interconnection member 5311 may be helically disposed in a non-overlapping manner (e.g., no portion of the electrical interconnection member 5311 may overlie itself in the region of the helix). It will be appreciated that in another embodiment, the pivot axis of the transducer array 5307 and accompanying structure may be offset from the central axis 5308. It will be further appreciated that in various embodiments, the axis of the helix, the pivot axis of the transducer array 5307, and the central axis 5308 may all be offset from each other, may all be coincidental, or two of the axes may be coincidental and offset from the third.

The electrical interconnection member 5311 may include ground and base layers. The ground and base layers may be configured differently than the other conductors of the electrical interconnection member 5311. For example, the ground layer may be in the form of a plane extending across the width of the electrical interconnection member 5311 and extending along the entire length of the electrical interconnection member 5311. Along the first portion of the electrical interconnection member 5312, the ground layer and/or the base layer may be separated from the remainder of the first portion of the electrical interconnection member 5312. Accordingly, the ground layer and/or base layer may be in the form of separate conductors (not shown) between the first end 5332 and the interconnection support 5334. Such an arrangement may result in a more flexible structure than that illustrated in FIG. 53 where the first portion of the electrical interconnection member 5312 includes the ground and base layers.

The first portion of the electrical interconnection member 5312 disposed within the enclosed volume 5317 may include additional layers of insulation relative to the second portion 5313. Such additional layers may provide protection against the fluid occupying the enclosed volume and/or such additional layers may provide protection against wear due to the first portion of the electrical interconnection member 5312 contacting other components (e.g., the case 5305). The additional layers may, for example, be in the form of one or more coatings and/or laminates.

The portion of the case 5305 that surrounds the enclosed volume 5317 in the region of the first portion of the electrical interconnection member 5312 may be structurally reinforced to resist kinking. Such reinforcement may be in the form of additional layers laminated to the inner and/or outer surface of the case 5305 or in the form of a structural support member secured to the case 5305.

In an embodiment, the first portion 5312 of the electrical interconnection member 5311 may include a total of about three revolutions about the central axis 5308. The total length of the catheter tip case 5305 may be selected to accommodate the number of revolutions needed for the first portion 5312 of the electrical interconnection member 5311. The total number of helical revolutions for the first portion 5312 of the electrical interconnection member 5311 may be determined based at least partially on desired coil expansion and contraction during pivotal movement, the desired level of counteracting torque imparted on the motor 5306 by the first portion 5312 during reciprocal movement, and the desired overall length of the catheter tip case 5305. Within the enclosed volume 5317, the first portion 5312 of the electrical interconnection member 5311 may be helically disposed such that there is a clearance between the outer diameter of the helix of the first portion 5312 and the inner surface of the catheter tip case 5305 as shown in FIG. 53.

The helically disposed first portion 5312 of the electrical interconnection member 5311 may be disposed such that a volume within the helically disposed first portion 5312 may contain a tube or other component with a lumen therethrough or other appropriate component. Such lumens may accommodate any appropriate use such as, for example, catheter insertion, drug delivery, device retrieval, and/or guidewire tracking. For example, a tube with a lumen therethrough may be disposed within the helically disposed first portion 5312. Such a tube may extend form the proximal end of the catheter probe assembly 5300, pass through the enclosed volume end wall 5318 (in embodiments including the enclosed volume end wall 5318) and past the bubble trap 5322 (in embodiments including the bubble trap 5322). In such an embodiment, the bubble trap 5322 may be offset from the central axis 5308 to accommodate the tube. A portion of such a lumen may extend through at least a portion of the first portion of the electrical interconnection member 5312. In an embodiment, the tube and lumen may terminate in a side port. For example, the lumen may terminate at the sidewall of the case in the region where the helically disposed first portion 5312 is located.

The interconnection support 5334 may serve to support an interconnection between the electrical interconnection member 5311 and a flexboard 5335. As noted, the second end 5333 of the first portion 5312 of the electrical interconnection member 5311 may be fixedly secured to the interconnection support 5334. Additionally, the flexboard 5335 may be fixedly secured to the interconnection support 5334. The individual conductors of the electrical interconnection member 5311 may be electrically connected to individual conductors of the flexboard 5335. The flexboard 5335 may serve to electrically interconnect the electrical interconnection member 5311 to the transducer array 5307. Insulative material may be disposed over the electrical interconnections between the electrical interconnection member 5311 and the flexboard 5335. The insulative material may be laminated over the electrical interconnections. In another embodiment, a rigid interconnection member may be used in place of the above-described flexboard 5335. Such a rigid interconnection member may serve to electrically interconnect the electrical interconnection member 5311 to the transducer array 5307.

The interconnection support 5334 may be configured as a hollow cylinder operable to be disposed about the outer surface of the motor 5306. Alternatively, the interconnection support 5334 may be configured as a curved plane that is not wrapped completely around the outer surface of the motor 5306. In either circumstance (e.g., hollow cylinder or curved plane), the interconnection support 5334 may be operable to rotate about a portion of the outer surface of the motor 5306. In this regard, as the motor 5306 reciprocally pivots the transducer array 5307, the transducer array backing 5328 by virtue of its fixed connection to the transducer array 5307 will also reciprocally pivot. In turn, by virtue of its fixed connection to the transducer array backing 5328, the flexboard 5335 will also reciprocally pivot. In turn, by virtue of their fixed connection to the flexboard 5335, the interconnection support 5334 and the second end 5333 of first portion 5312 the electrical interconnection member 5311 will also reciprocally pivot along with the transducer array 5307.

In another embodiment, the interconnection support 5334 and the flexboard 5335 may be constructed from a single flexboard. In such an embodiment, the interconnection support 5334 portion of the single flexboard may be formed into at least a portion of a cylinder such that it may be disposed at least partially about the outer surface of the motor 5306.

Although the transducer array 5307 and associated members are generally described herein as being disposed in a catheter tip 5301 at a distal end 5303 of the catheter probe assembly 5300, other configurations are contemplated. For example, in another embodiment, the members disposed within the catheter tip 5301 may be disposed at a point along the catheter shaft 5302 that is offset from the distal end 5303 of the catheter probe assembly 5300. In this regard, portions of the catheter shaft 5302 and/or other components may be disposed distal to the catheter tip 5301.

In an alternate embodiment, the catheter tip case 5305 may be in the form of a protective cage disposed about the electrical interconnection member 5311, motor 5306, array 5307, and other appropriate components of the catheter probe assembly 5300. Such a cage may allow blood (or other bodily fluid) into the volume corresponding to the enclosed volume 5317 of the embodiment of FIG. 53. Such an embodiment would not require the bellows member 5320 or the bubble trap 5322. The cage may be open enough to allow blood to flow throughout the volume corresponding to the enclosed volume 5317, yet have enough structure to assist in protecting blood vessels and/or other patient structures from damage from contact with the catheter probe assembly 5300. Moreover, in such an embodiment an acoustic structure may be interconnected to the array 5307. The acoustic structure may be made from a material or materials selected to maintain the imaging capabilities of the array 5307. The acoustic structure may be rounded in cross section to reduce turbulence in the surrounding blood, reduce damage to the surrounding blood cells, and aid in avoiding thrombus formation while the array is undergoing reciprocal pivotal movement. Other components may also be shaped to help reduce turbulence, avoid thrombus formation, and avoid damage to blood cells.

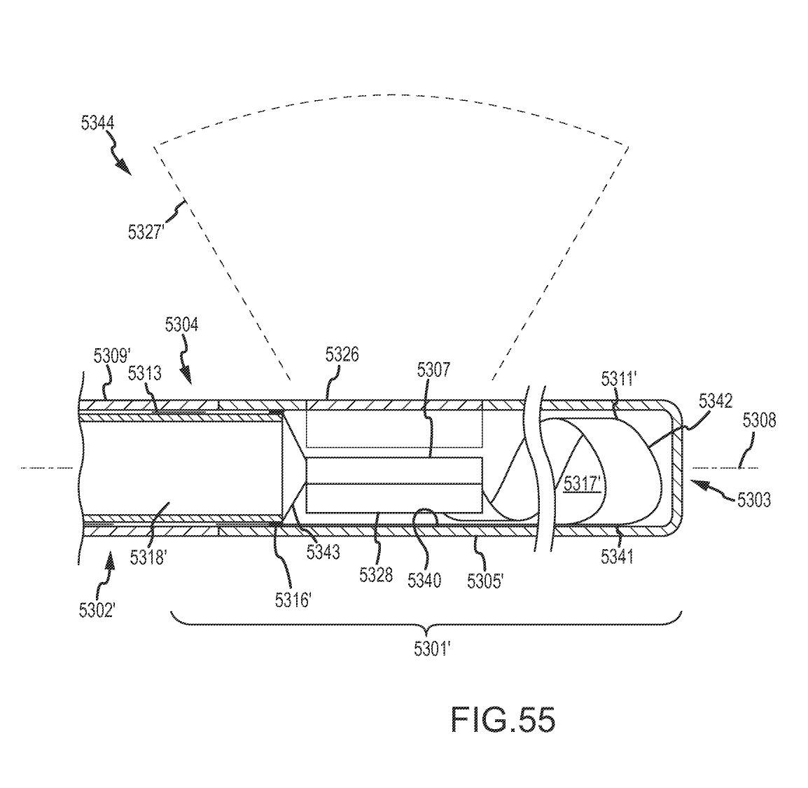

FIG. 55 is a partial cross-sectional view of an embodiment of an ultrasound catheter probe assembly 5344. Items similar to those of the embodiment of FIG. 53 are designated by a prime symbol (′) following the reference numeral. The catheter probe assembly 5344 includes a catheter tip 5301′ attached to a catheter shaft 5302′. Generally, the catheter probe assembly 5344 includes a driveshaft 5343 interconnected to the transducer array 5307. The driveshaft 5343 is operable to reciprocate and therefore reciprocate the transducer array 5307 interconnected to it. An electrical interconnection member 5311′ includes a first portion 5342 disposed in the distal end 5303 of the catheter probe assembly 5344 and operable to accommodate the reciprocal motion of the transducer array 5307. The electrical interconnection member 5311′ further includes a second portion 5313 disposed along the catheter shaft 5302′. The electrical interconnection member 5311′ further includes a third portion 5340 disposed along the catheter tip case 5305′ and operable to electrically interconnect the first portion 5342 to the second portion 5313.

The catheter probe assembly 5344 may generally be sized and shaped for insertion into a patient and subsequent imaging of an internal portion of the patient. The catheter probe assembly 5344 may generally include the distal end 5303 and a proximal end (not shown). During imaging, the distal end 5303 of the catheter probe assembly 5344 may be disposed within the body of a patient. A catheter tip 5301′ may be disposed between the distal end 5303 and a proximal end 5304 of the catheter tip 5301′. The catheter tip 5301′ may include a catheter tip case 5305′. The catheter tip 5301′ may include a central axis 5308. An enclosed volume 5317′ may be defined by the catheter tip case 5305′ and the driveshaft 5343. The enclosed volume 5317′ may be fluid-filled and sealed.

The catheter shaft 5302′ may use any appropriate guidance method such as, but not limited to, a set of control wires and associated controls to actively steer the catheter shaft 5302′. The catheter shaft 5302′ may be flexible and therefore be operable to be guided through and follow contours of the structure of the patient, such as the contours of the vasculature system.

The catheter probe assembly 5344 includes the transducer array 5307 and the array backing 5328. Generally, the transducer array 5307 is operable to transmit ultrasonic energy through the acoustic window 5326 of the catheter tip case 5305′. In general, the images generated by the catheter probe assembly 5344 may be of a subject (e.g., internal structure of a patient) within an image volume 5327′.

The transducer array 5307 may be interconnected to the driveshaft 5343, and the driveshaft 5343 may be operable to reciprocally pivot the transducer array 5307 about the central axis 5308 such that the image plane is swept about the central axis 5308 to form the image volume 5327′ as shown in FIG. 55. The sweeping of the image plane about the central axis 5308 enables the transducer array 5307 to scan the entire image volume 5327′ and thus a three dimensional image of the image volume 5327′ may be generated. The driveshaft 5343 may be operable to reciprocally pivot the transducer array 5307 at a rate sufficient enough to generate real-time or near real-time three-dimensional images of the image volume 5327′. The transducer array 5307 may be interconnected to the driveshaft at a proximal end of the transducer array 5307.

The driveshaft 5343, and therefore the transducer array 5307 interconnected to the driveshaft 5343, may be reciprocated using any appropriate means. For example, the proximal end of the catheter probe assembly 5344 may include a motor capable of reciprocally driving the driveshaft 5343 in both clockwise and counterclockwise directions. In this regard, the motor may be operable to reciprocally pivot the driveshaft 5343 and therefore reciprocally pivot the transducer array 5307 interconnected to the driveshaft 5343.

When driveshaft 5343 position is known, the corresponding position of the transducer array 5307 will be known. Driveshaft 5343 position may be tracked in any appropriate manner, such as through the use of an encoder and/or a magnetic position sensor.

The electrical interconnection member 5311′ may electrically interconnect the transducer array 5307 with the ultrasound imaging apparatus. The electrical interconnection member 5311′ may be a multi-conductor cable comprising of a plurality of conductors arranged side-by-side with electrically nonconductive material between the conductors.

The electrical interconnection member 5311′ may be anchored such that a portion of it is fixed relative to the catheter tip case 5305′. As noted above, the second portion 5313 of the electrical interconnection member 5311′ may be secured to the catheter shaft 5302′. Within the enclosed volume 5317′, the third portion 5340 of the electrical interconnection member 5311′ may be secured to the inner surface of the catheter tip case 5305′. The third portion 5340 of the electrical interconnection member 5311′ may be secured to the catheter tip case 5305′ in a region corresponding to the position of the transducer array 5307. In this regard, the third portion 5340 of the electrical interconnection member 5311′ may be disposed such that it does not interfere with the reciprocal movement of the transducer array 5307. Any appropriate method of anchoring the electrical interconnection member 5311′ to the catheter tip case 5305′ may be used. For example, adhesive may be used.

The first portion 5342 of the electrical interconnection member 5311′ is operable to maintain an electrical connection to the transducer array 5307 while the transducer array 5307 is pivoting relative to the catheter tip case 5305′. This may be achieved by coiling the first portion 5342 of the electrical interconnection member 5311′ within the enclosed volume 5317′. One end of the first portion 5342 of the electrical interconnection member 5311′ may be anchored to the catheter tip case 5305′ at an anchor point 5341 that is distal to the transducer array 5307. The other end of the first portion 5342 of the electrical interconnection member 5311′ may be electrically interconnected to the array backing 5328 or to a flexboard or other electrical member (not shown) that is in turn electrically interconnected to the transducer array 5307. Where the electrical interconnection member 5311′ is ribbon shaped, the first portion 5342 of the electrical interconnection member 5311′ may be disposed such that a top or bottom side of the ribbon faces and wraps about the central axis 5308.

FIG. 55 illustrates a configuration where the first portion 5342 of the electrical interconnection member 5311′ is helically disposed within the portion of the enclosed volume 5317′ distal to the transducer array 5307. The first portion 5342 of the electrical interconnection member 5311′ may be coiled about the central axis 5308 a plurality of times. The first portion 5342 of the electrical interconnection member 5311′ may be coiled about the central axis 5308 such that the first portion 5342 of the electrical interconnection member 5311′ forms a helix about the central axis 5308. As in the embodiment of FIG. 53, by coiling the electrical interconnection member 5311′ about the central axis 5308 a plurality of times, undesirable counteracting torque on the pivoting of the transducer array 5307 may be significantly avoided.

In an embodiment, the first portion 5342 of the electrical interconnection member 5311′ may include a total of about three revolutions about the central axis 5308. The total length of the catheter tip case 5305′ may be selected to accommodate the number of revolutions needed for the first portion 5342 of the electrical interconnection member 5311′.

A distal end of the driveshaft 5343 may be sealed along its outer perimeter using a sealing material 5316′. The sealing material 5316′ may be disposed as illustrated between the driveshaft 5343 and an inner surface of the catheter tip case 5305′. In another embodiment, the outer layer 5309′ of the catheter shaft 5302′ may extend to or beyond the distal end of the driveshaft 5343 and in such an embodiment, the sealing material 5316′ may be disposed between the driveshaft 5343 and an inner surface of the outer layer 5309′. The sealing material 5316′ may include any appropriate material and/or structure that allows relative rotational movement between the driveshaft 5343 and the outer layer 5309′ while substantially preventing the flow of fluid from the enclosed volume 5317′ past the sealing material 5316′. In another embodiment, the catheter shaft 5302′ may include an inner layer (similar to the inner layer 5310 of FIG. 53) and the driveshaft 5343 may be disposed within the inner layer. In such an embodiment, the inner layer, the outer layer 5309′, a volume between the inner layer and the outer layer 5309′, or any combination thereof, may house additional components, such as, for example, pull wires, reinforcing members and/or additional electrical conductors.

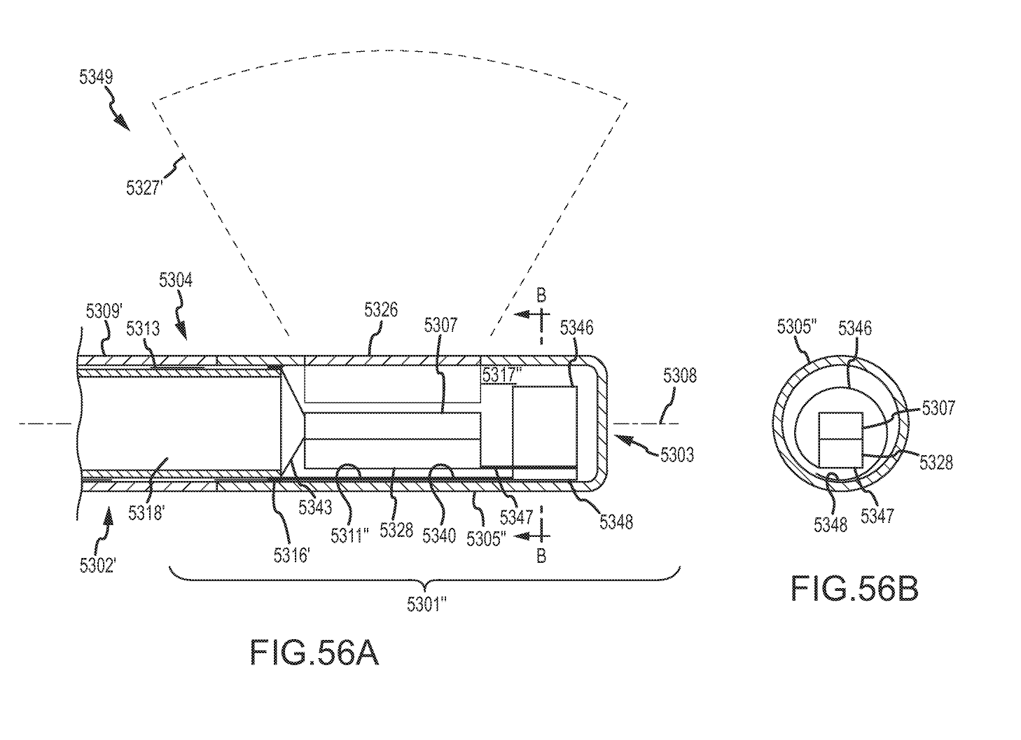

FIGS. 56A and 56B illustrate another embodiment of an ultrasound catheter probe assembly 5349. Items similar to those of the embodiment of FIG. 55 are designated by a double prime symbol (″) following the reference numeral. The catheter probe assembly 5349 includes a catheter tip 5301″ attached to a catheter shaft 5302′. In this embodiment, the catheter probe assembly 5349 includes a driveshaft 5343 interconnected to the transducer array 5307. An electrical interconnection member 5311″ includes a first portion 5346 disposed in the distal end 5303 of the catheter probe assembly 5349 and operable to accommodate the reciprocal motion of the transducer array 5307. The electrical interconnection member 5311″ further includes a second portion 5313 disposed along the catheter shaft 5302″. The electrical interconnection member 5311″ further includes a third portion 5340 disposed along the catheter tip case 5305″ and operable to electrically interconnect the first portion 5346 to the second portion 5313. An enclosed volume 5317″ may be defined by a catheter tip case 5305″ and the driveshaft 5343. The enclosed volume 5317″ may be fluid-filled and sealed.

The catheter probe assembly 5349 includes the transducer array 5307 and the array backing 5328. The transducer array 5307 may be interconnected to the driveshaft 5343, and the driveshaft 5343 may be operable to reciprocally pivot the transducer array 5307 about the central axis 5308 such that the image plane is swept about the central axis 5308 to form a three dimensional image volume 5327′ as shown in longitudinal cross section in FIG. 56A.

The electrical interconnection member 5311″ may electrically interconnect the transducer array 5307 with the ultrasound imaging apparatus (not shown). The electrical interconnection member 5311″ may include a portion including a multi-conductor cable comprising of a plurality of conductors arranged side-by-side with electrically nonconductive material between the conductors. The electrical interconnection member 5311″ may further include a portion including flexboard.

The electrical interconnection member 5311″ may be anchored such that a portion of it is fixed relative to the catheter tip case 5305″. As noted above, the second portion 5313 of the electrical interconnection member 5311″ may be secured to the catheter shaft 5302′. Within the enclosed volume 5317″, the third portion 5340 of the electrical interconnection member 5311″ may be secured to the inner surface of the catheter tip case 5305″. The third portion 5340 of the electrical interconnection member 5311″ may be secured to the catheter tip case 5305″ in a region corresponding to the position of the transducer array 5307. In this regard, the third portion 5340 of the electrical interconnection member 5311″ may be disposed such that it does not interfere with the reciprocal movement of the transducer array 5307. Any appropriate method of anchoring the third portion 5340 of the electrical interconnection member 5311″ to the catheter tip case 5305″ may be used. For example, adhesive may be used.

The first portion 5346 of the electrical interconnection member 5311″ is operable to maintain an electrical connection to the transducer array 5307 while the transducer array 5307 is pivoting relative to the catheter tip case 5305″. This may be achieved by coiling the first portion 5346 of the electrical interconnection member 5311″ within the enclosed volume 5317″. One end of the first portion 5346 of the electrical interconnection member 5311″ may be anchored to the catheter tip case 5305″ at an anchor point 5348 that is distal to the transducer array 5307. The other end of the first portion 5346 of the electrical interconnection member 5311″ may be electrically interconnected to a coil-to-backing portion 5347 of the electrical interconnection member 5311″. The coil-to-backing portion 5347 of the electrical interconnection member 5311″ may electrically interconnect the first portion 5346 of the electrical interconnection member 5311″ to the array backing 5328. The first portion 5346 of the electrical interconnection member 5311″ may have a generally flat cross-section and be disposed such that a top or bottom side of the first portion 5346 faces and wraps about the central axis 5308. The first portion 5346 of the electrical interconnection member 5311″ may be coiled in a “clock spring” arrangement where, as illustrated in FIGS. 56A and 56B, substantially the entirety of the first portion 5346 of the electrical interconnection member 5311″ is positioned at the same point along the central axis 5308. In this regard, a center line of the first portion 5346 of the electrical interconnection member 5311″ may generally occupy a single plane that is disposed perpendicular to the central axis 5308. One end of the clock spring of the first portion 5346 of the electrical interconnection member 5311″ may be electrically interconnected to the third portion 5340, while the other end may be electrically interconnected to the coil-to-backing portion 5347. Although FIGS. 56A and 56B illustrates the clock spring of the first portion 5346 as having a single coil, the clock spring of the first portion 5346 may be comprised of more or less than a single coil. For example, in an embodiment, the clock spring of the first portion 5346 may include 1.5 or 2 concentric coils (i.e., the clock spring of the first portion 5346 may wrap around 1.5 or 2 times). In an arrangement, the clock spring of the first portion 5346, the third portion 5340, and the coil-to-backing portion 5347 of the electrical interconnection member 5311″ may be constructed from a single flexboard or other conductor such as a GORE™ Micro-Miniature Ribbon Cable.

Similar to the embodiments of FIGS. 53 and 55, by coiling the clock spring of the first portion 5346 the electrical interconnection member 5311″ (e.g., about an axis parallel to the central axis 5308), undesirable counteracting torque on the pivoting of the transducer array 5307 may be significantly avoided. In this regard, pivoting of the transducer array 5307 about the central axis 5308 in such a configuration may result in a slight tightening, or slight loosening, of the turns of the clock spring of the first portion 5346 of the electrical interconnection member 5311″. Such a slight tightening and loosening may result in each coil (e.g., each individual rotation of the clock spring about the central axis 5308) producing only a small lateral displacement and corresponding displacement of fluid.

In alternate configurations of the catheter probe assemblies 5344, 5349 of FIGS. 55 and 56A, motors (not shown) may be used in place of the driveshafts 5343. Such motors may be located near the proximal ends of the catheter tips 5301′, 5301″. Such motors may be disposed within the enclosed volumes 5317′, 5317″, or they may be disposed outside of the enclosed volumes 5317′, 5317″.

Similar to as described above with reference to FIG. 53, in alternate embodiments, the catheter tip cases 5305′, 5305″ of the embodiments of FIGS. 55 and 56A may be in the form of a protective cages disposed about the electrical interconnection members 5311′, 5311″, arrays 5307, and other appropriate components of the catheter probe assemblies 5344, 5349. Such cages may allow blood (or other bodily fluid) into the volumes corresponding to the enclosed volumes 5317′, 5317″, of the embodiments of FIGS. 55 and 56A. The cages may be open enough to allow blood to flow throughout the volumes corresponding to the enclosed volumes 5317′, 5317″, yet have enough structure to assist in protecting tissues from damage due to contact with the catheter probe assemblies 5344, 5349 or components thereof. Moreover, and similar to as discussed above, acoustic structures, such as lenses or covers, may be interconnected to the signal emitting face of arrays 5307. Other components may also be shaped to help reduce turbulence, avoid thrombus formation, and avoid damage to tissue or blood cells.

In embodiments that include an enclosed volume within a catheter tip case, and embodiments where the catheter tip case is a cage that is open to the surrounding environment, the portion of the catheter tip case in the region of the helically coiled electrical interconnect (e.g., the first portion of the electrical interconnect 5312) may be steerable and/or flexible. In such a steerable and/or flexible configuration, the mechanical stresses due to steering and/or flexing on the electrical interconnect may be distributed over substantially the entire the helically coiled portion.

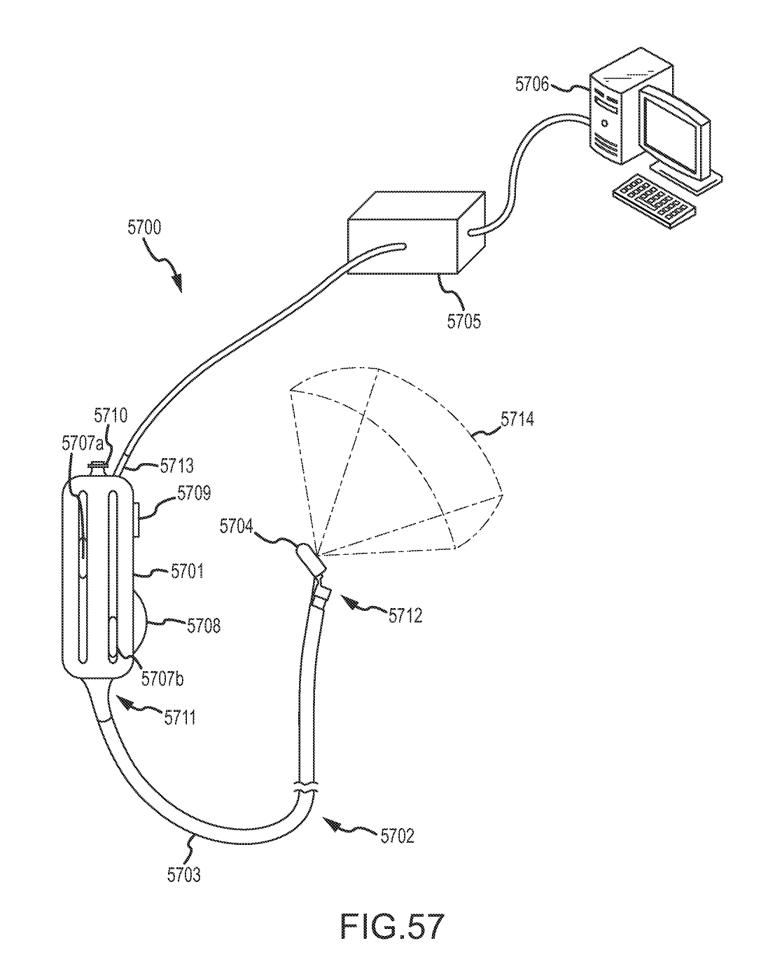

FIG. 57 illustrates an ultrasound imaging system 5700 suitable for real-time three dimensional imaging with a handle 5701 and a catheter 5702. The catheter 5702 includes a catheter body 5703 and a deflectable member 5704. The deflectable member 5704 may be hingedly connected to a distal end 5712 of the catheter body 5703. The deflectable member 5704 may have a hinge. The catheter body 5703 may be flexible and capable of bending to follow the contours of a body vessel into which it is being inserted or track over a guidewire or through a sheath.

The ultrasound imaging system 5700 may further include a motor controller 5705 and an ultrasound console 5706. The motor controller 5705 may be operable to control a motor (embodiments of which are discussed below) that may be disposed within or interconnected to an ultrasound array within the deflectable member 5704. The ultrasound console 5706 may include an image processor, operable to process signals from the ultrasound array, and a display device, such as a monitor. The various functions described with reference to the motor controller 5705 and ultrasound console 5706 may be performed by a single component or by any appropriate number of discrete components.

Hinges described herein may rely on bending (e.g., living hinges) and/or a pivot (e.g., where the hinge includes a pin along a pivot axis) to define the relative motion between the deflectable member and the catheter body. Such hinges may include a non-tubular portion that allows the deflectable member and the catheter body to move relative to each other. Thus, a typical catheter steering arrangement that relies on one side of a tubular portion of the catheter being compressed to a greater degree than an opposing side of the tubular portion to achieve catheter bending is not typically considered a hinge.

The handle 5701 may be disposed at a proximal end 5711 of the catheter 5702. The user (e.g., clinician, technician, interventionalist) of the catheter 5702 may control the steering of the catheter body 5703, deflection of the deflectable member, and various other functions of the catheter 5702. In this regard, the handle 5701 includes two sliders 5707a, 5707b for steering the catheter body 5703. These sliders 5707a, 5707b may be interconnected to control wires such that when the sliders 5707a, 5707b are moved relative to each other, a portion of the catheter body 5703 may be curved in a controlled manner. Any other appropriate method of controlling control wires within the catheter body 5703 may be utilized. For example, the sliders could be replaced with alternative means of control such as turnable knobs or buttons. Any appropriate number of control wires within the catheter body 5703 may be utilized.

The handle 5701 further includes a deflection controller 5708. The deflection controller 5708 may be used to control the deflection of the deflectable member 5704 relative to the catheter body 5703. The illustrated deflection controller 5708 is in the form of a rotatable knob, where a rotation of the deflection controller 5708 will produce a corresponding deflection of the deflectable member 5704. Other configurations of the deflection controller 5708 are contemplated, including, for example, a slider similar to slider 5707a.

The handle 5701 may further include a motor activation button 5709 in embodiments of the ultrasound imaging system 5700 that include a motor within the deflectable member 5704. The motor activation button 5709 may be used to activate and/or deactivate the motor. The handle 5701 may further include a port 5710 in embodiments of the ultrasound imaging system 5700 that include a lumen within the catheter body 5703. The port 5710 is in communication with the lumen such that the lumen may be used for conveyance of a device and/or material.

In use, the user may hold the handle 5701 and manipulate one or both sliders 5707a, 5707b to steer the catheter body 5703 as the catheter 5702 is moved to a desired anatomical position. The handle 5701 and sliders 5707a, 5707b may be configured such that the position of the sliders 5707a, 5707b relative to the handle 5701 may be maintained, thereby maintaining or “locking” the selected position of the catheter body 5703. The deflection controller 5708 may then be used to deflect the deflectable member 5704 to a desired position. The handle 5701 and deflection controller 5708 may be configured such that the position of the deflection controller 5708 relative to the handle 5701 may be maintained, thereby maintaining or “locking” the selected deflection of the deflectable member 5704. In this regard, the deflectable member 5704 may be selectively deflectable, and the catheter body 5703 may be selectively steered, independently. Also, the deflection of the deflectable member 5704 may be selectively locked, and the shape of the catheter body 5703 may be selectively locked, independently. Such maintenance of position may at least partially be achieved by, for example, friction, detents, and/or any other appropriate means. The controls for the steering, deflection, and motor may all be independently operated and controlled by the user.

The ultrasound imaging system 5700 may be used to capture images of a three dimensional imaging volume 5714 and/or capture 3D images in real-time 5714. The deflectable member 5704 may be positioned by steering the catheter body 5703, articulating the deflectable member 5704, or by a combination of steering the catheter body 5703 and articulating the deflectable member 5704. Moreover, in embodiments with a lumen, the ultrasound imaging system 5700 may further be used, for example, to deliver devices and/or materials to a selected region or selected regions within a patient.

The catheter body 5703 may have at least one electrically conductive wire that exits the catheter proximal end 5711 through a port or other opening in the catheter body 5703 and is electrically connected to a transducer driver and image processor (e.g., within the ultrasound console 5706).

Furthermore, in embodiments with a lumen, the user may insert an interventional device (e.g., a diagnostic device and/or therapeutic device) or material, or retrieve a device and/or material through the port 5710. The user may then feed the interventional device through the catheter body 5703 to move the interventional device to the distal end 5712 of the catheter body 5703. Electrical interconnections between the ultrasound console 5706 and the deflectable member 5704 may be routed through an electronics port 5713 and through the catheter body 5703 as described above.

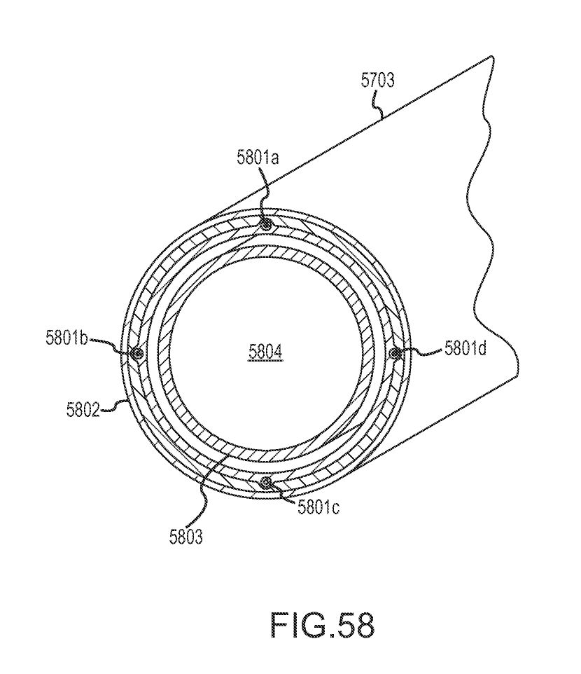

FIG. 58 is a cross-sectional view of the catheter body 5703 of FIG. 57. The catheter body 5703 includes four wires 5801a through 5801d disposed at equal intervals within catheter body 5703 for use in steering a steerable segment of the catheter body 5703 (also known as 4-way steering) for guiding the catheter 5702 to the appropriate anatomy. The steering may be by selective flexure along a steerable segment of the catheter body 5703. In this regard, two control wires 5801a, 5801c may be interconnected to slider 5707a such that moving the slider 5707a in a first direction causes the distal portion of the control wire 5801a to be pulled toward the handle 5701. Similar manipulation of the control wires 5801b through 5801d or appropriate combinations thereof may cause the steerable section of the catheter body 5703 to bend in a desired direction. Alternatively, in some embodiments, fewer or more than four control wires may be used. Control wires may also comprise cables or flat-sided ribbons.

Catheter body 5703 incorporates a tube-in-tube design where an inner tube 5803 with a lumen 5804 is disposed within an outer tube 5802 and the inner tube 5803 is movable relative to the outer tube 5802 to control the deflection of the deflectable member 5704 (e.g., in a manner such as described with reference to FIGS. 5C and 5D). The outer tube 5802 may include multiple layers and the wires 5801a through 5801d may be disposed within control wire lumens disposed within the layers of the outer tube 5802.

Alternatively, deflection of the deflectable member 5704 may be achieved by rotating the inner tube 5803 relative to the outer tube 5802 (e.g., in a manner such as described with reference to FIGS. 35A and 35B).

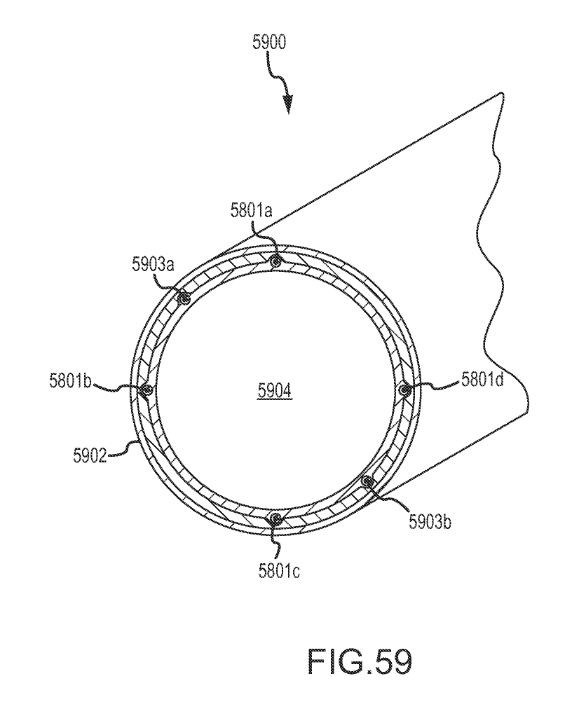

FIG. 59 illustrates an embodiment of a catheter body 5900 that may be used in the ultrasound imaging system 5700 in place of catheter body 5703. The catheter body 5900 includes control wires 5801a through 5801d to steer the catheter body 5900 in a similar manner as described with respect to FIG. 58. In place of the tube-in-tube design of FIG. 58, the catheter body 5900 may include a single tube 5902, and control wires 5903a and 5903b disposed therein that may be used to control the deflection of the deflectable member 5704. The control wires 5903a and 5903b may be similar in construction to control wires 5801a through 5801d. In other embodiments, electrically conductive elements (e.g., a flex circuit or wires connected to a motor) may be disposed along and/or within the catheter body 5900 and may be used to control the deflection of the deflectable member 5704 (e.g., by pulling and/or pushing on such electrically conductive elements). Catheter body 5900 may include a lumen 5904.

Any other appropriate system for steering a catheter may be used in place of the 4-way steering illustrated in FIGS. 58 and 59. For example, additional control wires (and appropriate additional controls) may be used, or fewer control wires may be used to steer the catheter. Other appropriate types of steering systems may be employed, such as electrically activated members (e.g., electropolymers) and thermally activated members (e.g., comprising shape memory material).

Moreover, any other appropriate system for controlling the deflection of the deflectable members may be used in place of the tube-in-tube system or control wires 5903a, 5903b illustrated in FIGS. 58 and 59, respectively. For example, electrically activated members (e.g., electropolymers) and/or thermally activated members (e.g., comprising shape memory material) may be employed.

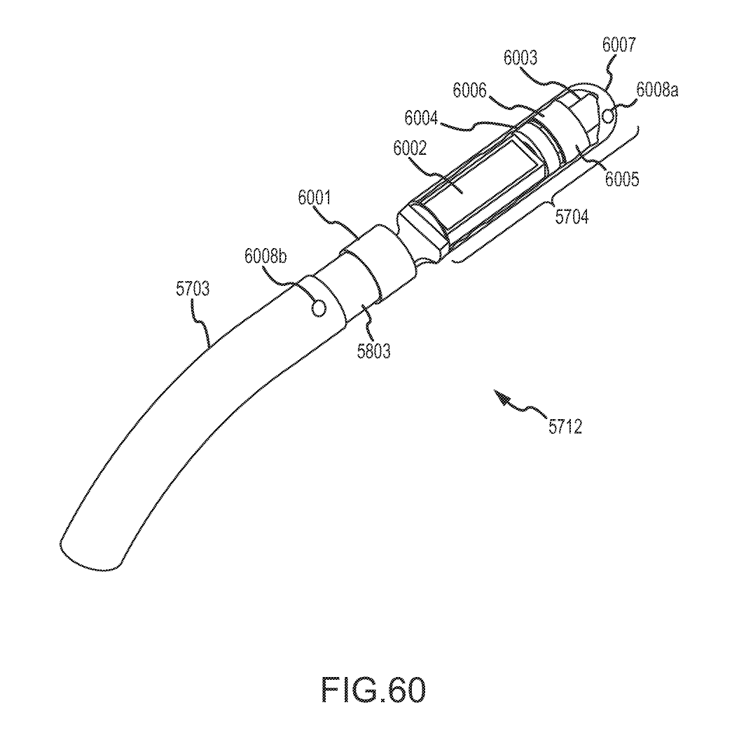

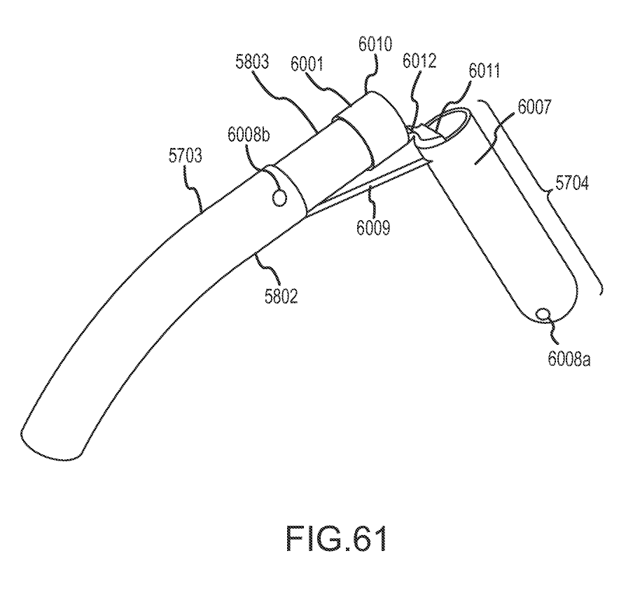

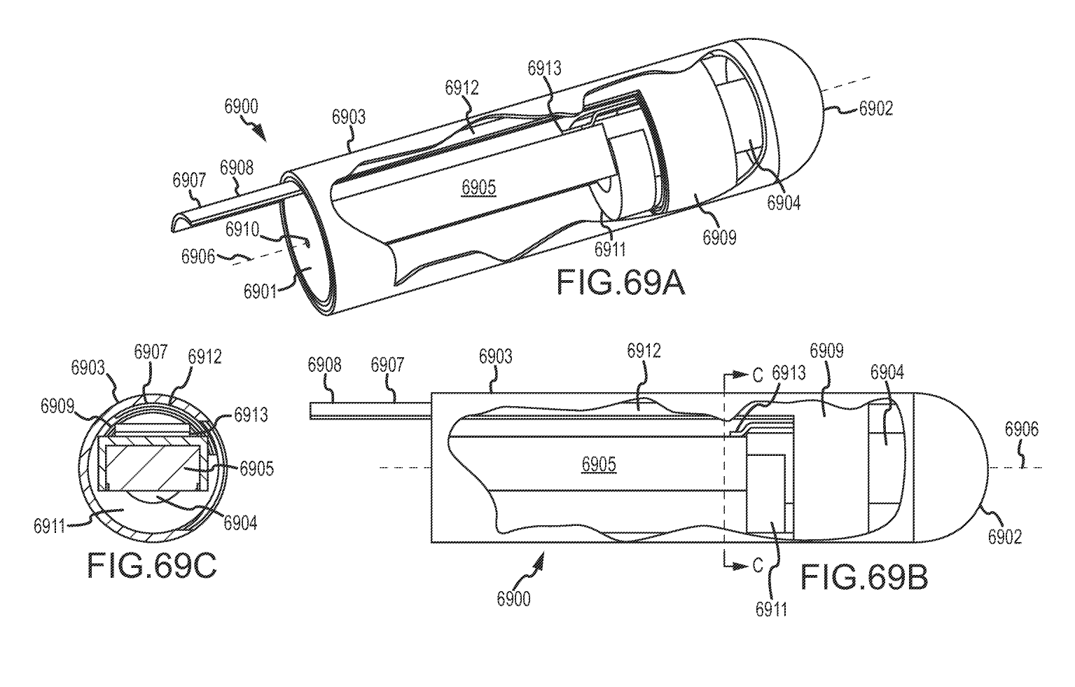

FIGS. 60 and 61 illustrate the distal end 5712 of catheter 5702. In the illustrated embodiment, the catheter body 5703 is connected by a hinge 6001 to the deflectable member 5704 (with a cutaway portion to reveal components within the deflectable member 5704). As illustrated in FIG. 60, a one dimensional transducer array 6002, motor 6003, motor mount 6004, and electrical interconnection member 6005 (that includes a clock spring portion 6006) may be disposed within a casing 6007 of the deflectable member 5704. The deflectable member 5704 and the components therein are described in detail with reference to FIGS. 69A through 69C. It is noted that other embodiments of deflectable members and/or other embodiments of structures that enable deflection of the various other embodiments of deflection members may be substituted for the deflectable member 5704 and/or the hinge 6001 illustrated in FIGS. 57, 60 and 61.

FIG. 61 illustrates the deflectable member 5704 in a position where it is deployed at about a +90 degree, forward-facing angle with respect to the end of the catheter body 5703. For explanatory purposes only, an angular value (e.g., the +90 degree angle of deflection shown in FIG. 61) may be used herein to describe the amount of rotation of a deflectable member with respect to a central axis of a catheter body away from a position where the deflectable member and catheter body are aligned. A positive value will generally be used to describe a rotation where the deflectable member is moved such that it is at least partially forward-facing (e.g., such that an ultrasound transducer array within the deflectable member is facing forward), and a negative value will generally be used to describe a rotation where the deflectable member is moved such that it is at least partially rearward-facing.

To deflect the deflectable member 5704 from the position of FIG. 60 to the position of FIG. 61, the inner tube 5803 may be advanced relative to the outer tube 5802. By virtue of the deflectable member 5704 being tethered to the outer tube 5703 by a tether 6009, the advancement may cause the deflectable member 5704 to rotate in a positive direction. The tether 6009 may be anchored to the deflectable member 5704 on one end and to the outer tube 5802 on the other end. The tether 6009 may be operable to prevent the tether anchor points from moving a distance away from each other greater than the length of the tether 6009. In this regard, through the tether 6009, the deflectable member 5704 may be restrainably interconnected to the outer tube 5802. Similarly, where the tether 6009 has adequate stiffness, retraction of the inner tube 5803 relative to the outer tube 5802 from the position shown in FIG. 60 may cause the deflectable member 5704 to rotate in a negative direction.

The tether 6009 may be a discrete device whose primary function is to control the deflection of the deflectable member 5704. In another embodiment, the tether 6009 may be a flexboard or other multiple conductor component that, in addition to providing the tethering function, electrically interconnects components within the deflectable member 5704 (e.g., the transducer array 6002) with components within the catheter body 5703 (e.g., similar to electrical interconnection member 104 of FIG. 5E) or elsewhere within the ultrasound imaging system 5700. In another embodiment, the tether 6009 may be a wire or wires used to electrically interconnect one or more components (e.g., sensors, motor 6003) within the deflectable member 5704 with the motor controller 5705, ultrasound console 5706, and/or other appropriate component of the ultrasound imaging system 5700.

FIGS. 60 and 61 illustrate a configuration using the living hinge 6001. A live or living hinge is a compliant hinge (flexure bearing) made from a flexible or compliant material, such as polymer. Generally, a living hinge joins two parts together, allowing them to pivot relative to each other along a bend line of the hinge. Living hinges are typically manufactured by injection molding. Polyethylenes, polypropylenes, polyurethanes, or polyether block amides such as PEBAX® are possible polymers for living hinges, due to their fatigue resistance.

The hinge 6001 allows for relative hinged movement between a first portion 6010 of the hinge 6001 and a second portion 6011 of the hinge 6001. The two portions 6010, 6011 are joined along a hinge line 6012 and the deflectable member 5704 and inner tube 5803 move relative to each other about the hinge line 6012. In this regard, the relative motion between the deflectable member 5704 and inner tube 5803 is constrained by a non-tubular element. This is in contrast to the relative movement between different sections of the catheter body 5703 that may occur due to manipulation of the wires 5801a through 5801d to steer the catheter body 5703, where the relative motion between the different sections of the catheter body 5703 is constrained by a tubular element (e.g., by the compression and/or elongation of the outer tube 5802 and/or the inner tube 5803).

The hinge 6001 may be a unitary part, such as a single molded part. Moreover, the hinge 6001 may be in direct contact with, and fixedly connected to, the parts whose relative motion is desired to be constrained. In this regard, the first portion of the hinge 6010 may in direct contact with and fixedly connected to the inner tube 5803, while the second portion 6011 of the hinge 6010 may be in direct contact with and fixedly connected to the deflectable member 5704.

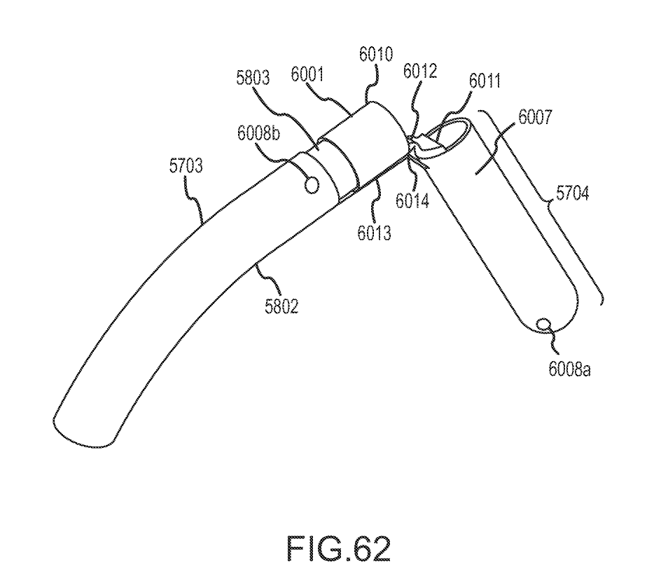

FIG. 62 illustrates a variation of the embodiment illustrated in FIGS. 60 and 61. In FIG. 62, the tether 6009 of FIGS. 60 and 61 is replaced with an actuation member 6013 that includes a hinge line 6014, thus the embodiment may use two living hinges (hinge 6001 with hinge line 6012 and hinge line 6014 of actuation member 6013) placed parallel to each other with tension applied to one as compression is applied to the other (e.g., by moving inner tube 5803 relative to outer tube 5802) to cause bending along both hinge lines 6012, 6014 in the same direction. By alternating which member (hinge 6001, actuation member 6013) is in tension and compression, the bend direction may be reversed. The hinge 6001 may be attached to the inner tube 5803 and may provide support for the deflectable member 5704. A flexboard (not shown) may be placed between the hinge 6001 and the actuation member 6013 or external to the hinge 6001 and the actuation member 6013. The actuation member 6013 may be attached to the deflectable member 5704 and the outer tube 5802 of the catheter body 5703. Alternatively, the actuation member 6013 may include a reinforced flexboard (not shown) that may act as a living hinge as well as an electrical interconnect member between the transducer array 6002 and an electrical conductor within the catheter body 5703. As compared to the embodiment of FIGS. 60 and 61, the embodiment of FIG. 62 may provide for a relatively large deflection angle of the deflectable member 5704 for a relatively small displacement between the outer tube 5802 and the inner tube 5803.

Embodiments of catheters described herein may also include one or more sensors for determining spatial positioning of the various components that may be inserted into a patient. For example, in concert with the imaging capability (e.g., 4D ultrasound imaging) of some of the embodiments, appropriately placed sensors may allow for the accurate identification of the spatial positions (e.g., within the cardiac chambers) of the various components (or portions thereof) of the embodiments. For example, relative positioning information provided by sensors facilitates the guidance of more complex ablation procedures, where electrical activity of the heart indicating treatment targets can be mapped to the catheter body and deflectable member positions.

An exemplary implementation of such sensors is illustrated in FIGS. 60 and 61 where a sensor 6008a placed at the distal end of the deflectable member 5704 may be used to accurately identify the spatial position and angular orientation of the deflectable member 5704 (e.g., when it is positioned within a cardiac chamber of a patient). Similarly, as illustrated in FIGS. 60 and 61, an optional second sensor 6008b placed at the distal end of the catheter body 5703 may be used to accurately identify the spatial position of the catheter body 5703. The use of two sensors allows the orientation of the catheter body 5703 relative to the deflectable member 5704 to be fully defined. The sensors 6008a, 6008b may be six degree of freedom (DOF) sensors that have the capability to pinpoint a relative position of a device with a high degree of accuracy. Recent advances in sensor design have reduced the size of such sensors to a diameter of about 0.94 mm (2.8 Fr). This profile provides the capability for these sensors to fit within the profile of, for example, a 9 to 10 Fr diameter catheter embodiment. Such 3D guidance sensors are available from Ascension Technology Corporation, Burlington, Vt., USA.

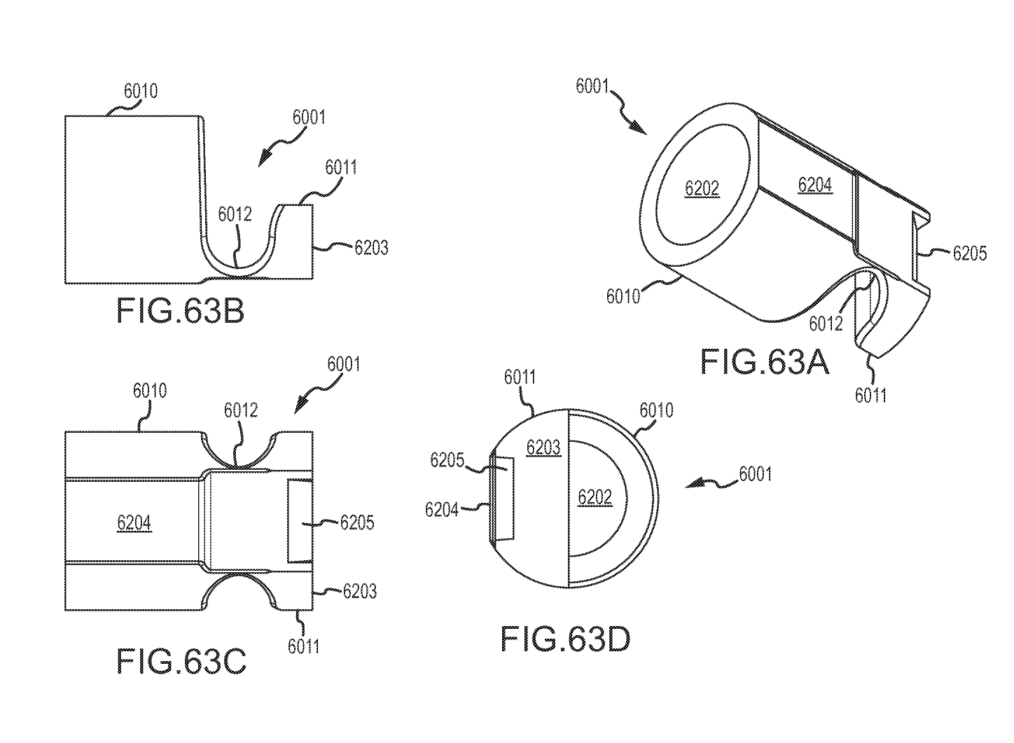

FIGS. 63A through 63D show the living hinge 6001 of FIGS. 60 through 62 isolated from the catheter 5702. The first portion 6010 of the living hinge 6001 is tubular to interface with the inner tube 5803. In alternate configurations, the first portion 6010 may be sized to interface with an outer wall of a distal end of a catheter body or with any other appropriate portion of a catheter body. The first portion 6010 may be sized such that a portion of a catheter body may be wrapped about the outer surface of the first portion 6010 to secure the first portion 6010 to the catheter body. The first portion 6010 may include a lumen 6202 which may provide access to a lumen of a catheter body (e.g., lumen 5804 of FIG. 58) to which the first portion 6010 is attached.

The second portion 6011 of the living hinge 6001 may be semicircular in shape and may be configured to interface with a deflectable member, such as deflectable member 5704 of FIGS. 60 through 62, or other appropriate member. The second portion 6011 may include an end wall 6203 that may interconnect to a deflectable member in any appropriate manner. For example, the end wall 6203 may interconnect to a deflectable member using adhesive, welds, pins, fasteners, or any combination thereof. Portions of the deflectable member may be overmolded or formed onto or over second portion 6011.

The second portion 6011 may neck down to a predetermined thickness at the hinge line 6012 to achieve a desired hinge strength while also achieving a desired level of resistance to bending.

The living hinge 6001 may include a flattened region 6204 disposed along an outer surface of the living hinge 6001. The flattened region 6204 may be sized to accept a flexboard or other electrical interconnection member that may connect electrical conductors in a catheter body to electrical components in a deflectable member. The living hinge 6001 may include a ramp 6205 which may allow clearance for an electrical interconnection member to pass into an attached deflectable member while not presenting a sharp edge against which the electrical interconnection member could contact when the deflectable member is deflected.

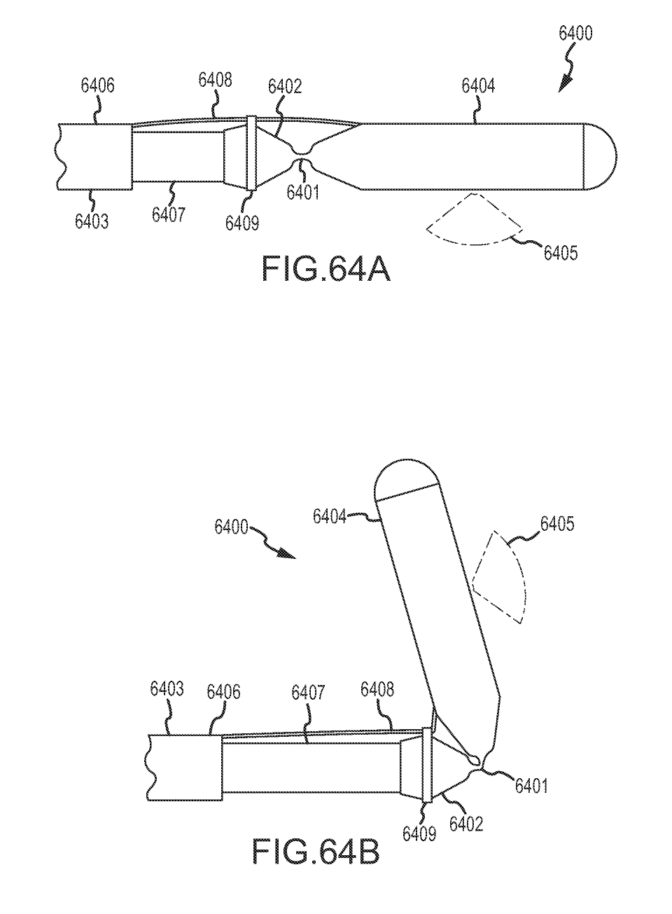

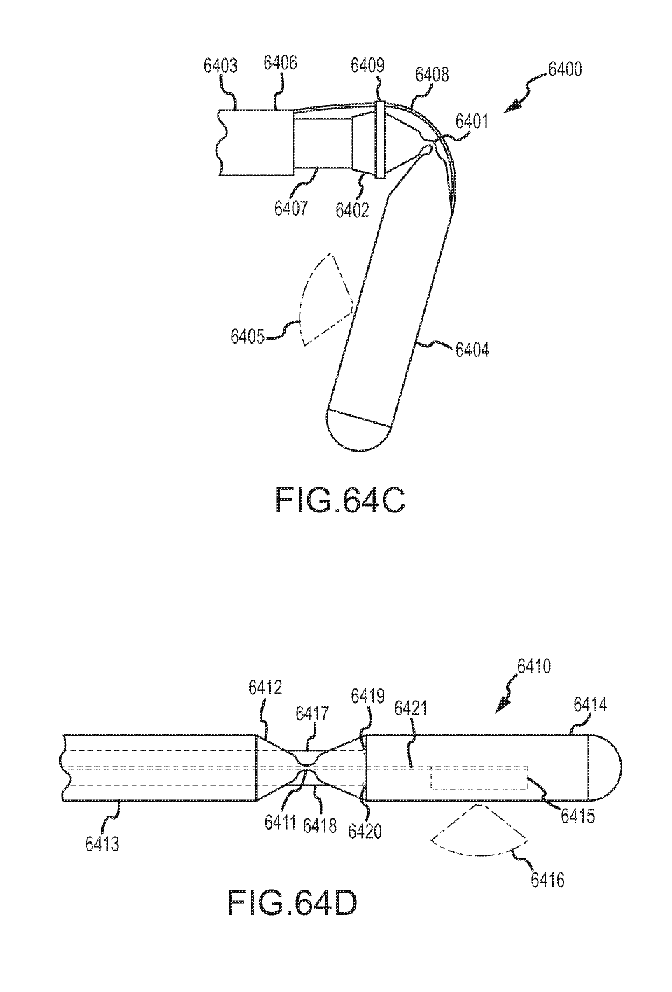

FIGS. 64A through 64C illustrate an embodiment of a catheter 6400 that includes a centrally disposed living hinge 6401 positioned between a distal end 6402 of a catheter body 6403 and a deflectable member 6404. The deflectable member 6404 may contain a transducer array (e.g., fixed one dimensional array, pivotable one dimensional array, two-dimensional array) capable of imaging a plane or volume 6405 (schematically represented) disposed proximate to the deflectable member 6404.

As illustrated in FIGS. 64B and 64C, the deflectable member 6404 may have a total range of motion of at least about 200 degrees. FIG. 64B shows the deflectable member 6404 pivoted about +100 degrees from the aligned position (FIG. 64A), and FIG. 64C shows the deflectable member 6404 pivoted about −100 degrees from the aligned position. This range of motion is achieved by displacing an outer tube 6406 of the catheter body 6403 relative to an inner tube 6407. A tether 6408 is interconnected to the outer tube 6406 and the deflectable member 6404. The tether 6408 may be restrained by a restraining member 6409 such that a portion of the tether 6408 remains proximate to the distal end 6402.

Accordingly, when the outer tube 6406 is moved proximally relative to the inner tube 6407 as illustrated in FIG. 64B, the tether 6408 pulls proximally on the deflectable member 6404 causing it to pivot in a positive direction. Similarly, when the outer tube 6406 is moved distally relative to the inner tube 6407 as illustrated in FIG. 64C, the tether 6408 pushes distally on the deflectable member 6404 causing it to pivot in a negative direction. The tether 6408 must possess an appropriate stiffness to enable it to push the deflectable member 6404 in a negative direction. The tether 6408 may be made to any appropriate flexibility and configuration to take the desired shape such as a flexible push bar or shape memory material. In an embodiment, the tether 6408 may be a flexboard or other electrical interconnection member that also serves to electrically interconnect the deflectable member 6404 to the catheter body 6403. In such a configuration, the flexboard may be reinforced to achieve adequate stiffness.

In an alternate embodiment, the catheter body 6403 may be constructed from a single tube and the tether 6408 may be a push/pull wire activated by a user of the catheter 6400. In such an embodiment, a user would pull on the push/pull wire to pull the deflectable member 6404 in a positive direction as illustrated in FIG. 64B, and push on the push/pull wire to push the deflectable member 6404 in a negative direction as illustrated in FIG. 64C.

FIG. 64D illustrates a catheter 6410, which is a variation of the catheter 6400. Catheter 6410 includes a centrally disposed living hinge 6411 positioned between a distal end 6412 of a catheter body 6413 and a deflectable member 6414. The deflectable member 6414 may contain a transducer array 6415 (e.g., fixed one dimensional array, pivotable one dimensional array, two-dimensional array) capable of imaging a plane or volume 6416 (schematically represented) disposed proximate to the deflectable member 6414.

The catheter 6410 may have a total range of motion comparable to that illustrated with respect to catheter 6400 (e.g., at least about 200 degrees). The catheter 6410 may include a first actuation member 6417 and a second actuation member 6418 that may be used to deflect the deflectable member 6414. The first and second activation members 6417, 6418 may be in the form of wires. The first and second activation members 6417, 6418 may run along the length of the catheter body 6413 to a point where a user operating the catheter 6410 may be able to selectively pull either actuation member 6417, 6418 to control the deflection of the deflectable member 6414.

The first actuation member 6417 may be fixed to the deflectable member 6414 at a first anchor point 6419 that is disposed on a side of the deflectable member 6414 opposite from a front face of the transducer array 6415. In this regard, pulling on the first actuation member 6417 may cause the deflectable member 6414 to rotate in a positive direction (upward as shown in FIG. 64D). The second actuation member 6418 may be fixed to the deflectable member 6414 at a second anchor point 6420 that is disposed on the same side of the deflectable member 6414 as the front face of the transducer array 6415. Pulling on the second actuation member 6418 may cause the deflectable member to rotate in a negative direction (downward as shown in FIG. 64D).

An electrical interconnection member 6421 may pass through the centrally disposed living hinge 6411. The electrical interconnection member 6421 may, for example, include a flexboard.

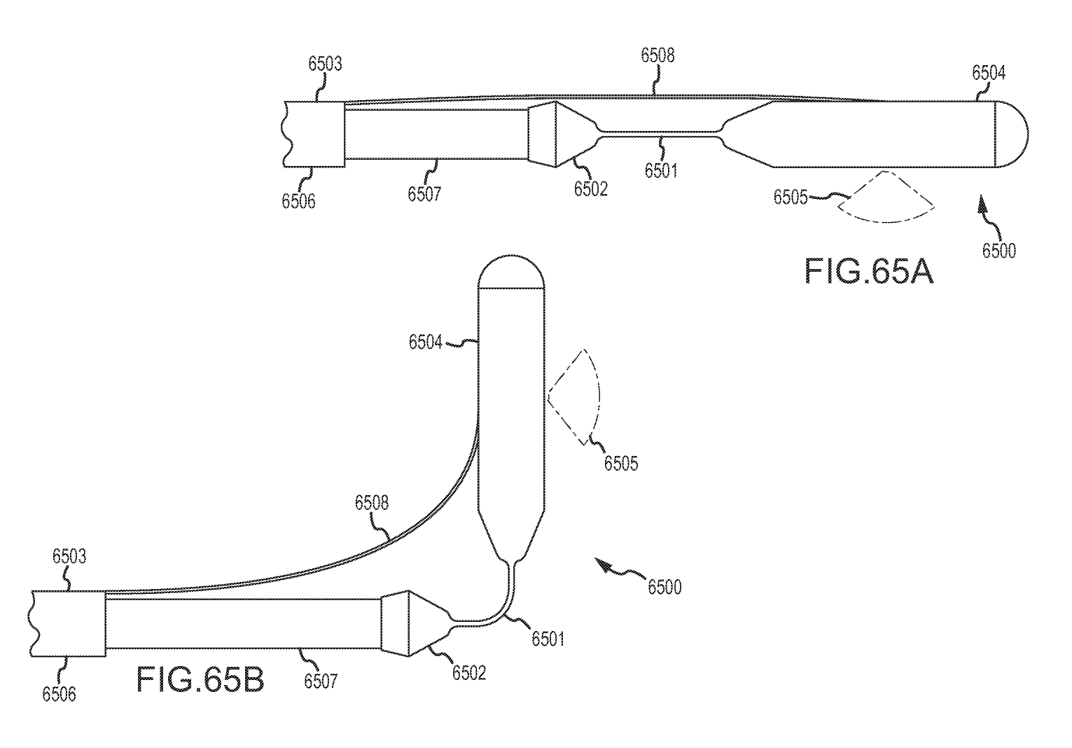

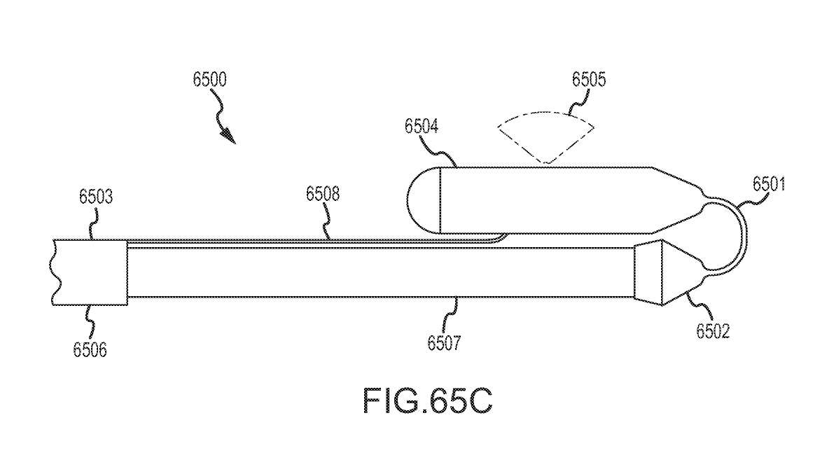

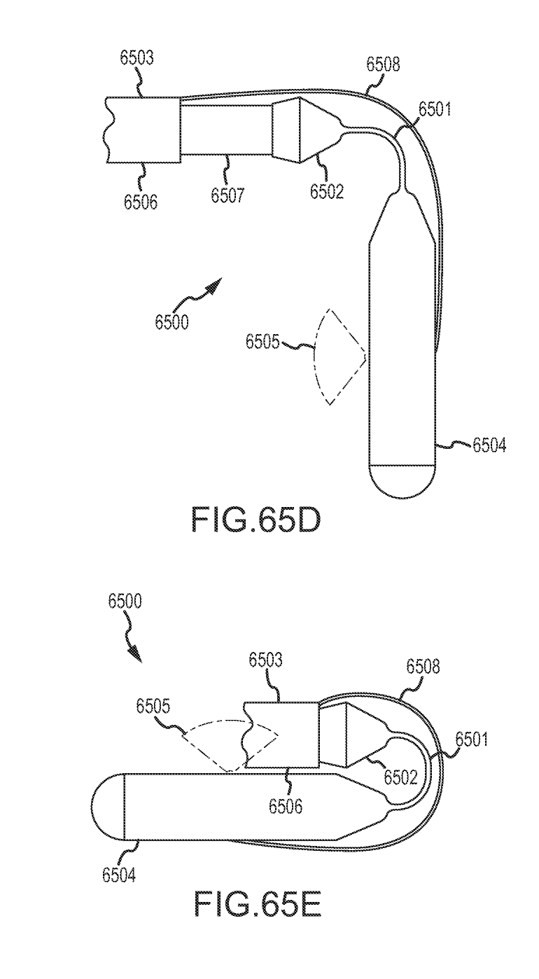

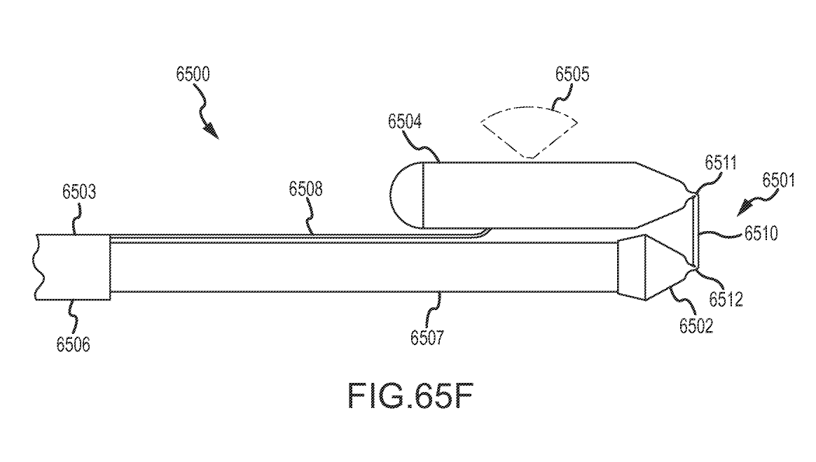

FIGS. 65A through 65E illustrate an embodiment of a catheter 6500 that includes a centrally disposed hinge 6501 positioned between a distal end 6502 of a catheter body 6503 and a deflectable member 6504. The deflectable member 6504 may contain a transducer array (e.g., fixed one dimensional array, pivotable one dimensional array, two-dimensional array) capable of imaging a plane or volume 6505 (schematically represented) disposed proximate to the deflectable member 6504.

As illustrated in FIGS. 65B through 65E, the deflectable member 6504 may have a total range of motion of about 360 degrees. FIG. 65C illustrates the deflectable member 6504 deflected about +180 degrees from the aligned position (FIG. 65A), and FIG. 65E shows the deflectable member 6504 deflected about −180 degrees from the aligned position. This range of motion is achieved by displacing an outer tube 6506 of the catheter body 6503 relative to an inner tube 6507. A tether 6508 is interconnected to the outer tube 6506 and the deflectable member 6504.

To achieve the 360 degrees of motion of the deflectable member 6504, the hinge 6501 may have a total length of at least the sum of one half the diameter of the deflectable member 6504 plus one half the diameter of the catheter body 6503 (e.g., about the distance between the center lines of the catheter body 6503 and the deflectable member 6504). In the illustrated embodiment, where the hinge 6501 is a single bendable member that generally bends uniformly as the deflectable member 6504 is deflected, the length of the hinge 6501 may be about one half the circumference of the deflectable member 6504 to allow the hinge 6501 to achieve the position illustrated in FIGS. 65C and 65E.

In an alternative configuration illustrated in FIG. 65F, the hinge 6501 may be a relatively stiff member 6510 with two living hinges 6511, 6512 disposed along its length. The distance between the two hinges 6511, 6512 may be about the distance between the center lines of the catheter body 6503 and the deflectable member 6504 when positioned as shown in FIG. 65F. In another alternative (not shown), the hinge 6501 may include a single living hinge with remaining portions of the hinge 6501 compliant enough to allow for positive or negative 180 degrees movement by the deflectable member 6504.

In the embodiments illustrated in FIGS. 65A through 65F, when the outer tube 6506 is moved proximally relative to the inner tube 6507 as illustrated in FIGS. 65B, 65C and 65F, the tether 6508 pulls proximally on the deflectable member 6504 causing it to deflect in a positive direction. Moving the outer tube 6506 proximally a first distance may deflect the deflectable member 6504 to a forward-looking position as illustrated in FIG. 65B. Continuing to move the outer tube proximally may cause the deflectable member 6504 to move into a side-facing position as illustrated in FIGS. 65C and 65F. Similarly, the deflectable member 6504 may be moved into a rearward-looking position (FIG. 65D) or a side-facing position (FIG. 65E) by moving the outer tube 6506 distally relative to the inner tube 6507.

The tether 6508 must possess an appropriate stiffness to enable it to push the deflectable member 6504 in the negative direction shown in FIGS. 65D and 65E. The tether 6508 may be made to any appropriate flexibility and configuration to take the desired shape such as a flexible push bar or shape memory material. In an embodiment, the tether 6508 may be a flexboard or other electrical interconnection member that also serves to electrically interconnect the deflectable member 6504 to the catheter body 6503. In such a configuration, the flexboard may be reinforced to achieve adequate stiffness.

A sheath or other mechanical support (not shown) may be used to secure the deflectable member 6504 in the aligned position shown in FIG. 65A while the catheter 6500 is being moved in the body. Once positioned, the sheath or other mechanical support may be removed (e.g., retracted) to allow for the deflection of the deflectable member.

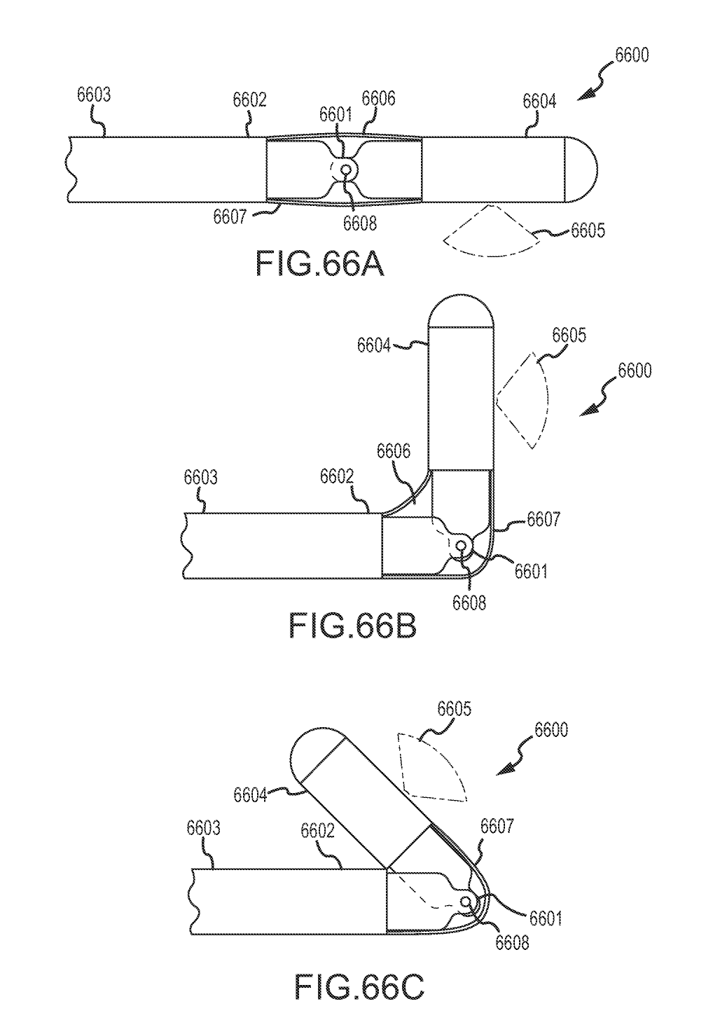

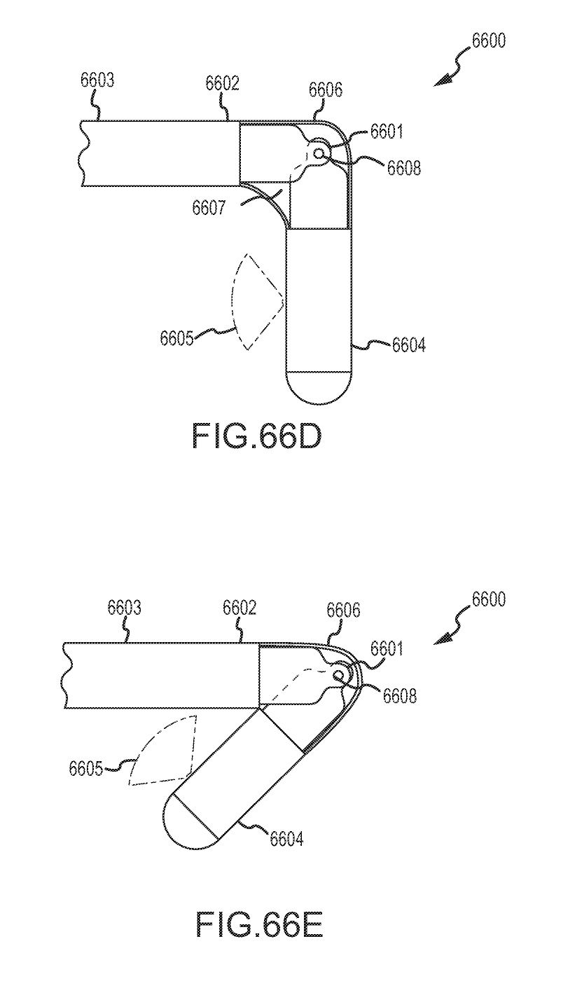

FIGS. 66A through 66E illustrate an embodiment of a catheter 6600 that includes a centrally disposed hinge 6601 positioned between a distal end 6602 of a catheter body 6603 and a deflectable member 6604. The deflectable member 6604 may contain a transducer array (e.g., fixed one dimensional array, pivotable one dimensional array, two-dimensional array) capable of imaging a plane or volume 6605 (schematically represented) disposed proximate to the deflectable member 6604.

As illustrated in FIGS. 66B through 66E, the deflectable member 6604 may have a total range of motion of at least about 270 degrees. FIG. 66C shows the deflectable member 6604 pivoted about +135 degrees from the aligned position (FIG. 66A), and FIG. 66E shows the deflectable member 6604 pivoted about −135 degrees from the aligned position. This range of motion is achieved through manipulation of a first actuation member 6606 and/or a second actuation member 6607. The actuation members 6606 and 6607 may, for example, be in the form of pull wires. The first and second actuation members 6606, 6607 may run along the length of the catheter body 6603 to a point where a user operating the catheter 6600 may be able to selectively pull either actuation member 6606, 6607 to control the deflection of the deflectable member 6604.

The first actuation member 6606 may be fixed to the deflectable member 6604 on a side of the deflectable member 6604 opposite from a front face of the transducer array. In this regard, pulling on the first actuation member 6606 may cause the deflectable member 6604 to rotate in a positive direction (upward as shown in FIG. 66B). In this regard, the deflectable member 6604 may be pivoted to achieve a desired angle, such as a forward-facing +90 degrees (FIG. 66B) or a positive 135 degrees (FIG. 66C). Such displacement through pulling on the first actuation member 6606 may be accompanied by relaxing tension on or feeding the second actuation member 6607 to allow for the longer portion of the second actuation member 6607 disposed distal to the distal end 6602 when the deflectable member 6604 is displaced in a positive direction as shown in FIGS. 66B and 66C.

The second actuation member 6607 may be fixed to the deflectable member 6604 on the same side of the deflectable member 6604 as the front face of the transducer array. In this regard, pulling on the second actuation member 6607 may cause the deflectable member 6604 to rotate in a negative direction (downward as shown in FIG. 66D). In this regard, the deflectable member 6604 may be pivoted to achieve a desired angle, such a rearward-facing −90 degrees (FIG. 66D) or −135 degrees (FIG. 66E). Such displacements may be accompanied by appropriate feeding of the first actuation member 6606 similar to that described above with respect to a positive displacement.

The catheter 6600 includes an electrical interconnection member (not shown) to electrically interconnect the deflection member 6604 with conductors running along the catheter body 6603. Such an electrical interconnection member may be in the form of a flexboard.

The hinge 6601 may include a pin 6608 and the deflectable member 6604 may pivot relative to the distal end 6602 about a central axis of the pin 6608. The pin 6608 may, for example, be integral with, or pressed into a corresponding hole of, the deflectable member 6604 such that the pin 6608 is fixed to the deflectable member 6604. The pin 6608 may fit within a hole in the distal end 6602 such that it is free to rotate within the hole as the deflectable member 6604 pivots relative to the distal end 6602. In this regard, the hinge 6601 may include a pair of surfaces (e.g., the outside surface of the pin 6608 and the inside surface of the hole in the distal end 6602) that may slide relative to each other to allow the deflectable member 6604 to deflect. Any other appropriate hinge, including a hinge where the pin 6608 is fixed to the distal end 6602 and free to pivot relative to the deflectable member 6604, may be used in place of the described hinge 6608.

The embodiments of FIGS. 64A through 64C and 65A through 65F are illustrated using a single tether 6408, 6408 and tube-in-tube actuation to effectuate deflection of the corresponding deflectable members. The embodiments of FIGS. 64D and 66A through 66E are each illustrated using two actuation members 6417, 6418, 6606, 6607 to effectuate deflection of the corresponding deflectable members. Such arrangements are for illustrative purposes only, and any appropriate deflection control system may be used with any appropriate hinge arrangement. For example, a tube-in-tube actuation system with a single tether may be used in the hinge embodiment of FIGS. 66A through 66E, while two actuation member systems may be employed with the embodiment of FIGS. 65A through 65F.

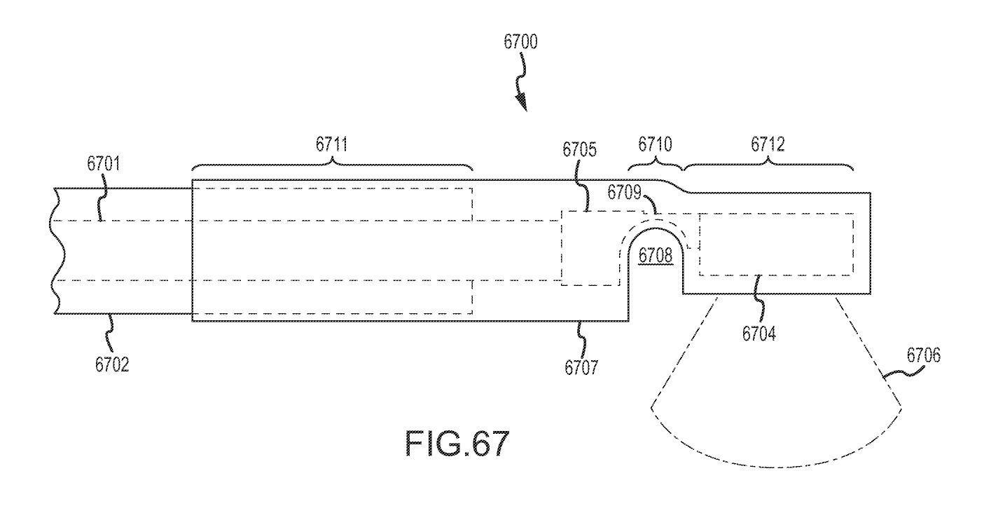

FIG. 67 illustrates a catheter 6700 that includes an inner tubular body 6701 and an outer tubular body 6702. Attached to the inner tubular body 6701 is living hinge 6705 similar to living hinge 6001. Attached to the living hinge 6705 is a deflectable member 6704. The deflectable member 6704 may contain a transducer array (e.g., fixed one dimensional array, pivotable one dimensional array driven by a motor, two-dimensional array) capable of imaging a plane or volume 6706 (schematically represented) disposed proximate to the deflectable member 6704.

The catheter 6700 may further include a tube tether 6707. The tube tether 6707 may be a piece of shrink tube (e.g., fluorinated ethylene propylene (FEP) shrink tube) or other bondable tubing with a portion 6708 removed so that the region 6710 of the tube tether 6707 proximate to a hinge line 6709 of the living hinge 6705 is non-tubular and may act as a tether (e.g., in a manner similar to the tether 6009 of FIG. 61). The tube tether 6707 may be secured to the outer tubular body 6702 in the region 6711 at the distal end of the outer tubular body 6702 via the application of heat, to cause the shrink tube to shrink, or application of adhesive and thereby become fixed to the outer tubular body 6702. Moreover, the tube tether 6707 may be secured to the deflectable member 6704 in the region 6712 via the application of heat, to cause the shrink tube to shrink, or application of adhesive and thereby become fixed to the deflectable member 6704.

The tube tether 6707 functions to cause the deflectable member 6704 to pivot in a positive direction (e.g., upward as shown in FIG. 67) relative to the inner tubular body 6701 when the inner tubular body 6701 is moved distally (e.g., to the right in FIG. 67) relative to the outer tubular body 6702. In this regard, the region 6710 of the tube tether 6707 performs a similar function as tether 6009 of FIG. 61. The tube tether 6707 may also cause the deflectable member 6704 to pivot in a negative direction (e.g., downward as shown in FIG. 67) when the inner tubular body 6701 is moved proximally (e.g., to the left in FIG. 67) relative to the outer tubular body 6702. Any appropriate electrical interconnection scheme, such as those described herein, may be used with the catheter 6700 of FIG. 67.

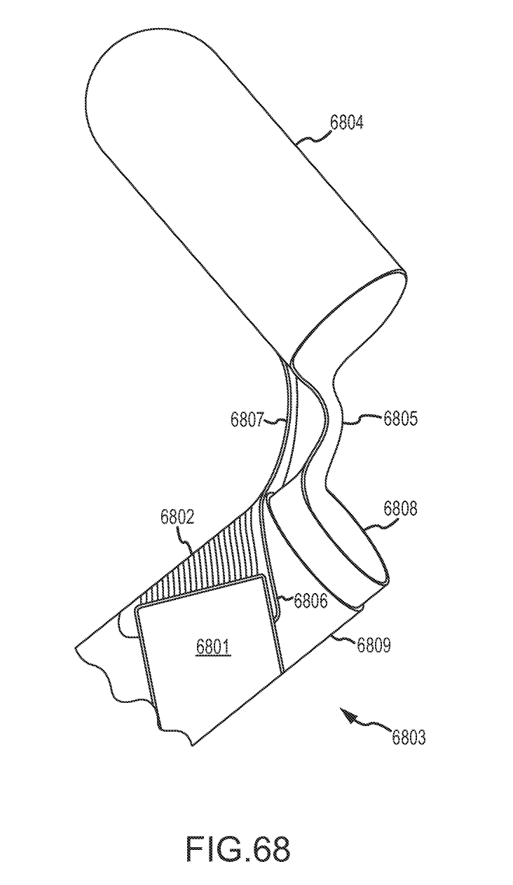

FIG. 68 shows an embodiment of an electrical interconnection between a helically disposed electrical interconnection member 6801 and a flexboard 6802 (a flexible/bendable electrical member). The electrical interconnection member 6801 is helically wrapped about a portion of a catheter body 6803. Additional layers of the catheter body 6803 disposed over the helically disposed electrical interconnection member 6801 are not shown in FIG. 68. The catheter body 6803 is hingedly interconnected to a deflectable member 6804 via a hinge 6805. The deflectable member 6804 and hinge 6805 may be similar to any appropriate member and hinge described herein. The deflectable member 6804 may contain a transducer array capable of imaging a plane or volume.

The flexboard 6802 may have an interconnection section 6806 where the conductors on the flexboard 6802 are spaced to coincide with the spacing of the conductors on the electrical interconnection member 6801. At the interconnection section 6806, the electrically conductive portions (e.g., traces, conductive paths) of the flexboard 6802 may be interconnected to the electrically conductive portions (e.g., wires) of the electrical interconnection member 6801. This electrical interconnection may be achieved by peeling back or removing some of the insulative material of the electrical interconnection member 6801 and contacting the exposed electrically conductive portions to corresponding exposed electrically conductive portions on the flexboard 6802.

As illustrated in FIG. 68, the flexboard 6802 may comprise a flexing or bending region 6807 that has a width narrower than the width of the interconnection section 6806. As will be appreciated, the width of each individual electrically conductive path through the flexing region 6807 may be smaller to the width of each electrically conductive member within the interconnection section 6806. Furthermore the pitch between each electrically conductive member within the flexing region 6807 may be smaller than the pitch of the interconnection section 6806. The flexing region 6807 may be interconnected to a transducer array (not shown) within the deflectable member 6804.

As illustrated in FIG. 68, the flexing region 6807 of the flexboard 6802 may be operable to flex during deflection of the deflectable member 6804. In this regard, the flexing region 6807 may be bendable in response to deflection of the deflectable member 6804. The individual conductors of the electrical interconnection member 6801 may remain in electrical communication with the individual transducers of the transducer array during deflection of the deflectable member 6804. Moreover, the flexing region 6807 of the flexboard 6802 may be operable to act as a tether such that when an inner tube 6808 is advanced relative to an outer tube 6809, the flexing region 6807, by virtue of its fixed length between the outer tube 6809 and the deflectable member 6804, causes the deflectable member 6804 to pivot in a positive direction as shown in FIG. 68. Additional wires, such as wires interconnected to a motor or sensors in the deflectable member 6804, may be run between the catheter body 6803 and the deflectable member 6804. Such wires may disposed such that they are not put in tension and do not serve as a tether when the deflectable member 6804 is pivoted.

The electrical interconnection member 6801 may comprise members that extend from a distal end to a proximal end of the catheter body 6803 or the electrical interconnection member 6801 may comprise a plurality of discrete, serially interconnected members that together extend from the distal end to the proximal end of the catheter body 6803. In an embodiment, the flexboard 6802 may include the electrical interconnection member 6801. In such an embodiment, the flexboard 6802 may have a helically wrapped portion extending from the distal end to the proximal end of the catheter body 6803. In such an embodiment, no electrical conductor interconnections (e.g., between the flexboard 6802 and a flat cable) may be required between the flexing region 6807 and the proximal end of the catheter body 6803.

In a variation of the configuration of the electrical interconnections illustrated in FIG. 68, a single (e.g., not constructed from a series of members subsequently interconnected to each other) electrical interconnection member may be used that runs from the proximal end of the catheter body 6803 or beyond (e.g., extending to a connection within ultrasound console 5706), all the way to an electrical interconnection with a transducer array disposed within the deflectable member 6804

In a first implementation, the single electrical interconnection member may be a flexboard or flex circuit. An exemplary route that may be followed by such a flex circuit would be to run from the proximal end of the catheter (or beyond), turn at an angle to accommodate wrapping in the catheter body wall, turn again at the distal end of the catheter body to run straight through the hinge, turn at a 90 degree angle to be wound as a clock spring within the deflectable member (e.g., to accommodate the reciprocal pivotal motion of a transducer array), and then turn at another 90 degree angle to run over the back of the transducer array and be connected thereto. In a variation, the flex circuit may travel down an interior portion of the catheter body instead of being wrapped in the catheter body wall.

A flex circuit of such a length may be produced from a sheet where the conductors are laid out in a back and forth pattern. The sheet may then be cut such that the conductive strip is configured in an accordion-like pattern. The conductive strip may then be folded at each bend to form a substantially straight single electrical interconnection member (apart from the end features to accommodate the deflectable member and/or connection to the ultrasound console 5706) of a desired length.

Such a single flex circuit configuration may be used with any appropriate embodiment described herein.

In a second implementation, the single electrical interconnection member may be a ribbon cable such as a GORE™ Micro-Miniature Ribbon Cable. Such a cable could be routed from the proximal end of the catheter (or beyond), down an interior portion of the catheter body, and continue through the hinge and then be attached to the back of the array. In such an embodiment, a backplane removed may be removed to increase the flexibility of the ribbon cable in specific areas, such as at the hinge and/or within the deflectable member. To further increase flexibility, the individual conductors of the ribbon cable may be separated in these areas. An example of a ribbon cable where the individual conductors are separated in the region of the hinge is illustrated in FIG. 50.

In an alternative arrangement of the second implementation, the individual conductors may be separated proximal to the hinge and may remain separated all the way to a transducer array disposed within the deflectable member (similar to the “flying leads” arrangement as discussed with respect to FIG. 50).

Such a single ribbon cable configuration may be used with any appropriate embodiment described herein.

FIGS. 69A through 69C are partial cross-sectional views of a deflectable member 6900 that may be connected to any appropriate hinge and catheter body described herein. For example, an end wall 6901 of deflectable member 6900 may be fixedly interconnected to end wall 6203 of hinge 6001. The deflectable member 6900 may generally be sized and shaped for insertion into a patient and subsequent imaging of an internal portion of the patient. The deflectable member 6900 may include a distal end 6902.

The deflectable member 6900 may include a case 6903. The case 6903 may be a relatively rigid member housing a motor 6904 and a transducer array 6905, both of which are discussed below. The deflectable member 6900 may include a central axis 6906.

An electrical interconnection member 6907 may be partially disposed within the deflectable member 6900. The electrical interconnection member 6907 may include a first portion 6908 disposed outside of the case 6903 (partially illustrated in FIGS. 69A and 69B). The first portion 6908 of the electrical interconnection member 6907 may be operable to electrically interconnect members within the deflectable member 6900 to electrical conductors in a catheter to which the deflectable member 6900 is attached (e.g., in a manner as discussed with reference to flexboard 6802 of FIG. 68). The first portion 6908 may also serve as a tether.

The case 6903 may be sealed, and an enclosed volume may be defined by the case 6903 and the end wall 6901. The enclosed volume may be fluid-filled. The transducer array 6905 and an associated backing may be similar to the transducer array 5307 and the associated array backing 5328 discussed with reference to FIG. 53. The case 6903 may include an acoustic window (not shown) similar to the acoustic window 5326 described with reference to FIG. 53.

As shown in FIG. 69C, the case 6903 may have a generally circular cross section. Moreover, the outer surface of the case 6903 may be smooth. Such a smooth, circular exterior profile may help in reducing thrombus formation and/or tissue damage as the deflectable member 6900 is moved (e.g., rotated, translated) within a patient.

In general, the images generated by the deflectable member 6900 may be of a subject (e.g., internal structure of a patient) within an image volume similar to the image volume 5327 discussed with reference to FIG. 53. The transducer array 6905 may be disposed on a mechanism operable to reciprocally pivot the transducer array 6905 about the central axis 6906, or an axis parallel to the central axis 6906, such that the image plane is swept about the central axis 6906, or an axis parallel to the central axis 6906, to form the image volume. In this regard, the deflectable member 6900 may be used in a system (e.g., ultrasound imaging system 5700) to display live or near-live video of the image volume.

The transducer array 6905 may be interconnected at a distal end to an output shaft of the motor 6904. Furthermore, the transducer array 6905 may be supported on a proximal end of the transducer array 6905 by a pivot 6910. The interface between the pivot 6910 and the transducer array 6905 may allow for the transducer array 6905 to reciprocally pivot about its rotational axis while substantially preventing any lateral movement of the transducer array 6905 relative to the case 6903. Accordingly, the transducer array 6905 may be operable to be reciprocally pivoted about its rotational axis.

The motor 6904 may be disposed at the distal end 6902 of the deflectable member 6900. The motor 6904 may be an electrically powered motor operable to selectively rotate the transducer array 6905 in both clockwise and counterclockwise directions. In this regard, the motor 6904 may be operable to reciprocally pivot the transducer array 6905.

The motor 6904 may be fixedly mounted to a motor mount 6911 that is in turn fixedly disposed relative to the case 6903. The motor mount 6911 may be interconnected to the motor 6904 at or near where the output shaft of the motor 6904 is interconnected to the transducer array 6905. Electrical interconnections to the motor 6904 may be achieved through a dedicated set of electrical interconnections (e.g., wires) separate from the electrical interconnection member 6907.

The electrical interconnection member 6907 may be anchored such that a portion of it is fixed relative to the case 6903. The electrical interconnection member 6907 includes a second portion 6909 disposed in the distal end 6902 of the deflectable member 6900 and operable to accommodate the reciprocal motion of the transducer array 6905. The electrical interconnection member 6907 further includes a third portion 6912 disposed along the case 6903 and operable to electrically interconnect the first portion 6908 to the second portion 6909.

The third portion 6912 of the electrical interconnection member 6907 may be anchored such that at least a portion of it is fixed relative to the case 6903. The third portion 6912 of the electrical interconnection member 6907 may be secured to the case 6903 in a region corresponding to the position of the transducer array 6905. In this regard, the third portion 6912 of the electrical interconnection member 6907 may be disposed such that it does not interfere with the reciprocal movement of the transducer array 6905. Any appropriate method of anchoring the third portion 6912 of the electrical interconnection member 6907 to the case 6903 may be used. For example, adhesive may be used.

The second portion 6909 of the electrical interconnection member 6907 is operable to maintain an electrical connection to the transducer array 6905 while the transducer array 6905 is pivoting. This may be achieved by coiling the second portion 6909 of the electrical interconnection member 6907 about the motor 6904 in an area distal to the motor mount 6911. In this regard, the electrical interconnection member 6907 may be coiled about an axis aligned with the axis of rotation of the rotational output of the motor 6904. One end of the second portion 6909 of the electrical interconnection member 6907 may be anchored to the case 6903 and the other end 6913 of the second portion 6909 of the electrical interconnection member 6907 may be electrically interconnected to the transducer array 6905 (through an array backing).

The second portion 6909 of the electrical interconnection member 6907 may have a generally flat cross-section and be disposed such that a top or bottom side of the second portion 6909 faces and wraps about the central axis 6906. The second portion 6909 of the electrical interconnection member 6907 may be coiled in a “clock spring” arrangement where, as illustrated in FIGS. 69A through 69C, substantially the entirety of the second portion 6909 of the electrical interconnection member 6907 is positioned at the same point along the central axis 6906.

One end of the clock spring of the second portion 6909 of the electrical interconnection member 6907 may be electrically interconnected to the third portion 6912, while the other end 6913 may be electrically interconnected to the transducer array 6905 (through the array backing). The clock spring of the second portion 6909 may be comprised of a partial coil or any appropriate number of coils.

Similar to the embodiments of FIGS. 53 and 55, by coiling the clock spring of the second portion 6909 of the electrical interconnection member 6907 (e.g., about an axis parallel to the central axis 6906), undesirable counteracting torque on the pivoting of the transducer array 6905 may be significantly avoided. In this regard, pivoting of the transducer array 6905 about the central axis 6906 in such a configuration may result in a slight tightening, or slight loosening, of the turns of the clock spring of the second portion 6909 of the electrical interconnection member 6907. Such a slight tightening and loosening may result in each coil producing only a small lateral displacement and corresponding displacement of fluid.

The clock spring of the second portion 6909, and other clock spring arrangements discussed herein, may provide for increased durability in comparison to a configuration where an electrical interconnection is twisted along its length. The clock spring of the second portion 6909, and other clock spring arrangements discussed herein, may be configured such that when the transducer array 6905 is positioned at the center of its desired range of motion, the clock spring of the second portion 6909 imparts little or no torque on the transducer array 6905. In such a configuration, when the motor 6904 moves the transducer array 6905 from the center position, the clock spring of the second portion 6909 may impart a torque on the transducer array 6905 that urges the transducer array 6905 back toward the center position. Such torque imparted on the transducer array 6905 may be selected to be minimal or it may be selected to assist the motor 6904 in returning the transducer array 6905 to the center position. In another arrangement, the clock spring of the second portion 6909 may be configured to urge the transducer array 6905 to one end of its desired range of motion. The configuration of the clock spring of the second portion 6909 also saves space within the deflectable member 6900 in that the pivoting of the transducer array 6905 may be accommodated by a portion of the electrical interconnection member 6907 (e.g., the second portion 6909) wrapped about a single point along the central axis 6906.

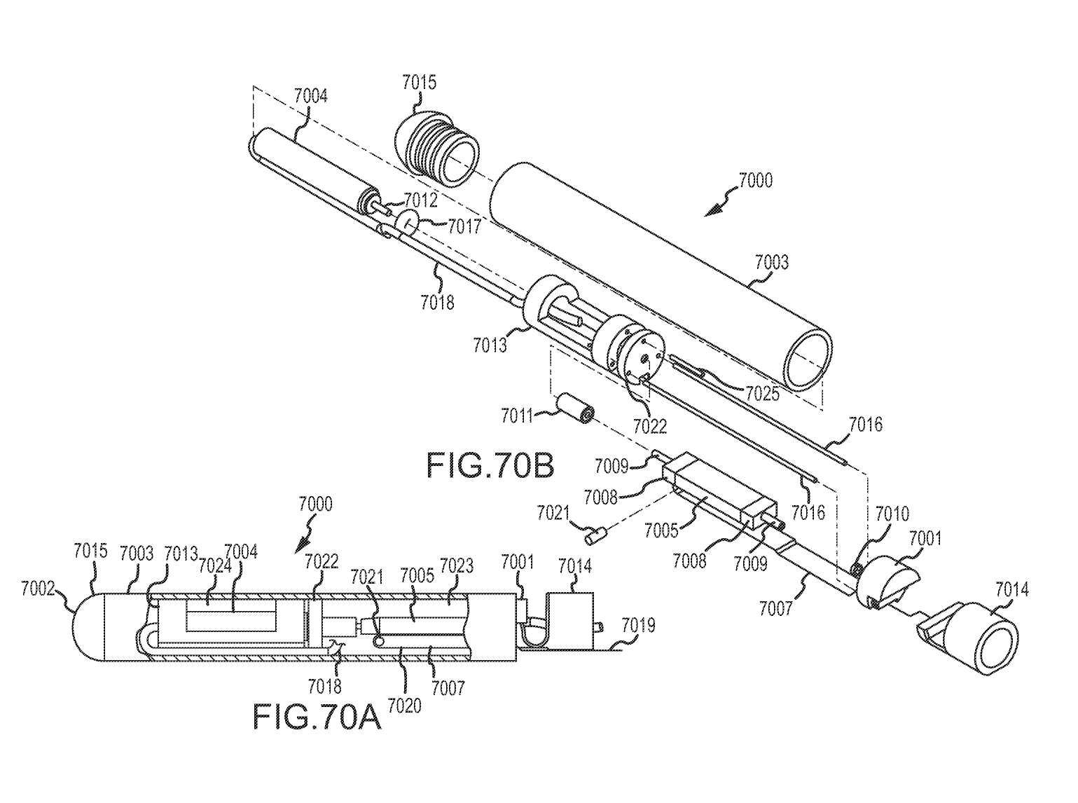

FIG. 70A is a partial cross-sectional view of a deflectable member 7000. FIG. 70B is an exploded view of the deflectable member 7000. Deflectable member 7000 may be connected to any appropriate hinge and catheter body described herein. For example, as illustrated, an end cap 7001 of deflectable member 7000 may be fixedly interconnected to hinge 7014. Hinge 7014 may be configured similarly to hinge 6001. The deflectable member 7000 may generally be sized and shaped for insertion into a patient and subsequent imaging of an internal portion of the patient. The deflectable member 7000 may include a distal end 7002.

The deflectable member 7000 may include a case 7003 and an end cap 7015. The end cap 7015 may be sized to fit within and seal the distal end 7002 of the case 7003. The case 7003 may be a relatively rigid member housing a motor 7004 and a transducer array 7005, both of which are discussed below.

An electrical interconnection member 7007 may be partially disposed within the deflectable member 7000. The electrical interconnection member 7007 may include a first portion 7019 disposed outside of the case 7003 that may be operable to electrically interconnect members within the deflectable member 7000 to electrical conductors in a catheter to which the deflectable member 7000 is attached (e.g., in a manner as discussed with reference to flexboard 6802 of FIG. 68).

In general, the deflectable member 7000 may be used in the process of generating images similar to as described above with reference to the deflectable member 6900. In this regard, the transducer array 7005 may be disposed on a mechanism operable to reciprocally pivot the transducer array 7005.

The transducer array 7005 may be fixed to and supported by a pair of array end caps 7008 disposed at opposing ends of the transducer array 7005. In turn, a pair of shafts 7009 may be fixedly inserted into corresponding holes in the array end caps 7008. One of the shafts 7009 may be disposed within a bearing 7010 that may be mounted to the end cap 7001. The bearing may allow the shaft 7009 disposed therein (and therefore the transducer array 7005 that is interconnected to the shaft 7009) to pivot relative to the end cap 7001. The other shaft 7009, disposed at a distal end of the transducer array 7005, may be fixed to a coupling 7011 that is in turn fixed to an output shaft 7012 of the motor 7004. Thus the transducer array 7005 may be fixed relative to the output shaft 7012 of the motor 7004 such that the motor 7004 may reciprocally pivot the transducer array 7005 about an array rotational axis defined by the output shaft 7012 and shafts 7009.

The motor 7004 may be disposed at the distal end 7002 of the deflectable member 7000. The motor 7004 may be an electrically powered motor operable to selectively pivot the transducer array 7005 in both clockwise and counterclockwise directions.

The motor 7004 may be disposed within a motor mount 7013 that is in turn fixedly disposed relative to the end cap 7001 via a pair of rods 7016. The pair of rods 7016 fix the motor mount 7013 to the end cap 7001 such that the motor mount 7013 is at a fixed distance from the end cap 7001 such that the transducer array 7005, array end caps 7008, and shafts 7009 may be disposed between the motor mount 7013 and the end cap 7001. Electrical interconnections to the motor 7004 may be achieved through a dedicated set of electrical interconnections 7018 (e.g., wires) separate from the electrical interconnection member 7007. It will be appreciated that such construction allows for the transducer array 7005, motor mount 7013, and motor 7004 to be mounted to the end cap 7001 in a sub-assembly. Subsequently, the case 7003 may be installed over such a sub-assembly.

An o-ring 7017 may be disposed about the output shaft 7012 of the motor 7004. The o-ring 7017 may be sandwiched between a proximal end of the motor mount 7013 and a plate 7022. Moreover, the proximal end of the motor 7004 (i.e., the end of the motor 7004 with the output shaft 7012) may also be disposed in the region between the proximal end of the motor mount 7013 and the plate 7022. Grease may be inserted in the region between the proximal end of the motor mount 7013 and the plate 7022 and on the o-ring 7017. The grease may restrict liquids from entering the region between the proximal end of the motor mount 7013 and the plate 7022 and therefore help to prevent liquids from entering the motor 7004 through the proximal end of the motor 7004. The motor mount 7013 and the plate 7022 may be sized to assist in restricting liquid from entering the region between the proximal end of the motor mount 7013 and the plate 7022. The plate 7022 may be fixed relative to the motor mount 7013 by the rods 7016 and a pin 7025.

The case 7003 may be sealed, and an enclosed volume may be defined by the case 7003, the end cap 7015, and the end cap 7001. The enclosed volume may include a proximal enclosed volume 7023 in the region between the plate 7022 and the end cap 7001 and a distal enclosed volume in the region between the proximal end of the motor mount 7013 and the end cap 7015.

The proximal enclosed volume 7023 may be fluid-filled. The transducer array 7005 and an associated backing may be similar to the transducer array 6905 and the associated array backing discussed with reference to FIGS. 69A through 69C. The case 7003 may include an acoustic window (not shown) in the region of the case 7003 corresponding to the transducer array 7005. Such an acoustic window may be similar to the acoustic window 5326 described with reference to FIG. 53. The fluid in the proximal enclosed volume 7023 may be selected to provide an acoustic coupling medium between the transducer array 7005 and the case 7003 or acoustic window (if present).

The distal enclosed volume 7024 may be fluid-filled. The fluid in the distal enclosed volume 7024 may be selected to provide a heat dissipation medium to cool the motor 7004. A sealant, such as an ultraviolet (UV) cured epoxy, may be placed around the portion of the motor 7004 where the electrical connections 7018 enter into the motor 7004 to restrict the ability of liquid to enter into the motor 7004. In this regard, through the use of the UV cured epoxy and the above-described grease, the motor 7004 may be of a type not specifically designed to be operable in a liquid-filled environment. Alternatively, a sealed motor designed to be operable in a liquid-filled environment may be used.

The electrical interconnection member 7007 may be a flexboard or other appropriate flexible multiple conductor member. The first portion 7019 may also serve as a tether. The electrical interconnection member 7007 may pass between the end cap 7001 and the case 7003 as it passes from the area proximate to the hinge 7014 to the interior of the deflectable member 7000. In this regard, the electrical interconnection member 7007 may be securely held between the end cap 7001 and the case 7003.

A second portion of the electrical interconnection member 7007 may be disposed within the deflectable member 7000 and may run from the end cap 7001 to the back side of the transducer array 7005. In particular, the second portion 7020 may run along the length of the transducer array 7005 in the space between the back side of the transducer array 7005 and the case 7003. At the distal end of the transducer array 7005, the second portion 7020 may wrap around a pin 7021 and then run along, and be in contact with, the backside of the transducer array 7005 to electrically interconnect to the transducer array 7005 (through a backing of the transducer array 7005).

The pin 7021 may be secured to the second portion 7020 and the second portion may be secured to the back side of the transducer array 7005. Thusly, the portion of the second portion 7020 in contact with the pin 7021 and the portion of the second portion 7020 in contact with the back side of the transducer array 7005 may be fixedly interconnected to the transducer array 7005. With the second portion 7020 secured to the pin 7021, the reciprocal pivotal motion of the transducer array 7005 may cause the second portion 7020 to flex in the region between where it is secured to the pin 7021 and where the second portion is secured between the end cap 7001 and the case 7003. Accordingly, the second portion 7020 of the electrical interconnection member 7007 is operable to maintain an electrical connection to the transducer array 7005 while the transducer array 7005 is pivoting.

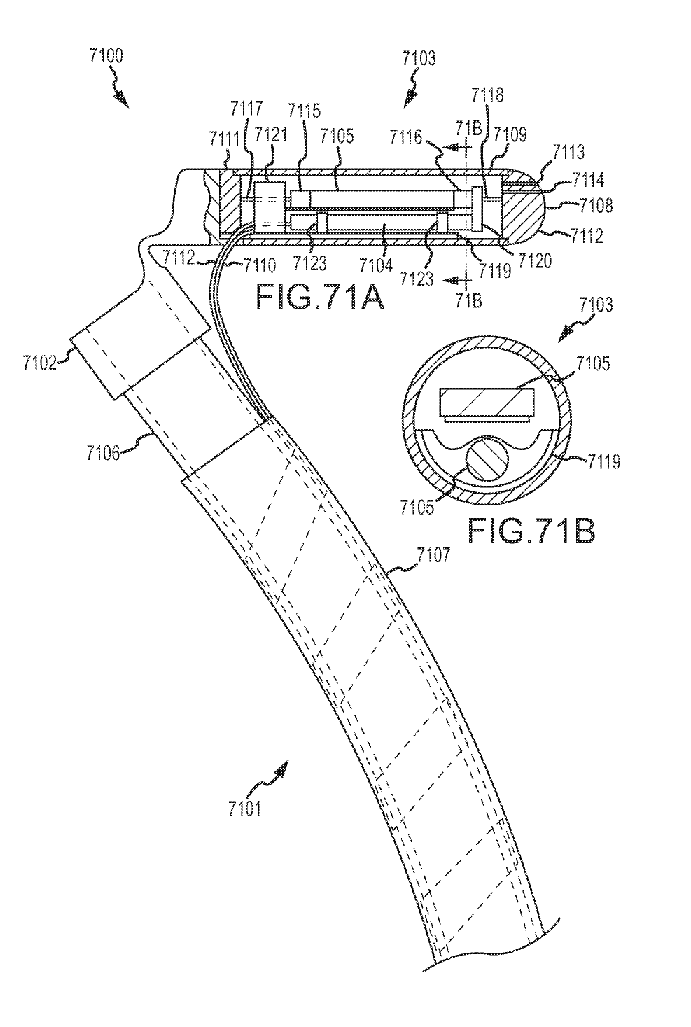

FIGS. 71A and 71B illustrate a distal end of a catheter 7100 that includes a catheter body 7101 connected by a living hinge 7102 (similar to the living hinge 6001 of FIGS. 60, 61, and 62), to a deflectable member 7103. The distal end of a catheter 7100 is illustrated in a steered state. The living hinge 7102 is supportably interconnected to the deflectable member 7103 and an inner tubular body 7106 of the catheter body 7101. An electrical interconnection member 7110 is flexible and acts as a restraining member interconnected to an outer tubular body 7107 of the catheter body 7101 and the deflectable member 7103. Selective relative movement between the inner tubular body 7106 and the outer tubular body 7107 causes the deflectable member 7103 to selectively deflect in a predetermined manner. The deflectable member 7103 in FIG. 71 is deflected to a forward-looking position.

FIG. 71A illustrates the deflectable member 7103 in partial cross-section. FIG. 71B is a cross sectional view of the deflectable member 7103 of FIG. 71A taken along line 71A-71A. The deflectable member 7103 may generally be sized and shaped for insertion into a patient and subsequent imaging of an internal portion of the patient. The deflectable member 7103 may include a distal end 7108. The deflectable member 7103 may include a case 7109. The case 7109 may be a relatively rigid member housing a motor 7104 and a transducer array 7105, both of which are discussed below.

The electrical interconnection member 7110 may be partially disposed within the deflectable member 7103. The electrical interconnection member 7110 may be fixed relative to deflectable member 7103 where the electrical interconnection member 7110 enters the deflectable member 7103. In this regard, stresses on the electrical interconnection member 7110 (e.g., due to its tethering function) may not be translated into the interior of the deflectable member 7103.

The case 7109 may be sealed, and an enclosed volume may be defined by the case 7109, an end wall 7111, and an end cap 7112. The enclosed volume may be fluid-filled. The enclosed volume may be filled by inserting fluid through a fluid port 7113 while allowing air within the enclosed volume to escape through an air vent 7114. Both the fluid port 7113 and the air vent 7114 may be sealed after the enclosed volume if filled with fluid. The case 7109 may include an acoustic window.

The transducer array 7105 and an associated backing may be similar to the transducer array 6905 and backing discussed with reference to FIG. 69. As shown in FIG. 71A, the transducer array 7105 is oriented with an active, front face facing upward, away from the motor 7104. In general, the image generation capabilities of the deflectable member 7103 are also similar to those discussed with reference to the deflectable member 6900 of FIG. 69.

The transducer array 7105 may be fixed to and supported by a proximal array end cap 7115 and a coaxial distal array end cap 7116 disposed at opposing ends of the transducer array 7105. A proximal shaft 7117 may be fixedly inserted into the proximal array end cap 7115. A distal shaft 7118 may be fixedly inserted into the distal array end cap 7116. The proximal shaft 7117 may be pivotably disposed within the end wall 7111 (e.g., within a bearing). The distal shaft 7118 may be pivotably disposed within the end cap 7112 (e.g., within a bearing). Thus, the transducer array 7105 may be operable to pivot about an axis defined by the distal shaft 7118 and the proximal shaft 7117.

The motor 7104 is disposed between a back side of the transducer array 7105 and a sled 7119 that is adjacent to a portion of the case 7109. In this regard, the motor 7104 and transducer array 7105 may be co-located at a common point along a longitudinal axis of the deflectable member 7103. The sled 7119 may support a pair of motor mounts 7123 that in turn, support the motor 7104. In this regard, the position of the motor 7104 may be fixed relative to the case 7109 and therefore also relative to the transducer array 7105. A transmission 7120 may operatively interconnect an output shaft (not shown) of the motor 7104 to the transducer array 7105 such that the motor 7004 may cause the transducer array 7105 to reciprocally pivot about the axis defined by the shafts 7117, 7118. The transmission 7120 may include any appropriate mechanism, such as two or more gears, a belt, a cam, or rigid links, that is able to communicate the output of the motor 7104 to a reciprocal pivotal motion of the transducer array 7105. In this regard, the motor 7104 may be operable to reciprocally pivot the transducer array 7105. The motor 7104 may be operable to be reciprocally driven, and the transmission 7120 may transmit such reciprocal motion of the output of the motor 7104 to reciprocally pivot the transducer array 7105. In another arrangement, the motor 7104 may be operable to be continuously driven in a selected direction, and the transmission 7120 may convert such continuous rotation of the output of the motor 7104 to a motion for reciprocally pivoting the transducer array 7105. Electrical interconnections to the motor 7104 may be achieved through a dedicated set of electrical interconnections 7112 (e.g., wires) separate from the electrical interconnection member 7110.

As noted, the electrical interconnection member 7110 may be fixed relative to deflectable member 7103 where the electrical interconnection member 7110 enters the deflectable member 7103. Within the deflectable member 7103, the electrical interconnection member 7110 may include a clock spring portion 7121 similar to the clock spring arrangement of the second portion 6909 of the embodiment of FIGS. 69A through 69C. In this regard, the clock spring portion 7121 of the electrical interconnection member 7110 may be disposed such that undesirable counteracting torque on the pivoting of the transducer array 7105 may be significantly avoided. The clock spring portion 7121 of the electrical interconnection member 7110 is operable to maintain an electrical connection to the transducer array 7105 while the transducer array 7105 is pivoting. The configuration of the clock spring portion 7121 also saves space within the deflectable member 7103, allowing an advantageously smaller deflectable member.

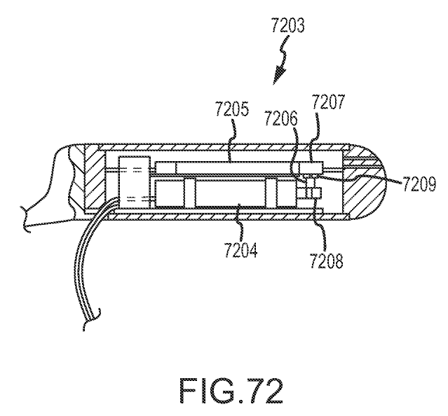

FIG. 72 illustrates a deflectable member 7203 in partial cross-section. The deflectable member 7203 is similar to the deflectable member 7103 of FIG. 71A. The deflectable member 7203 includes a transducer array 7205 and a motor 7204 disposed behind a back side of the transducer array 7105. However, in the deflectable member 7203, the motor 7204 is operatively interconnected to the transducer array 7205 via a cable 7206 partially wrapped about an output shaft 7208 of the motor 7204. Both ends of the cable 7206 are secured to a distal array end cap 7207 fixed to the transducer array 7205. Accordingly, as the motor 7204 rotates the output shaft 7208, a portion of the cable 7206 will be wound about the output shaft 7208 while simultaneously another portion of the cable 7206 will be unwound from the output shaft 7208. By attaching the ends of the cable 7206 to the transducer array 7205 on opposite sides of a rotational axis of the transducer array 7205, the winding and unwinding of the cable 7206 may be used to pivot the transducer array 7205.

Springs 7209 may be disposed between the ends of the cable 7206 and the distal array end cap 7207. Such springs 7209 may compensate for the non-linear variations in the distances between the anchor points of the cable 7206 to the distal array end cap 7207 as the transducer array 7205 pivots relative to the motor 7204. The springs may include a resilient polymer portion disposed between a top plate (to which the cable 7206 may be secured) and the distal array end cap 7207.

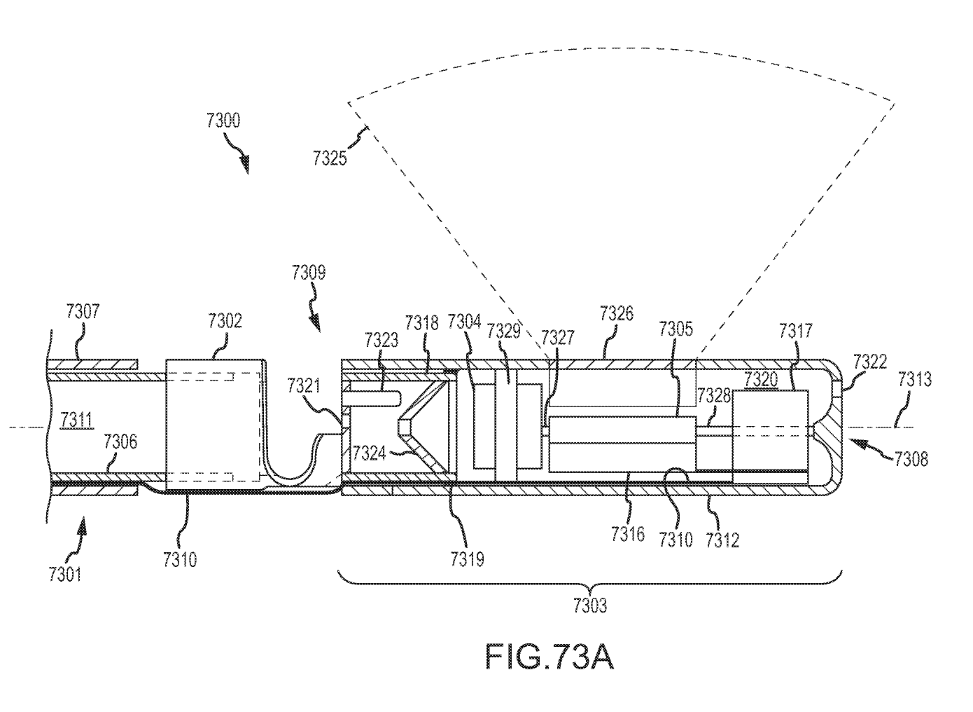

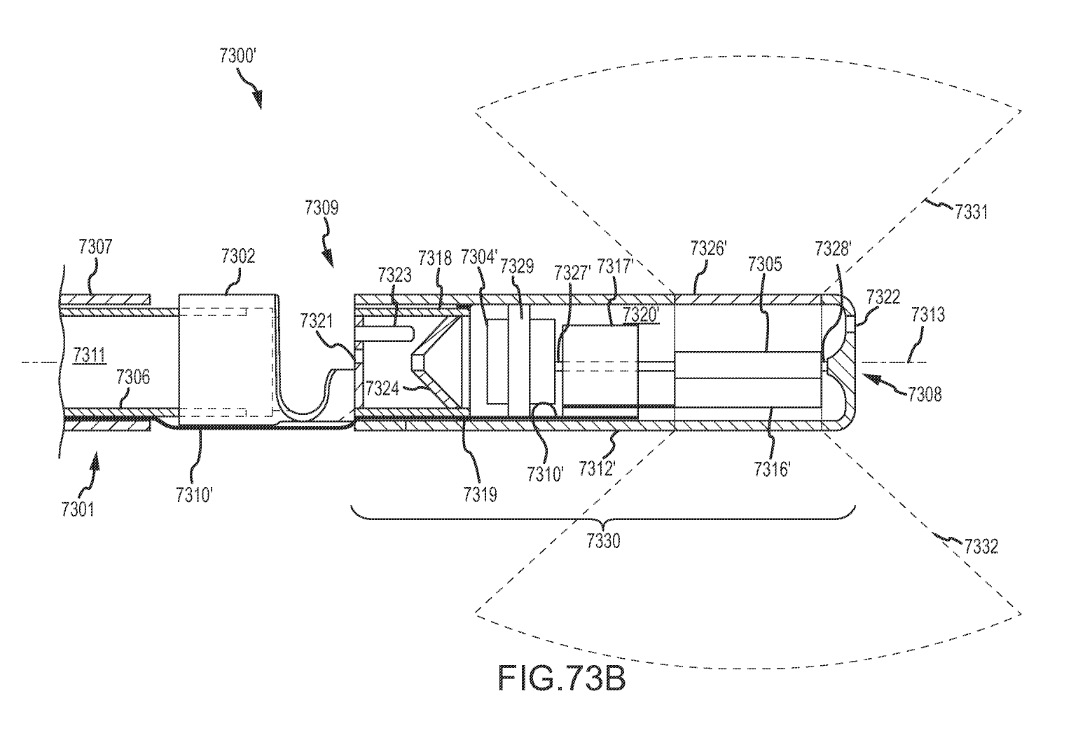

FIG. 73A illustrates a distal end of a catheter 7300 that includes a catheter body 7301 connected by a living hinge 7302 (similar to the living hinge 6001 of FIGS. 60, 61, and 62), to a deflectable member 7303. The living hinge 7302 is supportably interconnected to the deflectable member 7303 and an inner tubular body 7306 of the catheter body 7301. An electrical interconnection member 7310 is flexible and acts as a restraining member interconnected to an outer tubular body 7307 of the catheter body 7301 and the deflectable member 7303. Selective relative movement between the inner tubular body 7306 and the outer tubular body 7307 causes the deflectable member 7303 to selectively deflect in a predetermined manner. The deflectable member 7303 in FIG. 73 is illustrated in a non-deflected position. The inner tubular body 7306 may include a lumen 7311.

The deflectable member 7303 may generally include a distal end 7308 and a proximal end 7309. The deflectable member 7303 may include a case 7312. The case 7312 may be a relatively rigid (as compared to the catheter body 7301) member housing a motor 7304 and a transducer array 7305, both of which are discussed below. The deflectable member 7303 may include a longitudinal axis 7313.

Within the deflectable member 7303, the electrical interconnection member 7310 may run from the proximal end 7309 along the case 7312 between an array backing 7316 and the inner wall of the case 7312, to a clock spring portion 7317 of the electrical interconnection member 7310. From the clock spring portion 7317, the electrical interconnection member 7310 may interconnect to the array backing 7316. This configuration is similar to the configuration of the electrical interconnection member 5311″ of FIGS. 56A and 56B. In an arrangement, the electrical interconnection member 7310 may be constructed from a single flexboard.

The proximal end 7309 of the deflectable member 7303 may include an end member 7318 sealably disposed therein. The end member 7318 may be sealed along its outer perimeter using a sealing material 7319. The sealing material 7319 may be disposed as illustrated between the outer perimeter of the end member 7318 and an inner surface of the case 7312. The sealing material 7319 may be similar to the sealing material 5316 of FIG. 53. An enclosed volume 7320 may be defined by the case 7312 and the end member 7318. The enclosed volume 7320 may be fluid-filled and sealed.

The deflectable member 7303 may be filled using any appropriate method. The deflectable member 7303 may include a pair of sealable ports 7321, 7322 disposed on opposite ends of the deflectable member 7303. The sealable ports 7321, 7322 may allow for filling of the deflectable member 7303 in a manner similar to as described with reference to the catheter tip 5301 of FIG. 53. The deflectable member 7303 may include a bellows member 7323 that may function similarly to the bellows member 5320 of FIG. 53, with the exception that the bellows member 7323 may equalize or partially equalize pressure within the enclosed volume 7320 with the environment surrounding the deflectable member 7303.

The deflectable member 7303 may include a bubble-trap 7324, shown in cross section in FIG. 73. The bubble-trap 7324 may be configured, and function in a manner, similar to the bubble-trap 5324 described with reference to FIG. 53.

The deflectable member 7303 may be operable to reciprocally pivot the transducer array 7305 at a rate sufficient enough to generate 3D or 4D images of an image volume 7325. In this regard, the ultrasound imaging apparatus may be operable to display live video of the image volume. Generally, the transducer array 7305 is operable to transmit ultrasonic energy through an acoustic window 7326 of the case 7312.

The transducer array 7305 may be interconnected to an output shaft 7327 of the motor 7304 at a proximal end of the transducer array 7305. Furthermore, the transducer array 7305 may be supported on a distal end of the transducer array 7305 by a shaft 7328 that is supported at the distal end of the case 7312. The motor 7304 may be operable to reciprocally pivot the output shaft 7327 of the motor 7304 and therefore reciprocally pivot the transducer array 7305 interconnected to the output shaft 7327. The outer portion of the motor 7304 may be fixedly mounted to the inner surface of the case 7312 by one or more motor mounts 7329. Electrical interconnections (not shown) to the motor 7304 may be achieved through a dedicated set of electrical interconnections (e.g., wires) separate from the electrical interconnection member 7310. Alternatively, electrical interconnections to the motor 7304 may be made using a portion of the conductors of the electrical interconnection member 7310.

The positions of the motor 7304, the clock spring portion 7317, and the transducer array 7305 may be rearranged in any appropriate manner. For example, FIG. 73B illustrates a distal end of a catheter 7300′ that is similar to the catheter 7300 of FIG. 73A with the positions of the clock spring portion 7317 and transducer array 7305 swapped.

The catheter 7300′ of FIG. 73B includes a deflectable member 7330 that is deflectable in the same manner as the deflectable member 7303 of FIG. 73A. Within the deflectable member 7330, the electrical interconnection member 7310′ may run from the proximal end 7309 along the case 7312 between the motor 7304′ and the inner wall of the case 7312′, to the clock spring portion 7317′ of the electrical interconnection member 7310′. From the clock spring portion 7317′, the electrical interconnection member 7310′ may continue in a distal direction and interconnect to the array backing 7316. In an arrangement, the electrical interconnection member 7310′ may be constructed from a single flexboard.

The transducer array 7305 may be interconnected to an output shaft 7327′ of the motor 7304′ at a proximal end of the transducer array 7305. The output shaft 7327′ may extend through the clock spring portion 7317′. Furthermore, the transducer array 7305 may be supported on a distal end of the transducer array 7305 by a shaft 7328′ that is supported at the distal end of the case 7312′. The motor 7304′ may be operable to reciprocally pivot the output shaft 7327′ of the motor 7304 and therefore reciprocally pivot the transducer array 7305 interconnected to the output shaft 7327′. The acoustic window 7326′ may encircle the entire circumference of the case 7312′ or a portion thereof in the area of the transducer array 7305 to allow for imaging in directions as discussed below.

The motor 7304′ may be operable to reciprocally pivot the transducer array 7305 from the position illustrated in FIG. 73B a selected amount, such as +/−30 degrees. Thus the motor 7304′ may be operable to reciprocally pivot the transducer array 7305 through an angle large enough and at a rate sufficient enough to generate real-time or near real-time three-dimensional images of an image volume 7331 that is similar to the image volume 7325 of FIG. 73A.

The motor 7304′ may also be operable to first pivot the transducer array 7305 to a selected orientation and then reciprocally pivot the transducer array 7305 about the selected orientation a chosen distance. For example, the motor 7304′ may be operable to pivot the transducer array 7305 180 degrees from the position shown in FIG. 73B such that it is pointing downward in FIG. 73B, and then the motor 7304′ may be operable to reciprocally pivot the transducer array 7305 about the downward pointing position through an angle large enough and at a rate sufficient enough to generate real-time or near real-time three-dimensional images of an image volume 7332. In this regard, the motor 7304′ may initially pivot the transducer array 7305 and then reciprocate the transducer array 7305 around any chosen angle to image an imaging volume in any chosen direction, thus reducing the need to reposition the catheter 7300′ to achieve desired imaging volumes.

The motor 7304′ may be operable to reciprocally pivot the transducer array 7305 through 360 degrees or more. In this regard, the deflectable member 7330 may be operable to reciprocally pivot the transducer array 7305 through an angle large enough and at a rate sufficient enough to generate real-time or near real-time three-dimensional images of an image volume that completely encircles the deflectable member 7330.

The clock spring portion 7317′ may be configured to accommodate 360 degrees or more of rotation of the transducer array 7305. Such accommodation may be achieved by a single clock spring portion 7317′ or by multiple clock spring portions arranged in series with each portion accommodating a portion of the total pivoting of the transducer array 7305. In an arrangement, the clock spring portion 7317′, the motor 7304′, and the acoustic window 7326′ may be configured to accommodate a range of angular motion less than 360 degrees (e.g., 270 degrees, 180 degrees).

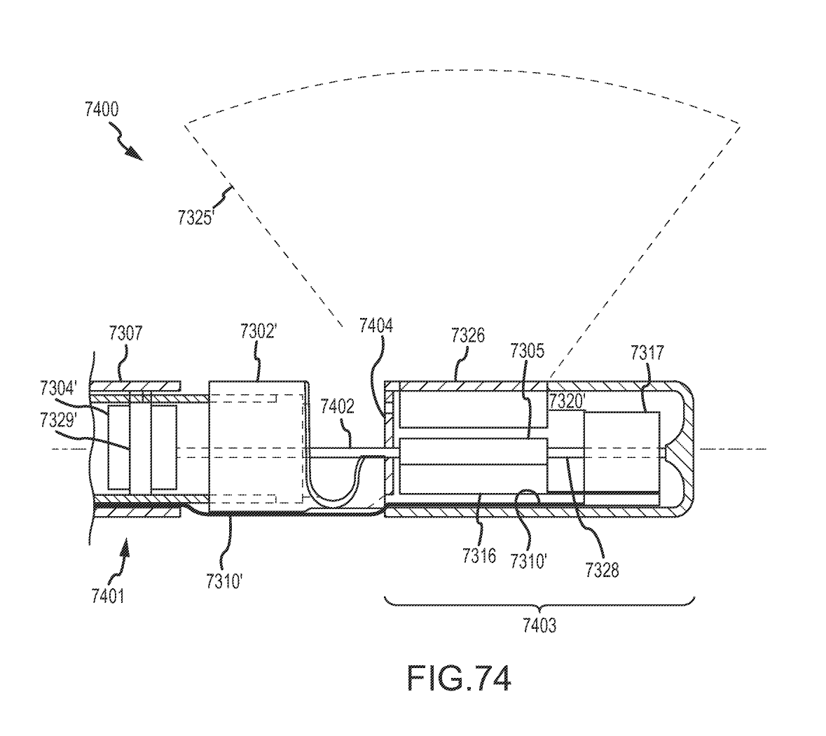

FIG. 74 is a partial cross-sectional view of an embodiment of a catheter 7400 that is similar to the catheter 7300 of FIG. 73. Items similar to those of the embodiment of FIG. 73 are designated by a prime symbol (′) following the reference numeral. A difference between the catheter 7400 of FIG. 74 and the catheter 7300 of FIG. 73 is that, in catheter 7400 a motor 7304′ for driving the transducer array 7305 is located in a distal end of a catheter body 7401 on an opposing side of the hinge 7302′ instead of in a deflectable member 7403. By moving the motor from the deflectable member 7403 to the catheter body 7401, the length of the deflectable member 7403 may be reduced. The motor 7304′ may be operable to drive the transducer array 7305 via a flexible drive member 7402 that may, on one end, be interconnected to an output shaft of the motor 7304′. On the other end, the flexible drive member 7402 may be interconnected to the transducer array 7305. The flexible drive member 7402 may be sealed along its outer perimeter where it passes through a proximal wall 7404 of the deflectable member 7403.

The motors driving motion (e.g., pivotal reciprocal) of transducer arrays discussed herein may be integrated into any appropriate embodiment discussed herein. The motors discussed herein (e.g., motor 6904) may be brushless DC motors. Wherein the motor used is a brushless DC motor, there are three wires driving three phases of motor current. The motor may be driven using pulse width modulation. In such a case the driver sends out pulses at, for example, a 40 KHz rate to keep the current at the desired level. Because of the sharp edges on the pulses this kind of driver can cause interference with the ultrasound system. To avoid this, a shield may be disposed around the motor wires to keep the interfering signal from passing to the conductors electrically connected to the transducer array. In another implementation, the pulse width modulation may be filtered to reduce the signal in the frequency band used by the transducer array (e.g., in the ultrasound frequency band). In a particular implementation, both the shielding and the filtering may be used. The motor may alternatively be driven by an analog driver that produces a continuous current (without pulses) to drive the motor.

Acoustic, capacitive, electromagnetic and optical sensor techniques may be utilized as means for detecting the angular position of any appropriate pivotable transducer array discussed herein. Based upon the data from the sensors, operation of the pivotable transducer array may be adaptively adjusted in order to compensate for variations in angular velocity of the pivotable transducer array. For example, adaptive compensation may be performed by adjusting the pulse repetition rate of transmitted ultrasonic energy, by adjusting the scan conversion algorithm, or by varying control of the motor to vary control of the rotation of the pivotable transducer array.

Any known sensor may be utilized in the embodiments discussed herein, including encoding by optical means including rotational encoders, distance by interferometry and/or brightness proximity, capacitive encoders, magnetic encoders, ultrasonic encoders, flexure of a flexible encoder membrane, and utilization of accelerometers.

One embodiment may use the sensor positioning data in comparison with a desired position utilizing a software program in a feedback system. If the actual position is behind the desired position (e.g., the angular position of pivotable transducer array is behind the desired angular position of the pivotable transducer array), a servo system may compensate by increasing the motor or drive operation. Conversely, if the actual position is ahead, the servo system may compensate by slowing the motor or drive.

Embodiments discussed herein of deflectable members may have an enclosed portion which may or may not contain a fluid. This fluid provides an acoustic coupling medium between the ultrasound transducer array and the acoustic window or tip. An additional benefit may be to provide cooling for the motor. Generally, the maximum desired temperature of a catheter operating in the body is about 41° C. Normal blood temperature is about 38° C. Under such circumstances, there may be a need to balance the power dissipation in the tip and the heat flow out of the tip such that the tip does not exceed a rise of about 3° C. above 38° C. Actual temperature monitoring near the distal end of the catheter body and in the deflectable member is desirable, with feedback to a controller with an automatic warning or shut down based upon some upper pre-determined temperature limit. A thermistor may be mounted within the tip to monitor the internal temperature so that the system may shut down operations before the temperature exceeds the pre-determined temperature limit. A thermocouple would be a suitable alternative to the use of a thermistor.

Active cooling methods such as thermoelectric cooling or passive conduction along metallic components may also be used in the embodiments discussed herein. Other types of thermal management systems, such as those disclosed in U.S. Patent Publication No. 2007/0167826, may be used in the embodiments discussed herein.

Fluid selected for use in the enclosed portion may provide: desired acoustic properties, desired thermal properties, appropriate low viscosity to not impede oscillatory motion of the array or other components, non-corrosiveness to components, and compatibility with the circulatory system and the rest of the human body in case of leakage. Fluids may be selected to avoid or minimize evaporation or development of bubbles over time. Embodiments discussed herein may have the fluid injected at the time of manufacture or at the point of use. In either case, the fluids may be sterile and miscible with water. Sterile saline is an example of a fluid that may be used in the embodiments discussed herein.

Embodiments discussed herein may include a deflectable member having a cylindrical shape or other shape designed to minimize vascular or bodily injury when moved (e.g., rotated, translated) or operated within a patient. Moreover, the outer surface of the deflectable members may be smooth. Such a smooth, atraumatic exterior profile may help in reducing thrombus formation and/or tissue damage. Such atraumatic shapes may be beneficial in reducing turbulence which may cause injury to blood cells.

Embodiments discussed herein generally described as including transducer arrays, ultrasound transducer arrays, or the like. However, it is also contemplated that the catheters discussed herein may include other appropriate devices in place of or in addition to such devices. For example, embodiments discussed herein may include ablation or other therapeutic devices in place of or in addition to the transducer arrays, ultrasound transducer arrays, or the like.

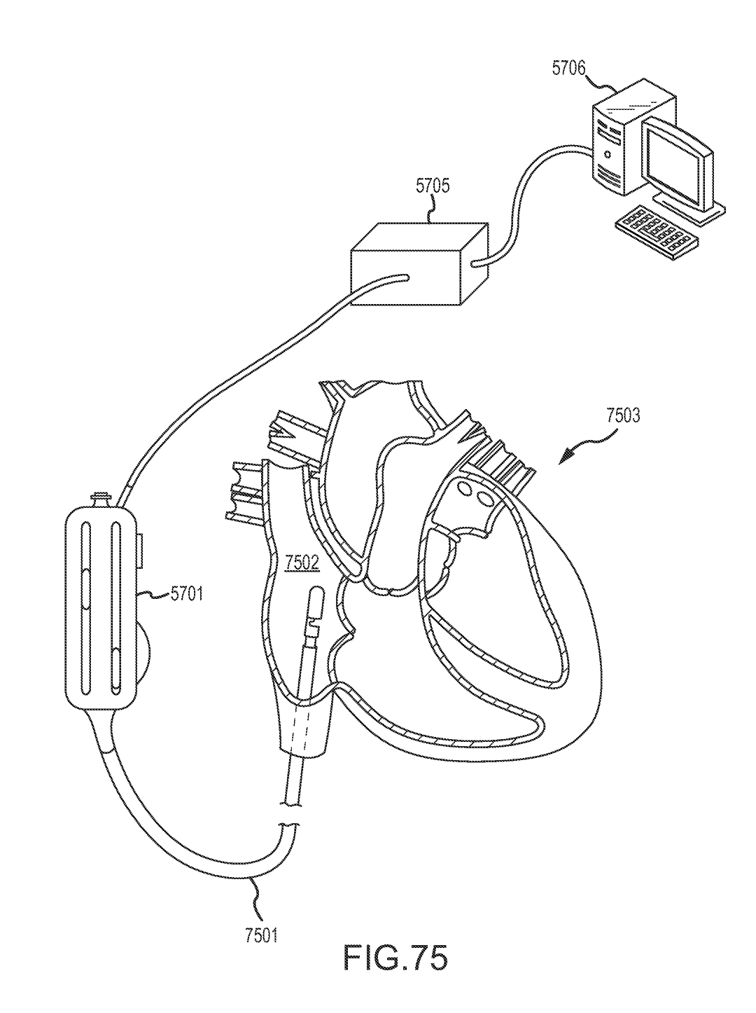

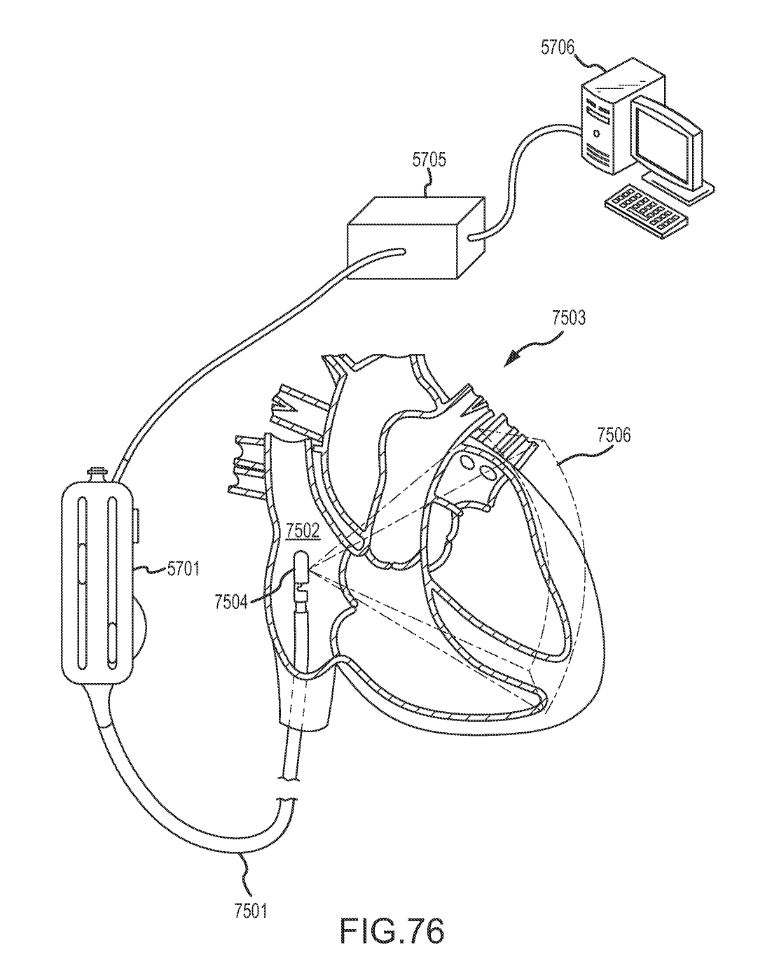

One difficulty associated with the use of conventional ICE catheters is the need to steer the catheter to multiple points within the heart in order to capture the various imaging planes needed during the procedure. FIG. 75 shows placement of a steerable catheter 7501 for intracardiac echocardiography within the right atrium 7502 of the heart 7503. FIG. 76 shows placement of the steerable catheter 7501 within the right atrium 7502 of the heart 7503 after the catheter has been repositioned (through steering of the catheter 7501) to place a deflectable member 7504 disposed at a distal end of the catheter 7501 at a desired position. The clinician may establish and then set the catheter 7501 position within the heart 7503 by locking the catheter 7501 position (locking mechanism on handle not shown). In this regard, once set, the catheter 7501 position may remain substantially unchanged while the deflectable member 7504 is deflected.

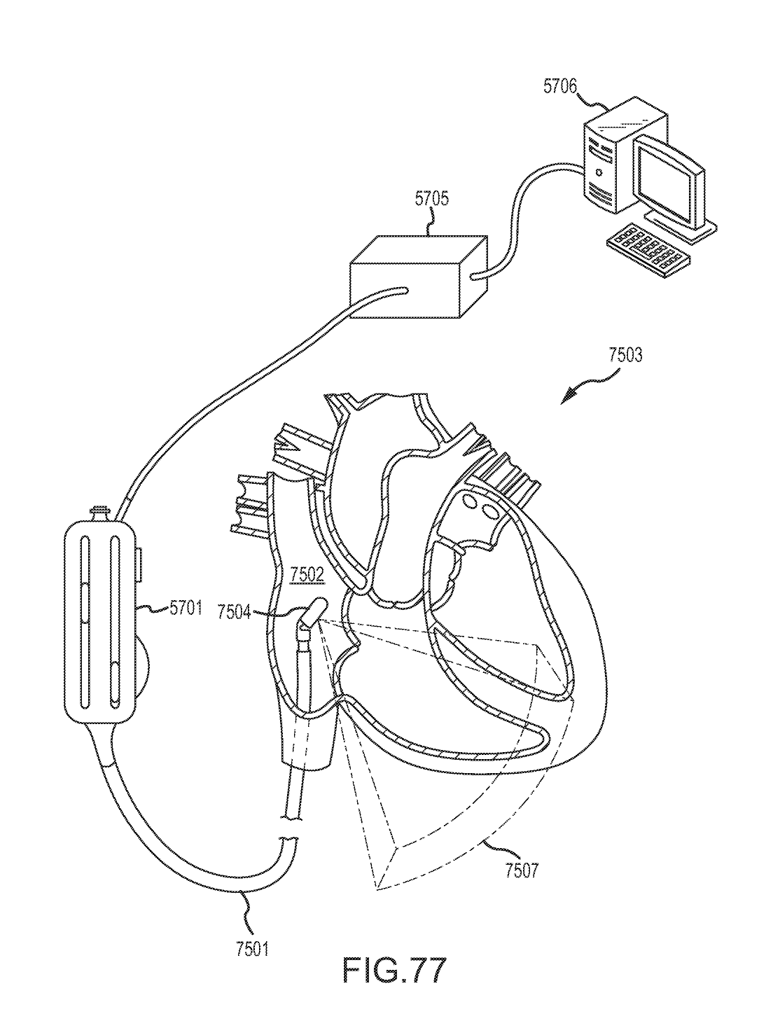

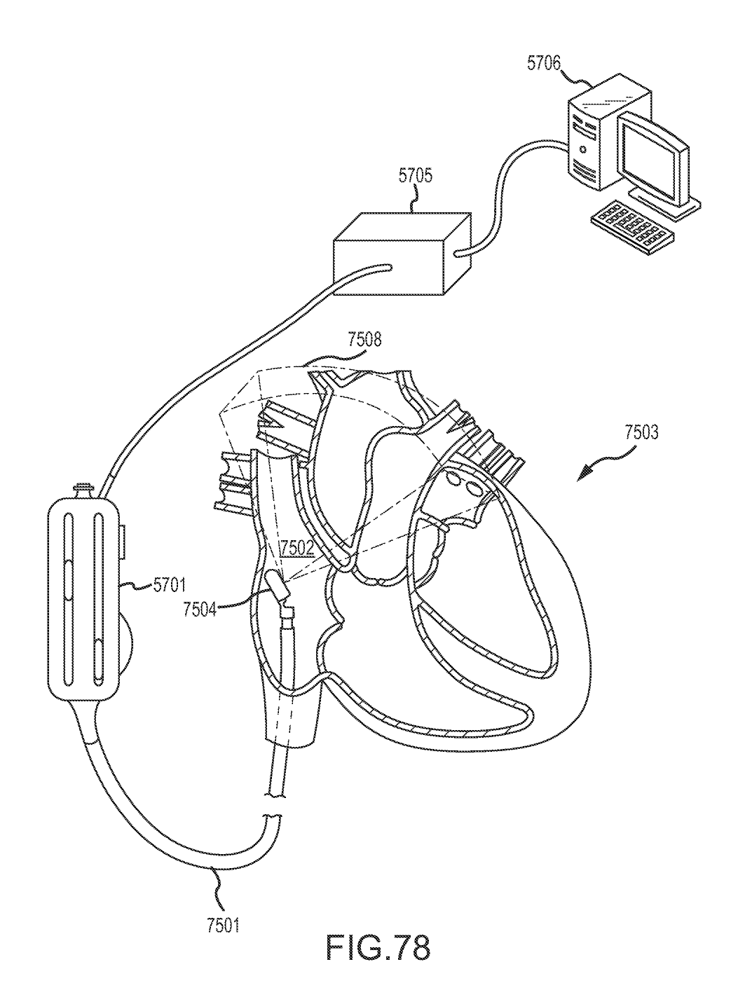

With the deflectable member positioned as illustrated in FIG. 76, a volumetric image may be generated from the three dimensional volume 7506 of a first portion of the heart 7503. The clinician may then manipulate the deflectable member 7504 orientation in order to capture the range of imaging volumes required. For example, FIG. 77 shows the deflectable member 7504 deflected to a second position to capture a volumetric image of the three dimensional volume 7507 of a second portion of the heart 7503. FIG. 78 shows the deflectable member 7504 deflected to a third position to capture a volumetric image of the three dimensional volume 7508 of a third portion of the heart 7503. Embodiments of deflectable members described herein may be operable to achieve such positions and more within the right atrium 7502 of the heart 7503 that may have an intracardiac volume with cross dimension of about 3 cm. Volumetric images of such three dimensional volumes 7506, 7507, and 7508 are obtainable by deflection of the deflectable member and operation of the motor to effectuate reciprocal pivoting of the ultrasound transducer array with the deflectable member while the distal end of the catheter 7501 remains in the position as shown in FIG. 75.

Clinical procedures that may be performed with embodiments disclosed herein include without limitation septal puncture and septal occluder deployment. A method for right atrial imaging utilizing embodiments may include advancing the catheter body to the right atrium, steering the distal end of the catheter body to a desired position, operating the motor to effectuate movement of the ultrasound transducer, and while maintaining the fixed catheter body position, deflect the deflectable member comprising the ultrasound transducer about the hinge to capture at least one image over at least one viewing plane.

Clinical procedures that may be performed from the left atrium include without limitation, left atrial appendage occluder placement, mitral valve replacement, aortic valve replacement, and cardiac ablation for atrial fibrillation. A method for left atrium imaging utilizing embodiments described herein may include advancing the catheter body to the right atrium, steering the distal end of the catheter body to a desired position, and while maintaining the fixed catheter body position, deflect the deflectable member comprising the ultrasound transducer about a hinge to achieve a desired position, operating the motor to effectuate movement of the ultrasound transducer to capture at least one image over at least one viewing plane of the intra-atrial septum, identify the anatomical region for septal puncture, advance a septal puncture tool through a lumen of the catheter, advance a guidewire, advance the catheter body to the left atrium, steer the catheter body to the desired position, and while maintaining the fixed catheter body position, deflect the deflectable member comprising the ultrasound transducer about the hinge to a desired position, and operate the motor to effectuate movement of the ultrasound transducer to capture at least one image over at least one viewing plane.

Additional modifications and extensions to the embodiments described above will be apparent to those skilled in the art. Such modifications and extensions are intended to be within the scope of the present invention as defined by the claims that follow.

What is claimed is:

1. Catheter comprising:
a catheter body having a proximal end and a distal end; and
a deflectable member hingedly connected to the distal end of said catheter body and operable for positioning across a range of angles relative to said catheter body;
wherein said deflectable member includes a component and a motor to effectuate movement of said component.

2. Catheter according to claim 1, wherein said component is an ultrasound transducer array.

3. Catheter according to claim 2, wherein said ultrasound transducer array is configured for at least one of two dimensional imaging, three dimensional imaging or real-time three dimensional imaging.

4. Catheter according to claim 1, wherein a minimum presentation width of said catheter is less than about 3 cm.

5. Catheter according to claim 1, wherein a length of a region in which deflection occurs when said deflectable member is deflected 90 degrees relative to said catheter body is less than a maximum cross dimension of said catheter body.

6. Catheter according to claim 1, wherein said catheter body comprises at least one steerable segment.

7. Catheter according to claim 6, wherein said one steerable segment is located at the distal end of the catheter body.

8. Catheter according to claim 1, wherein said deflectable member is operable for deflection across a range of angles relative to the longitudinal axis of the catheter body and said range is from about −90 degrees to about +180 degrees.

9. Catheter according to claim 1, wherein said deflectable member is operable for deflection across an arc of at least about 270 degrees relative to the longitudinal axis of the catheter body.

10. Catheter according to claim 1, wherein said catheter body comprises a lumen extending from the distal end of said catheter body to a point proximal thereto.

11. Catheter according to claim 10, wherein said lumen is for conveyance of at least one of a device and material.

12. Catheter according to claim 1, further comprising an actuation device operable for active deflection of said deflectable member.

13. Catheter according to claim 1, further comprising a distensible channel interconnected to said catheter body for conveyance of at least one of a device and material.

14. Catheter according to claim 1, wherein said catheter body comprises an invaginated portion for conveyance of at least one of a device and material.

15. Catheter according to claim 6, further comprising a hinge interconnecting the deflectable member and the catheter body.

16. Catheter according to claim 15, wherein said hinge is selected from the group consisting of living hinges, true hinges and combinations thereof, wherein upon deflection of said hinge a displacement arc is defined and the ratio of a maximum cross-dimension of the distal end of the catheter body to the displacement arc radius is at least about 1.

17. Catheter according to claim 15, wherein said hinge is a living hinge.

18. Catheter according to claim 15, wherein said hinge is an ideal hinge.

19. Catheter according to claim 15, wherein said hinge comprises a first cylindrical surface and a second cylindrical surface disposed about a common central axis, wherein upon deflection of said deflectable member, said first surface moves relative to said second surface.

20. Catheter according to claim 15, wherein said hinge comprises a non-tubular bendable portion.

21. Catheter according to claim 15, wherein upon deflection of said hinge a displacement arc is defined and the ratio of a maximum cross-dimension of the distal end of the catheter body to the displacement arc radius is at least about 1.

22. Catheter according to claim 15, further comprising an electrical interconnection interconnecting the ultrasound transducer array and the distal end of the catheter body.

23. Catheter according to claim 2, wherein said deflectable member comprises a portion comprising an enclosed volume, wherein a high viscosity non-water soluble couplant is disposed between a gap between a structure fixed to said ultrasound transducer array and an inner wall of said enclosed volume.

24. Catheter comprising:
a catheter body comprising a proximal end and a distal end; and
a deflectable member connected to the distal end of said catheter body and operable for positioning across a range of angles relative to a longitudinal axis of said catheter body at said distal end;
wherein said deflectable member includes a motor to effectuate movement of a component within said deflectable member.

25. Catheter comprising:
an outer tubular body;
a deflectable member comprising a motor; and
a hinge interconnecting said deflectable member and said outer tubular body.

26. Catheter according to claim 25, wherein said deflectable member further comprises an ultrasound transducer array.

27. Catheter according to claim 25, wherein said outer tubular body comprises at least one steerable segment.

28. Catheter according to claim 27, further comprising an actuation device operable for active deflection of said deflectable member.

29. Catheter according to claim 28, wherein said actuation device is a device selected from a group consisting of an electro-thermally activated shape memory material hinge, a wire, a tube, an electro-active material, fluid, stylet, permanent magnet, and electromagnet.

30. Catheter according to claim 28, wherein said actuation device extends from the proximal end to the distal end, wherein the actuation device and the outer tubular body are disposed for relative movement, and wherein the deflectable member is deflectable to a range of viewing angles from a forward-looking position to a rearward-looking position in response to a deflection force applied to the hinge upon applied relative movement between the actuation device and the outer tubular body.

31. Catheter according to claim 30, wherein the actuation device is an inner tubular body disposed within the outer tubular body.

32. Catheter according to claim 28, wherein the actuation device is a pull wire disposed along the outer tubular body.

33. Catheter according to claim 30, further comprising a handle disposed at the proximal end, wherein the handle comprises:
a handle body; and
a moving member movable relative to the body,
wherein the actuation device is interconnected to the moving member, wherein selected movement of the moving member relative to the handle body affects deflection of the deflectable member.

34. Catheter according to claim 33, wherein the handle further comprises a steering control for controlling the at least one steerable segment wherein said steering control is independently operable from said actuation device.

35. Catheter comprising:
a catheter body having at least one steerable segment and having a proximal end and a distal end; and
a deflectable member;
wherein said deflectable member includes a component, and wherein said deflectable member comprises a motor to effectuate movement of said component.

36. Catheter according to claim 35, further comprising:
a hinge interconnecting said deflectable member and said catheter body; and
an actuation device for selectively positioning said deflectable member;
wherein said component is an ultrasound transducer array, wherein said ultrasound transducer array is configured for use in at least one of two dimensional imaging, three dimensional imaging, or real-time three dimensional imaging.

37. Catheter according to claim 35, wherein said catheter body comprises a lumen extending from the distal end of said catheter body to a point proximal thereto for conveyance of at least one of a device and material.

38. Catheter according to claim 35, wherein said deflectable member is operable for positioning through a range of angles of greater than about 200 degrees relative to the longitudinal axis of said catheter body.

39. Catheter comprising:
a catheter body having a proximal end, a distal end, and at least one steerable segment;
a deflectable member supportably disposed at said distal end of said catheter body and operable for selective deflectable positioning across a range of angles relative to the longitudinal axis of said catheter body at said distal end;
a component supportably disposed on said deflectable member; and,
a motor supportably disposed on said deflectable member and operable for selective movement of said component.

40. Catheter according to claim 39, wherein said component is an ultrasound transducer array.

41. Catheter according to claim 39, wherein said steerable segment is steerable independent from said selective deflectable positioning of said deflectable member and independent from said selective movement of said component.

42. Catheter according to claim 41, wherein said deflectable member is operable for said selective deflectable positioning, independent from steering of said steerable segment and independent from said selective movement of said component.

43. Catheter according to claim 41, wherein said motor is operable for said selective movement of said component, independent from said deflectable positioning of said deflectable member and independent from steering of said steerable segment.

44. Catheter according to claim 40, further comprising:
a hinge interconnecting said distal end of said catheter body and said deflectable member.

45. Catheter according to claim 44, further comprising an electrical connection between the deflectable member and the catheter body.

46. Catheter according to claim 39, wherein a plane that is perpendicular to a longitudinal axis of the deflectable member intersects both said component and said motor.

47. Catheter according to claim 46, further comprising:
at least a first electrical interconnection member having a first portion coiled within said deflectable member and electrically interconnected to said component.

48. Catheter according to claim 47, wherein said first portion of said first electrical interconnection member is disposed in a clock spring arrangement.

49. Catheter according to claim 48, wherein said first portion of said first electrical interconnection member extends about said motor.

50. Catheter according to claim 39, wherein said catheter body comprises a lumen, for conveyance of at least one of a device and material, extending through at least a portion of the catheter body.

51. Catheter comprising:
a catheter body comprising a proximal end and a distal end;
a deflectable member supportably disposed at a said distal end of said catheter body and operable for selective deflectable positioning across a range of angles relative to the longitudinal axis of said catheter body; and
a component disposed in said deflectable member;
wherein said component is operable to move independently of said deflectable member, and wherein said deflectable member is operable to move independently from said catheter body.

52. Catheter comprising:
catheter body having a proximal end and a distal end;
lumen, for conveyance of at least one of a device and material, extending through at least a portion of the catheter body to a port located distal to the proximal end;
deflectable member, located at the distal end, wherein the deflectable member comprises a motor and a component; and
electrical conductor member comprising a plurality of electrical conductors in an arrangement extending from the component to the catheter body, wherein the arrangement is bendable in response to deflection of the deflectable member.

53. Catheter according to claim 52, wherein said arrangement is a flexboard arrangement.

54. Catheter according to claim 52, wherein said component is an ultrasound transducer array, wherein said ultrasound transducer array is configured for use in at least one of: two dimensional imaging, three dimensional imaging, or real-time three dimensional imaging and wherein said motor is operable to effectuate oscillatory movement of said ultrasound transducer array.

55. Catheter according to claim 53, wherein the flexboard arrangement is bendable in response to said oscillatory movement of said ultrasound transducer array.

56. Catheter comprising:
catheter body having a proximal end and a distal end;
lumen, for conveyance of at least one of a device and material extending through at least a portion of the catheter body to a port located distal to the proximal end; and
deflectable member located at said distal end, said deflectable member comprising a motor operable to effectuate movement of a component of said deflectable member.

57. Catheter according to claim 56, further comprising:
first electrical conductor portion comprising a plurality of electrical conductors arranged with electrically non-conductive material therebetween, the first electrical conductor portion extending from the proximal end to the distal end; and
second electrical conductor portion, electrically interconnected to the first electrical conductor portion at the distal end, comprising a plurality of electrical conductors;
wherein the component is an ultrasound transducer array, wherein the second electrical conductor portion is electrically interconnected to the ultrasound transducer array and is bendable in response to deflection of the deflectable member, wherein said ultrasound transducer array is configured for use in at least one of: two dimensional imaging, three dimensional imaging, or real-time three dimensional imaging.

58. Catheter according to claim 56, wherein the second electrical conductor portion is bendable in response to oscillatory movement of said ultrasound transducer array.

59. Catheter according to claim 58, wherein the catheter body comprises at least one steerable segment.

60. Catheter according to claim 59, further comprising a first electrical conductor portion to second electrical conductor portion junction.

61. Catheter according to claim 59, wherein the second electrical conductor portion comprises electrically conductive traces disposed on a flexible substrate.

62. Catheter according to claim 61, wherein the second electrical conductor portion aids in the deflection of the deflectable imaging device by operating as a flexible tether between the deflectable imaging device and the catheter body.

63. Catheter comprising:
outer tubular body extending from about a proximal end to a distal end of the catheter;
inner tubular body, extending from the proximal end to the distal end within the outer tubular body, the inner tubular body defining a lumen therethrough, for conveyance of at least one of a device and material, extending from proximate the proximal end to a port located proximate the distal end, wherein the outer tubular body and the inner tubular body are disposed for selective relative movement there between; and
deflectable member, at least a portion of which is permanently located outside of the outer tubular body at the distal end, supportability interconnected to one of the inner tubular body and the outer tubular body, wherein upon the selective relative movement the deflectable member is selectively deflectable in a predetermined manner;
wherein said deflectable member comprises a component and a motor operable for movement of said component.

64. Catheter according to claim 63, wherein said component is an ultrasound transducer array.

65. Catheter according to claim 63, wherein engagement between surfaces of the inner tubular body and the outer tubular body provides an interface sufficient to maintain a selected relative position between the inner tubular body and the outer tubular body and corresponding deflected position of the deflectable member.

66. Catheter according to claim 63, further comprising:
hinge located at the distal end, wherein the deflectable member is supportably interconnected to the hinge.

67. Catheter according to claim 66, wherein the hinge is supportably interconnected to the inner tubular body and restrainably interconnected to the outer tubular body.

68. Catheter according to claim 66, further comprising a restraining member interconnected to the deflectable member and the outer tubular body, wherein upon advancement of the inner tubular body relative to the outer tubular body, a deflection force is communicated to the deflectable member by the restraining member.

69. Catheter according to claim 63, wherein any movement of the inner tubular body relative to the outer tubular body produces a corresponding deflection of the deflectable member.

70. Catheter according to claim 68, wherein the restraining member is also a flexible electrical interconnection member.

71. Catheter according to claim 66, wherein at least one of the outer tubular body and the inner tubular body is steerable.

72. Catheter comprising:
catheter body having a proximal end, a distal end, and at least one steerable segment; and
deflectable member, located at said distal end, selectively deflectable from a first position to a second position, the deflectable member being interconnected to the catheter body and the deflectable member comprising a motor.

73. Catheter according to claim 72, wherein said deflectable member further comprises an ultrasound transducer array.

74. Catheter according to claim 72, wherein the deflectable member is deflectable about a deflection axis that is offset from a center axis of the catheter body.

75. Catheter according to claim 74, wherein the deflection axis lies in a plane transverse to the center axis.

76. Catheter according to claim 75, wherein the deflection axis lies in a plane orthogonal to the center axis.

77. Catheter according to claim 74, wherein the deflection axis lies in a plane that is parallel to the center axis.

78. Catheter according to claim 72, wherein the deflectable member is interconnected to the catheter body by a tether, wherein the tether restrainably interconnects the deflectable member to the catheter body.

79. Catheter according to claim 78, further comprising a flexible electrical interconnection member partially disposed between the deflectable member and the catheter body, wherein the portion of the flexible electrical interconnection member partially disposed between the deflectable member and the catheter body operates as a tether.

80. Catheter according to claim 78, further comprising a tether disposed between the deflectable member and the catheter body, wherein the tether includes a flexible electrical interconnection member.

81. Catheter according to claim 72, wherein the deflectable member comprises a tip, wherein the tip at least partially encases an ultrasound transducer array.

82. Catheter according to claim 71, further comprising a lumen, for conveyance of at least one of a device and material, extending through at least a portion of the catheter body from the proximal end to a port located distal to the proximal end.

83. Catheter comprising:
a catheter body,
a deflectable member,
an ultrasound transducer array disposed for pivotal movement about a pivot axis, and
at least a first electrical interconnection member having a first portion coiled and electrically interconnected to said ultrasound transducer array;
a motor operable to produce said pivotal movement; and
a hinge disposed between said catheter body and said deflectable member.

84. Catheter according to claim 83, said deflectable member having a portion having an enclosed volume, wherein said ultrasound transducer array is disposed for pivotal movement about said pivot axis within said enclosed volume, wherein said first portion is coiled within said enclosed volume.

85. Catheter according to claim 84, wherein said first portion of said first electrical interconnection member is helically disposed within said enclosed volume about a helix axis.

86. Catheter according to claim 85, wherein upon said pivotal movement said helically wrapped first portion of said first electrical interconnection member tightens and loosens about said helix axis.

87. Catheter according to claim 86, wherein said pivot axis is coincident with said helix axis.

88. Catheter according to claim 84, wherein said first electrical interconnection member is ribbon-shaped and comprises a plurality of conductors arranged side-by-side with electrically non-conductive material therebetween.

89. Catheter according to claim 88, wherein said first portion of said first electrical interconnection member is helically disposed within said enclosed volume about a helix axis.

90. Catheter according to claim 89, wherein upon said pivotal movement said helically wrapped first portion of said first electrical interconnection member tightens and loosens about said helix axis.

91. Catheter according to claim 84, wherein said first portion of said first electrical interconnection member is coiled a plurality of times within said enclosed volume.

92. Catheter according to claim 83, wherein said first portion of said first electrical interconnection member is helically disposed about said pivot axis.

93. Catheter according to claim 83, wherein said deflectable member is disposed at a distal end of said catheter body.

94. Catheter according to claim 83, wherein at least a portion of said deflectable member comprises a substantially round cross-sectional profile.

95. Catheter according to claim 83, further comprising a sealable port.

96. Catheter according to claim 84, wherein said motor is disposed within said enclosed volume and operatively interconnected to said ultrasound transducer array.

97. Catheter according to claim 83, further comprising a driveshaft operatively interconnected to said ultrasound transducer array, wherein said driveshaft drives said array for said pivotal movement.

98. Catheter according to claim 84, wherein said deflectable member comprises a distal end and a proximal end, wherein said first portion is disposed closer to said distal end than said ultrasound transducer array, and wherein said first portion is helically disposed within said enclosed volume about a helix axis.

99. Catheter according to claim 83, wherein said first portion of said first electrical interconnection member is disposed in a clock spring arrangement.

100. Catheter according to claim 99, wherein a midline of said first portion of said first electrical interconnection member is disposed within a single plane that is disposed perpendicular to said pivot axis.

101. Catheter according to claim 100, wherein said deflectable member comprises a distal end and a proximal end, wherein said first portion of said first electrical interconnection member is disposed closer to said distal end than said ultrasound transducer array.

102. Catheter according to claim 100, wherein said deflectable member comprises a distal end and a proximal end, wherein said ultrasound transducer array is disposed closer to said distal end than said first portion of said first electrical interconnection member.

103. Catheter according to claim 102, wherein said motor is operable to pivot said ultrasound transducer array through at least about 360 degrees.

104. Catheter according to claim 101, wherein said first portion of said first electrical interconnection member comprises a flexboard.

105. Catheter according to claim 83, further comprising a lumen, wherein a portion of said lumen is disposed within a coil of said first portion of said first electrical interconnection member.

106. Catheter according to claim 84, further comprising a fluid disposed within said enclosed volume.

107. Catheter comprising:
a catheter body with a proximal end and a distal end;
a deflectable member supportably disposed on the distal end of said catheter body and having a portion having a first volume, wherein said deflectable member is deflectable relative to a longitudinal axis of said catheter body at said distal end;
an ultrasound transducer array disposed for pivotal movement about a pivot axis within said first volume; and
at least a first electrical interconnection member having a first portion coiled within said first volume and electrically interconnected to said ultrasound transducer array.

108. Catheter according to claim 107, wherein said first volume is open to an environment surrounding at least a portion of said deflectable member.

109. Catheter according to claim 107, wherein said first portion of said first electrical interconnection member is helically disposed within said first volume about a helix axis.

110. Catheter according to claim 109, wherein said first electrical interconnection member further comprises a second portion adjoining said first portion, wherein said second portion is fixedly positioned relative to a case partially surrounding said first volume, wherein upon said pivotal movement, said coiled first portion of said first electrical interconnection member tightens and loosens.

111. Catheter according to claim 110, wherein said first electrical interconnection member is ribbon-shaped and comprises a plurality of conductors arranged with electrically non-conductive material therebetween.

112. Catheter according to claim 107, further comprising a structure fixed to and at least partially surrounding said ultrasound transducer array.

113. Catheter according to claim 112, wherein said structure comprises a generally round cross-sectional profile.

114. Catheter according to claim 112, wherein said structure is configured to minimize tissue and cellular trauma.

115. Catheter according to claim 107, wherein said first portion of said first electrical interconnection member is disposed in a clock spring arrangement.

116. Catheter comprising:
a deflectable member having a portion having an enclosed volume;
a fluid disposed within said enclosed volume;
an ultrasound transducer array disposed for reciprocal pivotal movement within said enclosed volume;
at least a first electrical interconnection member having at least a portion helically disposed within said enclosed volume and fixedly interconnected to said ultrasound transducer array, wherein upon said reciprocal movement said helically disposed portion loosens and tightens along a length thereof; and
a hinge disposed between said deflectable member and said catheter body.

117. Catheter according to claim 116, wherein said helically disposed portion is disposed about a pivot axis of said ultrasound transducer array.

118. Catheter according to claim 116, wherein an entirety of said helically disposed portion is offset from said pivot axis.

119. Catheter according to claim 118, wherein said helically disposed portion is ribbon-shaped and comprises a plurality of conductors arranged with electrically non-conductive material therebetween.

120. Catheter comprising:
a deflectable member having a portion having an enclosed volume;
a fluid disposed within said enclosed volume;
a catheter body;
a hinge disposed between said deflectable member and said catheter body; and
a bubble-trap member fixedly positioned within said enclosed volume and having a distal-facing, concave surface,
wherein a distal portion of said enclosed volume is defined distal to said bubble-trap member and a proximal portion of said enclosed volume is defined proximal to said bubble-trap member, wherein an aperture is provided through said bubble-trap member to fluidly interconnect from said distal portion of said enclosed volume to said proximal portion of said enclosed volume.

121. Catheter according to claim 120, wherein said bubble-trap member is disposed proximate to a proximal end of said deflectable member.

122. Catheter according to claim 120, further comprising a filter disposed across said aperture.

123. Catheter according to claim 122, wherein said filter is configured such that air may pass through said aperture, and wherein said filter is configured such that said fluid is unable to pass through said aperture.

124. Catheter according to claim 120, further comprising an ultrasound transducer array disposed for movement within said enclosed volume, wherein a gap between a structure fixed to said ultrasound transducer array and an inner wall of said enclosed volume is sized such that said fluid is drawn into said gap via capillary forces.

125. Catheter comprising:
a deflectable member a portion having an enclosed volume;
a fluid disposed within said enclosed volume;
an ultrasound transducer array disposed for movement within said enclosed volume;
a hinge; and,
a bellows member having a flexible, closed-end portion located in said fluid disposed within said enclosed volume and an open-end isolated from said fluid, wherein said bellows member is collapsible and expansible in response to volumetric variations in said fluid.

126. A method for operating a catheter, comprising:
providing a catheter body with a proximal end, a distal end, and at least one steerable segment, a deflectable member hingedly connected to the distal end of said catheter body, and an actuation device operable for selective deflection of said deflectable member; wherein said deflectable member comprises an ultrasound transducer array and a motor to effectuate movement of said ultrasound transducer array;
advancing said catheter body through a natural or otherwise-formed passageway in a patient;
steering said distal end of said catheter body to a desired position;
selectively deflecting said deflectable member to one or more angles relative to said catheter body with the distal end of said catheter body maintained in the desired position; and
operating said motor to effectuate movement of said ultrasound transducer array to obtain at least two unique 2D images.

127. A method according to claim 126, wherein said selective deflection step is completed within a volume having a cross-dimension of about 3 cm or less.

128. A method for operating a catheter having a catheter body with at least one independently steerable segment and a deflectable member supportably disposed at a distal end of said catheter body, comprising:
advancing said catheter through a passageway in a patient to a desired position, wherein said distal end of said catheter body is located at a first position;
deflecting said deflectable member to a desired angular position within a range of viewing angles relative to said distal end of said catheter body with said distal end maintained in said first position; and,
operating a motor supportably disposed on said deflectable member, with said deflectable member in said desired angular position, for driven movement of an ultrasound transducer array supportably disposed on said deflectable member.

129. A method for operating a catheter according to claim 128, wherein said advancing step comprises:
steering said catheter body by flexure along a length thereof.

130. A method for operating a catheter according to claim 129, wherein said advancing step comprises:
locking the longitudinal location of the distal end of said catheter body in said first position after steering.

131. A method for operating a catheter according to claim 130, further comprising:
rotating said catheter body to rotate said deflectable member.

132. A method for operating a catheter according to claim 131, wherein said rotating step is at least partially completed after said advancing step.

133. A method for operating a catheter according to claim 128, wherein said range of viewing angles is at least an arc of about 200 degrees, and wherein said deflecting step is completable within a volume having a cross-dimension of about 3 cm or less.

134. A method for operating a catheter according to 128, wherein said deflecting step comprises:
deforming a hinge, interconnecting said distal end of said catheter body and said deflectable member, from a first configuration to a second configuration.

135. A method for operating a catheter according to claim 128, wherein the ultrasound transducer array is side-looking during said advancing step and forward-looking during said operating step.

136. A method for operating a catheter according to claim 128, further comprising:
advancing or retrieving a device or material through a port at said distal end of said catheter body and into an imaging volume of said ultrasound transducer array during said operating step.

137. A method for operating a catheter according to claim 128, wherein said operating step comprises:
first pivoting said ultrasound transducer array about a pivot axis in a first direction;
tightening a plurality of coils of an electrical interconnection member connected to said ultrasound transducer array about said pivot axis during said first pivoting step;
second pivoting said transducer array in a second direction, wherein said second direction is opposite to said first direction; and
loosening said plurality of coils about said pivot axis during said second pivoting step.
